(19) United States
(12) Patent Application Publication
(10) Pub. No.: US 2022/0002695 A1
(43) Pub. Date: Jan. 6, 2022

(54) TARGETED ACTIVE GENE EDITING AGENT AND METHODS OF USE

(71) Applicant: Spotlight Therapeutics, Hayward, CA (US)

(72) Inventors: Hariharan JAYARAM, Menlo Park, CA (US); Eric ESTRIN, Oakland, CA (US); Jillian ASTARITA, Oakland, CA (US)

(73) Assignee: Spotlight Therapeutics, Hayward, CA (US)

(21) Appl. No.: 17/481,056

(22) Filed: Sep. 21, 2021

Related U.S. Application Data
(63) Continuation of application No. PCT/US2020/024289, filed on Mar. 23, 2020.
(60) Provisional application No. 62/822,529, filed on Mar. 22, 2019.

(51) Int. Cl. C12N 9/22; C07K 16/28; C12N 15/11; C07K 14/31; C12N 15/90

(52) U.S. Cl. CPC C12N 9/22; C07K 16/2845; C12N 15/11; C07K 14/31; C07K 2319/00; C12N 15/907; C12N 2310/20; C12N 2800/80; C07K 16/2866

(57) ABSTRACT

Methods and compositions related to intracellular delivery of gene editing proteins are provided. The invention relates to compositions and methods for transporting gene editing polypeptides, such as Cas9 or Cas12, into a cell ex vivo or in vivo. The invention includes a targeted active gene editing (TAGE) agent that includes an antigen binding polypeptide that specifically binds to an extracellular cell membrane-bound molecule, and a site-directed modifying polypeptide that recognizes a nucleic acid sequence. The antigen binding polypeptide and the site-directed modifying polypeptide are stably associated such that the site-directed modifying polypeptide can be internalized into a cell displaying the extracellular cell membrane-bound molecule.

### RELATED APPLICATIONS

This application is a continuation of International Application No. PCT/US2020/024289, filed on Mar. 23, 2020, which claims priority to U.S. Provisional Application No. 62/822,529, filed on Mar. 22, 2019. The content of the priority application is incorporated by reference herein.

### SEQUENCE LISTING

The instant application contains a Sequence Listing which has been submitted electronically in ASCII format and is hereby incorporated by reference in its entirety. Said ASCII copy, created on Sep. 20, 2021, is named S106638_1030US_C1_Sequence_Listing.txt and is 238 kilobytes in size.

### FIELD

The present invention generally relates to methods and compositions for editing a nucleic acid within a cell using site-directed modifying polypeptides conjugated to an antigen binding polypeptide.

### BACKGROUND OF THE INVENTION

CRISPR-associated RNA-guided endonucleases, such as Cas9, have become a versatile tool for genome engineering in various cell types and organisms (see, e.g., U.S. Pat. No. 8,697,359). Guided by a guide RNA, such as a dual-RNA complex or a chimeric single-guide RNA, RNA-guided endonucleases (e.g., Cas9) can generate site-specific double-strand breaks (DSBs) or single-stranded breaks (SSBs) within target nucleic acids (e.g., double-stranded DNA (dsDNA), single-stranded DNA (ssDNA), or RNA). When cleavage of a target nucleic acid occurs within a cell (e.g., a eukaryotic cell), the break in the target nucleic acid can be repaired by nonhomologous end joining (NHEJ) or homology directed repair (HDR). In addition, catalytically inactive RNA-guided endonucleases (e.g., Cas9) alone or fused to transcriptional activator or repressor domains can be used to alter transcription levels at sites within target nucleic acids by binding to the target site without cleavage.

However, the ability to deliver and target RNA-guided endonucleases to specific cells or tissues remains a challenge. A variety of methods or vehicles for delivery of RNA-guided endonucleases have been utilized, such as electroporation, nucleofection, microinjection, adeno-associated vectors (AAV), lentivirus, and lipid nanoparticles (see, e.g., in Lino, C. A. et al., 2018. Drug delivery, 25(1), pp. 1234-1257). As described in Lino et al, certain methods, such as microinjection or electroporation, are limited primarily to in vitro applications. Other modes of delivery, such as AAVs or lipid nanoparticles, have been utilized for in vivo delivery of RNA-guided endonucleases, but these delivery methods have faced challenges in an in vivo setting. For example, AAV-based delivery vehicles present immunological barriers, packaging size limitations, and the risk for genotoxic genome integration events (see, e.g., Lino et al., 2018; and Wang, D, et al., 2019. Nature Reviews Drug Discovery, 18(5), pp. 358-378). Further, delivery of RNA-guided endonucleases by lipid nanoparticles has several drawbacks, including endosomal degradation of cargo, specific cell tropism, and bioaccumulation in the liver (see, e.g., Lino et al., 2018; and Finn, J. D., et al., 2018. Cell reports, 22(9), pp. 2227-2235).

Alternative methods have been attempted to improve target delivery of RNA-guided endonucleases by modifying the RNA-guided endonucleases themselves with a receptor. However, examples of such receptor-mediated RNA-guided endonucleases have shown limited editing in vitro and did not achieve in vivo editing (see, e.g., Rouet, R., et al., 2018. Receptor-mediated delivery of CRISPR-Cas9 endonuclease for cell-type-specific gene editing. J Am Chem, 140(21), pp. 6596-6603).

### SUMMARY OF THE INVENTION

There is an unmet need for RNA-guided endonucleases with the capability of targeting desired cells or tissues, especially for in vivo editing. There is a need in the art for effective delivery of gene editing therapies utilizing RNA-guided endonucleases with the capability of targeting desired cells or tissues. Further, there is an unmet need for compositions and methods that provide in vivo targeted gene editing.

Provided herein are Targeted Active Gene Editing (TAGE) agents comprising an antigen-binding polypeptide which are able to edit specific cell types both in vivo and ex vivo. The modular and programmable design of TAGE agents enables rapid re-targeting and multi-functionality to enable flexible targeting of a variety of cell types. Further, by editing specific nucleic acid sequences (e.g., genes and regulatory elements) in target cells, TAGE agents have dual specificity and have fewer off-target effects than DNA-based delivery approaches (Cameron, et al. Nature methods. 14.6 (2017): 600; Kim, et al. Genome research. 24.6 (2014): 1012-1019). TAGE agents include one or more antigen-binding polypeptides that promote cell binding and/or cellular internalization of the TAGE agent in the target cell. Further, in some instances, the antigen-binding polypeptides, not only allow for receptor-mediated entry of the TAGE agent, but in certain instances, the antigen-binding polypeptides also mediate the biology of the cell (e.g., by altering intracellular signal transduction pathways).

Accordingly, provided herein are methods and compositions relating to a gene editing cell internalizing agent (TAGE agent) comprising an antigen binding polypeptide that specifically binds to an extracellular cell membrane-bound molecule (e.g., a cell surface molecule), and a site-directed modifying polypeptide that recognizes a nucleic acid sequence, wherein the antigen binding polypeptide and the site-directed modifying polypeptide are stably associated such that the site-directed modifying polypeptide can be internalized into a cell displaying the extracellular cell membrane-bound molecule (e.g., a cell surface molecule).

In some embodiments, the antigen binding polypeptide is an antibody, an antigen-binding portion of an antibody, or an antibody-mimetic.

In some embodiments, the site-directed modifying polypeptide comprises a nuclease or a nickase. In certain embodiments, the nuclease is a DNA endonuclease, such as Cas9 or Cas12.

In some embodiments, the TAGE agent further comprises a guide RNA that specifically hybridizes to a target region of the genome of the cell, wherein the guide RNA and the site-directed modifying polypeptide form a ribonucleoprotein.

In another aspect, the invention provides a targeted active gene editing (TAGE) agent comprising an antigen binding polypeptide which specifically binds to an extracellular cell membrane-bound molecule, and a site-directed modifying polypeptide comprising an RNA-guided DNA endonuclease that recognizes a CRISPR sequence, wherein the antigen binding polypeptide and the site-directed modifying polypeptide are stably associated such that the site-directed modifying polypeptide can be internalized into a cell displaying the extracellular cell membrane-bound molecule, and wherein the antigen binding polypeptide is an antibody, an antigen-binding portion of an antibody, or an antibody-mimetic.

In some embodiments, the TAGE agent comprises a guide RNA that specifically hybridizes to a target region of the genome of the cell, wherein the guide RNA and the site-directed modifying polypeptide form a ribonucleoprotein.

In some embodiments, the RNA-guided DNA endonuclease is a Cas9 nuclease. In some embodiments, the Cas9 nuclease is wildtype Cas9 nuclease (e.g., Streptococcus pyogenes Cas9, SEQ ID NO: 119). In some embodiments, the Cas9 nuclease comprises an amino acid sequence having at least 85%, 90%, 95%, 97%, 98%, 99%, or 100% identity to SEQ ID NO: 119. In certain embodiments, the Cas9 nuclease comprises the amino acid substitution C80A (e.g., SEQ ID NO: 1). In another the Cas9 nuclease comprises an amino acid sequence having at least 85%, 90%, 95%, 97%, 98%, 99%, or 100% identity to SEQ ID NO: 1.

In some embodiments, the RNA-guided DNA endonuclease is a nuclease other than Cas9 (e.g., such as one described in Section III). In certain embodiments, the RNA-guided DNA endonuclease is a CRISPR Type V nuclease. In specific embodiments, the RNA-guided DNA endonuclease is a Cas12 nuclease. In some embodiments, the Cas12 nuclease is wildtype Cas12 nuclease (e.g., Acidaminococcus sp. Cas12a, SEQ ID NO: 120). In some embodiments, the Cas12 nuclease comprises an amino acid sequence having at least 85%, 90%, 95%, 97%, 98%, 99%, or 100% identity to SEQ ID NO: 120. Examples of Cas12a variants useful in the TAGE agents herein include, but are not limited to, Alt-R® Cas12a (Cpf1) Ultra (e.g., IDT Catalog No. 10001272) or Cas12a as described in Kleinstiver, et al. Nature Biotechnology 37.3 (2019): 276-282, which is hereby incorporated by reference.

In some embodiments, the site-directed modifying polypeptide further comprises at least one nuclear localization signal (NLS).

In some embodiments, the site-directed modifying polypeptide further comprises a conjugation moiety that binds to the antigen binding polypeptide. In certain embodiments, the conjugation moiety is a protein. In certain embodiments, the protein is Protein A, SpyCatcher, or a Halo-Tag.

In some embodiments, the site-directed modifying polypeptide and the antigen binding polypeptide are conjugated via a linker. In certain embodiments, the linker is cleavable.

In some embodiments, the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule.

In some embodiments, the antigen-binding portion of the antibody is a nanobody, a domain antibody, an scFv, a Fab, a diabody, a BiTE, a diabody, a DART, a minibody, a F(ab′)₂, or an intrabody.

In some embodiments, the antibody is an intact antibody or a bispecific antibody.

In some aspects, the invention provides a targeted active gene editing (TAGE) agent comprising an antibody, or an antigen-binding portion thereof, which specifically binds to an extracellular cell membrane-bound protein, and a site-directed modifying polypeptide comprising a Cas9 nuclease, wherein the antibody, or antigen-binding portion thereof, and the site-directed modifying polypeptide are stably associated via a conjugation moiety such that the site-directed modifying polypeptide can be internalized into the cell expressing the extracellular cell membrane-bound protein via the antibody, or the antigen binding portion thereof.

In some embodiments, the site-directed modifying polypeptide further comprises at least one nuclear localization signal (NLS). In certain embodiments, the at least one NLS comprises an SV40 NLS. In certain embodiments, the SV40 NLS comprises the amino acid sequence PKKKRKV (SEQ ID NO: 8). In certain embodiments, the at least one NLS is at the C-terminus, the N-terminus, or both of the site-directed modifying polypeptide. In certain embodiments, the TAGE agent comprises at least two NLSs.

In certain embodiments, the TAGE agent further comprises a guide RNA that specifically hybridizes to a target region of the genome of a cell expressing the extracellular cell membrane-bound protein, wherein the guide RNA and the site-directed modifying polypeptide form a nucleoprotein.

In certain embodiments, the site-directed modifying polypeptide further comprises a conjugation moiety that can bind to the antibody, or antigen-binding portion thereof. In certain embodiments, the conjugation moiety is a protein. In some embodiments, the protein is Protein A, SpyCatcher, or a Halo-Tag.

In some embodiments, the Cas9 nuclease is wildtype Cas9 nuclease (e.g., Streptococcus pyogenes Cas9, SEQ ID NO: 119). In some embodiments, the Cas9 nuclease comprises an amino acid sequence having at least 85%, 90%, 95%, 97%, 98%, 99%, or 100% identity to SEQ ID NO: 119.

In certain embodiments, the Cas9 nuclease comprises the amino acid substitution C80A (e.g., SEQ ID NO: 1). In certain embodiments, the Cas9 nuclease has an amino acid sequence that is at least 85%, 90%, 95%, 97%, 98%, 99%, or 100% identical to SEQ ID NO: 1.

In certain embodiments, the antigen-binding portion of the antibody is a nanobody, a domain antibody, an scFv, a Fab, a diabody, a BiTE, a diabody, a DART, a minibody, a F(ab′)₂, or an intrabody.

In certain embodiments, the antibody is an intact antibody or a bispecific antibody.

In certain embodiments, the extracellular cell membrane-bound molecule or protein (e.g., cell surface molecule or protein) is HLA-DR, CD44, CD11a, CD22, CD3, CD20, CD33, CD32, CD44, CD47, CD59, CD54, CD25, AchR, CD70, CD74, CTLA4, EGFR, HER2, EpCam, OX40, PD-1, PD-L1, GITR, CD52, CD34, CD27, CD30, ICOS, or RSV.

In some embodiments, the extracellular cell membrane-bound molecule or protein is CD11a. In some embodiments, the antigen binding polypeptide is an anti-CD11a antibody, or antigen binding fragment thereof. In certain embodiments, the anti-CD11a antibody is efalizumab.

In some embodiments, the extracellular cell membrane-bound molecule or protein is CD25. In some embodiments, the antigen binding polypeptide is an anti-CD25 antibody, or antigen binding fragment thereof. In certain embodiments, the anti-CD25 antibody is daclizumab.

In another aspect, the invention provides a site-directed modifying polypeptide comprising an RNA-guided DNA endonuclease that recognizes a CRISPR sequence and a conjugation moiety that binds to an antibody, an antigen-binding portion of an antibody, or an antibody mimetic that specifically binds to an extracellular cell membrane-bound molecule (e.g., cell surface molecule).

In certain embodiments, the site-directed modifying polypeptide further comprises a guide RNA that specifically hybridizes to a target region of the genome of a cell. In certain embodiments, the RNA-guided DNA endonuclease is a Cas9 nuclease. In some embodiments, the Cas9 nuclease is wildtype Cas9 nuclease (e.g., Streptococcus pyogenes Cas9, SEQ ID NO: 119). In some embodiments, the Cas9 nuclease comprises an amino acid sequence having at least 85%, 90%, 95%, 97%, 98%, 99%, or 100% identity to SEQ ID NO: 119. In certain embodiments, the Cas9 nuclease comprises the amino acid substitution C80A (e.g., SEQ ID NO: 1). In another the Cas9 nuclease comprises an amino acid sequence having at least 85%, 90%, 95%, 97%, 98%, 99%, or 100% identity to SEQ ID NO: 1.

In certain embodiments, the RNA-guided DNA endonuclease is a CRISPR Type V nuclease. In specific embodiments, the RNA-guided DNA endonuclease is a Cas12 nuclease. In some embodiments, the Cas12 nuclease is wildtype Cas12 nuclease (e.g., Acidaminococcus sp. Cas12a, SEQ ID NO: 120). In some embodiments, the Cas12 nuclease comprises an amino acid sequence having at least 85%, 90%, 95%, 97%, 98%, 99%, or 100% identity to SEQ ID NO: 120. Examples of Cas12a variants useful in the TAGE agents herein include, but are not limited to, Alt-R® Cas12a (Cpf1) Ultra (e.g., IDT Catalog No. 10001272) or Cas12a as described in Kleinstiver, et al. Nature Biotechnology 37.3 (2019): 276-282, which is hereby incorporated by reference.

In certain embodiments, the site-directed modifying polypeptide further comprises at least one nuclear localization signal (NLS). In certain embodiments, the at least one NLS comprises an SV40 NLS. In certain embodiments, the SV40 NLS comprises PKKKRKV (SEQ ID NO: 8). In certain embodiments, the site-directed modifying polypeptide comprises at least two NLSs. In certain embodiments, the at least one NLS is at the C-terminus, the N-terminus, or both of the site-directed modifying polypeptide.

In certain embodiments, the site-directed modifying polypeptide further comprises a conjugation moiety that can bind to the antibody, antigen-binding portion thereof, or antibody mimetic. In certain embodiments, the conjugation moiety is a protein. In certain embodiments, the protein is Protein A, SpyCatcher, or a Halo-Tag.

In certain embodiments, the extracellular cell membrane-bound molecule is a protein selected from the group consisting of HLA-DR, CD44, CD11a, CD22, CD3, CD20, CD33, CD32, CD44, CD47, CD59, CD54, CD25, AchR, CD70, CD74, CTLA4, EGFR, HER2, or EpCam, OX40, PD-1, PD-L1, GITR, CD52, CD34, CD27, CD30, COS, or RSV.

In some embodiments, the extracellular cell membrane-bound molecule or protein is CD11a. In some embodiments, the antigen binding polypeptide is an anti-CD11a antibody, or antigen binding fragment thereof. In certain embodiments, the anti-CD11a antibody is efalizumab.

In some embodiments, the extracellular cell membrane-bound molecule or protein is CD25. In some embodiments, the antigen binding polypeptide is an anti-CD25 antibody, or antigen binding fragment thereof. In certain embodiments, the anti-CD25 antibody is daclizumab.

In another aspect, the invention provides a nucleoprotein comprising a site-directed modifying polypeptide and a guide RNA, wherein the guide RNA specifically hybridizes to a target region of the genome of a cell displaying the extracellular cell membrane-bound protein.

In another aspect, the invention provides an isolated nucleic acid encoding a site-directed modifying polypeptide described herein. In one embodiment, a vector comprises the nucleic acid. In another embodiment, a cell comprises the site-directed modifying polypeptide.

In another aspect, the invention provides a method of modifying the genome of a target cell, the method comprising contacting the target cell with a targeted active gene editing (TAGE) agent described herein. In certain embodiments, the target cell is a eukaryotic cell. In certain embodiments, the eukaryotic cell is a mammalian cell. In certain embodiments, the mammalian cell is a mouse cell, a non-human primate cell, or a human cell. In certain embodiments, the site-directed modifying polypeptide produces a cleavage site at the target region of the genome, thereby modifying the genome. In certain embodiments, the target region of the genome is a target gene.

In certain embodiments, a method comprising the use of a TAGE agent described herein is effective to modify expression of the target gene. In certain embodiments, the method is effective to increase expression of the target gene relative to a reference level. In certain embodiments, the method is effective to decrease expression of the target gene relative to a reference level.

In another aspect, provided herein is a method of modifying a nucleic acid sequence within a target cell in a mammalian subject, the method comprising contacting the target cell in the subject with a targeted active gene editing (TAGE) agent comprising an antigen binding polypeptide that specifically binds to an extracellular cell membrane-bound molecule, and a site-directed modifying polypeptide that recognizes the nucleic acid sequence within the target cell, such that the nucleic acid sequence of the target cell is modified.

In another aspect, provided herein is a method of modifying a nucleic acid sequence within a target cell in a mammalian subject, the method comprising locally administering to the subject a targeted active gene editing (TAGE) agent comprising an antigen binding polypeptide that specifically binds to an extracellular cell membrane-bound molecule, and a site-directed modifying polypeptide that recognizes the nucleic acid sequence within the target cell, such that the nucleic acid sequence of the target cell is modified.

In some embodiments, the method comprises locally administering the TAGE agent to the subject by intramuscular injection, intraosseous injection, intraocular injection, intratumoral injection, or intradermal injection.

In some embodiments, the method is effective to increase the level of genetically modified target cells in the subject relative to the level achieved by treatment with a site-directed modifying polypeptide lacking the antigen binding polypeptide.

In some embodiments, the mammalian subject is a human subject.

In some embodiments, the subject has a disease selected from an eye disease, a stem cell disorder, and a cancer, and wherein the method is effective to treat the disease.

In another aspect, provided herein is a method of modifying a nucleic acid sequence within a target mammalian cell, the method comprising contacting the target mammalian cell with a targeted active gene editing (TAGE) agent under conditions in which the TAGE agent is internalized into the target cell, such that the nucleic acid sequence is modified, wherein the TAGE agent comprises an antigen binding polypeptide that specifically binds to an extracellular cell membrane-bound molecule, and a site-directed modifying polypeptide that recognizes the nucleic acid sequence within the target cell, wherein the internalization of the TAGE agent is not dependent on electroporation.

In some embodiments, the target mammalian cell is a hematopoietic cell (HSC), a neutrophil, a T cell, a B cell, a dendritic cell, a macrophage, or a fibroblast. In certain embodiments, the target mammalian cell is a hematopoietic stem cell (HSC). In certain embodiments the target mammalian cell is a cell in the bone marrow that is not a hematopoietic stem cell (e.g., fibroblast, macrophages, osteoblasts, ostclasts, or endothelial cells).

In some embodiments, the antigen binding polypeptide specifically binds an extracellular cell membrane-bound molecule on a human HSC. In certain embodiments, the extracellular cell membrane-bound molecule on the HSC is CD34, EMCN, CD59, CD90, ckit, CD45, or CD49F.

In some embodiments, the target mammalian cell is contacted with the TAGE agent by co-incubation ex vivo.

In some embodiments, the method provides a genetically-modified target cell which is administered to a subject in need thereof.

In some embodiments, the target mammalian cell is contacted with the TAGE agent in situ by injection into a tissue of a subject.

In some embodiments, the TAGE agent is administered to the subject by intramuscular injection, intraosseous injection, intraocular injection, intratumoral injection, or intradermal injection.

In some embodiments, the nucleic acid is a gene in the genome of the target cell, wherein the expression of said gene is altered following said modification.

In some embodiments, the target mammalian cell is a mouse cell, a non-human primate cell, or a human cell.

In some embodiments, the antigen binding polypeptide is an antibody, an antigen-binding portion of an antibody, or an antibody-mimetic.

In certain embodiments, the antibody mimetic is an adnectin (i.e., fibronectin based binding molecules), an affilin, an affimer, an affitin, an alphabody, an aptamer, an affibody, a DARPin, an anticalin, an avimer, a fynomer, a Kunitz domain peptide, a monobody, a nanoCLAMP, a unibody, a versabody, an aptamer, or a peptidic molecule.

In some embodiments, the antigen-binding portion of the antibody is a nanobody, a domain antibody, an scFv, a Fab, a diabody, a BiTE, a diabody, a DART, a mnibody, a F(ab′)2, or an intrabody.

In some embodiments, the antibody is an intact antibody or a bispecific antibody.

In some embodiments, the extracellular cell membrane-bound molecule bound by the antigen binding polypeptide is HLA-DR, CD44, CD11a, CD22, CD3, CD20, CD33, CD32, CD44, CD47, CD59, CD54, CD25, AchR, CD70, CD74, CTLA4, EGFR, HER2, EpCam, OX40, PD-1, PD-L1, GITR, CD52, CD34, CD27, CD30, COS, or RSV.

In certain embodiments, the extracellular cell membrane-bound molecule or protein is CD11a. In some embodiments, the antigen binding polypeptide is an anti-CD11a antibody, or antigen binding fragment thereof. In certain embodiments, the anti-CD11a antibody is efalizumab, or an antigen binding fragment thereof.

In some embodiments, the extracellular cell membrane-bound molecule or protein is CD25. In some embodiments, the antigen binding polypeptide is an anti-CD25 antibody, or antigen binding fragment thereof. In certain embodiments, the anti-CD25 antibody is daclizumab.

In some embodiments, the TAGE agent further comprises at least one nuclear localization signal (NLS). In some embodiments, the TAGE agent comprises at least two nuclear localization signals (NLSs). In certain embodiments, the TAGE agent comprises four nuclear localization signals (NLSs). In certain embodiments, the TAGE agent comprises six nuclear localization signals (NLSs). In some embodiments, the TAGE agent comprises seven nuclear localization signals (NLSs). In some embodiments, the TAGE agent comprises eight nuclear localization signals (NLSs).

In some embodiments, the NLS comprises an SV40 NLS. In certain embodiments, the SV40 NLS comprises the amino acid sequence PKKKRKV (SEQ ID NO: 8).

In some embodiments, the target mammalian cell is a population of target mammalian cells. In some embodiments, the method is effective to increase the level (number) of genetically modified target mammalian cells in the population. In certain embodiments, the increase is evidenced by a response (e.g., phenotypic) in the mammalian cells. In certain embodiments, an increase number of mammalian cells modified by the TAGE agent can be determined by comparing the level in a population of mammalian cells relative to a level achieved by treatment with a site-directed modifying polypeptide lacking the antigen binding polypeptide.

In some embodiments, the site-directed modifying polypeptide of the TAGE agent has increased cellular internalization in the target mammalian cell. In certain embodiments, the increase in internalization is evidenced by a response, e.g., phenotypic, in the mammalian cell. In certain embodiments, an increase in the internalization of the TAGE agent into a mammalian cell can be determined by comparing the internalization of the TAGE agent in a population of mammalian cells relative to cellular internalization achieved with a site-directed modifying polypeptide lacking the antigen binding polypeptide.

In some embodiments, the site-directed modifying polypeptide of the TAGE agent has increased nuclear internalization in the target mammalian cell relative to nuclear internalization achieved with a site-directed modifying polypeptide lacking the antigen binding polypeptide.

In some embodiments, the site-directed modifying polypeptide comprises a nuclease or a nickase.

In some embodiments, the site-directed modifying polypeptide is a nucleic acid-guided nuclease, and the TAGE agent further comprises a guide nucleic acid that specifically hybridizes to a target region of the nucleic acid sequence of the target mammalian cell, wherein the guide nucleic acid and the nucleic acid-guided nuclease form a nucleoprotein.

In certain embodiments, the site-directed modifying polypeptide is a RNA-guided nuclease, and the TAGE agent further comprises a guide RNA that specifically hybridizes to a target region of the nucleic acid sequence of the target mammalian cell, wherein the guide RNA and the RNA-guided nuclease form a ribonucleoprotein. In some embodiments, the guide RNA is a single guide RNA (sgRNA) or a cr:trRNA.

In some embodiments, the RNA-guided nuclease is a Class 2 Cas polypeptide.

In some embodiments, the Class 2 Cas polypeptide is a Type II Cas polypeptide. In some embodiments, the Type II Cas polypeptide is Cas9. In some embodiments, the Cas9 nuclease is wildtype Cas9 nuclease (e.g., Streptococcus pyogenes Cas9, SEQ ID NO: 119). In some embodiments, the Cas9 nuclease comprises an amino acid sequence having at least 85%, 90%, 95%, 97%, 98%, 99%, or 100% identity to SEQ ID NO: 119. In certain embodiments, the Cas9 nuclease comprises the amino acid substitution C80A (e.g., SEQ ID NO: 1). In another the Cas9 nuclease comprises an amino acid sequence having at least 85%, 90%, 95%, 97%, 98%, 99%, or 100% identity to SEQ ID NO: 1.

In some embodiments, the Class 2 Cas polypeptide is a Type V Cas polypeptide. IN certain embodiments, the Type V Cas polypeptide is Cas12. In some embodiments, the Cas12 nuclease is wildtype Cas12 nuclease (e.g., Acidaminococcus sp. Cas12a, SEQ ID NO: 120). In some embodiments, the Cas12 nuclease comprises an amino acid sequence having at least 85%, 90%, 95%, 97%, 98%, 99%, or 100% identity to SEQ ID NO: 120. Examples of Cas12a variants useful in the TAGE agents herein include, but are not limited to, Alt-R® Cas12a (Cpf1) Ultra (e.g., IDT Catalog No. 10001272) or Cas12a as described in Kleinstiver, et al. Nature Biotechnology 37.3 (2019): 276-282, which is hereby incorporated by reference.

In some embodiments, the site-directed modifying polypeptide further comprises a conjugation moiety that binds to the antigen binding polypeptide or a complementary binding moiety attached thereto.

In certain embodiments, the conjugation moiety is a protein. In some embodiments, the protein is SpyCatcher or a Halo-Tag.

In some embodiments, the site-directed modifying polypeptide and the antigen binding polypeptide are conjugated via a linker. In some embodiments, the linker is a cleavable linker.

In some embodiments, the TAGE agent further comprises an endosomal escape agent. In certain embodiments, the endosomal escape agent is TDP or TDP-KDEL.

### BRIEF DESCRIPTION OF THE DRAWINGS

FIG. 1 is a schematic of a nuclease antibody-binding agent described herein complexed with an antibody, antigen-binding agent, or antibody-like molecule to form a targeted active gene editing (TAGE) agent. In FIG. 1, the term “nuclease antibody-binding agent” refers to a site-directed modifying polypeptide including a nuclease.

FIG. 2 graphically depicts the results of an in vitro DNA cleavage assay assessing Cas9-2×NLS-ProteinA alone (“Cas9-pA”) or Cas9-2×NLS-ProteinA complexed with an anti-CD3 antibody (“Cas9-pA:α-CD3”), or Cas9(C80A)-2×NLS (“C80A”) with activity plotted relative to Cas9(C80A)-2×NLS activity.

FIG. 3 graphically depicts the results of an ex vivo editing assay assessing editing activity of Cas9-2×NLS-ProteinA (“Cas9-pA”) or Cas9 (C80A)-2×NLS (“C80A”) following nucleofection into stimulated human T cells. A guide RNA targeting CD47 was associated with the respective TAGE agents to form ribonucleoproteins, and the ribonucleoproteins were nucleofected into T cells to test for editing. Editing was measured using a phenotypic readout measuring the loss of surface CD47 using flow cytometry. Editing activity is plotted relative to Cas9 (C80A)-2×NLS activity.

FIG. 4 graphically depicts the results of an in vitro binding assay to assess binding of Cas9-2×NLS-ProteinA (“Cas9-pA”) to an anti-CD3 antibody. The results for Cas9-pA alone and anti-CD3 antibody alone are also shown.

FIGS. 5A and 5B graphically depict the results of a FACS-based internalization assay measuring the rate of PBMC internalization for anti-CD3 (18 nM) or anti-CD22 (100 nM) antibodies in CD8 T cells (FIG. 5A) and in CD19 B Cells (FIG. 5B).

FIGS. 6A-6C show results from binding and internalization studies of antibodies (huIgG1, CD22) complexed with Cas9-2×NLS-proteinA (“Cas9-pA”) to form TAGE agents. FIG. 6A graphically depicts the results of a FACS-based cell binding assay in which 10 nM of each indicated protein was added to PBMCs and stained for 30 minutes. FIG. 6B graphically depicts the results of a FACS-based internalization assay in which 10 nM of each indicated protein was added to PBMCs for the indicated temperature and time. Samples from each condition with and without quenching with an anti-A488 antibody were assessed by FACS analysis. FIG. 6C further illustrates internalization by T cells vs B cells in the pool of PBMCs.

FIGS. 7A-7D graphically depicts the results of a FACS-based internalization assays utilizing various quench methods (heparin wash, acid wash, anti-A488 antibody, no quench), in which internalization of a TAGE agent including Cas9-2×NLS-proteinA (“Cas9-pA”), an anti-CD3 antibody, or Cas9-pA complexed with an anti-CD3 antibody (“Cas9 pA:CD3”) was assessed in T cells (FIGS. 7A and 7B) or myeloid cells (FIG. 7C). FIG. 7A graphically depicts the results of the internalization assay with an anti-CD3 antibody labelled with A488 or Cas9-pA:anti-CD3 RNP with guide RNA labelled with A488. FIG. 7B graphically depicts the results of the internalization assay in T cells with Cas9-pA:anti-CD3 RNP or Cas9-pA with guide RNA labelled with ATTO550. FIG. 7C graphically depicts the results of the internalization assay in myeloid cells with Cas9-pA:anti-CD3 RNP or Cas9-pA with guide RNA labelled with ATTO550. FIG. 7D graphically depicts the results of a live dead FACS-based assay to evaluate the toxicity effects of each quench method.

FIG. 8 graphically depicts the results of an in vitro DNA cleavage assay assessing the DNA cleavage by the TAGE agent Cas9-2×NLS-DARPin(Ec1) (“Cas9-Darpin(EC1)”) (also referred to as Cas9-DARPin(EpCAM)) or Cas9(C80A)-2×NLS (“C80A”) with activity plotted relative to Cas9(C80A)-2×NLS activity.

FIG. 9 graphically depicts the results of an ex vivo editing assay assessing editing of the TAGE agents Cas9-2×NLS-ProteinA (Cas9-pA) or Cas9 (C80A) following nucleofection into stimulated human T cells. A guide RNA targeting CD47 was associated with the respective TAGE agents to form ribonucleoproteins, and the ribonucleoproteins were nucleofected into T cells to test for editing. Editing was measured using a phenotypic readout measuring the loss of surface CD47 using flow cytometry. Editing activity is plotted relative to C80A activity.

FIGS. 10A-10D graphically depict the results of a FACS-based binding assay to assess binding of the TAGE agents Cas9-2×NLS-DARPin(EpCAM) (“Darpin”) or Cas9(C80A)-2×NLS (“C80A”) on the cell surface of epithelial cell lines BT474 or SKBR3. FIGS. 10A and 10B graphically depict the results of the FACS-based binding assay for Cas9 (C80A)-2×NLS or Cas9-2×NLS-DARPin(EpCAM) at 10, 25, 50, 100, or 300 nM on BT474 cells (FIG. 10A) or SKBR3 cells (FIG. 10B). FIG. 11C graphically depicts the results of binding by a EpCAM antibody on SKBR3 cells or BT474 cells, demonstrating that both cell lines express EpCAM. FIG. 10D graphically depicts the results of the FACS-based binding assay for Cas9 (C80A)-2×NLS or Cas9-2×NLS-DARPin(EpCAM) at 25, 100, or 300 nM on BT474 cells or SKBR3 cells.

FIG. 11 graphically depicts the results of a FACS-based internalization assay in which 100 nM or 300 nM of the TAGE agent Cas9-DARPin (EpCAM) was incubated with BT474 cells or SKBR3 cells for the indicated time (60 min or 30 min) at 37° C. or 4° C. prior to assaying with FACS, with or without prior quenching.

FIG. 12 graphically depicts the results of an ex vivo editing assay assessing editing achieved by co-incubation of the TAGE agent Cas9-2×NLS-DARPin (EpCAM) RNP with huCD47 guide RNA in BT474 cells or SKBR3 cells after the indicated time (4 days or 7 days). Results obtained from control cells not exposed to an RNP are also shown. Editing was measured using a phenotypic readout measuring the loss of surface CD47 using flow cytometry. The percent of edited cells as determined by flow cytometry is indicated on each graph.

FIG. 13 graphically depicts the results of an ex vivo editing assay, as assessed by flow cytometry, following nucleofection of the TAGE agent Cas9-2×NLS-DARPin (EpCAM) RNP with huCD47 guide RNA in human T cells after the indicated time (4 days or 7 days). Editing was measured using a phenotypic readout measuring the loss of surface CD47 using flow cytometry.

FIGS. 14A and 14B graphically depict analyses of Cas9-2×NLS-Halo:anti-CD22 TAGE agents (“Cas9-Halo=mCD22”). FIG. 14A graphically depicts a chromatogram from size exchange chromatography (S200 10/300 Increase sizing column) of a Cas9-Halo:anti-CD22 antibody TAGE agent, in which peaks between 8.5-11 mL represent antibody-Cas9 conjugated material. FIG. 14B is an image of an SDS-PAGE used to identify the ratio of Cas9-Antibody conjugation. The lanes containing material from peaks 1 through peak 3 of the size exchange analysis are notated. “Ab-2×Cas9” refers to conjugates with two Cas9 molecules per antibody.

FIGS. 15A and 15B graphically depict the results of a FACS-based internalization assay in which 20 nM of the indicated TAGE agent RNP (Cas9-2×NLS-Halo:anti-CD22 antibody (“Cas9-Halo:mCD22”), Cas9-2×NLS-Halo:IgG1 (“Cas9-Halo-IgG1”), or Cas9-2×NLS-Halo (“Cas9-Halo”)) with an A488 guide RNA was incubated with total splenocytes (FIG. 15A) or tumor infiltrating lymphocytes (FIG. 15B) for the indicated time (15 min or 60 min) at 37° C. or 4° C. Samples from each condition with and without quenching were assessed by FACS analysis gated on CD19+ B cells.

FIGS. 16A and 16B graphically depict the results of an in vitro DNA cleavage assay (FIG. 16A) and an ex vivo nucleofection editing assay in human T cells (FIG. 16B) assessing DNA cleavage by Cas9-2×NLS-Halo alone (“Cas9-Halo”), or DNA cleavage by TAGE agents including Cas9-2×NLS-Halo complexed with an anti-CD22 antibody (“Cas9-Halo:mCD22”), an anti-CTLA4 antibody (“Cas9-Halo:mCTLA4”), IgG1 (“Cas9-Halo:IgG1”) with activity plotted relative to Cas9(C80A)-2×NLS activity. To assess ex vivo editing, a guide RNA targeting CD47 was associated with the respective TAGE agents to form ribonucleoproteins, and the ribonucleoproteins were nucleofected into T cells to test for editing. Editing was measured using a phenotypic readout measuring the loss of surface CD47 using flow cytometry. FIG. 16B additionally shows editing by Halo-30a.a.-Cas9, Halo-3a.a.-Cas9, and hIgG1:Halo-3a.a.-Cas9, where 30 a.a. and 3 a.a. refers to the amino acid (“a.a.”) length of the peptide linker in the construct.

FIG. 17 graphically depicts the results from a FACS-based internalization assay in which the indicated TAGE agent RNPs (Cas9(C80A)-2×NLS (“C80A”), Cas9-2×NLS-Halo alone (“Cas9-Halo”), or Cas9-2×NLS-Halo complexed with an anti-CD22 antibody (“Halo-mCD22”), an anti-CTLA4 antibody (“Halo-mCTLA4”), MHCII-Nb (“MHCII-Nb”), or IgG1 (“Halo-IgG1”) were assessed for internalization into a mixed cell population isolated from B116F110 tumors. Results are shown for gated DC cells, non-DC myeloid cells, B cells, T cells, non-T/B cells, and CD45− PDPN+ cells.

FIGS. 18A-18C graphically depict results from an in vitro binding assay with a TAGE agent including Cas9-2×NLS-Halo (“Cas9-Halo”) conjugated to an anti-CD22 antibody (FIG. 18A; binding to mouse splenocytes), an anti-FAP antibody (FIG. 18B; binding to human fibroblasts), or an anti-CTLA-4 antibody (FIG. 18C; binding to T cells). FIG. 18A: 20 nM of either RNP with A488-labeled guide or A488-labeled antibody was incubated with total mouse splenocytes for 30 minutes on ice. FIG. 18B: Human fibroblasts were incubated with 20 nM protein for 30 minutes on ice. Antibody is labeled with A488 (1:1 dye:antibody) and each RNP contains a A488-labeled guide. FIG. 18C: Stimulated mouse T cells were incubated with 20 or 100 nM protein for 15 minutes at 37 C. Antibody was labeled with A488 (1:1 dye:antibody) and each RNP contains a A488-labeled guide.

FIG. 18D graphically depicts the results of an ex vivo editing assay with a TAGE agent including human anti-FAP antibody conjugated to Cas9-2×NLS-Halo and co-incubated with human dermal fibroblasts. Human dermal fibroblasts were plated overnight. A guide RNA targeting CD47 was associated with the respective TAGE agents to form ribonucleoproteins, and the ribonucleoproteins were co-incubated with fibroblasts to test for editing. Editing was measured using a phenotypic readout measuring the loss of surface CD47 using flow cytometry. 37.5 uM of RNP was incubated with the cells in 2.5% FBS for 1 hour. Then complete media was added, diluting the RNP to 300 nM. Samples were analyzed for CD47 expression on day 6 post incubation.

FIGS. 18E and 18F graphically depict the results of an ex vivo editing assay with a TAGE agent including mouse anti-CTLA-4 antibody conjugated to Cas9-Halo-2×NLS and co-incubated with regulatory T cells (FIG. 18E) or total stimulated T cells (FIG. 18F). Gene editing was measured using the TdTomato florescence reporter system. Induced Tregs or total splenocytes were stimulated for 3 days. 250,000 cells were incubated with 75 pmoles of RNP (3.75 uM) for one hour with 2.5% serum. After one hour, complete media was added to dilute RNP to 300 nM. Cells were analyzed by FACS on Day 6 post incubation to measure tdTomato signal.

FIGS. 19A-19F graphically depict the results of ex vivo editing and binding assays with a TAGE agent including a human anti-FAP antibody conjugated to Cas9. The antibody was conjugated via a spytag (ST) moiety to SpyCatcher-Cas9(WT)-2×NLS (“FAP=SC-Cas9”). A guide RNA targeting CD47 was associated with the respective TAGE agents to form ribonucleoproteins, and the ribonucleoproteins were co-incubated with fibroblasts to test for editing. Editing was measured using a phenotypic readout measuring the loss of surface CD47 using flow cytometry. FIG. 19A graphically depicts results of an FAP=SC-Cas9 editing assay in human dermal fibroblasts (“C80A” refers to Cas9(C80A)-2×NLS; “FAP-LL” refers to FAP-ST-long linker; “FAP-SL” refers to FAP-ST-short linker). FIGS. 19B and 19C graphically depicts results of an FAP=(4×-SC-2×)₂ editing assay in human dermal fibroblasts at 3750 nM (FIG. 19B) or 5850 nm (FIG. 19C) (“C800A+FAP” refers to added FAP-ST antibody added in trans during editing to rule out effects of unconjugated antibody, “2×” refers to 2×NLS, “4×” refers to 4×NLS). FIG. 19D graphically depicts the results comparing editing by hCTLA4=Cas9 (“Ipi”) vs FAP=Cas9 in human dermal fibroblasts (“No RNP” refers to a condition where no Cas9 was added; “C80A:BFP” refers to Cas9(C80A)-2×NLS added with a non-targeting guide: All other conditions used sgCD47 as a targeting gRNA; FAP=(SC-Cas9)2 refers to a positive control for targeting Cas9 to FAP+fibroblasts; Ipi=(SC-Cas9)2 refers to a negative control for Ab-Cas9; shouldn't bind fibroblasts). FIG. 19E show results of a fibroblast binding assay with the indicated molecules. FIG. 19F show results of a competition assay with excess Fc=SC-Cas9 and the indicated molecules on human dermal fibroblasts. “Pali” refers to palivizumab, an antibody against respiratory syncytial virus (RSV), used as negative control; “Ipi” refers to ipilimumab, antibody against CTLA-4, negative control; “Fc=(SC-Cas9)₂” refers to negative control for the Fc portion of antibody and 2 Cas9s linked together, “FAP=(SC-Cas9)₂” refers to full-length antibody, positive control; “FAP-F(ab′)2=(SC-Cas9)₂” refers to only F(ab′)2, no Fc domain; positive control; “FAP-Fab=(SC-Cas9)₂” refers to only Fab, single binding domain and no Fc domain; positive control; “FAP=(SC-Cas9)₂+excess FAP” refers to additional control where excess FAP antibody was added to block binding of FAP=Cas9 conjugate (demonstrates FAP-mediated specificity).

FIG. 20A-20C graphically depict the results of an in vitro screen for TAGE agents including antibody-Cas9 conjugates (“Ab=Cas9”) that bind T cells. Each antibody was conjugated via a SpyTag (“ST) to Cas9(WT)-2×NLS-Spycatcher-HTN (“AC28”). FIG. 20A graphically depicts the level of CD4+ T cell binding by the indicated RNPs. Total PBMCs were activated for 2 days and were then stained with Ab=Cas9 conjugates at 7 or 70 nM. The A550 signal comes from an A550-labeled guide. Pali=palivizumab, negative control. An ANOVA with multiple comparisons was conducted to compare each antibody to palivizumab (“Pali”); antibodies were moved to next step if they had significantly more staining than Pali. FIGS. 20B and 20C graphically depict the results of a blocking assay to assess whether T cell binding by the indicated Antibody=Cas9 TAGE agents was blocked by unconjugated (“cold”) antibody in CD8+ T cells (FIG. 20B) or CD4+ T cells (FIG. 20C). The TAGE agents were complexed with a A550-labeled guide, which generated the A550 signal notated on the Y-axis. FIGS. 20D and 20E graphically depict the percent of Ab=Cas9 binding that is blocked by an unconjugated antibody in CD4+ T cells (FIG. 20D) and CD8+ T cells (FIG. 20E).

FIGS. 21A and 21B graphically depict the results of an ex vivo editing assay in human CD4+ T cells (FIG. 21A) and CD8+ T cells (FIG. 21B) with TAGE agents identified in Example 19 including an antibody conjugated to Cas9 (Ab=Cas9). Anti-CD11a and anti-CD25a antibodies (as identified in the T cell screen described in Example 21) were conjugated to Cas9 (CD11a=Cas9 and CD25a=Cas9). Each antibody was conjugated via a SpyTag (“ST) to Cas9(WT)-2×NLS-Spycatcher-HTN (“AC28”) or Cas9(WT)-2×NLS-Spycatcher-4×NLS (“AC26”) to form antibody-based TAGE agents. A guide RNA targeting CD47 was associated with the respective TAGE agents, and the TAGE agents were co-incubated with T cells to test for editing. Editing was measured using a phenotypic readout measuring the loss of surface CD47 using flow cytometry. “2 step” indicates that 3750 nM RNP was added for 1 hour, then diluted until 300 nM, and incubated until readout. Antibody=AC26 (or AC28) refers to a test article including a full-length antibody; Pali=AC26 or Pali=AC28 was used as a negative control as it does not bind T cells. F(ab′)2 refers to an antibody fragment without the Fc domain.

FIGS. 22A and 22B graphically depict the results of an assay comparing two different methods for detecting ex vivo editing of T cells or fibroblasts: (1) editing measurements obtained by flow cytometry (e.g., to detect a phenotypic readout, i.e., loss of cell surface expression of CD47 or CD44) or (2) editing measurements obtained by next generation sequencing (NGS) to detect editing of the genes encoding CD47 or CD44. The same samples were analyzed by each approach and the measurements were compared. FIG. 22A graphically depicts a comparison between editing measurements by flow cytometry (y-axis) vs NGS (x-axis) for samples with 0% to 50% editing. FIG. 22A graphically depicts a comparison between editing measurements by flow cytometry (y-axis) vs NGS (x-axis) for samples with 0% to 2% editing (same samples as in FIG. 22B, but with different x-axis scale).

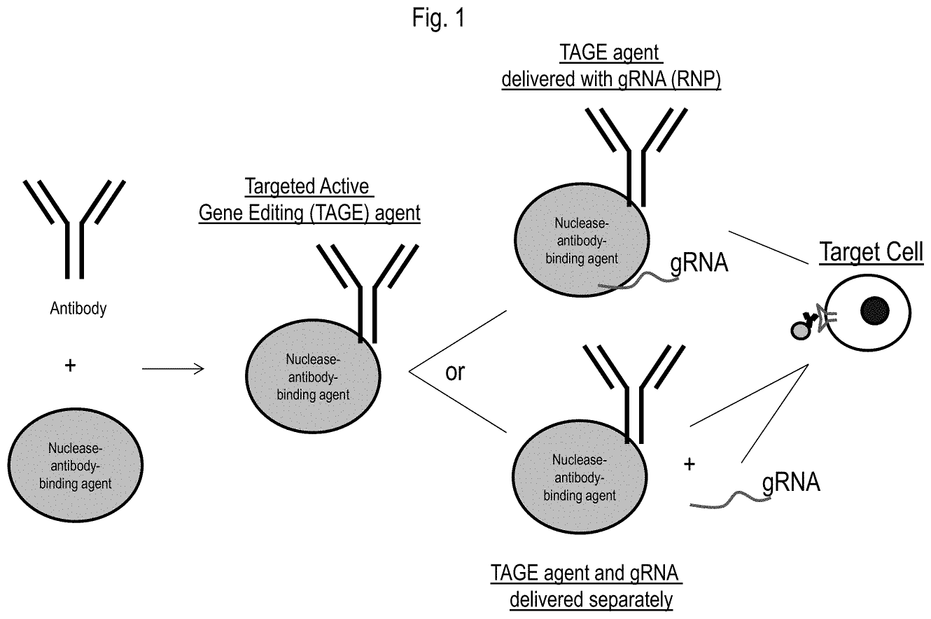

### DETAILED DESCRIPTION OF THE INVENTION

Provided herein are compositions and methods relating to Targeted Active Gene Editing (TAGE) agents that can edit nucleic acids within specific cell types in vivo and ex vivo. Further, provided herein are compositions and methods for promoting cellular internalization of site-directed modifying polypeptides within cells in vivo and ex vivo. The modular and programmable design of TAGE agents enables rapid re-targeting and multi-functionality to enable flexibility targeting of a variety of desired cell types. Further, by editing specific nucleic acids in specific target cells, TAGE agents have dual specificity and have fewer off-target effects than DNA-based delivery approaches. To achieve this, TAGE agents include one or more antigen-binding polypeptides that promote cell binding and/or cellular internalization. The TAGE agents of the present compositions and methods can thereby promote delivery and internalization of site-directed modifying polypeptides (e.g. gene editing polypeptides), such as Cas9, into target cell types. Further, antigen-binding polypeptides not only allow for receptor-mediated entry of the TAGE agent, but in certain instances, the antigen-binding polypeptides also mediate the biology of the cell (e.g., by altering intracellular signal transduction pathways). TAGE agents described herein are particularly suited for systemic delivery.

Accordingly, provided herein are methods and compositions relating to a TAGE agent comprising an antigen-binding polypeptide and a site-directed modifying polypeptide that recognizes a nucleic acid sequence within a cell, wherein the antigen-binding polypeptide and the site-directed modifying polypeptide are stably associated such that the site-directed modifying polypeptide can be internalized into a cell.

In one aspect, provided herein is a targeted active gene editing (TAGE) agent that comprises an antigen binding polypeptide that specifically binds to an extracellular cell membrane-bound molecule (e.g., a cell surface molecule), and a site-directed modifying polypeptide that recognizes a nucleic acid sequence within a target cell. The antigen binding polypeptide and the site-directed modifying polypeptide are stably associated such that the site-directed modifying polypeptide can be internalized into the target cell displaying the extracellular cell membrane-bound molecule.

Further, provided herein are methods of modifying a genome of a cell ex vivo or in vivo, and methods of delivering a site-directed modifying polypeptide to a subject via a TAGE agent. Targeted ex vivo editing by TAGE agents enables genetic modification of cells (e.g., hematopoietic stem cells) for use in a variety of cellular therapies. Additionally, administration of a TAGE agent to a subject enables targeted editing of desired cell types in vivo.

### 1. Definitions

The term “targeted active gene editing” or “TAGE” agent refers to a complex of molecules including an antigen binding polypeptide (e.g., an antibody or an antigen-binding portion thereof) that specifically binds to an extracellular target molecule (e.g., an extracellular protein or glycan, such as an extracellular protein on the cell surface) displayed on a cell membrane, and a site-directed modifying polypeptide (such as, but not limited to, an endonuclease) that recognizes a nucleic acid sequence. The antigen binding polypeptide of a TAGE agent is associated with the site-directed modifying polypeptide such that at least the site-directed modifying polypeptide is internalized by a target cell, i.e., a cell expressing an extracellular molecule bound by the antigen binding polypeptide. An example of a TAGE agent is an active CRISPR targeting (TAGE) agent where the site directed polypeptide is a nucleic acid-guided DNA endonuclease (e.g., RNA-guided endonuclease or DNA-guided endonuclease), such as Cas9 or Cas12. In some embodiments, the TAGE agent includes at least one NLS. Notably, a TAGE agent can target any nucleic acid within a cell, including, but not limited to, a gene.

The term “antigen binding polypeptide” as used herein refers to a protein that binds to a specified target antigen, such as an extracellular cell-membrane bound protein (e.g., a cell surface protein). Examples of an antigen binding polypeptide include an antibody, antigen-binding fragments of an antibody, and an antibody mimetic. In certain embodiments, an antigen-binding polypeptide is an antigen binding peptide.

As used herein, a “site-directed modifying polypeptide” refers to a protein that is targeted to a specific nucleic acid sequence or set of similar sequences of a polynucleotide chain via recognition of the particular sequence(s) by the modifying polypeptide itself or an associated molecule (e.g., RNA), wherein the polypeptide can modify the polynucleotide chain.

The terms “polypeptide” or “protein”, as used interchangeably herein, refer to any polymeric chain of amino acids. The term “polypeptide” encompasses native or artificial proteins, protein fragments and polypeptide analogs of a protein sequence.

The term “conjugation moiety” as used herein refers to a moiety that is capable of conjugating two more or more molecules, such as an antigen binding protein and a site-directed modifying polypeptide. The term “conjugation,” as used herein, refers to the physical or chemical complexation formed between a molecule (for e.g. an antibody) and the second molecule (e.g. a site-directed modifying polypeptide, therapeutic agent, drug or a targeting molecule). The chemical complexation constitutes specifically a bond or chemical moiety formed between a functional group of a first molecule (e.g., an antibody) with a functional group of a second molecule (e.g., a site-directed modifying polypeptide, therapeutic agent or drug). Such bonds include, but are not limited to, covalent linkages and non-covalent bonds, while such chemical moieties include, but are not limited to, esters, carbonates, imines phosphate esters, hydrazones, acetals, orthoesters, peptide linkages, and oligonucleotide linkages. In one embodiment, conjugation is achieved via a physical association or non-covalent complexation.

As used herein, the term “target cell” refers to a cell or population of cells, such as mammalian cells (e.g., human cells), which includes a nucleic acid sequence in which site-directed modification of the nucleic acid is desired (e.g., to produce a genetically-modified cell in vivo or ex vivo). In some instances, a target cell displays on its cell membrane an extracellular molecule (e.g., an extracellular protein such as a receptor or a ligand, or glycan) specifically bound by an antigen binding polypeptide of the TAGE agent.

As used herein, the term “genetically-modified cell” refers to a cell, or an ancestor thereof, in which a DNA sequence has been deliberately modified by a site-directed modifying polypeptide.

As used herein, the term “nucleic acid” refers to a molecule comprising nucleotides, including a polynucleotide, oligonucleotide, or other DNA or RNA. In one embodiment, a nucleic acid is present in a cell and can be transmitted to progeny of the cell via cell division. In some instances, the nucleic acid is a gene (e.g., an endogenous gene) found within the genome of the cell within its chromosomes. In other instances, a nucleic acid is a mammalian expression vector that has been transfected into a cell. DNA that is incorporated into the genome of a cell using, e.g., transfection methods, is also considered within the scope of an “nucleic acid” as used herein, even if the incorporated DNA is not meant to be transmitted to progeny cells.

As used herein, the term “endosomal escape agent” or “endosomal release agent” refers to an agent (e.g., a peptide) that, when conjugated to a molecule (e.g., a polypeptide, such as a site-directed modifying polypeptide), is capable of promoting release of the molecule from an endosome within a cell. Polypeptides that remain within endosomes can eventually be targeted for degradation or recycling rather than released into the cytoplasm or trafficked to a desired subcellular destination. Accordingly, in some embodiments, a TAGE agent comprises an endosomal escape agent.

As used herein, the term “stably associated” when used in the context of a TAGE agent refers to the ability of the antigen binding polypeptide and the site-directed modifying polypeptide to complex in such a way that the complex can be internalized into a target cell such that nucleic acid editing can occur within the cell. Examples of ways to determine if a TAGE agent is stably associated include in vitro assays whereby association of the complex is determined following exposure of a cell to the TAGE agent, e.g., by determining whether gene editing occurred using a standard gene editing system. Examples of such assays are known in the art, such as SDS-PAGE, western blot analysis, size exclusion chromatography, and electrophoretic mobility shift assay to determine protein complexes and PCR amplification, direct sequencing (e.g., next-generation sequencing or Sanger sequencing), enzymatic cleavage of a locus with a nuclease (e.g., Celery) to confirm editing; and indirect phenotypic assays that measure the downstream effects of editing a specific gene, such as loss of a protein as measured by Western blot or flow cytometry or generation of a functional protein, as measured by functional assays.

As used herein, the term “modifying a nucleic acid” refers to any modification to a nucleic acid targeted by the site-directed modifying polypeptide. Examples of such modifications include any changes to the amino acid sequence including, but not limited to, any insertion, deletion, or substitution of an amino acid residue in the nucleic acid sequence relative to a reference sequence (e.g., a wild-type or a native sequence). Such amino acid changes may, for example, may lead to a change in expression of a gene (e.g., an increase or decrease in expression) or replacement of a nucleic acid sequence. Modifications of nucleic acids can further include double stranded cleavage, single stranded cleavage, or binding of any RNA-guided endonuclease disclosed herein to a target site. Binding of a RNA-guided endonuclease can inhibit expression of the nucleic acid or can increase expression of any nucleic acid in operable linkage to the nucleic acid comprising the target site.

The term “cell-penetrating peptide” (CPP) refers to a peptide, generally of about 5-60 amino acid residues (e.g., 5-10, 10-15, 15-20, 20-25, 25-30, 30-35, 35-40, 40-45, 45-50, 50-55, or 55-60 amino acid resides) in length, that can facilitate cellular uptake of a conjugated molecule, particularly one or more site-specific modifying polypeptides. A CPP can also be characterized in certain embodiments as being able to facilitate the movement or traversal of a molecular conjugate across/through one or more of a lipid bilayer, micelle, cell membrane, organelle membrane (e.g., nuclear membrane), vesicle membrane, or cell wall. A CPP herein can be cationic, amphipathic, or hydrophobic in certain embodiments. Examples of CPPs useful herein, and further description of CPPs in general, are disclosed in Borrelli, Antonella, et al. Molecules 23.2 (2018): 295; Milletti, Francesca. Drug discovery today 17.15-16 (2012): 850-860, which are incorporated herein by reference. Further, there exists a database of experimentally validated CPPs (CPPsite, Gautam et al., 2012). The CPP of a TAGE agent can be any known CPP, such as a CPP shown in the CPPsite database.

As used herein, the term “nuclear localization signal” or “NLS” refers to a peptide that, when conjugated to a molecule (e.g., a polypeptide, such as a site-directed modifying polypeptide), is capable of promoting import of the molecule into the cell nucleus by nuclear transport. The NLS can, for example, direct transport of a protein with which it is associated from the cytoplasm of a cell across the nuclear envelope barrier. The NLS is intended to encompass not only the nuclear localization sequence of a particular peptide, but also derivatives thereof that are capable of directing translocation of a cytoplasmic polypeptide across the nuclear envelope barrier. In some embodiments, one or more NLSs (e.g., 1, 2, 3, 4, 5, 6, 7, 8, 2-6, 3-7, 4-8, 5-9, 6-10, 7-10, 8-10 NLSs) can be attached to the N-terminus, the C-terminus, or both the N- and C-termini of the polypeptide of a TAGE agent herein.

The term “TAT-related peptide” as used herein, refers to a CPP that is derived from the transactivator of transcription (TAT) of human immunodeficiency virus. The amino acid sequence of a TAT peptide comprises RKKRRQRRR (SEQ ID NO: 9). Thus, a TAT-related peptide includes any peptide comprising the amino acid sequence of RKKRRQRRR (SEQ ID NO: 9), or an amino acid sequence having conservative amino acid substitutions wherein the peptide is still able to internalize into a cell. In certain embodiments, a TAT-related peptide includes 1, 2, or 3 amino acid substitutions, wherein the TAT-related peptide is able to internalize into a target cell.

As used herein, the term “specifically binds” refers an antigen binding polypeptide which recognizes and binds to an antigen present in a sample, but which antigen binding polypeptide does not substantially recognize or bind other molecules in the sample. In one embodiment, an antigen binding polypeptide that specifically binds to an antigen, binds to an antigen with an Kd of at least about 1×10⁻⁴, 1×10⁻⁵, 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more as determined by surface plasmon resonance or other approaches known in the art (e.g., filter binding assay, fluorescence polarization, isotheramal titration calorimetry), including those described further herein. In one embodiment, an antigen binding polypeptide specifically binds to an antigen if the antigen binding polypeptide binds to an antigen with an affinity that is at least two-fold greater as determined by surface plasmon resonance than its affinity for a nonspecific antigen. When used in the context of a ligand, the term “specifically binds” refers to the ability of a ligand to recognize and bind to its respective receptor(s). When used in the context of a CPP, the term “specifically binds” refers to the ability of CPPs to translocate a cell's membrane. In some instances, when a CPP(s) and either an antibody or a ligand are combined as a TAGE agent, the TAGE agent may display the specific binding properties of both the antibody or ligand and the CPP(s). For example, in such instances, the antibody or ligand of the TAGE agent may confer specific binding to an extracellular cell surface molecule, such as a cell surface protein, while the CPP(s) confers enhanced ability of the TAGE agent to translocate across a cell membrane.

The term “antibody” is used herein in the broadest sense and encompasses various antibody structures, including but not limited to monoclonal antibodies, polyclonal antibodies, multispecific antibodies (e.g., bispecific antibodies), nanobodies, monobodies, and antibody fragments so long as they exhibit the desired antigen-binding activity.

The term “antibody” includes an immunoglobulin molecule comprising four polypeptide chains, two heavy (H) chains and two light (L) chains inter-connected by disulfide bonds, as well as multimers thereof (e.g., IgM). Each heavy chain (HC) comprises a heavy chain variable region (or domain) (abbreviated herein as HCVR or VH) and a heavy chain constant region (or domain). The heavy chain constant region comprises three domains, CH1, CH2 and CH3. Each light chain (LC) comprises a light chain variable region (abbreviated herein as LCVR or VL) and a light chain constant region. The light chain constant region comprises one domain (CL1). Each VH and VL is composed of three complementarity determining regions (CDRs) and four framework regions (FRs), arranged from amino-terminus to carboxy-terminus in the following order: FR1, CDR1, FR2, CDR2, 1-R3, CDR3, FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass. Thus, the VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

As used herein, the term “CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

The term “Fc domain” is used to define the C-terminal region of an immunoglobulin heavy chain, which may be generated by papain digestion of an intact antibody. The Fc domain may be a native sequence Fc domain or a variant Fc domain. The Fc domain of an immunoglobulin generally comprises two constant domains, a CH2 domain and a CH3 domain, and optionally comprises a CH4 domain Replacements of amino acid residues in the Fc portion to alter antibody effector function are known in the art (Winter, et al. U.S. Pat. Nos. 5,648,260; 5,624,821). The Fc domain of an antibody mediates several important effector functions e.g. cytokine induction, ADCC, phagocytosis, complement dependent cytotoxicity (CDC) and half-life/clearance rate of antibody and antigen-antibody complexes. In certain embodiments, at least one amino acid residue is altered (e.g., deleted, inserted, or replaced) in the Fc domain of an Fc domain-containing binding protein such that effector functions of the binding protein are altered.

An “intact” or a “full length” antibody, as used herein, refers to an antibody comprising four polypeptide chains, two heavy (H) chains and two light (L) chains. In one embodiment, an intact antibody is an intact IgG antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “human antibody”, as used herein, refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “humanized antibody” is intended to refer to antibodies in which CDR sequences derived from the germline of one mammalian species, such as a mouse, have been grafted onto human framework sequences. Additional framework region modifications may be made within the human framework sequences. A “humanized form” of an antibody, e.g., a non-human antibody, refers to an antibody that has undergone humanization.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

An “antibody fragment”, “antigen-binding fragment” or “antigen-binding portion” of an antibody refers to a molecule other than an intact antibody that comprises a portion of an intact antibody and that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv); and multispecific antibodies formed from antibody fragments.

A “multispecific antigen binding polypeptide” or “multispecific antibody” is an antigen binding polypeptide that targets and binds to more than one antigen or epitope. A “bispecific,” “dual-specific” or “bifunctional” antigen binding polypeptide or antibody is a hybrid antigen binding polypeptide or antibody, respectively, having two different antigen binding sites. Bispecific antigen binding polypeptides and antibodies are examples of a multispecific antigen binding polypeptide or a multispecific antibody and may be produced by a variety of methods including, but not limited to, fusion of hybridomas or linking of Fab′ fragments. See, e.g., Songsivilai and Lachmann, 1990, Clin. Exp. Immunol. 79:315-321; Kostelny et al., 1992, J. Immunol. 148:1547-1553, Brinkmann and Kontermann. 2017. MABS. 9(2):182-212. The two binding sites of a bispecific antigen binding polypeptide or antibody, for example, will bind to two different epitopes, which may reside on the same or different protein targets.

The term “antibody mimetic” or “antibody mimic” refers to a molecule that is not structurally related to an antibody but is capable of specifically binding to an antigen. Examples of antibody mimetics include, but are not limited to, an adnectin (i.e., fibronectin based binding molecules), an affilin, an affimer, an affitin, an alphabody, an affibody, DARPins, an anticalin, an avimer, a fynomer, a Kunitz domain peptide, a monobody, a nanoCLAMP, a nanobody, a unibody, a versabody, an aptamer, and a peptidic molecule all of which employ binding structures that, while they mimic traditional antibody binding, are generated from and function via distinct mechanisms.

Amino acid sequences described herein may include “conservative mutations,” including the substitution, deletion or addition of nucleic acids that alter, add or delete a single amino acid or a small number of amino acids in a coding sequence where the nucleic acid alterations result in the substitution of a chemically similar amino acid. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N) and glutamine (Q); N, Q, serine (S), threonine (T), and tyrosine (Y); K, R, H, D, and E; D, E, N, and Q; alanine (A), valine (V), leucine (L), isoleucine (I), proline (P), phenylalanine (F), tryptophan (W), methionine (M), cysteine (C), and glycine (G); F, W, and Y; H, F, W, and Y; C, S and T; C and A; S and T; C and S; S, T, and Y; V, I, and L; V, I, and T. Other conservative amino acid substitutions are also recognized as valid, depending on the context of the amino acid in question. For example, in some cases, methionine (M) can substitute for lysine (K). In addition, sequences that differ by conservative variations are generally homologous.

The term “isolated” refers to a compound, which can be e.g. an antibody or antibody fragment, that is substantially free of other cellular material. Thus, in some aspects, antibodies provided are isolated antibodies which have been separated from antibodies with a different specificity.

Additional definitions are described in the sections below.

Various aspects of the invention are described in further detail in the following subsections.

### II. Targeted Active Gene Editing (TAGE) Agent

The present invention includes a targeted active gene editing (TAGE) agent that is useful for delivering a gene editing polypeptide (i.e., a site-directed modifying polypeptide) to a target cell. In some embodiments, the TAGE agent can be a biologic. In particular embodiments, the site-directed modifying polypeptide contains a conjugation moiety that allows the protein to be conjugated to an antigen binding protein that binds to an antigen associated with the extracellular region of a cell membrane. This target specificity allows for delivery of the site-directed modifying polypeptide only to cells displaying the antigen (e.g., hematopoietic stem cells (HSCs), hematopotic progenitor stem cells (HPSCs), natural killer cells, macrophages, DC cells, non-DC myeloid cells, B cells, T cells (e.g., activated T cells), fibroblasts, or other cells). Such cells may be associated with a certain tissue or cell-type associated with a disease. The TAGE agent thus provides a means by which the genome of a target cell can be modified.

In one embodiment, a TAGE agent comprises a nucleic acid-guided endonuclease (e.g., RNA-guided endonuclease or DNA-guided endonuclease), such as Cas9, that recognizes a CRISPR sequence, and an antigen binding protein that specifically binds to an extracellular molecule (e.g., protein, glycan, lipid) localized on a target cell membrane. Examples of antigen binding proteins that can be used in the TAGE agent of the invention include, but are not limited to, an antibody, an antigen-binding portion of an antibody, or an antibody mimetic. The types of antigen binding proteins that can be used in the compositions and methods described herein are described in more detail in Section IV.

Proteins within the TAGE agent (i.e., at least a site-directed polypeptide and an antigen binding polypeptide) are stably associated such that the antigen binding protein directs the site-directed modifying polypeptide to the cell surface and the site-directed modifying polypeptide is internalized into the target cell. In certain embodiments, the antigen binding protein binds to the antigen on the cell surface such that the site-directed modifying polypeptide is internalized by the target cell but the antigen binding protein is not internalized. In some embodiments, the site-directed modifying polypeptide and the antigen binding protein are both internalized into the target cell.

As described in more detail in Section III, in certain embodiments, when the site-directed modifying polypeptide is a nucleic acid-guided endonuclease, such as Cas9, the nucleic acid-guided endonuclease is associated with a guide nucleic acid to form a nucleoprotein. For example, the guide RNA (gRNA) binds to a RNA-guided nuclease to form a ribonucleoprotein (RNP) or a guide DNA binds to a DNA-guided nuclease to form a deoxyribonucleoprotein (DNP). In other embodiments, the nucleic acid-guided endonuclease is associated with a guide nucleic acid that comprises a DNA:RNA hybrid. In such instances, the ribonucleoprotein (i.e., the RNA-guided endonuclease and the guide RNA), deoxyribonucleoprotein (i.e., the DNA-guided endonuclease and the guide DNA), or the nucleic acid-guided endonuclease bound to a DNA:RNA hybrid guide are internalized into the target cell. In a separate embodiment, the guide nucleic acid (e.g., RNA, DNA, or DNA:RNA hybrid) is delivered to the target cell separately from the nucleic acid-guided endonuclease into the same cell. The guide nucleic acid (e.g., RNA,DNA, or DNA:RNA hybrid) may already be present in the target cell upon internalization of the nucleic acid-guided endonuclease upon contact with the TAGE agent.

A TAGE agent specifically binds to an extracellular molecule (e.g., protein, glycan, lipid) localized on a target cell membrane. The target molecule can be, for example, an extracellular membrane-bound protein, but can also be a non-protein molecule such as a glycan or lipid. In one embodiment, the extracellular molecule is an extracellular protein that is expressed by the target cell, such as a ligand or a receptor. The extracellular target molecule may be associated with a specific disease condition or a specific tissue within in an organism. Examples of extracellular molecular targets associated with the cell membrane are described in the sections below.

The site-directed modifying polypeptide comprises a conjugation moiety such that the antigen binding protein can stably associate with the site-directed modifying polypeptide (thus forming a TAGE agent). The conjugation moiety provides for either a covalent or a non-covalent linkage between the antigen binding protein and the site-directed modifying polypeptide.

In certain embodiments, the conjugation moiety useful for the present TAGE agents are stable extracellularly, prevent aggregation of TAGE agents, and/or keep the TAGE agents freely soluble in aqueous media and in a monomeric state. Before transport or delivery into a cell, the TAGE agent is stable and remains intact, e.g., the antibody or antigen binding protein thereof remains linked to the nucleic acid-guided endonuclease.

In one embodiment, the conjugation moiety is Protein A, wherein the site-directed modifying polypeptide comprises Protein A and the antigen binding protein comprises an Fc region that can be bound by Protein A, e.g., an antibody comprising an Fc domain. In one embodiment, a site-directed modifying polypeptide comprises SEQ ID NO: 2, or an Fc binding portion thereof (SEQ ID NO: 2 corresponds to the amino acid sequence of Protein A).

In another embodiment, the conjugation moiety is Spycatcher/SpyTag peptide system. For example, in certain embodiments, the site-directed modifying polypeptide comprises SpyCatcher (e.g., at the N-terminus or C-terminus) and the antigen binding polypeptide comprises a SpyTag. For example, in instances where the site-directed modifying polypeptide comprises Cas9, the Cas9 may be conjugated to SpyCatcher to form SpyCatcher-Cas9 (SEQ ID NO: 6) or Cas9-SpyCatcher (SEQ ID NO: 7). In one embodiment, the SpyTag peptide sequence is VPTIVMVDAYKRYK (SEQ ID NO:116).

Other conjugation moieties useful in the TAGE agents provided herein include, but are not limited to, a Spycatcher tag, Snoop tag, haloalkane dehalogenase (Halo-tag), Sortase, mono-avidin, ACP tag, a SNAP tag, or any other conjugation moieties known in the art. In one embodiment, the antibody binding moiety is selected from Protein A, CBP, MBP, GST, poly(His), biotin/streptavidin, V5-tag, Myc-tag, HA-tag, NE-tag, His-tag, Flag tag, Halo-tag, Snap-tag, Fc-tag, Nus-tag, BCCP, thioredoxin, SnooprTag, SpyTag, SpyCatcher, Isopeptag, SBP-tag, S-tag, AviTag, and calmodulin.

In some embodiments, the antibody binding moiety is a chemical tag. For example, a chemical tag may be SNAP tag, a CLIP tag, a HaloTag or a TMP-tag. In one example, the chemical tag is a SNAP-tag or a CLIP-tag. SNAP and CLIP fusion proteins enable the specific, covalent attachment of virtually any molecule to a protein of interest. In another example, the chemical tag is a HaloTag. HaloTag involves a modular protein tagging system that allows different molecules to be linked onto a single genetic fusion, either in solution, in living cells, or in chemically fixed cells. In another example, the chemical tag is a TMP-tag.

In some embodiments, the antibody binding moiety is an epitope tag. For example, an epitope tag may be a poly-histidine tag such as a hexahistidine tag (SEQ ID NO: 25) or a dodecahistidine (SEQ ID NO: 126), a FLAG tag, a Myc tag, a HA tag, a GST tag or a V5 tag.

Depending on the conjugation approach, the site-directed modifying polypeptide and the antigen binding protein may each be engineered to comprise complementary binding pairs that enable stable association of the antibody-binding agent with the corresponding antibody, antigen-binding fragment thereof, or antibody mimetic upon contact. Exemplary binding moiety pairings include (i) streptavidin-binding peptide (streptavidin binding peptide; SBP) and streptavidin (STV), (ii) biotin and EMA (enhanced monomeric avidin), (iii) SpyTag (ST) and SpyCatcher (SC), (iv) Halo-tag and Halo-tag ligand, (v) and SNAP-Tag, (vi) Myc tag and anti-Myc immunoglobulins (vii) FLAG tag and anti-FLAG immunoglobulins, and (ix) ybbR tag and coenzyme A groups. In some embodiments, the antibody binding unit is selected from SBP, biotin, SpyTag, SpyCatcher, halo-tag, SNAP-tag, Myc tag, or FLAG tag.

In certain embodiments, the site-directed modifying polypeptide can alternatively be associated with the antigen binding protein via one or more linkers as described herein wherein the linker is a conjugation moiety.

The term “linker” as used herein means a divalent chemical moiety comprising a covalent bond or a chain of atoms that covalently attaches an antigen binding protein to a site-directed modifying polypeptide to form an TAGE agent. Any known method of conjugation of peptides or macromolecules can be used in the context of the present disclosure. Generally, covalent attachment of the antigen binding protein and the site-directed modifying polypeptide requires the linker to have two reactive functional groups, i.e., bivalency in a reactive sense. Bivalent linker reagents which are useful to attach two or more functional or biologically active moieties, such as peptides, nucleic acids, drugs, toxins, antibodies, haptens, and reporter groups are known, and methods for such conjugation have been described in, for example, Hermanson, G. T. (1996) Bioconjugate Techniques; Academic Press: New York, p 234-242, the disclosure of which is incorporated herein by reference as it pertains to linkers suitable for covalent conjugation. Further linkers are disclosed in, for example, Tsuchikama, K. and Zhiqiang, A. Protein and Cell, 9(1), p. 33-46, (2018), the disclosure of which is incorporated herein by reference as it pertains to linkers suitable for covalent conjugation.

Generally, linkers suitable for use in the compositions and methods disclosed are stable in circulation, but allow for release of the antigen binding protein and/or the site-directed modifying polypeptide in the target cell or, alternatively, in close proximity to the target cell. Linkers suitable for the present disclosure may be broadly categorized as non-cleavable or cleavable, as well as intracellular or extracellular, each of which is further described herein below.

Non-Cleavable Linkers

In some embodiments, the linker conjugating the antigen binding protein and the site-directed modifying polypeptide is non-cleavable. Non-cleavable linkers comprise stable chemical bonds that are resistant to degradation (e.g., proteolysis). Generally, non-cleavable linkers require proteolytic degradation inside the target cell, and exhibit high extracellular stability. Non-cleavable linkers suitable for use herein further may include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include alkylene (CH₂)ₚ, (C═O)(CH₂)ₚ, and polyethyleneglycol (PEG; (CH₂CH₂O)ₚ), units, wherein p is an integer from 1-6, independently selected for each occasion. Non-limiting examples of non-cleavable linker utilized in antibody-drug conjugation include those based on maleimidomethylcyclohexanecarboxylate, caproylmaleimide, and acetylphenylbutanoic acid.

Cleavable Linkers

In some embodiments, the linker conjugating the antigen binding protein and the site-directed modifying polypeptide is cleavable, such that cleavage of the linker (e.g., by a protease, such as metalloproteases) releases the CRISPR targeting element or the antibody or the antigen binding protein thereof, or both, from the TAGE agent in the intracellular or extracellular (e.g., upon binding of the molecule to the cell surface) environment. Cleavable linkers are designed to exploit the differences in local environments, e.g., extracellular and intracellular environments, for example, pH, reduction potential or enzyme concentration, to trigger the release of an TAGE agent component (i.e., the antigen binding protein, the site-directed modifying polypeptide, or both) in the target cell. Generally, cleavable linkers are relatively stable in circulation in vivo, but are particularly susceptible to cleavage in the intracellular environment through one or more mechanisms (e.g., including, but not limited to, activity of proteases, peptidases, and glucuronidases). Cleavable linkers used herein are stable outside the target cell and may be cleaved at some efficacious rate inside the target cell or in close proximity to the extracellular membrane of the target cell. An effective linker will: (i) maintain the specific binding properties of the antigen binding protein, e.g., an antibody; (ii) allow intra- or extracellular delivery of the TAGE agent or a component thereof (i.e., the site-directed modifying polypeptide); (iii) remain stable and intact, i.e. not cleaved, until the TAGE agent has been delivered or transported to its targeted site; and (iv) maintain the gene targeting effect (e.g., CRISPR) of the site-directed modifying polypeptide. Stability of the TAGE agent may be measured by standard analytical techniques such as mass spectroscopy, size determination by size exclusion chromatography or diffusion constant measurement by dynamic light scattering, HPLC, and the separation/analysis technique LC/MS.

Suitable cleavable linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation). Suitable cleavable linkers may include, for example, chemical moieties such as a hydrazine, a disulfide, a thioether or a peptide.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome. Generally, linkers including such acid-labile functionalities tend to be relatively less stable extracellularly. This lower stability may be advantageous where extracellular cleavage is desired.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation. Disulfide-based linkers tend to be relatively unstable in circulation in plasma, however, this lower stability may be advantageous where extracellular cleavage is desired. Susceptibility to cleavage may also be tuned by e.g., introducing steric bulk near the disulfide moiety to hinder reductive cleavage.

Linkers susceptible to enzymatic hydrolysis can be, e.g., a peptide-containing linker that is cleaved by an intracellular peptidase or protease enzyme, including, but not limited to, a lysosomal or endosomal protease. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit. In certain embodiments, linkers comprising a peptide moiety may be susceptible to varying degrees of cleavage both intra- and extracellularly. Accordingly, in some embodiments, the linker comprises a dipeptide, and the TAGE agent is cleaved extracellularly. Accordingly, in some embodiments, the linker comprises a dipeptide, and the TAGE agent is stable extracellularly, and is cleaved intracellularly.

Linkers suitable for conjugating the antigen binding protein as disclosed herein to a site-directed modifying polypeptide, as disclosed herein, include those capable of releasing the antigen binding protein or the site-directed modifying polypepitde by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

Linkers suitable for use herein further may include one or more groups selected from C1-C₆ alkylene, C1-C6 heteroalkylene, C2-C6 alkenylene, C2-C6 heteroalkenylene, C2-C6 alkynylene, C2-C6 heteroalkynylene, C3-C6 cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ— units, wherein p is an integer from 1-6, independently selected for each occasion.

In some embodiments, the linker may include one or more of a hydrazine, a disulfide, a thioether, a dipeptide, a p-aminobenzyl (PAB) group, a heterocyclic self-immolative group, an optionally substituted C1-C6 alkyl, an optionally substituted C1-C6 heteroalkyl, an optionally substituted C2-C6 alkenyl, an optionally substituted C2-C6 heteroalkenyl, an optionally substituted C2-C6 alkynyl, an optionally substituted C2-C6 heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C1-C₆ alkylene and the like.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB. In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

Suitable linkers may be substituted with groups which modulate solubility or reactivity. Suitable linkers may contain groups having solubility enhancing properties. Linkers including the (CH₂CH₂O)ₚ unit (polyethylene glycol, PEG), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference as it pertains to linkers suitable for covalent conjugation. Linkers containing such groups are described, for example, in U.S. Pat. No. 9,636,421 and U.S. Patent Application Publication No. 2017/0298145, the disclosures of which are incorporated herein by reference as they pertain to linkers suitable for covalent conjugation.

Suitable linkers for covalently conjugating an antigen binding protein and a site-directed modifying polypeptide as disclosed herein can have two reactive functional groups (i.e., two reactive termini), one for conjugation to the antigen binding protein, and the other for conjugation to the site-directed modifying polypeptide. Suitable sites for conjugation on the antigen binding protein are, in certain embodiments, nucleophilic, such as a thiol, amino group, or hydroxyl group. Reactive (e.g., nucleophilic) sites that may be present within an antigen-binding protein as disclosed herein include, without limitation, nucleophilic substituents on amino acid residues such as (i) N-terminal amine groups, (ii) side chain amine groups, e.g. lysine, (iii) side chain thiol groups, e.g. cysteine, (iv) side chain hydroxyl groups, e.g. serine; or (iv) sugar hydroxyl or amino groups where the antibody is glycosylated. Suitable sites for conjugation on the antigen binding protein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Accordingly, the antibody conjugation reactive terminus on the linker is, in certain embodiments, a thiol-reactive group such as a double bond (as in maleimide), a leaving group such as a chloro, bromo, iodo, or an R-sulfanyl group, or a carboxyl group.

Suitable sites for conjugation on the site-directed modifying polypeptide can also be, in certain embodiments, nucleophilic. Reactive (e.g., nucleophilic) sites that may be present within a site-directed modifying polypeptide as disclosed herein include, without limitation, nucleophilic substituents on amino acid residues such as (i) N-terminal amine groups, (ii) side chain amine groups, e.g. lysine, (iii) side chain thiol groups, e.g. cysteine, (iv) side chain hydroxyl groups, e.g. serine; or (iv) sugar hydroxyl or amino groups where the antibody is glycosylated. Suitable sites for conjugation on the site-directed modifying polypeptide include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Accordingly, the site-directed modifying polypeptide conjugation reactive terminus on the linker is, in certain embodiments, a thiol-reactive group such as a double bond (as in maleimide), a leaving group such as a chloro, bromo, iodo, or an R-sulfanyl group, or a carboxyl group.

In some embodiments, the reactive functional group attached to the linker is a nucleophilic group which is reactive with an electrophilic group present on an antigen binding protein, the site-directed modifying polypeptide, or both. Useful electrophilic groups on an antigen binding protein or site-directed modifying polypeptide include, but are not limited to, aldehyde and ketone carbonyl groups. The heteroatom of a nucleophilic group can react with an electrophilic group on an antigen binding protein or site-directed modifying polypeptide and form a covalent bond to the antigen binding protein or the site-directed modifying polypeptide. Useful nucleophilic groups include, but are not limited to, hydrazide, oxime, amino, hydroxyl, hydrazine, thiosemicarbazone, hydrazine carboxylate, and arylhydrazide.

In some embodiments, the TAGE agent as disclosed herein comprises a nucleoside or a nucleotide. Suitable sites for conjugation on such nucleosides or nucleotides include —OH or phosphate groups, respectively. Linkers and conjugation methods suitable for use in such embodiments are disclosed in, for example, Wang, T. P., et al., Bioconj. Chem. 21(9), 1642-55, 2010, and Bernardinelli, G. and Hogberg, B. Nucleic Acids Research, 45(18), p. e160; published online 16 Aug. 2017, the disclosure of each of which is incorporated herein by reference as it pertains to linkers suitable for covalent conjugation.

When the term “linker” is used in describing the linker in conjugated form, one or both of the reactive termini will be absent, (having been converted to a chemical moiety) or incomplete (such as being only the carbonyl of a carboxylic acid) because of the formation of the bonds between the linker and the antigen binding protein, and/or between the linker and the site-directed modifying polypeptide. Accordingly, linkers useful herein include, without limitation, linkers containing a chemical moiety formed by a coupling reaction between a reactive functional group on the linker and a nucleophilic group or otherwise reactive substituent on the antigen binding protein, and a chemical moiety formed by a coupling reaction between a reactive functional group on the linker and a nucleophilic group on the site-directed modifying polypeptide.

Examples of chemical moieties formed by these coupling reactions result from reactions between chemically reactive functional groups, including a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β unsaturated carbonyl pair, among others), and the like. Coupling reactions between the reactive functional groups to form the chemical moiety include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Suitable linkers may contain an electrophilic functional group for reaction with a nucleophilic functional group on the antigen binding protein, the site-directed modifying polypeptide, or both.

In some embodiments, the reactive functional group present within antigen binding protein, the site-directed modifying polypeptide, or both as disclosed herein are amine or thiol moieties. Certain antigen binding proteins have reducible interchain disulfides, i.e. cysteine bridges. Antigen binding proteins may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antigen binding proteins through the reaction of lysines with 2-iminothiolane (Traut's reagent) resulting in conversion of an amine into a thiol. Reactive thiol groups may be introduced into the antigen binding protein by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Linkers suitable for the synthesis of the covalent conjugates as disclosed herein include, without limitation, reactive functional groups such as maleimide or a haloalkyl group. These groups may be present in linkers or cross linking reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, one or both of the reactive functional groups attached to the linker is a maleimide, azide, or alkyne. An example of a maleimide-containing linker is the non-cleavable maleimidocaproyl-based linker. Such linkers are described by Doronina et al., Bioconjugate Chem. 17:14-24, 2006, the disclosure of which is incorporated herein by reference as it pertains to linkers for chemical conjugation.

In some embodiments, the reactive functional group is —(C═O)— or —NH(C═O)—, such that the linker may be joined to the antigen binding protein or the site-directed modifying polypeptide by an amide or urea moiety, respectively, resulting from reaction of the —(C═O)— or —NH(C═O)— group with an amino group of the antigen binding protein or the site-directed modifying polypeptide, or both.

In some embodiments, the reactive functional group is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (het-ero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive functional group comprises a cycloalkene group, a cycloalkyne group, or an optionally substituted (hetero)cycloalkynyl group.

Examples of suitable bivalent linker reagents suitable for preparing conjugates as disclosed herein include, but are not limited to, N-succinimidyl 4-(maleimidomethyl)cyclohexanecarboxylate (SMCC), N-succinimidyl-4-(N-maleimidomethyl)-cyclohexane-1-carboxy-(6-amidocaproate), which is a “long chain” analog of SMCC (LC-SMCC), κ-maleimidoundecanoic acid N-succinimidyl ester (KMUA), γ-maleimidobutyric acid N-succinimidyl ester (GMBS), ε-maleimidocaproic acid N-hydroxysuccinimide ester (EMCS), m-maleimidobenzoyl-N-hydroxysuccinimide ester (MBS), N-(α-maleimidoacetoxy)-succinimide ester (AMAS), succinimidyl-6-(β-maleimidopropionamido)hexanoate (SMPH), N-succinimidyl 4-(p-maleimidophenyl)-butyrate (SMPB), and N-(p-maleimidophenyl)isocyanate (PMPI). Cross-linking reagents comprising a haloacetyl-based moiety include N-succinimidyl-4-(iodoacetyl)-aminobenzoate (SIAB), N-succinimidyl iodoacetate (SIA), N-succinimidyl bromoacetate (SBA), and N-succinimidyl 3-(bromoacetamido)propionate (SBAP).

It will be recognized by one of skill in the art that any one or more of the chemical groups, moieties and features disclosed herein may be combined in multiple ways to form linkers useful for conjugation of the antigen binding protein as disclosed herein to a site-directed modifying polypeptide, as disclosed herein. Further linkers useful in conjunction with the compositions and methods described herein, are described, for example, in U.S. Patent Application Publication No. 2015/0218220, the disclosure of which is incorporated herein by reference as is pertain to linkers suitable for covalent conjugation.

### III. Site-Directed Modifying Polypeptide of TAGE Agent

The TAGE agent comprises a site-directed modifying polypeptide, such as a nucleic acid-guided endonuclease (e.g., RNA-guided endonuclease (e.g., Cas9) or DNA-guided endonuclease) that recognizes a nucleic acid sequence in the target cell.

The site-directed modifying polypeptides used in the presently disclosed compositions and methods are site-specific, in that the polypeptide itself or an associated molecule recognizes and is targeted to a particular nucleic acid sequence or a set of similar sequences (i.e., target sequence(s)). In some embodiments, the site-directed modifying polypeptide (or its associated molecule) recognizes sequences that are similar in sequence, comprising conserved bases or motifs that can be degenerate at one or more positions.

In particular embodiments, the site-directed modifying polypeptide modifies the polynucleotide at particular location(s) (i.e., modification site(s)) outside of its target sequence. The modification site(s) modified by a particular site-directed modifying polypeptide are also generally specific to a particular sequence or set of similar sequences. In some of these embodiments, the site-directed modifying polypeptide modifies sequences that are similar in sequence, comprising conserved bases or motifs that can be degenerate at one or more positions. In other embodiments, the site-directed modifying polypeptide modifies sequences that are within a particular location relative to the target sequence(s). For example, the site-directed modifying polypeptide may modify sequences that are within a particular number of nucleic acids upstream or downstream from the target sequence(s).

As used herein with respect to site-directed modifying polypeptides, the term “modification” means any insertion, deletion, substitution, or chemical modification of at least one nucleotide the modification site or alternatively, a change in the expression of a gene that is adjacent to the target site. The substitution of at least one nucleotide in the modification site can be the result of the recruitment of a base editing domain, such as a cytidine deaminase or adenine deaminase domain (see, for example, Eid et al. (2018) Biochem J 475(11):1955-1964, which is herein incorporated in its entirety).

The change in expression of a gene adjacent to a target site can result from the recruitment of a transcriptional activation domain or transcriptional repression domain to the promoter region of the gene or the recruitment of an epigenetic modification domain that covalently modifies DNA or histone proteins to alter histone structure and/or chromosomal structure without altering the DNA sequence, leading to changes in gene expression of an adjacent gene. The term “modification” also encompasses the recruitment to a target site of a detectable label that can be conjugated to the site-directed modifying polypeptide or an associated molecule (e.g., gRNA) that allows for the detection of a specific nucleic acid sequence (e.g., a disease-associated sequence).

In some embodiments, the site-directed modifying polypeptide is a nuclease or variant thereof and the agent comprising the nuclease or variant thereof is thus referred to herein as a gene editing cell targeting (TAGE) agent. As used herein a “nuclease” refers to an enzyme which cleaves a phosphodiester bond in the backbone of a polynucleotide chain. Suitable nucleases for the presently disclosed compositions and methods can have endonuclease and/or exonuclease activity. An exonuclease cleaves nucleotides one at a time from the end of a polynucleotide chain. An endonuclease cleaves a polynucleotide chain by cleaving phosphodiester bonds within a polynucleotide chain, other than those at the two ends of a polynucleotide chain. The nuclease can cleave RNA polynucleotide chains (i.e., ribonuclease) and/or DNA polynucleotide chains (i.e., deoxyribonuclease).

Nucleases cleave polynucleotide chains, resulting in a cleavage site. As used herein, the term “cleave” refers to the hydrolysis of phosphodiester bonds within the backbone of a polynucleotide chain. Cleavage by nucleases of the presently disclosed TAGE agents can be single-stranded or double-stranded. In some embodiments, a double-stranded cleavage of DNA is achieved via cleavage with two nucleases wherein each nuclease cleaves a single strand of the DNA. Cleavage by the nuclease can result in blunt ends or staggered ends.

Non-limiting examples of nucleases suitable for the presently disclosed compositions and methods include meganucleases, such as homing endonucleases; restriction endonucleases, such as Type IIS endonucleases (e.g., FokI)); zinc finger nucleases; transcription activator-like effector nucleases (TALENs), and nucleic acid-guided nucleases (e.g., RNA-guided endonuclease, DNA-guided endonuclease, or DNA/RNA-guided endonuclease).

As used herein, a “meganuclease” refers to an endonuclease that binds DNA at a target sequence that is greater than 12 base pairs in length. Meganucleases bind to double-stranded DNA as heterodimers. Suitable meganucleases for the presently disclosed compositions and methods include homing endonucleases, such as those of the LAGLIDADG (SEQ ID NO: 127) family comprising this amino acid motif or a variant thereof.

As used herein, a “zinc finger nuclease” or “ZFN” refers to a chimeric protein comprising a zinc finger DNA-binding domain fused to a nuclease domain from an exonuclease or endonuclease, such as a restriction endonuclease or meganuclease. The zinc finger DNA-binding domain is bound by a zinc ion that serves to stabilize the unique structure.

As used herein, a “transcription activator-like effector nuclease” or “TALEN” refers to a chimeric protein comprising a DNA-binding domain comprising multiple TAL domain repeats fused to a nuclease domain from an exonuclease or endonuclease, such as a restriction endonuclease or meganuclease. TAL domain repeats can be derived from the TALE family of proteins from the Xanthomonas genus of Proteobacteria. TAL domain repeats are 33-34 amino acid sequences with hypervariable 12^{th} and 13^{th} amino acids that are referred to as the repeat variable diresidue (RVD). The RVD imparts specificity of target sequence binding. The TAL domain repeats can be engineered through rational or experimental means to produce variant TALENs that have a specific target sequence specificity (see, for example, Boch et al. (2009) Science 326(5959):1509-1512 and Moscou and Bogdanove (2009) Science 326(5959):1501, each of which is incorporated by reference in its entirety). DNA cleavage by a TALEN requires two DNA target sequences flanking a nonspecific spacer region, wherein each DNA target sequence is bound by a TALEN monomer. In some embodiments, the TALEN comprises a compact TALEN, which refers to an endonuclease comprising a DNA-binding domain with one or more TAL domain repeats fused in any orientation to any portion of a homing endonuclease (e.g., I-TevI, MmeI, EndA, End1, I-BasI, I-TevII, I-TevIII, I-TwoI, MspI, MvaI, NucA, and NucM). Compact TALENs are advantageous in that they do not require dimerization for DNA processing activity, thus only requiring a single target site.

As used herein, a “nucleic acid-guided nuclease” refers to a nuclease that is directed to a specific target sequence based on the complementarity (full or partial) between a guide nucleic acid (i.e., guide RNA or gRNA, guide DNA or gDNA, or guide DNA/RNA hybrid) that is associated with the nuclease and a target sequence. The binding between the guide RNA and the target sequence serves to recruit the nuclease to the vicinity of the target sequence. Non-limiting examples of nucleic acid-guided nucleases suitable for the presently disclosed compositions and methods include naturally-occurring Clustered Regularly Interspaced Short Palindromic Repeats (CRISPR)-associated (Cas) polypeptides from a prokaryotic organism (e.g., bacteria, archaea) or variants thereof. CRISPR sequences found within prokaryotic organisms are sequences that are derived from fragments of polynucleotides from invading viruses and are used to recognize similar viruses during subsequent infections and cleave viral polynucleotides via CRISPR-associated (Cas) polypeptides that function as an RNA-guided nuclease to cleave the viral polynucleotides. As used herein, a “CRISPR-associated polypeptide” or “Cas polypeptide” refers to a naturally-occurring polypeptide that is found within proximity to CRISPR sequences within a naturally-occurring CRISPR system. Certain Cas polypeptides function as RNA-guided nucleases.

There are at least two classes of naturally-occurring CRISPR systems, Class 1 and Class 2. In general, the nucleic acid-guided nucleases of the presently disclosed compositions and methods are Class 2 Cas polypeptides or variants thereof given that the Class 2 CRISPR systems comprise a single polypeptide with nucleic acid-guided nuclease activity, whereas Class 1 CRISPR systems require a complex of proteins for nuclease activity. There are at least three known types of Class 2 CRISPR systems, Type II, Type V, and Type VI, among which there are multiple subtypes (subtype II-A, II-B, II-C, V-A, V-B, V-C, VI-A, VI-B, and VI-C, among other undefined or putative subtypes). In general, Type II and Type V-B systems require a tracrRNA, in addition to crRNA, for activity. In contrast, Type V-A and Type VI only require a crRNA for activity. All known Type II and Type V RNA-guided nucleases target double-stranded DNA, whereas all known Type VI RNA-guided nucleases target single-stranded RNA. The RNA-guided nucleases of Type II CRISPR systems are referred to as Cas9 herein and in the literature. In some embodiments, the nucleic acid-guided nuclease of the presently disclosed compositions and methods is a Type II Cas9 protein or a variant thereof. Type V Cas polypeptides that function as RNA-guided nucleases do not require tracrRNA for targeting and cleavage of target sequences. The RNA-guided nuclease of Type VA CRISPR systems are referred to as Cpf1; of Type VB CRISPR systems are referred to as C2C1; of Type VC CRISPR systems are referred to as Cas12C or C2C3; of Type VIA CRISPR systems are referred to as C2C2 or Cas13A1; of Type VIB CRISPR systems are referred to as Cas13B; and of Type VIC CRISPR systems are referred to as Cas13A2 herein and in the literature. In certain embodiments, the nucleic acid-guided nuclease of the presently disclosed compositions and methods is a Type VA Cpf1 protein or a variant thereof. Naturally-occurring Cas polypeptides and variants thereof that function as nucleic acid-guided nucleases are known in the art and include, but are not limited to Streptococcus pyogenes Cas9, Staphylococcus aureus Cas9, Streptococcus thermophilus Cas9, Francisella novicida Cpf1, or those described in Shmakov et al. (2017) Nat Rev Microbiol 15(3):169-182; Makarova et al. (2015) Nat Rev Microbiol 13(11):722-736; and U.S. Pat. No. 9,790,490, each of which is incorporated herein in its entirety. Class 2 Type V CRISPR nucleases include Cas12 and any subtypes of Cas12, such as Cas12a, Cas12b, Cas12c, Cas12d, Cas12e, Cas12f, Cas12g, Cas12h, and Cas12i. Class 2 Type VI CRISPR nucleases including Cas13 can be included in the TAGE agent in order to cleave RNA target sequences.

The nucleic acid-guided nuclease of the presently disclosed compositions and methods can be a naturally-occurring nucleic acid-guided nuclease (e.g., S. pyogenes Cas9) or a variant thereof. Variant nucleic acid-guided nucleases can be engineered or naturally occurring variants that contain substitutions, deletions, or additions of amino acids that, for example, alter the activity of one or more of the nuclease domains, fuse the nucleic acid-guided nuclease to a heterologous domain that imparts a modifying property (e.g., transcriptional activation domain, epigenetic modification domain, detectable label), modify the stability of the nuclease, or modify the specificity of the nuclease.

In some embodiments, a nucleic acid-guided nuclease includes one or more mutations to improve specificity for a target site and/or stability in the intracellular microenvironment. For example, where the protein is Cas9 (e.g., SpCas9) or a modified Cas9, it may be beneficial to delete any or all residues from N175 to R307 (inclusive) of the Rec2 domain. It may be found that a smaller, or lower-molecular mass, version of the nuclease is more effective. In some embodiments, the nuclease comprises at least one substitution relative to a naturally-occurring version of the nuclease. For example, where the protein is Cas9 or a modified Cas9, it may be beneficial to mutate C80 or C574 (or homologs thereof, in modified proteins with indels). In Cas9, desirable substitutions may include any of C80A, C80L, C801, C80V, C80K, C574E, C574D, C574N, C574Q (in any combination) and in particular C80A. Substitutions may be included to reduce intracellular protein binding of the nuclease and/or increase target site specificity. Additionally or alternatively, substitutions may be included to reduce off-target toxicity of the composition.

The nucleic acid-guided nuclease is directed to a particular target sequence through its association with a guide nucleic acid (e.g., guideRNA (gRNA), guideDNA (gDNA)). The nucleic acid-guided nuclease is bound to the guide nucleic acid via non-covalent interactions, thus forming a complex. The polynucleotide-targeting nucleic acid provides target specificity to the complex by comprising a nucleotide sequence that is complementary to a sequence of a target sequence. The nucleic acid-guided nuclease of the complex or a domain or label fused or otherwise conjugated thereto provides the site-specific activity. In other words, the nucleic acid-guided nuclease is guided to a target polynucleotide sequence (e.g. a target sequence in a chromosomal nucleic acid; a target sequence in an extrachromosomal nucleic acid, e.g. an episomal nucleic acid, a minicircle; a target sequence in a mitochondrial nucleic acid; a target sequence in a chloroplast nucleic acid; a target sequence in a plasmid) by virtue of its association with the protein-binding segment of the polynucleotide-targeting guide nucleic acid.

Thus, the guide nucleic acid comprises two segments, a “polynucleotide-targeting segment” and a “polypeptide-binding segment.” By “segment” it is meant a segment/section/region of a molecule (e.g., a contiguous stretch of nucleotides in an RNA). A segment can also refer to a region/section of a complex such that a segment may comprise regions of more than one molecule. For example, in some cases the polypeptide-binding segment (described below) of a polynucleotide-targeting nucleic acid comprises only one nucleic acid molecule and the polypeptide-binding segment therefore comprises a region of that nucleic acid molecule. In other cases, the polypeptide-binding segment (described below) of a DNA-targeting nucleic acid comprises two separate molecules that are hybridized along a region of complementarity.

The polynucleotide-targeting segment (or “polynucleotide-targeting sequence” or “guide sequence”) comprises a nucleotide sequence that is complementary (fully or partially) to a specific sequence within a target sequence (for example, the complementary strand of a target DNA sequence). The polypeptide-binding segment (or “polypeptide-binding sequence”) interacts with a nucleic acid-guided nuclease. In general, site-specific cleavage or modification of the target DNA by a nucleic acid-guided nuclease occurs at locations determined by both (i) base-pairing complementarity between the polynucleotide-targeting sequence of the nucleic acid and the target DNA; and (ii) a short motif (referred to as the protospacer adjacent motif (PAM)) in the target DNA.

A protospacer adjacent motif can be of different lengths and can be a variable distance from the target sequence, although the PAM is generally within about 1 to about 10 nucleotides from the target sequence, including about 1, about 2, about 3, about 4, about 5, about 6, about 7, about 8, about 9, or about 10 nucleotides from the target sequence. The PAM can be 5′ or 3′ of the target sequence. Generally, the PAM is a consensus sequence of about 3-4 nucleotides, but in particular embodiments, can be 2, 3, 4, 5, 6, 7, 8, 9, or more nucleotides in length. Methods for identifying a preferred PAM sequence or consensus sequence for a given RNA-guided nuclease are known in the art and include, but are not limited to the PAM depletion assay described by Karvelis et al. (2015) Genome Biol 16:253, or the assay disclosed in Pattanayak et al. (2013) Nat Biotechnol 31 (9):839-43, each of which is incorporated by reference in its entirety.

The polynucleotide-targeting sequence (i.e., guide sequence) is the nucleotide sequence that directly hybridizes with the target sequence of interest. The guide sequence is engineered to be fully or partially complementary with the target sequence of interest. In various embodiments, the guide sequence can comprise from about 8 nucleotides to about 30 nucleotides, or more. For example, the guide sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the guide sequence is about 10 to about 26 nucleotides in length, or about 12 to about 30 nucleotides in length. In particular embodiments, the guide sequence is about 30 nucleotides in length. In some embodiments, the degree of complementarity between a guide sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the guide sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

In some embodiments, a guide nucleic acid comprises two separate nucleic acid molecules (an “activator-nucleic acid” and a “targeter-nucleic acid”, see below) and is referred to herein as a “double-molecule guide nucleic acid” or a “two-molecule guide nucleic acid.” In other embodiments, the subject guide nucleic acid is a single nucleic acid molecule (single polynucleotide) and is referred to herein as a “single-molecule guide nucleic acid,” a “single-guide nucleic acid,” or an “sgNA.” The term “guide nucleic acid” or “gNA” is inclusive, referring both to double-molecule guide nucleic acids and to single-molecule guide nucleic acids (i.e., sgNAs). In those embodiments wherein the guide nucleic acid is an RNA, the gRNA can be a double-molecule guide RNA or a single-guide RNA. Likewise, in those embodiments wherein the guide nucleic acid is a DNA, the gDNA can be a double-molecule guide DNA or a single-guide DNA.

An exemplary two-molecule guide nucleic acid comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA-like molecule (targeter-RNA) comprises both the polynucleotide-targeting segment (single stranded) of the guide RNA and a stretch (“duplex-forming segment”) of nucleotides that forms one half of the dsRNA duplex of the polypeptide-binding segment of the guide RNA, also referred to herein as the CRISPR repeat sequence.

The term “activator-nucleic acid” or “activator-NA” is used herein to mean a tracrRNA-like molecule of a double-molecule guide nucleic acid. The term “targeter-nucleic acid” or “targeter-NA” is used herein to mean a crRNA-like molecule of a double-molecule guide nucleic acid. The term “duplex-forming segment” is used herein to mean the stretch of nucleotides of an activator-NA or a targeter-NA that contributes to the formation of the dsRNA duplex by hybridizing to a stretch of nucleotides of a corresponding activator-NA or targeter-NA molecule. In other words, an activator-NA comprises a duplex-forming segment that is complementary to the duplex-forming segment of the corresponding targeter-NA. As such, an activator-NA comprises a duplex-forming segment while a targeter-NA comprises both a duplex-forming segment and the DNA-targeting segment of the guide nucleic acid. Therefore, a subject double-molecule guide nucleic acid can be comprised of any corresponding activator-NA and targeter-NA pair.

The activator-NA comprises a CRISPR repeat sequence comprising a nucleotide sequence that comprises a region with sufficient complementarity to hybridize to an activator-NA (the other part of the polypeptide-binding segment of the guide nucleic acid). In various embodiments, the CRISPR repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and the antirepeat region of its corresponding tracr sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

A corresponding tracrRNA-like molecule (i.e., activator-NA) comprises a stretch of nucleotides (duplex-forming segment) that forms the other part of the double-stranded duplex of the polypeptide-binding segment of the guide nucleic acid. In other words, a stretch of nucleotides of a crRNA-like molecule (i.e., the CRISPR repeat sequence) are complementary to and hybridize with a stretch of nucleotides of a tracrRNA-like molecule (i.e., the anti-repeat sequence) to form the double-stranded duplex of the polypeptide-binding domain of the guide nucleic acid. The crRNA-like molecule additionally provides the single stranded DNA-targeting segment. Thus, a crRNA-like and a tracrRNA-like molecule (as a corresponding pair) hybridize to form a guide nucleic acid. The exact sequence of a given crRNA or tracrRNA molecule is characteristic of the CRISPR system and species in which the RNA molecules are found. A subject double-molecule guide RNA can comprise any corresponding crRNA and tracrRNA pair.

A trans-activating-like CRISPR RNA or tracrRNA-like molecule (also referred to herein as an “activator-NA”) comprises a nucleotide sequence comprising a region that has sufficient complementarity to hybridize to a CRISPR repeat sequence of a crRNA, which is referred to herein as the anti-repeat region. In some embodiments, the tracrRNA-like molecule further comprises a region with secondary structure (e.g., stem-loop) or forms secondary structure upon hybridizing with its corresponding crRNA. In particular embodiments, the region of the tracrRNA-like molecule that is fully or partially complementary to a CRISPR repeat sequence is at the 5′ end of the molecule and the 3′ end of the tracrRNA-like molecule comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNC found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA-like molecule that is fully or partially complementary to the CRISPR repeat sequence comprises from about 8 nucleotides to about 30 nucleotides, or more. For example, the region of base pairing between the tracrRNA-like anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the degree of complementarity between a CRISPR repeat sequence and its corresponding tracrRNA-like anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA-like molecule can comprise from about 60 nucleotides to more than about 140 nucleotides. For example, the tracrRNA-like molecule can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA-like molecule is about 80 to about 100 nucleotides in length, including about 80, about 81, about 82, about 83, about 84, about 85, about 86, about 87, about 88, about 89, about 90, about 91, about 92, about 93, about 94, about 95, about 96, about 97, about 98, about 99, and about 100 nucleotides in length.

A subject single-molecule guide nucleic acid (i.e., sgNA) comprises two stretches of nucleotides (a targeter-NA and an activator-NA) that are complementary to one another, are covalently linked by intervening nucleotides (“linkers” or “linker nucleotides”), and hybridize to form the double stranded nucleic acid duplex of the protein-binding segment, thus resulting in a stem-loop structure. The targeter-NA and the activator-NA can be covalently linked via the 3′ end of the targeter-NA and the 5′ end of the activator-NA. Alternatively, the targeter-NA and the activator-NA can be covalently linked via the 5′ end of the targeter-NA and the 3′ end of the activator-NA.

The linker of a single-molecule DNA-targeting nucleic acid can have a length of from about 3 nucleotides to about 100 nucleotides. For example, the linker can have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt, including but not limited to about 3, about 4, about 5, about 6, about 7, about 8, about 9, about 10, about 11, about 12, about 13, about 14, about 15, about 16, about 17, about 18, about 19, about 20, or more nucleotides. In some embodiments, the linker of a single-molecule DNA-targeting nucleic acid is 4 nt.

An exemplary single-molecule DNA-targeting nucleic acid comprises two complementary stretches of nucleotides that hybridize to form a double-stranded duplex, along with a guide sequence that hybridizes to a specific target sequence.

Appropriate naturally-occurring cognate pairs of crRNAs (and, in some embodiments, tracrRNAs) are known for most Cas proteins that function as nucleic acid-guided nucleases that have been discovered or can be determined for a specific naturally-occurring Cas protein that has nucleic acid-guided nuclease activity by sequencing and analyzing flanking sequences of the Cas nucleic acid-guided nuclease protein to identify tracrRNA-coding sequence, and thus, the tracrRNA sequence, by searching for known antirepeat-coding sequences or a variant thereof. Antirepeat regions of the tracrRNA comprise one-half of the ds protein-binding duplex. The complementary repeat sequence that comprises one-half of the ds protein-binding duplex is called the CRISPR repeat. CRISPR repeat and antirepeat sequences utilized by known CRISPR nucleic acid-guided nucleases are known in the art and can be found, for example, at the CRISPR database on the world wide web at crispr.i2bc.paris-saclay.fr/crispr/.

The single guide nucleic acid or dual-guide nucleic acid can be synthesized chemically or via in vitro transcription. Assays for determining sequence-specific binding between a nucleic acid-guided nuclease and a guide nucleic acid are known in the art and include, but are not limited to, in vitro binding assays between an expressed nucleic acid-guided nuclease and the guide nucleic acid, which can be tagged with a detectable label (e.g., biotin) and used in a pull-down detection assay in which the nucleoprotein complex is captured via the detectable label (e.g., with streptavidin beads). A control guide nucleic acid with an unrelated sequence or structure to the guide nucleic acid can be used as a negative control for non-specific binding of the nucleic acid-guided nuclease to nucleic acids.

In some embodiments, the DNA-targeting RNA, gRNA, or sgRNA or nucleotide sequence encoding the DNA-targeting RNA, gRNA, or sgRNA comprises modifications of the nucleotide sequence. In some cases, the sgRNA (e.g., truncated sgRNA) comprises a first nucleotide sequence that is complementary to the target nucleic acid and a second nucleotide sequence that interacts with a Cas polypeptide. In other instances, the sgRNA comprises one or more modified nucleotides. In some cases, one or more of the nucleotides in the first nucleotide sequence and/or the second nucleotide sequence are modified nucleotides.

In some embodiments, the modified nucleotides comprise a modification in a ribose group, a phosphate group, a nucleobase, or a combination thereof. In some instances, the modification in the ribose group comprises a modification at the 2′ position of the ribose group. In some cases, the modification at the 2′ position of the ribose group is selected from the group consisting of 2′-O-methyl, 2′-fluoro, 2′-deoxy, 2′-O-(2-methoxyethyl), and a combination thereof. In other instances, the modification in the phosphate group comprises a phosphorothioate modification. In other embodiments, the modified nucleotides are selected from the group consisting of a 2′-ribo 3′-phosphorothioate (S), 2′-O-methyl (M) nucleotide, a 2′-O-methyl 3′-phosphorothioate (MS) nucleotide, a 2′-O-methyl 3′-thioPACE (MSP) nucleotide, and a combination thereof.

In certain embodiments, the site-directed modifying polypeptide of the presently disclosed compositions and methods comprise a nuclease variant that functions as a nickase, wherein the nuclease comprises a mutation in comparison to the wild-type nuclease that results in the nuclease only being capable of cleaving a single strand of a double-stranded nucleic acid molecule, or lacks nuclease activity altogether (i.e., nuclease-dead).

A nuclease, such as a nucleic acid-guided nuclease, that functions as a nickase only comprises a single functioning nuclease domain. In some of these embodiments, additional nuclease domains have been mutated such that the nuclease activity of that particular domain is reduced or eliminated.

In other embodiments, the nuclease (e.g., RNA-guided nuclease) lacks nuclease activity completely and is referred to herein as nuclease-dead. In some of these embodiments, all nuclease domains within the nuclease have been mutated such that all nuclease activity of the polypeptide has been eliminated. Any method known in the art can be used to introduce mutations into one or more nuclease domains of a site-directed nuclease, including those set forth in U.S. Publ. Nos. 2014/0068797 and U.S. Pat. No. 9,790,490, each of which is incorporated by reference in its entirety.

Any mutation within a nuclease domain that reduces or eliminates the nuclease activity can be used to generate a nucleic acid-guided nuclease having nickase activity or a nuclease-dead nucleic acid-guided nuclease. Such mutations are known in the art and include, but are not limited to the D10A mutation within the RuvC domain or H840A mutation within the HNH domain of the S. pyogenes Cas9 or at similar position(s) within another nucleic acid-guided nuclease when aligned for maximal homology with the S. pyogenes Cas9. Other positions within the nuclease domains of S. pyogenes Cas9 that can be mutated to generate a nickase or nuclease-dead protein include G12, G17, E762, N854, N863, H982, H983, and D986. Other mutations within a nuclease domain of a nucleic acid-guided nuclease that can lead to nickase or nuclease-dead proteins include a D917A, E1006A, E1028A, D1227A, D1255A, N1257A, D917A, E1006A, E1028A, D1227A, D1255A, and N1257A of the Francisella novicida Cpf1 protein or at similar position(s) within another nucleic acid-guided nuclease when aligned for maximal homology with the F. novicida Cpf1 protein (U.S. Pat. No. 9,790,490, which is incorporated by reference in its entirety).

Site-directed modifying polypeptides comprising a nuclease-dead domain can further comprise a domain capable of modifying a polynucleotide. Non-limiting examples of modifying domains that may be fused to a nuclease-dead domain include but are not limited to, a transcriptional activation or repression domain, a base editing domain, and an epigenetic modification domain. In other embodiments, the site-directed modifying polypeptide comprising a nuclease-dead domain further comprises a detectable label that can aid in detecting the presence of the target sequence.

The epigenetic modification domain that can be fused to a nuclease-dead domain serves to covalently modify DNA or histone proteins to alter histone structure and/or chromosomal structure without altering the DNA sequence itself, leading to changes in gene expression (upregulation or downregulation). Non-limiting examples of epigenetic modifications that can be induced by site-directed modifying polypeptides include the following alterations in histone residues and the reverse reactions thereof: sumoylation, methylation of arginine or lysine residues, acetylation or ubiquitination of lysine residues, phosphorylation of serine and/or threonine residues; and the following alterations of DNA and the reverse reactions thereof: methylation or hydroxymethylation of cystosine residues. Non-limiting examples of epigenetic modification domains thus include histone acetyltransferase domains, histone deacetylation domains, histone methyltransferase domains, histone demethylase domains, DNA methyltransferase domains, and DNA demethylase domains.

In some embodiments, the site-directed polypeptide comprises a transcriptional activation domain that activates the transcription of at least one adjacent gene through the interaction with transcriptional control elements and/or transcriptional regulatory proteins, such as transcription factors or RNA polymerases. Suitable transcriptional activation domains are known in the art and include, but are not limited to, VP16 activation domains.

In other embodiments, the site-directed polypeptide comprises a transcriptional repressor domain, which can also interact with transcriptional control elements and/or transcriptional regulatory proteins, such as transcription factors or RNA polymerases, to reduce or terminate transcription of at least one adjacent gene. Suitable transcriptional repression domains are known in the art and include, but are not limited to, IκB and KRAB domains.

In still other embodiments, the site-directed modifying polypeptide comprising a nuclease-dead domain further comprises a detectable label that can aid in detecting the presence of the target sequence, which may be a disease-associated sequence. A detectable label is a molecule that can be visualized or otherwise observed. The detectable label may be fused to the nucleic-acid guided nuclease as a fusion protein (e.g., fluorescent protein) or may be a small molecule conjugated to the nuclease polypeptide that can be detected visually or by other means. Detectable labels that can be fused to the presently disclosed nucleic-acid guided nucleases as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

The nucleic acid-guided nuclease can be delivered as part of a TAGE agent into a cell as a nucleoprotein complex comprising the nucleic acid-guided nuclease bound to its guide nucleic acid. Alternatively, the nucleic acid-guided nuclease is delivered as a TAGE agent and the guide nucleic acid is provided separately. In certain embodiments, a guide RNA can be introduced into a target cell as an RNA molecule. The guide RNA can be transcribed in vitro or chemically synthesized. In other embodiments, a nucleotide sequence encoding the guide RNA is introduced into the cell. In some of these embodiments, the nucleotide sequence encoding the guide RNA is operably linked to a promoter (e.g., an RNA polymerase III promoter), which can be a native promoter or heterologous to the guide RNA-encoding nucleotide sequence.

In certain embodiments, the site-directed polypeptide can comprise additional amino acid sequences, such as at least one nuclear localization sequence (NLS). Nuclear localization sequences enhance transport of the site-directed polypeptide into the nucleus of a cell. Proteins that are imported into the nucleus bind to one or more of the proteins within the nuclear pore complex, such as importin/karypherin proteins, which generally bind best to lysine and arginine residues. The best characterized pathway for nuclear localization involves short peptide sequence which binds to the importin-α protein. These nuclear localization sequences often comprise stretches of basic amino acids and given that there are two such binding sites on importin-α, two basic sequences separated by at least 10 amino acids can make up a bipartite NLS. The second most characterized pathway of nuclear import involves proteins that bind to the importin-β1 protein, such as the HIV-TAT and HIV-REV proteins, which use the sequences RKKRRQRRR (SEQ ID NO: 9) and RQARRNRRRRWR (SEQ ID NO: 13), respectively to bind to importin-β1. Other nuclear localization sequences are known in the art (see, e.g., Lange et al., J. Biol. Chem. (2007) 282:5101-5105). The NLS can be the naturally-occurring NLS of the site-directed polypeptide or a heterologous NLS. As used herein, “heterologous” in reference to a sequence is a sequence that originates from a foreign species, or, if from the same species, is substantially modified from its native form in composition and/or genomic locus by deliberate human intervention. Non-limiting examples of NLS sequences that can be used to enhance the nuclear localization of the site-directed polypeptides include the NLS of the SV40 Large T-antigen and c-Myc. In certain embodiments, the NLS comprises the amino acid sequence PKKKRKV (SEQ ID NO: 8).

The site-directed polypeptide can comprise more than one NLS, such as two, three, four, five, six, or more NLS sequences. Each of the multiple NLSs can be unique in sequence or there can be more than one of the same NLS sequence used. The NLS can be on the amino-terminal (N-terminal) end of the site-directed polypeptide, the carboxy-terminal (C-terminal) end, or both the N-terminal and C-terminal ends of the polypeptide. In certain embodiments, the site-directed polypeptide comprises four NLS sequences on its N-terminal end. In other embodiments, the site-directed polypeptide comprises two NLS sequences on the C-terminal end of the site-directed polypeptide. In still other embodiments, the site-directed polypeptide comprises four NLS sequences on its N-terminal end and two NLS sequences on its C-terminal end.

In certain embodiments, the site-directed polypeptide further comprises a cell penetrating peptide (CPP), which induces the absorption of a linked protein or peptide through the plasma membrane of a cell. Generally, CPPs induce entry into the cell because of their general shape and tendency to either self-assemble into a membrane-spanning pore, or to have several positively charged residues, which interact with the negatively charged phospholipid outer membrane inducing curvature of the membrane, which in turn activates internalization. Exemplary permeable peptides include, but are not limited to, transportan, PEP1, MPG, p-VEC, MAP, CADY, polyR (e.g., SEQ ID NO: 128), HIV-TAT (SEQ ID NO: 9), HIV-REV (SEQ ID NO: 13), Penetratin, R6W3, P22N, DPV3, DPV6, K-FGF, and C105Y, and are reviewed in van den Berg and Dowdy (2011) Current Opinion in Biotechnology 22:888-893 and Farkhani et al. (2014) Peptides 57:78-94, each of which is herein incorporated by reference in its entirety.

Along with or as an alternative to an NLS, the site-directed polypeptide can comprise additional heterologous amino acid sequences, such as a detectable label (e.g., fluorescent protein) described elsewhere herein, or a purification tag, to form a fusion protein. A purification tag is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

The presently disclosed compositions and methods can be used to edit genomes through the introduction of a sequence-specific, double-stranded break that is repaired (via e.g., error-prone non-homologous end-joining (NHEJ), microhomology-mediated end joining (MMEJ), or alternative end-joining (alt-EJ) pathway) to introduce a mutation at a specific genomic location. Due to the error-prone nature of repair processes, repair of the double-stranded break can result in a modification to the target sequence. Alternatively, a donor template polynucleotide may be integrated into or exchanged with the target sequence during the course of repair of the introduced double-stranded break, resulting in the introduction of the exogenous donor sequence. Accordingly, the compositions and methods can further comprise a donor template polynucleotide that may comprise flanking homologous ends. In some of these embodiments, the donor template polynucleotide is tethered to the TAGE agent via a linker as described elsewhere herein (e.g., the donor template polynucleotide is bound to the site-directed polypeptide via a cleavable linker).

In some embodiments, the donor sequence alters the original target sequence such that the newly integrated donor sequence will not be recognized and cleaved by the nucleic acid-guided nuclease. The donor sequence may comprise flanking sequences that have substantial sequence identity with the sequences flanking the target sequence to enhance the integration of the donor sequence via homology-directed repair. In particular embodiments wherein the nucleic acid-guided nuclease generates double-stranded staggered breaks, the donor polynucleotide can be flanked by compatible overhangs, allowing for incorporation of the donor sequence via a non-homologous repair process during repair of the double-stranded break.

### IV. Antigen Binding Polypeptide of the TAGE Agent

An antigen binding polypeptide targets an extracellular antigen associated with a cell membrane and provide specificity with which to deliver a site-directed modifying polypeptide. Examples of antigen binding polypeptides that may be included in the TAGE agent described herein include, but are not limited to, an antibody, an antigen-binding fragment of an antibody, or an antibody mimetic.

### Antibodies and Antigen Binding Fragments

In certain embodiments, a TAGE agent as provided herein comprises an antigen binding polypeptide that is an antibody, or an antigen-binding fragment thereof, that specifically binds to an extracellular molecule (e.g., protein, glycan, lipid) localized on a target cell membrane or associated with a specific tissue. The extracellular molecule specifically bound by the antibody, or antigen-binding fragment thereof, can be an antigen, such as, but not limited to, HLA-DR, CD3, CD11a, CD20, CD22, CD25, CD32, CD33, CD44, CD47, CD54, CD59, CD70, CD74, AchR, CTLA4, CXCR4, EGFR, Her2, EpCam, PD-1, or FAP1. In certain embodiments, the antigen is CD22. In on embodiment, the antibody or antigen binding portion thereof specifically binds to CD3. Other exemplary targets for the antibody, antigen-binding fragment thereof, in the TAGE agent of the present invention include: (i) tumor-associated antigens; (ii) cell surface receptors, (iii) CD proteins and their ligands, such as CD3, CD4, CD8, CD11a, CD19, CD20, CD22, CD25, CD32, CD33, CD34, CD40, CD44, CD47, CD54, CD59, CD70, CD74, CD79a (CD79a), and CD79P (CD79b); (iv) members of the ErbB receptor family such as the EGF receptor, HER2, HER3 or HER4 receptor; (v) cell adhesion molecules such as LFA-1, Mac1, p150,95, VLA-4, ICAM-1, VCAM and αv/β3 integrin including either alpha or beta subunits thereof (e.g. anti-CD11a, anti-CD18 or anti-CD11 b antibodies); and (vi) growth factors such as VEGF; IgE; blood group antigens; flk2/flt3 receptor; obesity (OB) receptor; mpl receptor; CTLA4; protein C, BR3, c-met, tissue factor, β7 etc. Other examples of antigens that can be targeted by the antibody, or an antigen-binding fragment thereof, include cell surface receptors such as those described in Chen and Flies. Nature reviews immunology. 13.4 (2013): 227, which is incorporated herein by reference.

Antigen binding polypeptides used in the TAGE agents described herein may also be specific to a certain cell type. For example, an antigen binding polypeptide, such as an antibody or antigen binding portion thereof, may bind to an antigen present on the cell surface of a hematopoietic cell (HSC). Examples of antigens found on HSCs include, but are not limited to, CD34, EMCN, CD59, CD90, c-KIT, CD45, or CD49F. Other cell types that may be bound by the antigen binding polypeptide via an antigen expressed or displayed on the cell's extracellular surface, and thus gene edited by the TAGE agent, include a neutrophil, a T cell, a B cell, a dendritic cell, a macrophage, and a fibroblast.

Exemplary antibodies (or antigen-binding fragments thereof) include those selected from, and without limitation, an anti-HLA-DR antibody, an anti-CD3 antibody, an anti-CD20 antibody, an anti-CD22 antibody, an anti-CD11a antibody, an anti-CD25 antibody, an anti-CD32 antibody, an anti-CD33 antibody, an anti-CD44 antibody, an anti-CD47 antibody, an anti-CD54 antibody, an anti-CD59 antibody, an anti-CD70 antibody, an anti-CD74 antibody, an anti-AchR antibody, an anti-CTLA4 antibody, an anti-CXCR4 antibody, an anti-EGFR antibody, an anti-Her2 antibody, an anti-EpCam antibody, an-anti-PD-1 antibody, or an anti-FAP1 antibody. Exemplary antibodies to these various targets are described in the sequence table below as SEQ ID Nos: 14 to 115.

In one embodiment, the TAGE agent includes an antigen binding polypeptide that is an anti-CD22 antibody, or antigen-binding fragment thereof. In certain embodiments, the anti-CD22 antibody is selected from epratuzumab (also known as hL22, see, e.g., U.S. Pat. No. 5,789,554; US. App. No. 20120302739; sold by Novus Biologicals, Cat No. NBP2-75189 (date Mar. 3, 2019), bectumomab (see, e.g., U.S. Pat. No. 8,420,086), RFB4 (see, e.g., U.S. Pat. No. 7,355,012), SM03(see, e.g., Zhao et al., Clin Drug Investig (2016) 36:889-902), NCI m972 (see,e.g., U.S. Pat. Nos. 8,591,889, 9,279,019, 9,598,492), or NCI m971 (see, e.g., U.S. Pat. Nos. 7,456,260, 8,591,889, 9,279,019, 9,598,492).

In one embodiment, the TAGE agent comprises an anti-CD22 antibody, or antigen-binding portion thereof. In some embodiments, the anti-CD22 antibody, or antigen-binding portion thereof, comprises a variable heavy chain region comprising the amino acid residues set forth in SEQ ID NO: 108, and a light chain variable region comprising the amino acid residues set forth in SEQ ID NO: 109. In one embodiment, the anti-CD22 antibody, or antigen binding fragment thereof, comprises a heavy chain variable region comprising CDR1, CDR2 and CDR3 domains as set forth in SEQ ID NO: 108, and a light chain variable region comprising CDR1, CDR2 and CDR3 domains as set forth in SEQ ID NO: 109. CDRs can be determined according to Kabat numbering.

In certain embodiments, the TAGE agent includes an antigen binding polypeptide that is an anti-CD11a antibody, or an antigen-binding fragment thereof. CD11a (also known as integrin, alpha L; lymphocyte function-associated antigen 1; alpha polypeptide; or ITGAL; Uniprot Accession No. P20701), is an integrin that is involved in cellular adhesion and lymphocyte costimulatory signaling. CD11a is one of the two components, along with CD18, which form lymphocyte function-associated antigen-1, which is expressed on leukocytes. In certain embodiments, the anti-CD11a antibody is efalizumab (described, e.g., in WO1998023761 or U.S. Pat. No. 6,652,855, each of which are hereby incorporated by reference).

In one embodiment, the anti-CD11a antibody comprises a heavy chain variable region comprising a CDR1, CDR2 and CDR3 of anti-CD11a antibody efalizumab, and a light chain variable region comprising a CDR1, CDR2 and CDR3 of anti-CD11a antibody efalizumab. In one embodiment, the anti-CD11a antibody comprises the heavy chain variable region of anti-CD11a antibody efalizumab, and the light chain variable region of anti-CD11a antibody efalizumab.

In certain embodiments, the TAGE agent includes an antigen binding polypeptide that is an anti-CD25 antibody, or antigen-binding fragment thereof. CD25 (also known as Interleukin-2 receptor alpha chain, IL2RA; Uniprot Accession No. P01589), is a type I transmembrane protein present on activated T cells, activated B cells, some thymocytes, myeloid precursors, and oligodendrocytes. The interleukin 2 (IL2) receptor alpha (IL2RA) and beta (IL2RB) chains, together with the common gamma chain (IL2RG), form the high-affinity IL2 receptor. In certain embodiments, the anti-CD25 antibody is daclizumab (described, e.g., in U.S. Pat. No. 7,361,740, which is hereby incorporated by reference).

In one embodiment, the anti-CD25 antibody comprises a heavy chain variable region comprising a CDR1, CDR2 and CDR3 of anti-CD25 antibody daclizumab, and a light chain variable region comprising a CDR1, CDR2 and CDR3 of anti-CD25 antibody daclizumab. In one embodiment, the anti-CD25 antibody comprises the heavy chain variable region of anti-CD25 antibody daclizumab, and the light chain variable region of anti-CD11a antibody daclizumab.

In certain embodiments, the TAGE agent includes an antigen binding polypeptide that is an anti-FAP antibody, or fragment thereof. Fibroblast activation protein (FAP), also known as Seprase, is a membrane-bound serine protease of the prolyl oligopeptidase family with post-prolyl endopeptidase activity. FAP's restricted expression to the tumor microenvironment (e.g., tumor stroma) makes it an attractive therapeutic candidate to target in the treatment of various tumors. In certain embodiments, the anti-FAP antibody is selected from Sibrotuzumab/BIBH1 (described in WO 99/57151, Mersmann et al., Int J Cancer 92, 240-248 (2001); Schmidt et al., Eur J Biochem 268, 1730-1738 (2001); WO 01/68708, WO 2007/077173), F19 (described in WO 93/05804, ATCC Number HB 8269, sold by R&D systems, Catalog No.: MAB3715), OS4 (described in Wuest et al., J Biotech 92, 159-168 (2001)). Other anti-FAP antibodies are described, for example, in U.S. Pat. Nos. 8,568,727; 8,999,342, US. App. No. 20160060356; US. App. No. 20160060357, and U.S. Pat. No. 9,011,847, each of which is incorporated by reference herein.

In one embodiment, the TAGE agent comprises an anti-FAP antibody, or antigen-binding portion thereof. In some embodiments, the anti-FAP antibody, or antigen-binding portion thereof, comprises a variable heavy chain region comprising the amino acid residues set forth in SEQ ID NO: 100, and a light chain variable region comprising the amino acid residues set forth in SEQ ID NO: 101. In one embodiment, the anti-FAP antibody, or antigen binding fragment thereof, comprises a heavy chain variable region comprising CDR1, CDR2 and CDR3 domains as set forth in SEQ ID NO: 100, and a light chain variable region comprising CDR1, CDR2 and CDR3 domains as set forth in SEQ ID NO: 101. CDRs can be determined according to Kabat numbering.

In certain embodiments, the TAGE agent includes an antigen binding polypeptide that is an anti-CTLA4 antibody, or fragment thereof. CTLA-4 (cytotoxic T-lymphocyte-associated protein 4), also known as CD152 (cluster of differentiation 152), is a member of the immunoglobulin superfamily of protein receptors and functions as an immune checkpoint to downregulate immune responses. CTLA4 expressed on the surface of T lymphocytes with transient expression on the surface of early activated CD8 T cells; and constitutive expression on regulatory T cells. In certain embodiments, the anti-CTLA4 antibody is selected from Ipilimumab (trade name: YERVOY®, described in U.S. Pat. Nos. 6,984,720; 605,238, 8,017,114, 8,318,916, and 8,784,815.) Other anti-CTLA4 antibodies are described, for example, in U.S. Pat. No. 9,714,290; U.S. patent Ser. No. 10/202,453, and US. Publication No. 20170216433, each of which is incorporated by reference herein.

In one embodiment, the anti-CTLA4 antibody, or antigen-binding portion thereof, comprises a variable heavy chain region comprising the amino acid residues set forth in SEQ ID NO: 102, and a light chain variable region comprising the amino acid residues set forth in SEQ ID NO: 103. In one embodiment, the anti-CTLA4 antibody, or antigen binding fragment thereof, comprises a heavy chain variable region comprising CDR1, CDR2 and CDR3 domains as set forth in SEQ ID NO: 102, and a light chain variable region comprising CDR1, CDR2 and CDR3 domains as set forth in SEQ ID NO: 103. CDRs can be determined according to Kabat numbering. The foregoing sequences correspond to anti-CTLA4 antibody ipilumimab.

In one embodiment, the anti-CTLA4 antibody, or antigen-binding portion thereof, comprises a variable heavy chain region comprising the amino acid residues set forth in SEQ ID NO: 104, and a light chain variable region comprising the amino acid residues set forth in SEQ ID NO: 105. In one embodiment, the anti-CTLA4 antibody, or antigen binding fragment thereof, comprises a heavy chain variable region comprising CDR1, CDR2 and CDR3 domains as set forth in SEQ ID NO: 104, and a light chain variable region comprising CDR1, CDR2 and CDR3 domains as set forth in SEQ ID NO: 105. CDRs can be determined according to Kabat numbering. The foregoing sequences correspond to anti-CTLA4 antibody tremelimumab.

In certain embodiments, the TAGE agent includes an antigen binding polypeptide that is an anti-CD44 antibody, or fragment thereof. CD44 is a ubiquitous cell surface glycoprotein that is highly expressed in many cancers and regulates metastasis via recruitment of CD44 to the cell surface. In certain embodiments, the anti-CD44 antibody is selected from RG7356 (described in PCT Publication: WO2013063498A1). Other anti-CTLA4 antibodies are described, for example, in US. Publication No. 20170216433, US. Publication No. 20070237761 A1, and US. Publication No. US20100092484, each of which is incorporated by reference herein.

In one embodiment, the TAGE agent comprises an anti-CD44 antibody, or antigen-binding portion thereof. In some embodiments, the anti-CD44 antibody, or antigen-binding portion thereof, comprises a variable heavy chain region comprising the amino acid residues set forth in SEQ ID NO: 30, and a light chain variable region comprising the amino acid residues set forth in SEQ ID NO: 31. In one embodiment, the anti-CD44 antibody, or antigen binding fragment thereof, comprises a heavy chain variable region comprising CDR1, CDR2 and CDR3 domains as set forth in SEQ ID NO: 30, and a light chain variable region comprising CDR1, CDR2 and CDR3 domains as set forth in SEQ ID NO: 31. CDRs can be determined according to Kabat numbering.

In certain embodiments, the TAGE agent includes an antigen binding polypeptide that is an anti-CD54 antibody, or fragment thereof. The CD54 is a cell surface glycoprotein that binds to the leucocyte function-associated antigen-1 (CD11a/CD18 [LFA-1]). CD54 modulates both LFA-1-dependent adhesion of leucocytes to endothelial cells and immune functions involving cell-to-cell contact. Anti-CD54 antibodies are described, for example, in U.S. Pat. Nos. 7,943,744, 5,773,293, 8,623,369, PCT Publication No. WO91/16928, and US. Publication No. US20100092484, each of which is incorporated by reference herein.

In one embodiment, the TAGE agent comprises an anti-CD54 antibody, or antigen-binding portion thereof. In some embodiments, the anti-CD54 antibody, or antigen-binding portion thereof, comprises a variable heavy chain region comprising the amino acid residues set forth in SEQ ID NO: 86, and a light chain variable region comprising the amino acid residues set forth in SEQ ID NO: 87. In one embodiment, the anti-CD54 antibody, or antigen binding fragment thereof, comprises a heavy chain variable region comprising CDR1, CDR2 and CDR3 domains as set forth in SEQ ID NO: 86, and a light chain variable region comprising CDR1, CDR2 and CDR3 domains as set forth in SEQ ID NO: 87. CDRs can be determined according to Kabat numbering.

In certain embodiments, the TAGE agent includes an antigen binding polypeptide that is an anti-CD33 antibody, or fragment thereof. CD33 or Siglec-3 (sialic acid binding Ig-like lectin 3, SIGLEC3, SIGLEC-3, gp67, p67) is a myeloid specific member of the sialic acid-binding receptor family and is expressed highly on myeloid progenitor cells but at much lower levels in differentiated cells. In certain embodiments, the anti-CD33 antibody is selected from lintuzumab (also known as clone HuM195, described in U.S. Pat. No. 9,079,958,) 2H12 (described in U.S. Pat. No. 9,587,019). Other CD33 antibodies have been described in, for example, U.S. Pat. Nos. 7,342,110, 7,557,189, 8,119,787, 8,337,855, 8,124,069, 5,730,982, U.S. Pat. No. 7,695,71, WO2012074097, WO2004043344, WO1993020848, WO2012045752, WO2007014743, WO2003093298, WO2011036183, WO1991009058, WO2008058021, WO2011038301, Hoyer et al., (2008) Am. J. Clin. Pathol. 129, 316-323, Rollins-Raval and Roth, (2012) Histopathology 60, 933-942), Pérez-Oliva et al., (2011) Glycobiol. 21, 757-770), Ferlazzo et al. (2000) Eur J Immunol. 30:827-833, Vitale et al., (2001) Proc Natl Acad Sci USA. 98:5764-5769, Jandus et al., (2011) Biochem. Pharmacol. 82, 323-332, O'Reilly and Paulson, (2009) Trends Pharmacol. Sci. 30, 240-248, Jurcic, (2012) Curr Hematol Malig Rep 7, 65-73, and Ricart, (2011) Clin. Cancer Res. 17, 6417-6427, each of which is incorporated by reference herein.

In certain embodiments, the TAGE agent includes an antigen binding polypeptide that is an anti-CD22 antibody, or fragment thereof. In certain embodiments, the anti-CD22 antibody is the anti-CD22 antibody epratuzumab (also known as hL22, see, e.g., U.S. Pat. No. 5,789,554; US. App. No. 20120302739; sold by Novus Biologicals, Cat No. NBP2-75189 (date Mar. 3, 2019) or an anti-CD22 antibody comprising antigen binding regions corresponding to the epratuzumab antibody. Epratuzumab antibody is a humanized antibody derived from antibody LL2 (EPB-2), a murine anti-CD22 IgG2a raised against Raji Burkitt lymphoma cells.

In one embodiment, the anti-CD22 antibody comprises a heavy chain comprising a CDR1, CDR2 and CDR3 of anti-CD22 antibody epratuzumab, and a light chain variable region comprising a CDR1, CDR2 and CDR3 of anti-CD22 antibody epratuzumab.

In one embodiment, the TAGE agent includes an antigen binding polypeptide that is an anti-CD3 antibody, or antigen binding fragment thereof. In certain embodiments, the anti-CD3 antibody is muromonab (also known as OKT3; sold by BioLegend, Cat. No. 317301 or 317302 (date Mar. 3, 2019)), visilizumab (see, e.g., U.S. Pat. Nos. 5,834,597, 7,381,803, US App. No. 20080025975), otelixizumab (see, e.g., WO2007145941), or Dow2 (see, e.g., WO2014129270).

In certain embodiments, the TAGE agent comprises an anti-CD3 antibody, wherein the anti-CD3 antibody is the anti-CD3 antibody muromonab (also known as OKT3; sold by BioLegend, Cat. No. 317301 or 317302 (date Mar. 3, 2019)) or an anti-CD3 antibody comprising antigen binding regions corresponding to muromonab.

In one embodiment, the anti-CD3 antibody comprises a heavy chain comprising a CDR1, CDR2 and CDR3 of anti-CD3 antibody muromonab, and a light chain variable region comprising a CDR1, CDR2 and CDR3 of anti-CD3 antibody muromonab.

In one embodiment, the TAGE agent comprises an anti-CD3 antibody, or antigen-binding portion thereof. In some embodiments, the anti-CD3 antibody, or antigen-binding portion thereof, comprises a variable heavy chain region comprising the amino acid residues set forth in SEQ ID NO: 76, and a light chain variable region comprising the amino acid residues set forth in SEQ ID NO: 77. In one embodiment, the anti-CD3 antibody, or antigen binding fragment thereof, comprises a heavy chain variable region comprising CDR1, CDR2 and CDR3 domains as set forth in SEQ ID NO: 76, and a light chain variable region comprising CDR1, CDR2 and CDR3 domains as set forth in SEQ ID NO: 77. CDRs can be determined according to Kabat numbering.

In one embodiment, the TAGE agent comprises an anti-CD45 antibody, or antigen-binding portion thereof. In some embodiments, the anti-CD45 antibody, or antigen-binding portion thereof, comprises a variable heavy chain region comprising the amino acid residues set forth in SEQ ID NO: 14, and a light chain variable region comprising the amino acid residues set forth in SEQ ID NO: 15. In one embodiment, the anti-CD45 antibody, or antigen binding fragment thereof, comprises a heavy chain variable region comprising CDR1, CDR2 and CDR3 domains as set forth in SEQ ID NO: 14, and a light chain variable region comprising CDR1, CDR2 and CDR3 domains as set forth in SEQ ID NO: 15. CDRs can be determined according to Kabat numbering.

In one embodiment, the TAGE agent comprises an anti-CD48 antibody, or antigen-binding portion thereof. In some embodiments, the anti-CD48 antibody, or antigen-binding portion thereof, comprises a variable heavy chain region comprising the amino acid residues set forth in SEQ ID NO: 16, and a light chain variable region comprising the amino acid residues set forth in SEQ ID NO: 17. In one embodiment, the anti-CD45 antibody, or antigen binding fragment thereof, comprises a heavy chain variable region comprising CDR1, CDR2 and CDR3 domains as set forth in SEQ ID NO: 16, and a light chain variable region comprising CDR1, CDR2 and CDR3 domains as set forth in SEQ ID NO: 17. CDRs can be determined according to Kabat numbering.

In one embodiment, the TAGE agent comprises an anti-TIM3 antibody, or antigen-binding portion thereof. In some embodiments, the anti-TIM3 antibody, or antigen-binding portion thereof, comprises a variable heavy chain region comprising the amino acid residues set forth in SEQ ID NO: 18, and a light chain variable region comprising the amino acid residues set forth in SEQ ID NO:193. In one embodiment, the anti-TIM3 antibody, or antigen binding fragment thereof, comprises a heavy chain variable region comprising CDR1, CDR2 and CDR3 domains as set forth in SEQ ID NO: 18, and a light chain variable region comprising CDR1, CDR2 and CDR3 domains as set forth in SEQ ID NO: 19. CDRs can be determined according to Kabat numbering.

In one embodiment, the TAGE agent comprises an anti-CD73 antibody, or antigen-binding portion thereof. In some embodiments, the anti-CD73 antibody, or antigen-binding portion thereof, comprises a variable heavy chain region comprising the amino acid residues set forth in SEQ ID NO: 20, and a light chain variable region comprising the amino acid residues set forth in SEQ ID NO: 21. In one embodiment, the anti-CD73 antibody, or antigen binding fragment thereof, comprises a heavy chain variable region comprising CDR1, CDR2 and CDR3 domains as set forth in SEQ ID NO: 20, and a light chain variable region comprising CDR1, CDR2 and CDR3 domains as set forth in SEQ ID NO: 21. CDRs can be determined according to Kabat numbering.

In one embodiment, the TAGE agent comprises an anti-TIGIT antibody, or antigen-binding portion thereof. In some embodiments, the anti-TIGIT antibody, or antigen-binding portion thereof, comprises a variable heavy chain region comprising the amino acid residues set forth in SEQ ID NO: 22, and a light chain variable region comprising the amino acid residues set forth in SEQ ID NO: 23. In one embodiment, the anti-TIGIT antibody, or antigen binding fragment thereof, comprises a heavy chain variable region comprising CDR1, CDR2 and CDR3 domains as set forth in SEQ ID NO: 22, and a light chain variable region comprising CDR1, CDR2 and CDR3 domains as set forth in SEQ ID NO: 23. CDRs can be determined according to Kabat numbering.

In one embodiment, the TAGE agent comprises an anti-CCR4 antibody, or antigen-binding portion thereof. In some embodiments, the anti-CCR4 antibody, or antigen-binding portion thereof, comprises a variable heavy chain region comprising the amino acid residues set forth in SEQ ID NO: 24, and a light chain variable region comprising the amino acid residues set forth in SEQ ID NO: 25. In one embodiment, the anti-CCR4 antibody, or antigen binding fragment thereof, comprises a heavy chain variable region comprising CDR1, CDR2 and CDR3 domains as set forth in SEQ ID NO: 24, and a light chain variable region comprising CDR1, CDR2 and CDR3 domains as set forth in SEQ ID NO: 25. CDRs can be determined according to Kabat numbering.

In one embodiment, the TAGE agent comprises an anti-IL-4R antibody, or antigen-binding portion thereof. In some embodiments, the anti-IL-4R antibody, or antigen-binding portion thereof, comprises a variable heavy chain region comprising the amino acid residues set forth in SEQ ID NO: 26, and a light chain variable region comprising the amino acid residues set forth in SEQ ID NO: 27. In one embodiment, the anti-IL-4R antibody, or antigen binding fragment thereof, comprises a heavy chain variable region comprising CDR1, CDR2 and CDR3 domains as set forth in SEQ ID NO: 26, and a light chain variable region comprising CDR1, CDR2 and CDR3 domains as set forth in SEQ ID NO: 27. CDRs can be determined according to Kabat numbering.

In one embodiment, the TAGE agent comprises an anti-CCR2 antibody, or antigen-binding portion thereof. In some embodiments, the anti-CCR2 antibody, or antigen-binding portion thereof, comprises a variable heavy chain region comprising the amino acid residues set forth in SEQ ID NO: 28, and a light chain variable region comprising the amino acid residues set forth in SEQ ID NO: 29. In one embodiment, the anti-CCR2 antibody, or antigen binding fragment thereof, comprises a heavy chain variable region comprising CDR1, CDR2 and CDR3 domains as set forth in SEQ ID NO: 28, and a light chain variable region comprising CDR1, CDR2 and CDR3 domains as set forth in SEQ ID NO: 29. CDRs can be determined according to Kabat numbering.

In one embodiment, the TAGE agent comprises an anti-CCR5 antibody, or antigen-binding portion thereof. In some embodiments, the anti-CCR5 antibody, or antigen-binding portion thereof, comprises a variable heavy chain region comprising the amino acid residues set forth in SEQ ID NO: 32, and a light chain variable region comprising the amino acid residues set forth in SEQ ID NO: 33. In one embodiment, the anti-CCR5 antibody, or antigen binding fragment thereof, comprises a heavy chain variable region comprising CDR1, CDR2 and CDR3 domains as set forth in SEQ ID NO: 32, and a light chain variable region comprising CDR1, CDR2 and CDR3 domains as set forth in SEQ ID NO: 33. CDRs can be determined according to Kabat numbering.

In one embodiment, the TAGE agent comprises an anti-CXCR4 antibody, or antigen-binding portion thereof. In some embodiments, the anti-CXCR4 antibody, or antigen-binding portion thereof, comprises a variable heavy chain region comprising the amino acid residues set forth in SEQ ID NO: 34, and a light chain variable region comprising the amino acid residues set forth in SEQ ID NO: 35. In one embodiment, the anti-CXCR4 antibody, or antigen binding fragment thereof, comprises a heavy chain variable region comprising CDR1, CDR2 and CDR3 domains as set forth in SEQ ID NO: 34, and a light chain variable region comprising CDR1, CDR2 and CDR3 domains as set forth in SEQ ID NO: 35. CDRs can be determined according to Kabat numbering.

In one embodiment, the TAGE agent comprises an anti-SLAMF7 antibody, or antigen-binding portion thereof. In some embodiments, the anti-SLAMF7 antibody, or antigen-binding portion thereof, comprises a variable heavy chain region comprising the amino acid residues set forth in SEQ ID NO: 36, and a light chain variable region comprising the amino acid residues set forth in SEQ ID NO: 37. In one embodiment, the anti-SLAMF7 antibody, or antigen binding fragment thereof, comprises a heavy chain variable region comprising CDR1, CDR2 and CDR3 domains as set forth in SEQ ID NO: 36, and a light chain variable region comprising CDR1, CDR2 and CDR3 domains as set forth in SEQ ID NO: 37. CDRs can be determined according to Kabat numbering.

In one embodiment, the TAGE agent comprises an anti-ICOS antibody, or antigen-binding portion thereof. In some embodiments, the anti-ICOS antibody, or antigen-binding portion thereof, comprises a variable heavy chain region comprising the amino acid residues set forth in SEQ ID NO: 38, and a light chain variable region comprising the amino acid residues set forth in SEQ ID NO: 39. In one embodiment, the anti-ICOS antibody, or antigen binding fragment thereof, comprises a heavy chain variable region comprising CDR1, CDR2 and CDR3 domains as set forth in SEQ ID NO: 38, and a light chain variable region comprising CDR1, CDR2 and CDR3 domains as set forth in SEQ ID NO: 39. CDRs can be determined according to Kabat numbering.

In one embodiment, the TAGE agent comprises an anti-PD-L1 antibody, or antigen-binding portion thereof. In some embodiments, the anti-PD-L1 antibody, or antigen-binding portion thereof, comprises a variable heavy chain region comprising the amino acid residues set forth in SEQ ID NO: 40, and a light chain variable region comprising the amino acid residues set forth in SEQ ID NO: 41. In one embodiment, the anti-PD-L1 antibody, or antigen binding fragment thereof, comprises a heavy chain variable region comprising CDR1, CDR2 and CDR3 domains as set forth in SEQ ID NO: 40, and a light chain variable region comprising CDR1, CDR2 and CDR3 domains as set forth in SEQ ID NO: 41. CDRs can be determined according to Kabat numbering.

In one embodiment, the TAGE agent comprises an anti-OX40 antibody, or antigen-binding portion thereof. In some embodiments, the anti-OX40 antibody, or antigen-binding portion thereof, comprises a variable heavy chain region comprising the amino acid residues set forth in SEQ ID NO: 42, and a light chain variable region comprising the amino acid residues set forth in SEQ ID NO: 43. In one embodiment, the anti-OX40 antibody, or antigen binding fragment thereof, comprises a heavy chain variable region comprising CDR1, CDR2 and CDR3 domains as set forth in SEQ ID NO: 42, and a light chain variable region comprising CDR1, CDR2 and CDR3 domains as set forth in SEQ ID NO: 43. CDRs can be determined according to Kabat numbering.

In one embodiment, the TAGE agent comprises an anti-CD11a antibody, or antigen-binding portion thereof. In some embodiments, the anti-CD11a antibody, or antigen-binding portion thereof, comprises a variable heavy chain region comprising the amino acid residues set forth in SEQ ID NO: 44, and a light chain variable region comprising the amino acid residues set forth in SEQ ID NO: 45. In one embodiment, the anti-CD11a antibody, or antigen binding fragment thereof, comprises a heavy chain variable region comprising CDR1, CDR2 and CDR3 domains as set forth in SEQ ID NO: 44, and a light chain variable region comprising CDR1, CDR2 and CDR3 domains as set forth in SEQ ID NO: 45. CDRs can be determined according to Kabat numbering.

In one embodiment, the TAGE agent comprises an anti-CD40L antibody, or antigen-binding portion thereof. In some embodiments, the anti-CD40L antibody, or antigen-binding portion thereof, comprises a variable heavy chain region comprising the amino acid residues set forth in SEQ ID NO: 46, and a light chain variable region comprising the amino acid residues set forth in SEQ ID NO: 47. In one embodiment, the anti-CD40L antibody, or antigen binding fragment thereof, comprises a heavy chain variable region comprising CDR1, CDR2 and CDR3 domains as set forth in SEQ ID NO: 46, and a light chain variable region comprising CDR1, CDR2 and CDR3 domains as set forth in SEQ ID NO: 47. CDRs can be determined according to Kabat numbering.

In one embodiment, the TAGE agent comprises an anti-IFNAR1 antibody, or antigen-binding portion thereof. In some embodiments, the anti-IFNAR1 antibody, or antigen-binding portion thereof, comprises a variable heavy chain region comprising the amino acid residues set forth in SEQ ID NO: 48, and a light chain variable region comprising the amino acid residues set forth in SEQ ID NO: 49. In one embodiment, the anti-IFNAR1 antibody, or antigen binding fragment thereof, comprises a heavy chain variable region comprising CDR1, CDR2 and CDR3 domains as set forth in SEQ ID NO: 48, and a light chain variable region comprising CDR1, CDR2 and CDR3 domains as set forth in SEQ ID NO: 49. CDRs can be determined according to Kabat numbering.

In one embodiment, the TAGE agent comprises an anti-transferrin antibody, or antigen-binding portion thereof. In some embodiments, the anti-transferrin antibody, or antigen-binding portion thereof, comprises a variable heavy chain region comprising the amino acid residues set forth in SEQ ID NO: 50, and a light chain variable region comprising the amino acid residues set forth in SEQ ID NO:51. In one embodiment, the anti-transferrin antibody, or antigen binding fragment thereof, comprises a heavy chain variable region comprising CDR1, CDR2 and CDR3 domains as set forth in SEQ ID NO: 50, and a light chain variable region comprising CDR1, CDR2 and CDR3 domains as set forth in SEQ ID NO: 51. CDRs can be determined according to Kabat numbering.

In one embodiment, the TAGE agent comprises an anti-CD80 antibody, or antigen-binding portion thereof. In some embodiments, the anti-CD80 antibody, or antigen-binding portion thereof, comprises a variable heavy chain region comprising the amino acid residues set forth in SEQ ID NO: 52, and a light chain variable region comprising the amino acid residues set forth in SEQ ID NO: 53. In one embodiment, the anti-CD80 antibody, or antigen binding fragment thereof, comprises a heavy chain variable region comprising CDR1, CDR2 and CDR3 domains as set forth in SEQ ID NO: 52, and a light chain variable region comprising CDR1, CDR2 and CDR3 domains as set forth in SEQ ID NO: 53. CDRs can be determined according to Kabat numbering.

In one embodiment, the TAGE agent comprises an anti-IL6-R antibody, or antigen-binding portion thereof. In some embodiments, the anti-IL6-R antibody, or antigen-binding portion thereof, comprises a variable heavy chain region comprising the amino acid residues set forth in SEQ ID NO: 54, and a light chain variable region comprising the amino acid residues set forth in SEQ ID NO: 55. In one embodiment, the anti-IL6-R antibody, or antigen binding fragment thereof, comprises a heavy chain variable region comprising CDR1, CDR2 and CDR3 domains as set forth in SEQ ID NO: 54, and a light chain variable region comprising CDR1, CDR2 and CDR3 domains as set forth in SEQ ID NO: 55. CDRs can be determined according to Kabat numbering.

In one embodiment, the TAGE agent comprises an anti-TCRb antibody, or antigen-binding portion thereof. In some embodiments, the anti-TCRb antibody, or antigen-binding portion thereof, comprises a variable heavy chain region comprising the amino acid residues set forth in SEQ ID NO: 56, and a light chain variable region comprising the amino acid residues set forth in SEQ ID NO: 57. In one embodiment, the anti-TCRb antibody, or antigen binding fragment thereof, comprises a heavy chain variable region comprising CDR1, CDR2 and CDR3 domains as set forth in SEQ ID NO: 56, and a light chain variable region comprising CDR1, CDR2 and CDR3 domains as set forth in SEQ ID NO: 57. CDRs can be determined according to Kabat numbering.

In one embodiment, the TAGE agent comprises an anti-CD59 antibody, or antigen-binding portion thereof. In some embodiments, the anti-CD59 antibody, or antigen-binding portion thereof, comprises a variable heavy chain region comprising the amino acid residues set forth in SEQ ID NO: 58, and a light chain variable region comprising the amino acid residues set forth in SEQ ID NO: 59. In one embodiment, the anti-CD59 antibody, or antigen binding fragment thereof, comprises a heavy chain variable region comprising CDR1, CDR2 and CDR3 domains as set forth in SEQ ID NO: 58, and a light chain variable region comprising CDR1, CDR2 and CDR3 domains as set forth in SEQ ID NO: 59. CDRs can be determined according to Kabat numbering.

In one embodiment, the TAGE agent comprises an anti-CD4 antibody, or antigen-binding portion thereof. In some embodiments, the anti-CD4 antibody, or antigen-binding portion thereof, comprises a variable heavy chain region comprising the amino acid residues set forth in SEQ ID NO: 60, and a light chain variable region comprising the amino acid residues set forth in SEQ ID NO: 61. In one embodiment, the anti-CD4 antibody, or antigen binding fragment thereof, comprises a heavy chain variable region comprising CDR1, CDR2 and CDR3 domains as set forth in SEQ ID NO: 60, and a light chain variable region comprising CDR1, CDR2 and CDR3 domains as set forth in SEQ ID NO: 61. CDRs can be determined according to Kabat numbering.

In one embodiment, the TAGE agent comprises an anti-HLA-DR antibody, or antigen-binding portion thereof. In some embodiments, the anti-HLA-DR antibody, or antigen-binding portion thereof, comprises a variable heavy chain region comprising the amino acid residues set forth in SEQ ID NO: 62, and a light chain variable region comprising the amino acid residues set forth in SEQ ID NO: 63. In one embodiment, the anti-HLA-DR antibody, or antigen binding fragment thereof, comprises a heavy chain variable region comprising CDR1, CDR2 and CDR3 domains as set forth in SEQ ID NO: 62, and a light chain variable region comprising CDR1, CDR2 and CDR3 domains as set forth in SEQ ID NO: 63. CDRs can be determined according to Kabat numbering.

In one embodiment, the TAGE agent comprises an anti-LAG3 antibody, or antigen-binding portion thereof. In some embodiments, the anti-LAG3 antibody, or antigen-binding portion thereof, comprises a variable heavy chain region comprising the amino acid residues set forth in SEQ ID NO: 64, and a light chain variable region comprising the amino acid residues set forth in SEQ ID NO: 65. In one embodiment, the anti-LAG3 antibody, or antigen binding fragment thereof, comprises a heavy chain variable region comprising CDR1, CDR2 and CDR3 domains as set forth in SEQ ID NO: 64, and a light chain variable region comprising CDR1, CDR2 and CDR3 domains as set forth in SEQ ID NO: 65. CDRs can be determined according to Kabat numbering.

In one embodiment, the TAGE agent comprises an anti-4-1 BB antibody, or antigen-binding portion thereof. In some embodiments, the anti-4-1 BB antibody, or antigen-binding portion thereof, comprises a variable heavy chain region comprising the amino acid residues set forth in SEQ ID NO: 66, and a light chain variable region comprising the amino acid residues set forth in SEQ ID NO: 67. In one embodiment, the anti-4-1 BB antibody, or antigen binding fragment thereof, comprises a heavy chain variable region comprising CDR1, CDR2 and CDR3 domains as set forth in SEQ ID NO: 66, and a light chain variable region comprising CDR1, CDR2 and CDR3 domains as set forth in SEQ ID NO: 67. CDRs can be determined according to Kabat numbering.

In one embodiment, the TAGE agent comprises an anti-GITR antibody, or antigen-binding portion thereof. In some embodiments, the anti-GITR antibody, or antigen-binding portion thereof, comprises a variable heavy chain region comprising the amino acid residues set forth in SEQ ID NO: 68, and a light chain variable region comprising the amino acid residues set forth in SEQ ID NO: 69. In one embodiment, the anti-GITR antibody, or antigen binding fragment thereof, comprises a heavy chain variable region comprising CDR1, CDR2 and CDR3 domains as set forth in SEQ ID NO: 68, and a light chain variable region comprising CDR1, CDR2 and CDR3 domains as set forth in SEQ ID NO: 69. CDRs can be determined according to Kabat numbering.

In one embodiment, the TAGE agent comprises an anti-CD27 antibody, or antigen-binding portion thereof. In some embodiments, the anti-CD27 antibody, or antigen-binding portion thereof, comprises a variable heavy chain region comprising the amino acid residues set forth in SEQ ID NO: 70, and a light chain variable region comprising the amino acid residues set forth in SEQ ID NO: 71. In one embodiment, the anti-CD27 antibody, or antigen binding fragment thereof, comprises a heavy chain variable region comprising CDR1, CDR2 and CDR3 domains as set forth in SEQ ID NO: 70, and a light chain variable region comprising CDR1, CDR2 and CDR3 domains as set forth in SEQ ID NO: 71. CDRs can be determined according to Kabat numbering.

In one embodiment, the TAGE agent comprises an anti-nkg2a antibody, or antigen-binding portion thereof. In some embodiments, the anti-nkg2a antibody, or antigen-binding portion thereof, comprises a variable heavy chain region comprising the amino acid residues set forth in SEQ ID NO: 72, and a light chain variable region comprising the amino acid residues set forth in SEQ ID NO: 73. In one embodiment, the anti-nkg2a antibody, or antigen binding fragment thereof, comprises a heavy chain variable region comprising CDR1, CDR2 and CDR3 domains as set forth in SEQ ID NO: 72, and a light chain variable region comprising CDR1, CDR2 and CDR3 domains as set forth in SEQ ID NO: 73. CDRs can be determined according to Kabat numbering.

In one embodiment, the TAGE agent comprises an anti-CD25 antibody, or antigen-binding portion thereof. In some embodiments, the anti-CD25 antibody, or antigen-binding portion thereof, comprises a variable heavy chain region comprising the amino acid residues set forth in SEQ ID NO: 74, and a light chain variable region comprising the amino acid residues set forth in SEQ ID NO: 75. In one embodiment, the anti-CD25 antibody, or antigen binding fragment thereof, comprises a heavy chain variable region comprising CDR1, CDR2 and CDR3 domains as set forth in SEQ ID NO: 74, and a light chain variable region comprising CDR1, CDR2 and CDR3 domains as set forth in SEQ ID NO: 75. CDRs can be determined according to Kabat numbering.

In one embodiment, the TAGE agent comprises an anti-TLR2 antibody, or antigen-binding portion thereof. In some embodiments, the anti-TLR2 antibody, or antigen-binding portion thereof, comprises a variable heavy chain region comprising the amino acid residues set forth in SEQ ID NO: 78, and a light chain variable region comprising the amino acid residues set forth in SEQ ID NO: 79. In one embodiment, the anti-TLR2 antibody, or antigen binding fragment thereof, comprises a heavy chain variable region comprising CDR1, CDR2 and CDR3 domains as set forth in SEQ ID NO: 78, and a light chain variable region comprising CDR1, CDR2 and CDR3 domains as set forth in SEQ ID NO: 79. CDRs can be determined according to Kabat numbering.

In one embodiment, the TAGE agent comprises an anti-PD1 antibody, or antigen-binding portion thereof. In some embodiments, the anti-PD1 antibody, or antigen-binding portion thereof, comprises a variable heavy chain region comprising the amino acid residues set forth in SEQ ID NO: 80, and a light chain variable region comprising the amino acid residues set forth in SEQ ID NO: 81. In one embodiment, the anti-PD1 antibody, or antigen binding fragment thereof, comprises a heavy chain variable region comprising CDR1, CDR2 and CDR3 domains as set forth in SEQ ID NO: 80, and a light chain variable region comprising CDR1, CDR2 and CDR3 domains as set forth in SEQ ID NO: 81. CDRs can be determined according to Kabat numbering.

In one embodiment, the TAGE agent comprises an anti-CD2 antibody, or antigen-binding portion thereof. In some embodiments, the anti-CD2 antibody, or antigen-binding portion thereof, comprises a variable heavy chain region comprising the amino acid residues set forth in SEQ ID NO: 82, and a light chain variable region comprising the amino acid residues set forth in SEQ ID NO: 83. In one embodiment, the anti-CD2 antibody, or antigen binding fragment thereof, comprises a heavy chain variable region comprising CDR1, CDR2 and CDR3 domains as set forth in SEQ ID NO: 82, and a light chain variable region comprising CDR1, CDR2 and CDR3 domains as set forth in SEQ ID NO: 83. CDRs can be determined according to Kabat numbering.

In one embodiment, the TAGE agent comprises an anti-CD52 antibody, or antigen-binding portion thereof. In some embodiments, the anti-CD52 antibody, or antigen-binding portion thereof, comprises a variable heavy chain region comprising the amino acid residues set forth in SEQ ID NO: 84, and a light chain variable region comprising the amino acid residues set forth in SEQ ID NO: 85. In one embodiment, the anti-CD52 antibody, or antigen binding fragment thereof, comprises a heavy chain variable region comprising CDR1, CDR2 and CDR3 domains as set forth in SEQ ID NO: 84, and a light chain variable region comprising CDR1, CDR2 and CDR3 domains as set forth in SEQ ID NO: 85. CDRs can be determined according to Kabat numbering.

In one embodiment, the TAGE agent comprises an anti-EGFR antibody, or antigen-binding portion thereof. In some embodiments, the anti-EGFR antibody, or antigen-binding portion thereof, comprises a variable heavy chain region comprising the amino acid residues set forth in SEQ ID NO: 88, and a light chain variable region comprising the amino acid residues set forth in SEQ ID NO: 89. In one embodiment, the anti-EGFR antibody, or antigen binding fragment thereof, comprises a heavy chain variable region comprising CDR1, CDR2 and CDR3 domains as set forth in SEQ ID NO: 88, and a light chain variable region comprising CDR1, CDR2 and CDR3 domains as set forth in SEQ ID NO: 89. CDRs can be determined according to Kabat numbering.

In one embodiment, the TAGE agent comprises an anti-IGF-1R antibody, or antigen-binding portion thereof. In some embodiments, the anti-IGF-1R antibody, or antigen-binding portion thereof, comprises a variable heavy chain region comprising the amino acid residues set forth in SEQ ID NO: 90, and a light chain variable region comprising the amino acid residues set forth in SEQ ID NO: 91. In one embodiment, the anti-IGF-1R antibody, or antigen binding fragment thereof, comprises a heavy chain variable region comprising CDR1, CDR2 and CDR3 domains as set forth in SEQ ID NO: 90, and a light chain variable region comprising CDR1, CDR2 and CDR3 domains as set forth in SEQ ID NO: 91. CDRs can be determined according to Kabat numbering.

In one embodiment, the TAGE agent comprises an anti-CD30 antibody, or antigen-binding portion thereof. In some embodiments, the anti-CD30 antibody, or antigen-binding portion thereof, comprises a variable heavy chain region comprising the amino acid residues set forth in SEQ ID NO: 92, and a light chain variable region comprising the amino acid residues set forth in SEQ ID NO: 93. In one embodiment, the anti-CD30 antibody, or antigen binding fragment thereof, comprises a heavy chain variable region comprising CDR1, CDR2 and CDR3 domains as set forth in SEQ ID NO: 92, and a light chain variable region comprising CDR1, CDR2 and CDR3 domains as set forth in SEQ ID NO: 93. CDRs can be determined according to Kabat numbering.

In one embodiment, the TAGE agent comprises an anti-CD19 antibody, or antigen-binding portion thereof. In some embodiments, the anti-CD19 antibody, or antigen-binding portion thereof, comprises a variable heavy chain region comprising the amino acid residues set forth in SEQ ID NO: 94, and a light chain variable region comprising the amino acid residues set forth in SEQ ID NO: 95. In one embodiment, the anti-CD19 antibody, or antigen binding fragment thereof, comprises a heavy chain variable region comprising CDR1, CDR2 and CDR3 domains as set forth in SEQ ID NO: 94, and a light chain variable region comprising CDR1, CDR2 and CDR3 domains as set forth in SEQ ID NO: 95. CDRs can be determined according to Kabat numbering.

In one embodiment, the TAGE agent comprises an anti-CD34 antibody, or antigen-binding portion thereof. In some embodiments, the anti-CD34 antibody, or antigen-binding portion thereof, comprises a variable heavy chain region comprising the amino acid residues set forth in SEQ ID NO: 96, and a light chain variable region comprising the amino acid residues set forth in SEQ ID NO: 97. In one embodiment, the anti-CD34 antibody, or antigen binding fragment thereof, comprises a heavy chain variable region comprising CDR1, CDR2 and CDR3 domains as set forth in SEQ ID NO: 96, and a light chain variable region comprising CDR1, CDR2 and CDR3 domains as set forth in SEQ ID NO: 97. CDRs can be determined according to Kabat numbering.

In one embodiment, the TAGE agent comprises an anti-CD59 antibody, or antigen-binding portion thereof. In some embodiments, the anti-CD59 antibody, or antigen-binding portion thereof, comprises a variable heavy chain region comprising the amino acid residues set forth in SEQ ID NO: 98, and a light chain variable region comprising the amino acid residues set forth in SEQ ID NO: 99. In one embodiment, the anti-CD59 antibody, or antigen binding fragment thereof, comprises a heavy chain variable region comprising CDR1, CDR2 and CDR3 domains as set forth in SEQ ID NO: 98, and a light chain variable region comprising CDR1, CDR2 and CDR3 domains as set forth in SEQ ID NO: 99. CDRs can be determined according to Kabat numbering.

In one embodiment, the TAGE agent comprises an anti-CD47 antibody, or antigen-binding portion thereof. In some embodiments, the anti-CD47 antibody, or antigen-binding portion thereof, comprises a variable heavy chain region comprising the amino acid residues set forth in SEQ ID NO: 114, and a light chain variable region comprising the amino acid residues set forth in SEQ ID NO: 115. In one embodiment, the anti-CD47 antibody, or antigen binding fragment thereof, comprises a heavy chain variable region comprising CDR1, CDR2 and CDR3 domains as set forth in SEQ ID NO: 114, and a light chain variable region comprising CDR1, CDR2 and CDR3 domains as set forth in SEQ ID NO: 115. CDRs can be determined according to Kabat numbering.

In some embodiments, the antibody, antigen binding fragment thereof, comprises variable regions having an amino acid sequence that is at least 95%, 96%, 97% or 99% identical to an antibody disclosed herein, including sequences in the cited references. Alternatively, the antibody, or antigen binding fragment thereof, comprises CDRs of an antibody disclosed herein with framework regions of the variable regions described herein having an amino acid sequence that is at least 95%, 96%, 97% or 99% identical to an antibody disclosed herein, including sequences in the cited references. The sequences and disclosure specifically recited herein are expressly incorporated by reference.

In some embodiments, the TAGE agent comprises an antigen binding polypeptide that binds to a protein expressed on the surface of cells selected from hematopoietic stem cells (HSCs), hematopoetic progenitor stem cells (HPSCs), natural killer cells, macrophages, DC cells, non-DC myeloid cells, B cells, T cells (e.g., activated T cells), fibroblasts, or other cells. In some embodiments, the T cells are CD4 or CD8 T cells. In certain embodiments, the T cells are regulatory T cells (T regs) or effector T cells. In some embodiments, the T cells are tumor infiltrating T cells. In some embodiments, the cell is a hematopoietic stem cell (HSCsO or a hematopoietic progenitor cells (HPSCs). In some embodiments, the macrophages are M1 or M2 macrophages.

In certain embodiments, the antigen binding protein of the TAGE agent is an antigen-binding fragment. Examples of such fragments include, but are not limited to, a domain antibody, a nanobody, a unibody, an scFv, a Fab, a BiTE, a diabody, a DART, a minibody, a F(ab′)₂, or an intrabody.

In one embodiment, the antigen binding polypeptide of the TAGE agent is a nanobody.

In one embodiment, the nanobody is an anti-MHCII nanobody. In one embodiment, the anti-MHCII nanobody comprises the amino acid sequence of SEQ ID NO: 110.

In one embodiment, the nanobody is an anti-EGFR nanobody. In one embodiment, the anti-EGFR nanobody comprises the amino acid sequence of SEQ ID NO: 111.

In one embodiment, the nanobody is an anti-HER2 nanobody. In one embodiment, the anti-HER2 nanobody comprises the amino acid sequence of SEQ ID NO: 112.

In one embodiment, a TAGE agent comprises a domain antibody and a site-directed modifying polypeptide. Domain antibodies (dAbs) are small functional binding units of antibodies, corresponding to the variable regions of either the heavy (VH) or light (VL) chains of human antibodies. Domain Antibodies have a molecular weight of approximately 13 kDa. Domantis has developed a series of large and highly functional libraries of fully human VH and VL dAbs (more than ten billion different sequences in each library), and uses these libraries to select dAbs that are specific to therapeutic targets. In contrast to many conventional antibodies, domain antibodies are well expressed in bacterial, yeast, and mammalian cell systems. Further details of domain antibodies and methods of production thereof may be obtained by reference to U.S. Pat. Nos. 6,291,158; 6,582,915; 6,593,081; 6,172,197; 6,696,245; U.S. Serial No. 2004/0110941; European patent application No. 1433846 and European Patents 0368684 & 0616640; WO05/035572, WO04/101790, WO04/081026, WO04/058821, WO04/003019 and WO03/002609, each of which is herein incorporated by reference in its entirety.

In one embodiment, a TAGE agent comprises a nanobody and a site-directed modifying polypeptide. Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a perfectly stable polypeptide harbouring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are extremely stable, can be administered by means other than injection (see, e.g., WO 04/041867, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and may be produced in prokaryotic or eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula or Pichia) (see, e.g., U.S. Pat. No. 6,838,254, which is herein incorporated by reference in its entirety). The production process is scalable and multi-kilogram quantities of Nanobodies have been produced. Because Nanobodies exhibit a superior stability compared with conventional antibodies, they can be formulated as a long shelf-life, ready-to-use solution.

The nanoclone method (see, e.g., WO 06/079372, which is herein incorporated by reference in its entirety) is a proprietary method for generating nanobodies against a desired target, based on automated high-throughout selection of B-cells and could be used in the context of the instant invention.

In one embodiment, a TAGE agent comprises a unibody and a site-directed modifying polypeptide. UniBodies are another antibody fragment technology, however this technology is based upon the removal of the hinge region of IgG4 antibodies. The deletion of the hinge region results in a molecule that is essentially half the size of traditional IgG4 antibodies and has a univalent binding region rather than the bivalent binding region of IgG4 antibodies. It is also well known that IgG4 antibodies are inert and thus do not interact with the immune system, which may be advantageous for the treatment of diseases where an immune response is not desired, and this advantage is passed onto UniBodies. For example, unibodies may function to inhibit or silence, but not kill, the cells to which they are bound. Additionally, unibody binding to cancer cells do not stimulate them to proliferate. Furthermore, because unibodies are about half the size of traditional IgG4 antibodies, they may show better distribution over larger solid tumors with potentially advantageous efficacy. UniBodies are cleared from the body at a similar rate to whole IgG4 antibodies and are able to bind with a similar affinity for their antigens as whole antibodies. Further details of UniBodies may be obtained by reference to patent application WO2007/059782, which is herein incorporated by reference in its entirety.

In one embodiment, a TAGE agent comprises an affibody and a site-directed modifying polypeptide. Affibody molecules represent a class of affinity proteins based on a 58-amino acid residue protein domain, derived from one of the IgG-binding domains of staphylococcal protein A. This three helix bundle domain has been used as a scaffold for the construction of combinatorial phagemid libraries, from which affibody variants that target the desired molecules can be selected using phage display technology (Nord K, Gunneriusson E, Ringdahl J, Stahl S, Uhlen M, Nygren P A, Binding proteins selected from combinatorial libraries of an α-helical bacterial receptor domain, Nat Biotechnol 1997; 15:772-7. Ronmark J, Gronlund H, Uhlen M, Nygren P A, Human immunoglobulin A (IgA)-specific ligands from combinatorial engineering of protein A, Eur J Biochem 2002; 269:2647-55). The simple, robust structure of Affibody molecules in combination with their low molecular weight (6 kDa), make them suitable for a wide variety of applications, for instance, as detection reagents (Ronmark J, Harmon M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren P A, Inhibition of the CD28-CD80 co-stimulation signal by a CD28-binding Affibody ligand developed by combinatorial protein engineering, Protein Eng 2003; 16:691-7). Further details of Affibodies and methods of production thereof may be obtained by reference to U.S. Pat. No. 5,831,012 which is herein incorporated by reference in its entirety.

In some embodiments, the antibody, antigen-binding fragment thereof, or antibody mimetic may specifically bind to an extracellular molecule (e.g., protein, glycan, lipid) localized on a target cell membrane or associated with a specific tissue with an Kd of at least about 1×10⁻⁴, 1×10⁻⁵, 1×10⁻⁶ M, 1×10⁻⁷ M, 1×10⁻⁸ M, 1×10⁻⁹ M, 1×10¹⁰ M, 1×10⁻¹¹ M, 1×10⁻¹² M, or more, and/or bind to an antigen with an affinity that is at least two-fold greater than its affinity for a nonspecific antigen. Such binding can result in antigen-mediated surface interactions. It shall be understood, however, that the binding protein may be capable of specifically binding to two or more antigens which are related in sequence. For example, the binding polypeptides of the invention can specifically bind to both human and a non-human (e.g., mouse or non-human primate) orthologs of an antigen.

In some embodiments, the antibody, antigen-binding fragment thereof, or antibody mimetic binds to a hapten which in turn specifically binds an extracellular cell surface protein (e.g., a Cas9-antibody-hapten targeting a cell receptor).

Binding or affinity between an antigen and an antibody can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet® analysis (forteBIO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmuno assay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

The antibody or antigen-binding fragment thereof, described herein can be in the form of full-length antibodies, bispecific antibodies, dual variable domain antibodies, multiple chain or single chain antibodies, and/or binding fragments that specifically bind an extracellular molecule, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the antibody is an IgG (e.g. IgG1, IgG2, IgG3 or IgG4).

In one embodiment, the antibody is Abciximab (ReoPro; CD41), alemtuzumab (Lemtrada, Campath; CD52), abrilumab (integrin α4β7), alacizumab pegol (VEGFR2), alemtuzumab (Lemtrada, Campath; CD52), anifrolumab (interferon α/β receptor), apolizumab (HLA-DR), aprutumab (FGFR2); aselizumab (L-selectin or CD62L), atezolizumab (Tecentriq; PD-L1), avelumab (Bavencio; PD-L1), azintuxizumab (CD319); basiliximab (Simulect; CD25), BCD-100 (PD-1), bectummomab (LymphoScan; CD22), belantamab (BCMA); belimumab (Benlysta; BAFF), bemarituzumab (FGFR2), benralizumab (Fasenra; CD125), bersanlimab (ICAM-1), bimagrumab (ACVR2B), bivatuzumab (CD44 v6), bleselumab (CD40), blinatumomab (Blincyto; CD19), blosozumab (SOST); brentuximab (Adcentris; CD30), brontictuzumab (Notch 1), cabiralizumab (CSF1R), camidanlumab (CD25), camrelizumab (PD-1), carotuximab (endoglin), catumaxomab (Removab; EpCAM, CD3), cedelizumab (CD4); cemipilimab (Libtayo; PCDC1), cetrelimab (PD-1), cetuximab (Erbitux; EGFR), cibisatamab (CEACAM5), cirmtuzumab (ROR1), cixutumumab (IGF-1 receptor, CD221), clenoliximab (CD4), coltuximab (CD19), conatumumab (TRAIL-R2), dacetuzumab (CD40), daclizumab (Zenapax; CD25), dalotuzumab (IGF-1 receptor, CD221), dapirolizumab pegol (CD154, CD40L), daratumumab (Darzalex; CD38), demcizumab (DLL4), denintuzumab (CD19), depatuxizumab (EGFR), drozitumab (DR5); DS-8201 (HER2), deligotuzumab (ERBB3, HER3), dupilumab (IL-4Rα), durvalumab (Imfinzi; PD-L1), duvortuxizumab (CD19, CD3E), efalizumab (CD11a), elgemtumab (ERBB3, HER3); elotuzumab (SLAMF7), emactuzumab (CSF1R), enapotamab (AXL), enavatuzumab (TWEAK receptor), enlimonomab pegol (ICAM-1, CD54), enoblituzumab (CD276), enoticumab (DLL4), epratuzumab (CD22), erlizumab (ITGB2, CD18), ertumaxomab (Rexomun; HER2/neu, CD3), etaracizumab (Abergin; integrin avP3), etigilimab (TIGIT), etrolizumab (integrin β₇), exbivirumab (hepatitis B surface antigen), fanolesomab (NeutroSpec; CD15), faralimomab (interferon receptor), farletuzumab (folate receptori), FBTA05 (Lymphomun, CD20), fibatuzumab (ephrin receptor A3), figitumumab (IGF-1 receptor, CD221), flotetuzumab (IL 3 receptor); foralumab (CD3 epsilon); futuximab (EGFR), galiximab (CD80), gancotamab (HER2/neu), ganitumab (IGF-1 receptor, CD221), gavilimomab (CD147, basigin), gemtuzumab (Mylotarg; CD33), gomiliximab (CD23, IgE receptor), ianalumab (BAFF-R), ibalizumab (Trogarzo; CD4), 1B1308 (PD-1), ibritumomab tiuxetan (CD20), icrucumab (VEGFR-1), ifabotuzumab (EPHA3), iladatuzumab (CD97B), imgatuzumab (EGFR), indusatumab (GUCY2C), inebilizumab (CD19), intetumumab (CD51), inolimomab (CD25), inotuzumab (Besponsa; CD22), ipilimumab (Yervoy; CD152), iomab-B (CD45), iratumumab (CD30), isatuximab (CD38), iscalimab (CD40), istiratumab (IGF1R, CD221), itolizumab (Alzumab, CD6), keliximab (CD4), laprituximab (EGFR), lemalesomab (NCA-90, granulocyte antigen), lenvervimab (hepatitis B surface antigen), leronlimab (CCR5), lexatumumab (TRAIL-R2), libivirumab (hepatitis B surface antigen), losatuxizumab (EGFR, ERBB1, HER1), lilotomab (CD37), lintuzumab (CD33), lirilumab (KIR2D), lorvotuzumab (CD56), lucatumumab (CD40), lulizumab pegol (CD28), lumiliximab (CD23, IgE receptor), lumretuzumab (ERBB3, HER3), lupartumab (LYPD3), mapatumumab (TRAIL-R1), margetuximab (HER2), maslimomab (T-cell receptor), mavrilimumab (GMCSF receptor α-chain), matuzumab (EGFR), mirvetuximab (folate receptor alpha), modotuximab (EGFR extracellular domain III), mogamulizumab (CCR4), monalizumab (NKG2A), mosunetuzumab (CD3E, MS4A1, CD20), moxetumomab pasudotox (CD22), muromonab-CD3 (CD3), nacolomab (C242 antigen), naratuximab (CD37), narnatumab (MST1R), natalizumab (Tysabri, integrin α₄), naxitamab (c-Met), necitumumab (EGFR), nemolizumab (IL31 RA), nimotuzumab (Theracim, Theraloc; EGFR), nirsevimab (RSVFR), nivolumab (PD-1), obinutuzumab (CD20), ocaratuzumab (CD20), ocrelizumab (CD20), odulimomab (LFA-1, CD11a), ofatumumab (CD20), olaratumab (PDGF-Rα), omburtamab (CD276), onartuzumab (human scatter factor receptor kinase), ontuxizumab (TEMi), onvatilimab (VSIR), opicinumab (LINGO-1), otelixizumab (CD3), otlertuzumab (CD37), oxelumab (OX-40), panitumumab (EGFR), patitumab (ERBB3, HER3), PDR001 (PD-1), pembrolizumab (Keytruda, PD-1), pertuzumab (Omnitarg, HER2/neu), pidilizumab (PD-1), pinatuzumab (CD22), plozalizumab (CCR2), pogalizumab (TNFR superfamily member 4), polatuzumab (CD79B), prilizimab (CD4), PRO 140 (CCR5), ramucirumab (Cyramza; VEGFR2), ravagalimab (CD40), relatlimab (LAG3), rinucumab (platelet-derived growth factor receptor beta); rituzimab (MabThera, Rituzan; CD20), robatumumab (IGF-1 receptor, CD221), roledumab (RHD), rovelizumab (LeukArrest; CD11, CD18), rozanolixizumab (FCGRT), ruplizumab (Antova; CD154, CD40L), SA237 (IL-6R), sacituzumab (TROP-2), samalizumab (CD200), samrotamab (LRRC15), satralizumab (IL6 receptor), seribantumab (ERBB3, HER3), setrusumab (SOST), SGN-CD19A (CD19), SHP647 (mucosal addressin cell adhesion molecule), siplizumab (CD2), sirtratumab (SLITRK6), spartalizumab (PDCD1, CD279), sulesomab (NCA-90, granulocyte antigen), suptavumab (RSVFR), tabalumab (BAFF), tadocizumab (integrin α_{IIb}β₃) talacotuzumab (CD123), taplitumomab paptox (CD19), tarextumab (Notch receptor), tavolimab (CD134), telisotuzumab (HGFR), teneliximab (CD40), tepoditamab (dendritic cell-associated lectin 2), teprotumomab (IGF-1 receptor, CD221), tetulomab (CD37), TGN1412 (CD28), tibulizumab (BAFF), tigatuzumab (TRAIL-R2), timigutuzumab (HER2), tiragotumab (TIGIT), tislelizumab (PCDC1, CD279), tocilizumab (Actemra, RoActemra; IL-6 receptor), tomuzotuximab (EGFR, HER1), toralizumab (CD154, CD40L), tositumomab (Bexxar; CD20), tovetumab (PDGFRA), trastuzumab (Herceptin; HER2/neu); trastuzumab (Kadcyla; HER2/neu); tregalizumab (CD4), tremelimumab (CTLA4), ublituximab (MS4A1), ulocuplumab (CXCR4, CD184), urelumab (4-1BB, CD137), utomilumab (4-1BB, CD137), vadastuximab talirine (CD33), vanalimab (CD40), vantictumab (Frizzled receptor), varlilumab (CD27), vatelizumab (ITGA2, CD49b), vedolizumab (Entyvio; integrin α₄β₇), veltuzumab (CD20), vesencumab (NRP1), visilizumab (Nuvion; CD3), vobarilizumab (IL6R), volociximab (integrin α₅β₁), vonlerolizumab (CD134), vopratelimab (CD278, ICOS), XMAB-5574 (CD19), zalutumumab (HuMax-EGFr; EGFR), zanolimumab (HuMax-CD4; CD4), zatuximab (HER1), zenocutuzumab (ERBB3, HER3), ziralimumab (CD147, basigin); zolbetuximab (Claudin 18 Isoform 2), zolimomab (CD5), 3F8 (GD2 ganglioside), adecatumumab (EpCAM), altumomab (Hybri-ceaker; CEA), amatuximab (mesothelin), anatumomab mafenatox (TAG-72), anetumab (MSLN), arcitumomab (CEA), atorolimumab (Rhesus factor); bavituximab (phosphatidylserine), besilesomab (Scintimun; CEA-related antigen), cantuzumab (MUC1), caplacizumab (Cablivi; VWF), clivatuzumab tetraxetan (hPAM4-Cide; MUC1), codrituzumab (glypican 3), crizanlizumab (selectin P), crotedumab (GCGR), dinutuximab (Unituxin; GD2 ganglioside), ecromeximab (GD3 ganglioside); edrecolomab (EpCAM); elezanumab (RGMA), fgatipotuzumab (MUC1), glembatumumab (GPNMB), igovomab (Indimacis-125; CA-125), IMAB362 (CLDN18.2), imaprelimab (MCAM), inclacumab (selectin P), indatuximab (SDC1), labetuzumab (CEA-Cide, CEA), lifastuzumab (phosphate-sodium co-transporter), minretumomab (TAG-72), mitumomab (GD3 ganglioside), morolimumab (Rhesus factor), naptumomab estafenatox (5T4), oportuzumab monatox (EpCAM), oregovomab (CA-125), pankomab (tumor specific glycosylation of MUC1), pemtumomab (Theragyn, MUC1), racotumomab (Vaxira, NGNA ganglioside), radretumab (fibronectina extra domain-B), refanezumab (myelin-associated glycoprotein), sontuzumab (episialin); TRBS07 (GD2 ganglioside), tucotuzumab celmoleukin (EpCAM), loncastuximab (CD19), milatuzumab (CD74), satumomab pendetide (TAG-72), sofituzumab (CA-125), solitomab (EpCAM), abituzumab (CD51), adalimumab (Humira; TNF-α), brodalumab (Siliq; IL-17 receptor), cergutuzumab amunaleukin (CEA), golimumab (Simponi; TNF-α), infliximab (Remicade; TNF-α), varisacumab (VEGFR2), sarilumab (Kevzara, IL-6R), siltuximab (Sylvant; soluble IL-6, IL-6R), or avicixizumab (DLL4, VEGFA). Antibodies or antigen binding proteins to cell surface targets disclosed in the previous sentence with respect to specific antibodies are also contemplated as a target on the cell surface, e.g., HER2.

In other embodiments, an antibody that can be used in the compositions and methods disclosed herein is an antibody known to internalize and be effective as an antibody drug conjugate (ADC). Examples of such antibodies, which can be used in TAGE agents described herein includes, but are not limited to, anetumab (mesothelin), aorutumab (FGFR2), azintuxizumab (SLAMF7), belantamab (TNFRSFi7), bivatuzumab (CD44v6), brentuximab (CD30), camidanlumab (CD25), cantuzumab (CanAg), cantuzumab (CanAg), clivatuzumab (MUC1), cofetuzumab (PTK7), coltuximab (CD19), denintuzumab (CD19), depatuxizumab (EGFR), enapotamab (AXL), enfortumab (Nectin-4), epratuzumab (CD22), gemtuzumab (CD33), glembatumumab (GPNMB), hertuzumab (HER2), iladatuzumab (CD79B), indatuximab (CD138), industuzumab (GCC), inotuzumab (CD22), labetuzumab (CEA-CAM4), ladiratuzumab (LIV-1), laprituximab (EGFR), lifastuzumab (SLC34A2), loncastuximab (CD19), lorvotuzumab (CD56), losatuximab (EGFR), lupartumab (LYPD3), iratumumab (CD30), milatuzumab (CD74), mirvetuximab (PSMA), naratuximab (CD37), pinatuzumab (CD22), polatuzumab (CD79B), rovalpituzumab (DLL3), sacituzumab (TACSTD2), samrotamab (LRRC15), sirtratumab (SLTRK6), sofituzumab (mucin 16), telisotuzumab (c-Met), tisotumab (TF), trastuzumab (ERBB2), vadastuximab (CD33), vandortuzumab (STEAPi), or vorsetuzumab (CD70). Antibodies directed to the targets referenced in the previous sentence are also contemplated herein. Additional cell surface targets that have been shown to be effective ADC targets include, but are not limited to, KAAG-1, PRLR, DLK1, ENPP3, FLT3, ADAM-9, CD248, endothelin receptor ETB, HER3, TM4SF1, SLC44A4, 5T4, AXL, Ror2, CA9, CFC1B, MT1-MMP, HGFR, CXCR4, TIM-1, CD166, CD163, GPC2, S. Aureus, folate receptor, FXYD5, CD20, CA125, AMHRII, BCMA, CDH-6, CD98, SAIL, CLDN6, CLDN18.2, EGFRviii, alpha-V integrin, CD123, HLA-DR, CD117, FGFR, EphA, CD205, CD276, CD99, Globo H, MTX3, MTX5, P-cadherin, SSTR2, EFNA4, Notch3, TROP2, Ganglioside GD3, FOLH1, LY6E, CEA-CAM5, LAMP1, Le(y), CD352, ER-alpha36, STn, folate receptor alpha, P. aeruginosa antigen, CD38, H-Ferritin, SLeA, NKA, CD147, OFP, SLITRK5, EphrinA4, VEGFR2, GCL, CEACAMi, CEACAM6, or NaPi2b.

The antibody, or antigen-binding fragment thereof, described herein can be in the form of full-length antibodies, bispecific antibodies, dual variable domain antibodies, multiple chain or single chain antibodies, and/or binding fragments that specifically bind an extracellular molecule, including but not limited to Fab, Fab′, (Fab′)2, Fv), scFv (single chain Fv), surrobodies (including surrogate light chain construct), single domain antibodies, camelized antibodies and the like. They also can be of, or derived from, any isotype, including, for example, IgA (e.g., IgA1 or IgA2), IgD, IgE, IgG (e.g. IgG1, IgG2, IgG3 or IgG4), or IgM. In some embodiments, the antibody is an IgG (e.g. IgG1, IgG2, IgG3 or IgG4). In certain embodiments, the antigen binding polypeptide is a multispecific protein, such as a multispecific (e.g., bispecific) antibody.

In one embodiments, the antigen binding protein is a bispecific molecule comprising a first antigen binding site from a first antibody that binds to a target on the extracellular cell membrane of a cell and a second antigen binding site with a different binding specificity, such as a binding specificity for a second target on the extracellular cell membrane of the cell, i.e. a bispecific antibody wherein the first and second antigen binding sites do not cross-block each other for binding to either the first or the second antigen. Examples of target antigens are provided above. Thus, it is contemplated that a TAGE agent comprises a bispecific molecule that binds to two antigens, including those described herein, e.g., CTLA4 and CD44.

Exemplary bispecific antibody molecules comprise (i) two antibodies, one with a specificity to a first antigen and another to a second target that are conjugated together, (ii) a single antibody that has one chain or arm specific to a first antigen and a second chain or arm specific to a second antigen, (iii) a single chain antibody that has specificity to a first antigen and a second antigen, e.g., via two scFvs linked in tandem by an extra peptide linker; (iv) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (v) a chemically-linked bispecific (Fab′)₂ fragment; (vi) a Tandab, which is a fusion of two single chain diabodies resulting in a tetravalent bispecific antibody that has two binding sites for each of the target antigens; (vii) a flexibody, which is a combination of scFvs with a diabody resulting in a multivalent molecule; (viii) a so called “dock and lock” molecule, based on the “dimerization and docking domain” in Protein Kinase A, which, when applied to Fabs, can yield a trivalent bispecific binding protein consisting of two identical Fab fragments linked to a different Fab fragment; (ix) a so-called Scorpion molecule, comprising, e.g., two scFvs fused to both termini of a human Fc-region; and (x) a diabody.

Examples of platforms useful for preparing bispecific antibodies include but are not limited to BITE (Micromet), DART (MacroGenics), Fcab and Mab.sup.2 (F-star), Fc-engineered IgG1 (Xencor) or DuoBody (based on Fab arm exchange, Genmab).

Examples of different classes of bispecific antibodies include but are not limited to asymmetric IgG-like molecules, wherein the one side of the molecule contains the Fab region or part of the Fab region of at least one antibody, and the other side of the molecule contains the Fab region or parts of the Fab region of at least one other antibody; in this class, asymmetry in the Fc region could also be present, and be used for specific linkage of the two parts of the molecule; symmetric IgG-like molecules, wherein the two sides of the molecule each contain the Fab region or part of the Fab region of at least two different antibodies; IgG fusion molecules, wherein full length IgG antibodies are fused to extra Fab regions or parts of Fab regions; Fc fusion molecules, wherein single chain Fv molecules or stabilized diabodies are fused to Fcgamma regions or parts thereof; Fab fusion molecules, wherein different Fab-fragments are fused together; ScFv- and diabody-based molecules wherein different single chain Fv molecules or different diabodies are fused to eachother or to another protein or carrier molecule.

Examples of asymmetric IgG-like molecules include but are not limited to the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (EMD Serono), the Biclonic (Merus) and the DuoBody (Genmab A/S).

Example of symmetric IgG-like molecules include but are not limited to Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

Examples of IgG fusion molecules include but are not limited to Dual Variable Domain (DVD)-Ig (Abbott), IgG-like Bispecific (ImClone/Eli Lilly), Ts2Ab (Medlmmune/AZ) and BsAb (Zymogenetics), HERCULES (Biogen Idec) and TvAb (Roche).

Examples of Fc fusion molecules include but are not limited to ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv) 2-Fab (National Research Center for Antibody Medicine—China).

Examples of class V bispecific antibodies include but are not limited to F(ab)₂ (Medarex/Amgen), Dual-Action or Bis-Fab (Genentech), Dock-and-Lock (DNL) (ImmunoMedics), Bivalent Bispecific (Biotecnol) and Fab-Fv (UCB-Celltech). Examples of ScFv- and diabody-based molecules include but are not limited to Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COM BODY (Epigen Biotech).

Antibodies, antigen-binding fragments, or an antibody mimetic that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive antigen binding assay.

The antibodies or binding fragments described herein may also include modifications and/or mutations that alter the properties of the antibodies and/or fragments. Methods of engineering antibodies to include any modifications are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

Antibodies or fragments thereof, may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CLL-1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody (or antibody fragment), nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR− CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

### Antibody Mimetic

The TAGE agent may include an antibody mimetic capable of binding an antigen of interest. As detailed below, a wide variety of antibody fragment and antibody mimetic technologies have been developed and are widely known in the art. Generally, an antibody mimetic, described herein, are not structurally related to an antibody, and include adnectins, affibodies, DARPins, anticalins, avimers, versabodies, aptamers and SMIPS. An antibody mimetic uses binding structures that, while mimicking traditional antibody binding, are generated from and function via distinct mechanisms. Some of these alternative structures are reviewed in Gill and Damle (2006) 17: 653-658.

In one embodiment, a TAGE agent comprises an adnectin molecule and a site-directed modifying polypeptide. Adnectin molecules are engineered binding proteins derived from one or more domains of the fibronectin protein. Fibronectin exists naturally in the human body. It is present in the extracellular matrix as an insoluble glycoprotein dimer and also serves as a linker protein. It is also present in soluble form in blood plasma as a disulphide linked dimer. The plasma form of fibronectin is synthesized by liver cells (hepatocytes), and the ECM form is made by chondrocytes, macrophages, endothelial cells, fibroblasts, and some cells of the epithelium. As mentioned previously, fibronectin may function naturally as a cell adhesion molecule, or it may mediate the interaction of cells by making contacts in the extracellular matrix. Typically, fibronectin is made of three different protein modules, type I, type II, and type III modules. For a review of the structure of function of the fibronectin, see Pankov and Yamada (2002) J Cell Sci.; 115 (Pt 20):3861-3, Hohenester and Engel (2002) 21:115-128, and Lucena et al. (2007) Invest Clin. 48:249-262.

In one embodiment, adnectin molecules are derived from the fibronectin type III domain by altering the native protein which is composed of multiple beta strands distributed between two beta sheets. Depending on the originating tissue, fibronectin may contain multiple type III domains which may be denoted, e.g., 1 Fn3, 2Fn3, 3Fn3, etc. The 10Fn3 domain contains an integrin binding motif and further contains three loops which connect the beta strands. These loops may be thought of as corresponding to the antigen binding loops of the IgG heavy chain, and they may be altered by methods discussed below to specifically bind a target of interest. Preferably, a fibronectin type III domain useful for the purposes of this invention is a sequence which exhibits a sequence identity of at least 30%, at least 40%, at least 50%, at least 60%, at least 70%, at least 80%, at least 90%, or at least 95% to the sequence encoding the structure of the fibronectin type III molecule which can be accessed from the Protein Data Bank (PDB, rcsb.org/pdb/home/home.do) with the accession code: 1ttg. Adnectin molecules may also be derived from polymers of 10Fn3 related molecules rather than a simple monomeric 10Fn3 structure.

Although the native 10Fn3 domain typically binds to integrin, 10Fn3 proteins adapted to become adnectin molecules are altered so to bind antigens of interest. In one embodiment, the alteration to the 10Fn3 molecule comprises at least one mutation to a beta strand. In a preferred embodiment, the loop regions which connect the beta strands of the 10Fn3 molecule are altered to bind to the antigen of interest.

The alterations in the 10Fn3 may be made by any method known in the art including, but not limited to, error prone PCR, site-directed mutagenesis, DNA shuffling, or other types of recombinational mutagenesis which have been referenced herein. In one example, variants of the DNA encoding the 10Fn3 sequence may be directly synthesized in vitro, and later transcribed and translated in vitro or in vivo. Alternatively, a natural 10Fn3 sequence may be isolated or cloned from the genome using standard methods (as performed, e.g., in U.S. Pat. Application No. 20070082365), and then mutated using mutagenesis methods known in the art.

In one embodiment, a target antigen may be immobilized on a solid support, such as a column resin or a well in a microtiter plate. The target is then contacted with a library of potential binding proteins. The library may comprise 10Fn3 clones or adnectin molecules derived from the wild type 10Fn3 by mutagenesis/randomization of the 10Fn3 sequence or by mutagenesis/randomization of the 10Fn3 loop regions (not the beta strands). In a preferred embodiment the library may be an RNA-protein fusion library generated by the techniques described in Szostak et al., U.S. Pat. Nos. 6,258,558 and 6,261,804; Szostak et al., WO989/31700; and Roberts & Szostak (1997) 94:12297-12302. The library may also be a DNA-protein library (e.g., as described in Lohse, U.S. Pat. No. 6,416,950, and WO 00/32823). The fusion library is then incubated with the immobilized target antigen and the solid support is washed to remove non-specific binding moieties. Tight binders are then eluted under stringent conditions and PCR is used to amply the genetic information or to create a new library of binding molecules to repeat the process (with or without additional mutagenesis). The selection/mutagenesis process may be repeated until binders with sufficient affinity to the target are obtained. Adnectin molecules for use in the present invention may be engineered using the PROfusion™ technology employed by Adnexus, a Briston-Myers Squibb company. The PROfusion technology was created based on the techniques referenced above (e.g., Roberts & Szostak (1997) 94:12297-12302). Methods of generating libraries of altered 10Fn3 domains and selecting appropriate binders which may be used with the present invention are described fully in the following U.S. patent and patent application documents and are incorporated herein by reference: U.S. Pat. Nos. 7,115,396; 6,818,418; 6,537,749; 6,660,473; 7,195,880; 6,416,950; 6,214,553; 6,623,926; 6,312,927; 6,602,685; 6,518,018; 6,207,446; 6,258,558; 6,436,665; 6,281,344; 7,270,950; 6,951,725; 6,846,655; 7,078,197; 6,429,300; 7,125,669; 6,537,749; 6,660,473; and U.S. Pat. Application Nos. 20070082365; 20050255548; 20050038229; 20030143616; 20020182597; 20020177158; 20040086980; 20040253612; 20030022236; 20030013160; 20030027194; 20030013110; 20040259155; 20020182687; 20060270604; 20060246059; 20030100004; 20030143616; and 20020182597. The generation of diversity in fibronectin type III domains, such as 10Fn3, followed by a selection step may be accomplished using other methods known in the art such as phage display, ribosome display, or yeast surface display, e.g., Lipovsek et al. (2007) Journal of Molecular Biology 368: 1024-1041; Sergeeva et al. (2006) Adv Drug Deliv Rev. 58:1622-1654; Petty et al. (2007) Trends Biotechnol. 25: 7-15; Rothe et al. (2006) Expert Opin Biol Ther. 6:177-187; and Hoogenboom (2005) Nat Biotechnol. 23:1105-1116.

It should be appreciated by one of skill in the art that the methods references cited above may be used to derive antibody mimics from proteins other than the preferred 10Fn3 domain. Additional molecules which can be used to generate antibody mimics via the above referenced methods include, without limitation, human fibronectin modules 1 Fn3-9Fn3 and 11 Fn3-17Fn3 as well as related Fn3 modules from non-human animals and prokaryotes. In addition, Fn3 modules from other proteins with sequence homology to 10Fn3, such as tenascins and undulins, may also be used. Other exemplary proteins having immunoglobulin-like folds (but with sequences that are unrelated to the VH domain) include N-cadherin, ICAM-2, titin, GCSF receptor, cytokine receptor, glycosidase inhibitor, E-cadherin, and antibiotic chromoprotein. Further domains with related structures may be derived from myelin membrane adhesion molecule P0, CD8, CD4, CD2, class I MHC, T-cell antigen receptor, CD1, C2 and I-set domains of VCAM-1, I-set immunoglobulin fold of myosin-binding protein C, I-set immunoglobulin fold of myosin-binding protein H, I-set immunoglobulin-fold of telokin, telikin, NCAM, twitchin, neuroglian, growth hormone receptor, erythropoietin receptor, prolactin receptor, GC-SF receptor, interferon-gamma receptor, beta-galactosidase/glucuronidase, beta-glucuronidase, and transglutaminase. Alternatively, any other protein that includes one or more immunoglobulin-like folds may be utilized to create a adnecting like binding moiety. Such proteins may be identified, for example, using the program SCOP (Murzin et al., J. Mol. Biol. 247:536 (1995); Lo Conte et al., Nucleic Acids Res. 25:257 (2000).

In one embodiment, a TAGE agent comprises an aptamer and a site-directed modifying polypeptide. An “aptamer” used in the compositions and methods disclosed herein includes aptamer molecules made from either peptides or nucleotides. Peptide aptamers share many properties with nucleotide aptamers (e.g., small size and ability to bind target molecules with high affinity) and they may be generated by selection methods that have similar principles to those used to generate nucleotide aptamers, for example Baines and Colas. 2006. Drug Discov Today. 11 (7-8):334-41; and Bickle et al. 2006. Nat Protoc. 1 (3):1066-91 which are incorporated herein by reference.

In certain embodiment, an aptamer is a small nucleotide polymer that binds to a specific molecular target. Aptamers may be single or double stranded nucleic acid molecules (DNA or RNA), although DNA based aptamers are most commonly double stranded. There is no defined length for an aptamer nucleic acid; however, aptamer molecules are most commonly between 15 and 40 nucleotides long.

Aptamers often form complex three-dimensional structures which determine their affinity for target molecules. Aptamers can offer many advantages over simple antibodies, primarily because they can be engineered and amplified almost entirely in vitro. Furthermore, aptamers often induce little or no immune response.

Aptamers may be generated using a variety of techniques, but were originally developed using in vitro selection (Ellington and Szostak. (1990) Nature. 346 (6287):818-22) and the SELEX method (systematic evolution of ligands by exponential enrichment) (Schneider et al. 1992. J Mol Biol. 228 (3):862-9) the contents of which are incorporated herein by reference. Other methods to make and uses of aptamers have been published including Klussmann. The Aptamer Handbook Functional Oligonucleotides and Their Applications. ISBN: 978-3-527-31059-3; Ulrich et al. 2006. Comb Chem High Throughput Screen 9 (8):619-32; Cerchia and de Franciscis. 2007. Methods Mol Biol. 361:187-200; Ireson and Kelland. 2006. Mol Cancer Ther. 2006 5 (12):2957-62; U.S. Pat. Nos. 5,582,981; 5,840,867; 5,756,291; 6,261,783; 6,458,559; 5,792,613; 6,111,095; and U.S. patent application U.S. Pub. No. US20070009476A1; U.S. Pub. No. US20050260164A1; U.S. Pat. No. 7,960,102; and U.S. Pub. No. US20040110235A1 which are all incorporated herein by reference.

The SELEX method is clearly the most popular and is conducted in three fundamental steps. First, a library of candidate nucleic acid molecules is selected from for binding to specific molecular target. Second, nucleic acids with sufficient affinity for the target are separated from non-binders. Third, the bound nucleic acids are amplified, a second library is formed, and the process is repeated. At each repetition, aptamers are chosen which have higher and higher affinity for the target molecule. SELEX methods are described more fully in the following publications, which are incorporated herein by reference: Bugaut et al. 2006. 4 (22):4082-8; Stoltenburg et al. 2007 Biomol Eng. 2007 24 (4):381-403; and Gopinath. 2007. Anal Bioanal Chem. 2007. 387 (1):171-82.

In one embodiment, a TAGE agent comprises a DARPin and a site-directed modifying polypeptide. DARPins (Designed Ankyrin Repeat Proteins) are one example of an antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains.

DARPins can be produced in bacterial expression systems at very high yields and they belong to the most stable proteins known. Highly specific, high-affinity DARPins to a broad range of target proteins, including human receptors, cytokines, kinases, human proteases, viruses and membrane proteins, have been selected. DARPins having affinities in the single-digit nanomolar to picomolar range can be obtained.

DARPins have been used in a wide range of applications, including ELISA, sandwich ELISA, flow cytometric analysis (FACS), immunohistochemistry (IHC), chip applications, affinity purification or Western blotting. DARPins also proved to be highly active in the intracellular compartment for example as intracellular marker proteins fused to green fluorescent protein (GFP). DARPins were further used to inhibit viral entry with IC50 in the pM range. DARPins are not only ideal to block protein-protein interactions, but also to inhibit enzymes. Proteases, kinases and transporters have been successfully inhibited, most often an allosteric inhibition mode. Very fast and specific enrichments on the tumor and very favorable tumor to blood ratios make DARPins well suited for in vivo diagnostics or therapeutic approaches.

Additional information regarding DARPins and other DRP technologies can be found in U.S. Patent Application Publication No. 2004/0132028 and International Patent Application Publication No. WO 02/20565, both of which are hereby incorporated by reference in their entirety.

In one embodiment, a TAGE agent comprises an anticalin and a site-directed modifying polypeptide. Anticalins are an additional antibody mimetic technology, however in this case the binding specificity is derived from lipocalins, a family of low molecular weight proteins that are naturally and abundantly expressed in human tissues and body fluids. Lipocalins have evolved to perform a range of functions in vivo associated with the physiological transport and storage of chemically sensitive or insoluble compounds. Lipocalins have a robust intrinsic structure comprising a highly conserved β-barrel which supports four loops at one terminus of the protein. These loops form the entrance to a binding pocket and conformational differences in this part of the molecule account for the variation in binding specificity between individual lipocalins.

While the overall structure of hypervariable loops supported by a conserved β-sheet framework is reminiscent of immunoglobulins, lipocalins differ considerably from antibodies in terms of size, being composed of a single polypeptide chain of 160-180 amino acids which is marginally larger than a single immunoglobulin domain.

In one embodiment, a TAGE agent comprises a lipocalin and a site-directed modifying polypeptide. Lipocalins are cloned and their loops are subjected to engineering in order to create anticalins. Libraries of structurally diverse anticalins have been generated and anticalin display allows the selection and screening of binding function, followed by the expression and production of soluble protein for further analysis in prokaryotic or eukaryotic systems. Studies have successfully demonstrated that anticalins can be developed that are specific for virtually any human target protein can be isolated and binding affinities in the nanomolar or higher range can be obtained.

Anticalins can also be formatted as dual targeting proteins, so-called duocalins. A duocalin binds two separate therapeutic targets in one easily produced monomeric protein using standard manufacturing processes while retaining target specificity and affinity regardless of the structural orientation of its two binding domains.

Modulation of multiple targets through a single molecule is particularly advantageous in diseases known to involve more than a single causative factor. Moreover, bi- or multivalent binding formats such as duocalins have significant potential in targeting cell surface molecules in disease, mediating agonistic effects on signal transduction pathways or inducing enhanced internalization effects via binding and clustering of cell surface receptors. Furthermore, the high intrinsic stability of duocalins is comparable to monomeric Anticalins, offering flexible formulation and delivery potential for Duocalins.

Additional information regarding anticalins can be found in U.S. Pat. No. 7,250,297 and International Patent Application Publication No. WO 99/16873, both of which are hereby incorporated by reference in their entirety.

Another antibody mimetic technology useful in the context of the instant invention are avimers. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. Other potential advantages include simple and efficient production of multitarget-specific molecules in Escherichia coli, improved thermostability and resistance to proteases. Avimers with sub-nanomolar affinities have been obtained against a variety of targets.

Additional information regarding avimers can be found in U.S. Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

In one embodiment, a TAGE agent comprises a versabody and a site-directed modifying polypeptide. Versabodies are another antibody mimetic technology that could be used in the context of the instant invention. Versabodies are small proteins of 3-5 kDa with >15% cysteines, which form a high disulfide density scaffold, replacing the hydrophobic core that typical proteins have. The replacement of a large number of hydrophobic amino acids, comprising the hydrophobic core, with a small number of disulfides results in a protein that is smaller, more hydrophilic (less aggregation and non-specific binding), more resistant to proteases and heat, and has a lower density of T-cell epitopes, because the residues that contribute most to MHC presentation are hydrophobic. All four of these properties are well-known to affect immunogenicity, and together they are expected to cause a large decrease in immunogenicity.

The inspiration for versabodies comes from the natural injectable biopharmaceuticals produced by leeches, snakes, spiders, scorpions, snails, and anemones, which are known to exhibit unexpectedly low immunogenicity. Starting with selected natural protein families, by design and by screening the size, hydrophobicity, proteolytic antigen processing, and epitope density are minimized to levels far below the average for natural injectable proteins.

Given the structure of versabodies, these antibody mimetics offer a versatile format that includes multi-valency, multi-specificity, a diversity of half-life mechanisms, tissue targeting modules and the absence of the antibody Fc region. Furthermore, versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life.

Additional information regarding versabodies can be found in U.S. Patent Application Publication No. 2007/0191272 which is hereby incorporated by reference in its entirety.

In one embodiment, a TAGE agent comprises an SMIP and a site-directed modifying polypeptide. SMIPs™ (Small Modular ImmunoPharmaceuticals-Trubion Pharmaceuticals) are engineered to maintain and optimize target binding, effector functions, in vivo half life, and expression levels. SMIPS consist of three distinct modular domains. First they contain a binding domain which may consist of any protein which confers specificity (e.g., cell surface receptors, single chain antibodies, soluble proteins, etc). Secondly, they contain a hinge domain which serves as a flexible linker between the binding domain and the effector domain, and also helps control multimerization of the SMIP drug. Finally, SMIPS contain an effector domain which may be derived from a variety of molecules including Fc domains or other specially designed proteins. The modularity of the design, which allows the simple construction of SMIPs with a variety of different binding, hinge, and effector domains, provides for rapid and customizable drug design.

More information on SMIPs, including examples of how to design them, may be found in Zhao et al. (2007) Blood 110:2569-77 and the following U.S. Pat. App. Nos. 20050238646; 20050202534; 20050202028; 20050202023; 20050202012; 20050186216; 20050180970; and 20050175614.

The detailed description of antibody fragment and antibody mimetic technologies provided above is not intended to be a comprehensive list of all technologies that could be used in the context of the instant specification. For example, and also not by way of limitation, a variety of additional technologies including alternative polypeptide-based technologies, such as fusions of complimentary determining regions as outlined in Qui et al., Nature Biotechnology, 25 (8) 921-929 (2007), which is hereby incorporated by reference in its entirety, as well as nucleic acid-based technologies, such as the RNA aptamer technologies described in U.S. Pat. Nos. 5,789,157, 5,864,026, 5,712,375, 5,763,566, 6,013,443, 6,376,474, 6,613,526, 6,114,120, 6,261,774, and 6,387,620, all of which are hereby incorporated by reference, could be used in the context of the instant invention.

TAGE Agent Constructs

In some embodiments, the TAGE agent comprises in order from N-terminus to C-terminus an antigen-binding polypeptide and a site-directed modifying polypeptide (e.g., Cas9).

In some embodiments, the TAGE agent comprises in order from N-terminus to C-terminus a site-directed modifying polypeptide (e.g., Cas9) and an antigen-binding polypeptide.

In some embodiments, the TAGE agent comprises in order from N-terminus to C-terminus a site-directed modifying polypeptide (e.g., Cas9), two nuclear localization signals (e.g., 2×SV40 NLSs), and SpyCatcher. For example, the TAGE agent may comprise a Cas9-2×NLS-SpyCatcher construct, which may in turn be conjugated to an antigen-binding polypeptide linked to a SpyTag.

In some embodiments, the TAGE agent comprises in order from N-terminus to C-terminus a SpyCatcher, a site-directed modifying polypeptide (e.g., Cas9), and two nuclear localization signals (e.g., 2×SV40 NLSs). For example, the TAGE agent may comprise a SpyCatcher-Cas9-2×NLS construct, which may in turn be conjugated to an antigen-binding polypeptide linked to a SpyTag.

In some embodiments, the TAGE agent comprises in order from N-terminus to C-terminus a series of polypeptides linked together by peptide linkers (e.g., a genetic fusion) or chemical linkers selected from Table 1. In some embodiments, a construct as set forth in Table 1 further includes one or more peptide linkers between the indicated polypeptides. In certain embodiments, a construct set forth in Table 1 further includes a peptide sequence corresponding to a HRV 3C Protease Cleavage site.

TABLE 1
Examples of TAGE Agents or Fragments Thereof

| | Other Constructs |
|---|---|
| | (e.g., for conjugation to a site-directed |
| Constructs including a Site-Directed | polypeptide via a conjugation moiety) |
| Modifying Polypeptide | “Ab” refers to antibody. |
| SpyCatcher-Cas9(WT)-2xNLS | Anti-CD11a Ab-SpyTag or |
| | SpyTag-Anti-CD11a Ab |
| Cas9(WT)-2xNLS-Spycatcher-4xNLS | Anti-CD11a F(ab′)2-SpyTag or |
| | SpyTag-Anti-CD11a F(ab′)2 |
| Cas9(WT)-2xNLS-Spycatcher-HTN | Anti-CD25 Ab-SpyTag or |
| | SpyTag-Anti-CD25 Ab |
| 4xNLS-Spycatcher-Cas9(WT)-2xNLS | Anti-CD25 F(ab′)2-SpyTag or |
| | SpyTag-Anti-CD25 F(ab′)2 |
| HTN-Spycatcher-Cas9(WT)-2xNLS | Anti-CD27-Ab SpyTag or |
| | SpyTag-Anti-CD27 Ab |
| SpyCatcher-Cas9(WT)-2xNLS | Anti-CD44-Ab SpyTag or |
| | SpyTag-Anti-CD44 Ab |
| Cas9(WT)-2xNLS-Spycatcher-4xNLS | Anti-CD52-Ab SpyTag or |
| | SpyTag-Anti-CD52 Ab |
| Cas9(WT)-2xNLS-Spycatcher-HTN | Anti-CD54-Ab SpyTag or |
| | SpyTag-Anti-CD54 Ab |
| (SpyCatcher-Cas9(WT)-2xNLS)₂ | Anti-GITR-Ab SpyTag or |
| | SpyTag-Anti-GITR Ab |
| SpyCatcher-TDP-Cas9 | Anti-HLA-DR-Ab SpyTag or |
| | SpyTag-Anti-HLA-DR Ab |
| SpyCatcher-TDP-Cas9-KDEL | Anti-ICOS-Ab SpyTag or |
| | SpyTag-Anti-ICOS Ab |
| Cas9(C80A)-MHCiiNb-2XNLS | Anti-OX40-Ab SpyTag or |
| | SpyTag-Anti-OX40 Ab |
| Cas9-2xNLS-Darpin(EpCam) | Anti-PD-L1-Ab SpyTag or |
| | SpyTag-Anti-PD-L2 Ab |
| Cas9-2xNLS-ProteinA | Anti-PD-1-Ab SpyTag or |
| | SpyTag-Anti-PD-1 Ab |
| | Anti-CTLA-4 Ab-SpyTag or |
| | SpyTag-Anti-CTLA-4 Ab |
| | Anti-FAP Ab-SpyTag or |
| | SpyTag-Anti-Fap Ab |
| | Anti-Fap(F(ab′)2-SpyTag or |
| | SpyTag-Anti-Fap(F(ab′)2 |
| | Anti-CD22 Ab-Halo Tag or |
| | Halo Tag-Anti-CD22 Ab |
| | Anti-Fap Ab-Halo Tag or |
| | Halo Tag-Anti-Fap Ab |
| | Anti-CTLA-4 Ab-Halo Tag or |
| | Halo Tag-Anti-CLTA-4 Ab |

In some embodiments, the TAGE agent comprises a first series of polypeptides (e.g., a first genetic fusion, such as a fusion selected from Table 1) and a second series of polypeptides (e.g., a second genetic fusion, such as a fusion selected from Table 1), wherein the first and second genetic fusions stably associate in a non-covalent manner or covalent manner, e.g., via complementary conjugation moieties, such as SpyCatcher/Spytag or Halo/Halo-Tag or ligand).

In some embodiments, the TAGE comprises an Antibody-SpyTag fusion (in order from N-terminus to C-terminus) conjugated to SpyCatcher-Cas9(WT)-2×NLS (in order form N-terminu to C-terminus).

In some embodiments, the TAGE comprises an Antibody-SpyTag fusion (in order from N-terminus to C-terminus) conjugated to (SpyCatcher-Cas9(WT)-2×NLS)₂ (in order form N-terminu to C-terminus).

In some embodiments, the TAGE comprises an Antibody-SpyTag fusion (in order from N-terminus to C-terminus) conjugated to Cas9(WT)-2×NLS-Spycatcher-4×NLS (in order form N-terminu to C-terminus).

In some embodiments, the TAGE comprises an Antibody-SpyTag fusion (in order from N-terminus to C-terminus) conjugated to Cas9(WT)-2×NLS-Spycatcher-HTN (in order form N-terminu to C-terminus).

In some embodiments, the TAGE comprises an Antibody-SpyTag fusion (in order from N-terminus to C-terminus) conjugated to 4×NLS-Spycatcher-Cas9(WT)-2×NLS (in order form N-terminu to C-terminus).

In some embodiments, the TAGE comprises an Antibody-SpyTag fusion (in order from N-terminus to C-terminus) conjugated to HTN-Spycatcher-Cas9(WT)-2×NLS (in order form N-terminu to C-terminus).

### III. Methods of Use

A TAGE agent described herein can be used to modify the genome of a target cell. The method comprises contacting the target cell with a TAGE agent disclosed herein, such that at least the site-directed modifying polypeptide is internalized into the cell and subsequently modifies the genome (or target nucleic acid) of the targeted cell. Such methods may be used in an in vitro setting, ex vivo, or in vivo, including for therapeutic use where the modification of the genome of a subject in need thereof results in treatment of a disease or disorder.

The TAGE agent described herein can be used to target a site-directed modifying polypeptide to any cell displaying an antigen of interest. The cell can be a eukaryotic cell, including, but not limited to, a mammalian cell. Examples of mammalian cells that can be targeted (and have their genome's modified) by the TAGE agent of the invention include, but are not limited to, a mouse cell, a non-human primate cell, or a human cell.

The TAGE agent, in certain instances, can be used to edit specific cell types ex vivo or in vivo, such as hematopoietic stem cells (HSCs), hematopoietic progenitor stem cells (HPSCs), natural killer cells, macrophages, DC cells, non-DC myeloid cells, B cells, T cells (e.g., activated T cells), fibroblasts, or other cells. In some embodiments, the T cells are CD4 or CD8 T cells. In certain embodiments, the T cells are regulatory T cells (T regs) or effector T cells. In some embodiments, the T cells are tumor infiltrating T cells. In some embodiments, the cell is a hematopoietic stem cell (HSC) or a hematopoietic progenitor cells (HPSCs). In some embodiments, the macrophages are M0, M1, or M2 macrophages. In some embodiments, the TAGE agent is used to edit multiple (e.g., two or more) cell types selected from hematopoietic stem cells, hematopoietic progenitor stem cells (HPSCs), natural killer cells, macrophages, DC cells, non-DC myeloid cells, B cells, T cells (e.g., activated T cells), and fibroblasts.

In some embodiments, the TAGE agent further comprises a CPP and the method comprises contacting a T cell (e.g., a human T cell) with the TAGE agent.

In some embodiments, the TAGE agent further comprises a CPP and the method comprises contacting a macrophage (e.g., a human macrophage) with the TAGE agent.

In some embodiments, the TAGE agent further comprises a CPP and the method comprises contacting an HSC (e.g., a human HSC) with the TAGE agent. In some embodiments, the TAGE agent comprises a CPP and the method comprises contacting a cell in the bone marrow of a subject with the TAGE agent. In some embodiments, the cell is not a hematopoietic stem cell (e.g., fibroblast, macrophages, osteoblasts, ostclasts, or endothelial cells).

In some embodiments, the TAGE agent further comprises at least four NLSs and the method comprises contacting a T cell (e.g., a human T cell) with the TAGE agent.

In some embodiments, the TAGE agent further comprises at least four NLSs and the method comprises contacting a macrophage (e.g., a human macrophage) with the TAGE agent.

In some embodiments, the TAGE agent further comprises a CPP and the method comprises contacting an HSC (e.g., a human HSC) with the TAGE agent.

In some embodiments, the TAGE agent further comprises at least six NLSs and the method comprises contacting a T cell (e.g., a human T cell) with the TAGE agent.

In some embodiments, the TAGE agent further comprises at least four NLSs and the method comprises contacting a macrophage (e.g., a human macrophage) with the TAGE agent.

In some embodiments, the TAGE agent further comprises at least six NLSs and the method comprises contacting an HSC (e.g., a human HSC) with the TAGE agent.

In some embodiments, the TAGE agent comprises at least six NLSs and the method further comprises contacting a fibroblast (e.g., a human fibroblast) with the TAGE agent.

In some embodiments, the TAGE agent further comprises a His-TAT-NLS (HTN) peptide and the method comprises contacting a T cell with the TAGE agent (e.g., a human T cell).

In some embodiments, the TAGE agent further comprises an HTN peptide and the method comprises contacting a macrophage with the TAGE agent (e.g., a human macrophage).

In some embodiments, the TAGE agent further comprises an HTN peptide and the method comprises contacting an HSC (e.g., a human HSC) with the TAGE agent. In some embodiments, the TAGE agent comprises an HTN peptide and the method comprises contacting a cell in the bone marrow of a subject with the TAGE agent. In some embodiments, the cell is not a hematopoietic stem cell (e.g., fibroblast, macrophages, osteoblasts, ostclasts, or endothelial cells).

In some embodiments, the TAGE agent further comprises an HTN peptide and the method comprises contacting a fibroblast (e.g., a human fibroblast) with the TAGE agent.

In some embodiments, the TAGE agent further comprises an antibody and the method comprises contacting a T cell (e.g., a human T cell) with the TAGE agent.

In some embodiments, the TAGE agent comprises an antibody and the method further comprises contacting a macrophage (e.g., a human macrophage) with the TAGE agent.

In some embodiments, the TAGE agent comprises an antibody and the method further comprises contacting an HSC (e.g., a human HSC) with the TAGE agent.

In some embodiments, the TAGE agent further comprises an antibody and the method comprises contacting a fibroblast (e.g., a human fibroblast) with the TAGE agent

In some embodiments, the TAGE agent further comprises an anti-FAP antibody and the method comprises contacting a fibroblast (e.g., a human fibroblast). with the TAGE

In some embodiments, the TAGE agent further comprises an anti-CTLA-4 antibody and the method comprises contacting a T cell (e.g., a human T cell) with the TAGE agent.

In some embodiments, the TAGE agent further comprises an anti-CD25 antibody and the method comprises contacting a T cell (e.g., a human T cell) with the TAGE agent.

In some embodiments, the TAGE agent further comprises an anti-CD11a antibody and the method comprises contacting a T cell e.g., a human T cell). with the TAGE agent.

In certain embodiments, the site-directed modifying polypeptide of the TAGE agent produces a cleavage site at the target region of the genome of the target cell, subsequently modifying the genome of the cell and impacting gene expression. Thus, in one embodiment, the target region of the genome is a target gene. The site-directed modifying polypeptide's ability to modify the genome of the target cell provides, in certain embodiments, a way to modify expression of the target gene. Expression levels of a target nucleic acid, e.g., a gene, can be determined according to standard methods, where in certain circumstances, the method disclosed herein is effective to increase expression of the target gene relative to a reference level. Alternatively, in other circumstances, the method disclosed herein is able to decrease expression of the target gene relative to a reference level. Reference levels can be determined in standard assays using a non-specific guide RNA/site-directed modifying polypeptide, where increases or decreases in the target nucleic acid, e.g., gene, may be measured relative to the control.

Internalization of the site-directed modifying polypeptide can be determined according to standard internalization assays, as well as those described in the Examples below. In one embodiment, at least 1%, at least 2%, at least 3%, at least 4%, at least 5%, at least 8%, at least 9%, at least 10%, or at least 15% of the site-directed modifying polypeptide is internalized by the cell within a given time (e.g., one hour, two hours, three hours, or more than three hours) of contact of the TAGE agent with the extracellular cell-bound antigen. For instance, in certain embodiments, the site-directed modifying polypeptide is internalized by a target cell within one hour of contact of the TAGE agent with the extracellular cell-bound antigen at a higher efficiency versus a control agent, e.g., an unconjugated (i.e., without the antigen binding polypeptide) site-directed modifying polypeptide.

Internalization of the TAGE agent, or a component thereof, can be assessed using any internalization assays known in the art. For example, internalization of a TAGE agent, or a component thereof, can be assessed by attaching a detectable label (e.g. a fluorescent dye) to the peptide (and/or to the cargo to be transfected) or by fusing the peptide with a reporter molecule, thus enabling detection once cellular uptake has occurred, e.g., by means of FACS analysis or via specific antibodies. In some embodiments, one or more components of the TAGE agent is conjugated to a reporter molecule having a quenchable signal. For example, as described in Example 5, a FACS-based internalization assay can be utilized based on the detection of Alexa-488 labeled TAGE components (e.g., a protein component or nucleic acid guide) following incubation of the labeled component with cells for a given period of time, after which the results achieved with or without quenching with an anti-A488 antibody are compared. Labeled molecules that are internalized by a target cell are protected from quenching by the anti-A488 antibody and therefore retain a stronger Alexa488 signal relative to a control following quenching. In contrast, labeled molecules that are not internalized, and therefore remain on the cell surface, are susceptible to quenching by the anti-A488 antibody and therefore display a reduced Alexa488 signal relative to an unquenched control.

The TAGE agent described herein can be used to target a site-directed modifying polypeptide to any cell that can be targeted by a given extracellular cell membrane binding protein (e.g., antigen binding protein, ligand, or CPP). The cell can be a eukaryotic cell, including, but not limited to, a mammalian cell. Examples of mammalian cells that can be targeted (and have their genome's modified) by the TAGE agent of the invention include, but are not limited to, a mouse cell, a non-human primate cell, or a human cell. The eukaryotic cell can be one that exists (i) in an organism/tissue in vivo, (ii) in a tissue or group of cells ex vivo, or (iii) in an in vitro state. In certain instances, the eukaryotic cell herein can be as it exists in an isolated state (e.g., in vitro cells, cultured cells) or a non-isolated state (e.g., in a subject, e.g., a mammal, such as a human, non-human primate, or a mouse). A eukaryotic cell in certain embodiments is a mammalian cell, such as a human cell.

The ability of a TAGE agent to edit a target nucleic acid, e.g., gene, in a target cell can be determined according to methods known in the art, including, for example, phenotypic assays or sequencing assays. Such assays may determine the presence or absence of a marker associated with the gene or nucleic acid of the target cell that is being edited by the TAGE agent. For example, as described in the examples below, a CD47 flow cytometry assay can be used to determine the efficacy of a TAGE agent for gene editing. In the CD47 flow cytometry assay, an endogenous CD47 gene sequence in the target cell is targeted by the TAGE agent, where editing is evidenced by a lack of CD47 expression on the cell surface of the target cell. Levels of CD47 can be measured in a population of cells and compared to a control TAGE agent where a non-targeting guide RNA is used as a negative control in the same type of target cell. Decreases in the level of CD47, for example, relative to the control indicates gene editing of the TAGE agent. In certain instances, a decrease of at least 1%, at least 2%, at at least 5%, at least 10%, at least 15%, at least 20%, at least 25%, at least 30%, at least 35%, and so forth, relative to a control in a testing assay indicates nucleic acid, e.g., gene, editing by the TAGE agent. Ranges of the foregoing percentages are also contemplated herein. Other ways in which nucleic acid, e.g., gene, editing activity of a TAGE agent can be determined include sequence based assays, e.g., amplicon sequencing, known in the art.

In alternative embodiments, an endogenous sequence in the target cell is targeted by the TAGE agent, where editing is evidenced by an increase in expression of a marker on the cell surface of the target cell or intracellular (e.g., to account for intracellular tDtomato or fluorescent (e.g., GFP), etc., reporters). In such embodiments, increases in the level of a marker as detected by flow cytometry, for example, relative to the control indicates gene editing of the TAGE agent. In certain instances, an increase of the cell surface marker of at least 1%, at least 2%, at least 5%, at least 10%, at least 15%, at least 20%, at least 25%, at least 30%, at least 35%, and so forth, relative to a control in a testing assay indicates nucleic acid, e.g., gene, editing by the TAGE agent. In certain instances, an increase in expression of the cell surface marker of at least 2-fold, 3-fold, 4-fold, 5-fold, 6-fold, 7-fold, 8-fold, 9-fold, 10-fold, or more, and so forth, relative to a control in a testing assay indicates nucleic acid, e.g., gene, editing by the TAGE agent. For example, an increase in the expression of a fluorescent marker (e.g., TdTomato fluorescent system) can be used to measure an increase of editing by the TAGE agent. Ranges of the foregoing percentages are also contemplated herein. Other ways in which nucleic acid, e.g., gene, editing activity of a TAGE agent can be determined include sequence based assays, e.g., amplicon sequencing, known in the art.

In some embodiments, the TAGE agent targets an endogenous gene sequence (e.g., CD47) encoding a cell surface protein in the target cell, and editing is evidenced by the percentage of target cells that lack expression of the cell surface protein on the cell surface of the target cell. In some embodiments, the percentage of target cells that lack expression of the cell surface protein, as detected by flow cytometry, for example, relative to the control indicates gene editing of the TAGE agent. In certain instances, absence of a cell surface protein (e.g., CD47) in at least 0.05%, at least 0.1%, at least 0.2%, at least 0.3%, at least 0.4%, at least 0.5%, at least 0.6%, at least 0.7%, at least 0.8%, at least 0.9%, at least 1%, at least 2%, at least 5%, at least 10%, at least 15%, at least 20%, at least 25%, at least 30%, at least 35%, and so forth of target cells in a population of target cells as detected by a testing assay indicates nucleic acid, e.g., gene, editing by the TAGE agent. Ranges of the foregoing percentages are also contemplated herein. In some instances, the percentage of target cells with an absence of a cell surface protein (e.g., CD47) is increased by at least 2-fold, 3-fold, 4-fold, 5-fold, 6-fold, 7-fold, 8-fold, 9-fold, 10-fold, or more, relative to a control in a testing assay indicates nucleic acid, e.g., gene, editing by the TAGE agent. Other ways in which nucleic acid, e.g., gene, editing activity of a TAGE agent can be determined include sequence based assays, e.g., amplicon sequencing, known in the art.

In alternative embodiments, an endogenous sequence in the target cell is targeted by the TAGE agent, where editing is evidenced by a change in fold of the level of gene editing relative to a control (e.g., a non-edited target cell). In one embodiment, a certain fold increase or decrease of a cell surface marker as detected by flow cytometry, would indicate nucleic acid, e.g., gene, editing relative to a control, e.g., a TAGE agent with a non-targeting guide RNA, or a TAGE agent which lacks the antigen binding polypeptide as a negative control. In certain instances, the fold increase of the cell surface marker is at least 1 fold, at least 2 fold, at least 3 fold, at least 4 fold, at least 5 fold, at least 1-5 fold, at least 1-4 fold, at least 2-5 fold higher in level, and so forth, relative to a control. In certain instances, an increase in expression of the cell surface marker of at least 2-fold, 3-fold, 4-fold, 5-fold, 6-fold, 7-fold, 8-fold, 9-fold, 10-fold, or more, and so forth, relative to a control in a testing assay indicates nucleic acid, e.g., gene, editing by the TAGE agent. In certain instances, a decrease in expression of the cell surface marker of at least 2-fold, 3-fold, 4-fold, 5-fold, 6-fold, 7-fold, 8-fold, 9-fold, 10-fold, or more, and so forth, relative to a control in a testing assay indicates nucleic acid, e.g., gene, editing by the TAGE agent. Such a fold increase or decrease (depending on result of nucleic acid editing facilitated by the TAGE agent) would indicate nucleic acid, e.g., gene, editing by the TAGE agent. In certain instances, an increase in expression of the cell surface marker of at least 2-fold, 3-fold, 4-fold, 5-fold, 6-fold, 7-fold, 8-fold, 9-fold, 10-fold, or more, and so forth, relative to a control in a testing assay indicates nucleic acid, e.g., gene, editing by the TAGE agent. Ranges of the foregoing fold changes are also contemplated herein. Other ways in which nucleic acid, e.g., gene, editing activity of a TAGE agent can be determined include sequence based assays, e.g., amplicon sequencing, known in the art.

For methods in which the proteins (e.g., antibody binding polypeptide) are delivered to cells, the proteins can be produced using any method known in the art, e.g., through covalent or non-covalent linkages, or expression in a suitable host cell from nucleic acid encoding the variant protein. A number of methods are known in the art for producing proteins. For example, the proteins can be produced in and purified from yeast, bacteria, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the antigen binding polypeptide can be linked to a moiety that facilitates transfer into a cell, e.g., a lipid nanoparticle, optionally with a linker that is cleaved once the protein is inside the cell.

In some embodiments, the antigen binding polypeptide may deliver a site-specific modifying polypeptide into a cell via an endocytic process. Examples of such a process might include macropinocytosis, clathrin-mediated endocytosis, caveolae/lipid raft-mediated endocytosis, and/or receptor mediated endocytosis mechanisms (e.g., scavenger receptor-mediated uptake, proteoglycan-mediated uptake).

Once a site-specific modifying polypeptide is inside a cell, it may traverse an organelle membrane such as a nuclear membrane or mitochondrial membrane, for example. In certain embodiments, the site-specific modifying polypeptide includes at least one (e.g., at least 1, 2, 3, 4, or more) nuclear-targeting sequence (e.g., NLS). In other embodiments, the ability to traverse an organelle membrane such as a nuclear membrane or mitochondrial membrane does not depend on the presence of a nuclear-targeting sequence. Accordingly, in some embodiments, the site-specific modifying polypeptide does not include an NLS.

In some embodiments, the TAGE agent is administered to cells ex vivo, such as hematopoietic stem cells (HSCs) or hematopoietic progenitor stem cells (HSPCs). For example, upon administering a TAGE agent provided herein (e.g., an anti-CD34 TAGE agent, or a TAGE-CPP agent) to HSCs ex vivo, TAGE-edited HSCs may then be transplanted into a subject in need of a hematopoietic stem cell transplant.

In certain embodiments, the TAGE agent described herein may be administered to a subject, e.g., by local administration. In some embodiments, the TAGE agent may be administered to the subject transdermally, subcutaneously, intravenously, intramuscularly, intraocularly, intraosseously, or intratumorally.

The TAGE agent may be administered to a subject in a therapeutically effective amount (e.g., in an amount to achieve a level of genome editing that treats or prevents a disease in a subject). For example, a therapeutically effective amount of a TAGE agent may be administered to a subject having a cancer (e.g., a colon carcinoma or a melanoma), an eye disease, or a stem cell disorder. A therapeutically effective amount may depend on the mode of delivery, e.g., whether the TAGE agent is administered locally (e.g., by intradermal (e.g., via the flank or ear in the case of a mouse), intratumoral, intraosseous, intraocular, or intramuscular injection) or systemically.

The TAGE agents described herein may be formulated to be compatible with the intended route of administration, such as by intradermal, intratumoral, intraosseous, intraocular, or intramuscular injection. Solutions, suspensions, dispersions, or emulsions may be used for such administrations and may include a sterile diluent, such as water for injection, saline solution, polyethylene glycols, glycerine, propylene glycol or other synthetic solvents; anti-bacterial agents such as benzyl alcohol or methylparabens; antioxidants such as ascorbic acid or sodium bisulfate; buffers such as acetates, citrates or phosphates, and agents for the adjustment of tonicity such as sodium chloride or dextrose. The pH may be adjusted with acids or bases, such as hydrochloric acid or sodium hydroxide. Preparations may be enclosed in ampules, disposable syringes or multiple dose vials made of glass or plastic. In certain embodiments, a pharmaceutical composition comprises a TAGE agent and a pharmaceutically acceptable carrier.

The TAGE agents can be included in a kit, container, pack or dispenser, together with medical devices suitable for delivering the compositions to a subject, such as by intradermal, intratumoral, intraosseous, intraocular, or intramuscular injection. The compositions included in kits may be supplied in containers of any sort such that the life of the different components may be preserved and may not be adsorbed or altered by the materials of the container. For example, sealed glass ampules or vials may contain the compositions described herein that have been packaged under a neutral non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include bottles that are fabricated from similar substances as ampules, and envelopes that consist of foil-lined interiors, such as aluminum or an alloy. Other containers include test tubes, vials, flasks, bottles, syringes, etc. Some containers may have a sterile resealable access port, such as a bottle having a stopper that may be pierced repeatedly by a hypodermic injection needle.

A TAGE agent may be administered to a subject by a route in accordance with the therapeutic goal. A variety of routes may be used to deliver a TAGE agent to desired cells or tissues, including systemic or local delivery.

In certain embodiments, a TAGE agent may be administered to a subject having a cancer, such as a colon carcinoma or a melanoma. In some embodiments, the cancer is, for example, a melanoma, a urogenetical cancer, a non-small cell lung cancer, a small-cell lung cancer, a lung cancer, a leukemia, a hepatocarcinoma, a retinoblastoma, an astrocytoma, a glioblastoma, a gum cancer, a tongue cancer, a neuroblastoma, a head cancer, a neck cancer, a breast cancer, a pancreatic cancer, a prostate cancer, a renal cancer, a bone cancer, a testicular cancer, an ovarian cancer, a mesothelioma, a cervical cancer, a gastrointestinal cancer, a lymphoma, a myeloma, a brain cancer, a colon cancer, a sarcoma or a bladder cancer. The cancer may be a primary cancer or a metastasized cancer. In certain embodiments, the TAGE agent may be injected directly into a tumor (i.e., by intratumoral injection) in a subject, for instance, in an amount effective to edit one or more cell types in the tumor (e.g., macrophages, CD4+ T cells, CD8+ T cells, or fibroblasts). For example, TAGE agents of the present disclosure may be used to treat a solid tumor in subject (e.g., a human) by administering the TAGE agent intratumorally.

In some embodiments, a TAGE agent may be injected directly into a solid tumor with a needle, such as a Turner Biopsy Needle or a Chiba Biopsy Needle. When treating a solid tumor in the lung, for example, a TAGE agent may be administered within the thorax using a bronchoscope or other device capable of cannulating the bronchial. Masses accessible via the bronchial tree may be directly injected by using a widely available transbronchial aspiration needles. A TAGE agent may also be implanted within a solid tumor using any suitable method known to those skilled in the art of penetrating tumor tissue. Such techniques may include creating an opening into the tumor and positioning a TAGE agent in the tumor.

In other embodiments, a TAGE agent may be injected into the bone marrow (i.e., intraosseous injection) of a subject. Intraosseous delivery may be used to edit bone marrow cells (e.g., hematopoietic stem cells (HSCs)) in a subject. When delivered intraosseously, a TAGE agent of the present disclosure may be used to treat a stem cell disorder in a subject (e.g., a human) where bone marrow cells, e.g., HSCS, are modified in such a way as to provide treatment for a stem cell disorder.

In yet further embodiments, a TAGE agent may be injected directly into the ocular compartment of a subject, e.g., a human, in an amount effective to edit subretinal cells (e.g., retinal pigment epithelium (RPE) or photoreceptors). For example, TAGE agents of the present disclosure may be used to treat an eye disease in a subject (e.g., a human) by administering the TAGE agent intraocularly (e.g., by subretinal injection).

In one embodiment, a TAGE agent comprising an antigen binding polypeptide (e.g., antibody), may be administered to a human subject via local delivery. Local delivery refers to delivery to a specific location on a body where the TAGE agent will act within the region it is delivered to, and not systemically. Examples of local delivery for a TAGE agent including an antigen binding polypeptide, include topical administration, ocular delivery, intra-articular delivery, intra-cardiac delivery, intradermal, intracutaneous delivery, intraosseous delivery, intrathecal delivery, or inhalation.

In one embodiment, a TAGE agent comprising an antigen binding polypeptide (e.g., an antibody or an antigen-binding fragment thereof) is administered to a human subject via systemic administration. Examples of systemic delivery for a TAGE agent containing an antigen binding polypeptide (e.g., an antibody or an antigen-binding fragment thereof) include intravenous injection or intraperitoneal injection.

### EXAMPLES

The invention will be more fully understood by reference to the following examples. They should not, however, be construed as limiting the scope of the invention. All literature and patent citations are incorporated herein by reference.

As used throughout the Examples, the symbol “-” in a name of a construct (e.g., Cas9-2×NLS) refers to a genetic fusion, unless otherwise indicated. The symbol “=” or “:” in the name of a construct (e.g., Cas9-proteinA:Antibody; Antibody-SpyTag=SpyCatcher-Cas9) refers to conjugation mediated by interaction between two conjugation moieties (e.g., ProteinA and the Fc region of an antibody, SpyCatcher and SpyTag; or Halo and Halo-tag).

### Example 1. Design and Production of Cas9-2×NLS-ProteinA

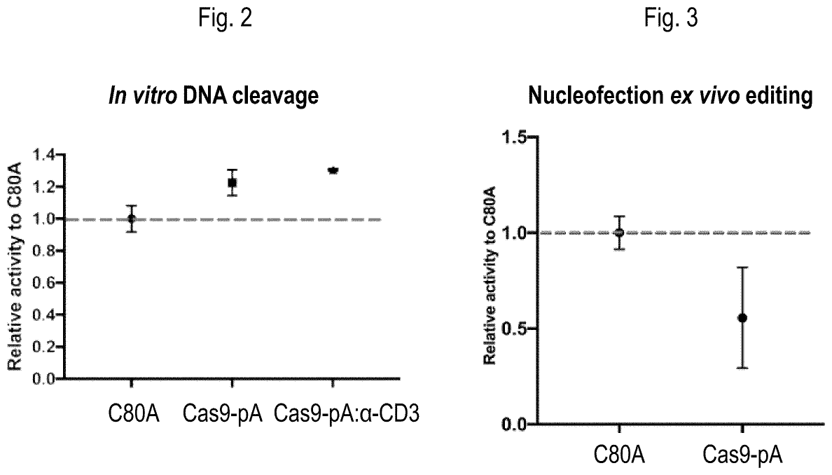

A Cas9 fusion including a 2× Nuclear Localization Signal and Protein A (Cas9 (C80A)-2×NLS-ProteinA, also referred to as “Cas9-2×NLS-ProteinA” or “Cas9-pA” hereinafter unless otherwise indicated; SEQ ID NO: 3; FIG. 2A) was constructed and purified from E. coli according to the following steps.

E. coli containing a vector expressing Cas9-2×NLS-ProteinA was cultured in selective TB media at 37° C. with shaking at >200 rpm. At an OD600 of 0.6-0.8, expression of Cas9-2×NLS-ProteinA was induced with 1 mM IPTG overnight at 16° C. or 3 hr at 37° C. The culture was subsequently harvested by centrifugation at 4000×g for 20 min at 4° C. Each liter of cells was resuspended with 20 ml of cold lysis buffer (50 mM Tris pH 8, 500 mM NaCl, 10 mM imidazole, 1× protease inhibitors, 0.025% TX-100) and cells were lysed by sonication. Debris was pelleted at 15000×g for 40 min at 4° C.

The lysate was applied to a 5 ml NiNTA Fastflow prepacked column. The column was washed with at least 5 volumes of NiNTA wash buffer (50 mM Tris pH 8, 500 mM NaCl, 10 mM imidazole). The column was then washed with at least 5 volumes of TX-100 buffer (50 mM Tris pH8, 500 mM NaCl, 10 mM imidazole, 0.025% TX-100). The column was subsequently washed with NiNTA wash buffer until complete. Washing was monitored by Bradford reagent. The sample was eluted in NiNTA elution buffer (50 mM Tris pH 8, 500 mM NaCl, 300 mM imidazole) and monitored by Bradford reagent. Typically, all protein was eluted with 4 column volumes of NiNTA Elution buffer.

The protein concentration was measured in the eluent and HRV 3C protease was added at 1:90 w/w of protease:eluent. The eluent was transferred to dialysis cassettes, and dialyzed overnight in 1 L of dialysis buffer (50 mM Tris pH 8, 300 mM NaCl) at 4 C. The dialysate was applied to a 5 ml NiNTA column equilibrated in overnight dialysis buffer, and the flowthrough was collected. This step was repeated a second time. The column was washed with ˜5 ml of overnight dialysis buffer to ensure all flowthrough protein was collected. The sample was then diluted with 1:1 v/v with no salt buffer (20 mM Hepes pH 7.5, 10% glycerol) to bring the salt concentration down to ˜150 mM, and centrifuged for 10 min and 4000 rpm to pellet any precipitated protein.

Soluble protein was applied to a HiTrap SP column equilibrated in ion exchange (IEX) buffer A (20 mM Hepes pH 7.5, 150 mM KCL, 10% glycerol) and eluted over a linear gradient of 20 CV from IEX buffer A to B (20 mM Hepes pH 7.5, 1.5M KCl, 10% glycerol) at a rate of 5 ml/min (Akta Pure). The SP column was washed in 0.5M NaOH to ensure no endotoxin carryover from other purifications.

Cas9-2×NLS-ProteinA eluted off the SP column with a peak at ˜33 mS/cm or about 22% IEX Buffer B. Fractions were pooled and concentrated to ˜0.5 ml with a 30 kDa spin concentrator.

Protein was separated on an S200 Increase 10/300 column equilibrated in Size Exclusion Buffer (20 mM Hepes pH 7.5, 200 mM KCl, 10% glycerol). The S200 column was washed in 0.5M NaOH to ensure there was no endotoxin carryover from other purifications. Cas9-ProteinA was eluted with a peak at ˜12 ml. Protein was concentrated and stored at −80C.

Following purification, the sample was incubated with selective endotoxin-removal resin until the endotoxin levels are appropriately low (e.g., generally 0.1 EU/dose).

The Cas9-2×NLS-ProteinA fusion was purified at a final concentration of approximately 1 mg/L.

### Example 2. In Vitro DNA Cleavage by Cas9-2×NLS-ProteinA

DNA cleavage by Cas9-2×NLS-ProteinA alone (Cas9-pA) or Cas9-2×NLS-ProteinA bound to an anti-CD3 antibody (“Cas9-pA-α-CD3”) was assessed by an in vitro DNA-cleavage assay.

500 nM of Cas9-pA:α-CD3 was reconstituted by combining 1 ul of 5× Buffer (100 mM HEPES pH 7.5, 1 M KCl, 25% glycerol, 25 mM MgCl2), 2.5 ul of 1 uM Cas9, 0.6 ul of 5 uM refolded guide RNA (gRNA; 0.6 nM final concentration), and 0.9 ul water. The reconstituted Cas9 RNP was incubated for 10 minutes at 37° C. to allow for Cas9 gRNA binding. To assess DNA cleavage, 100 nM of each Cas9 RNP was incubated for 30 minutes at 37° C. with 100 nM of a dsDNA target. Cas9 (C80A)-2×NLS (“C80A”) was assessed as a control.

1 ul of 20 mg/ml proteinase K was added to the reaction and incubated for 15 min at 50° C. The quenching reaction was held at 4° C. prior to separation on a Fragment Analyzer capillary electrophoresis (CE) instrument. 2 ul of the reaction was diluted with 22 ul of TE buffer and analyzed by capillary electrophoresis, per the manufacturer's recommendations. Cleavage reactions were run in triplicate, and background was subtracted from the band intensities. Percent cleavage was quantified with the following equation: % cleavage=(total moles cleaved product)/(total moles of substrate). The results are expressed as % cleavage relative to the Cas9 (C80A) internal control.

As shown in FIG. 2, Cas9-pA:α-CD3 achieved similar levels of DNA cleavage as Cas9 (C80A)-2×NLS.

### Example 3. Ex Vivo DNA Editing by Cas9-2×NLS-ProteinA Following Nucleofection

To assess the capacity of Cas9-2×NLS-ProteinA (“Cas9-pA”) to edit DNA ex vivo, 25 pM of Cas9-2×NLS-ProteinA or Cas9(C80A)-2×NLS (“C80A”) were introduced into stimulated human T cells by nucleofection.

To isolate stimulated human T cells, PBMCs were first isolated from buffy coat (SepMate isolation protocol from StemCell). T cells were then isolated from PBMCs (EasySep isolation protocol from StemCell) into T cell media (X-Vivo-15 media, 5% FBS, 50 uM 2-mercaptoethanol, 10 uM N-acetyl L-cysteine, and 1% Penn-Strepp). To stimulate the T cells, T cells were transferred to a flask at a concentration of 1×10⁶ cells/mL in T cell media and stimulation reagents (200 U ml⁻¹ IL-2, 5 ng ml⁻¹ IL-7, 5 ng ml⁻¹ IL-15, and immunocult soluble CD3/CD28 25 ul per ml) were added to the T cells. After 72 hours of stimulation, T cells were prepared for nucleofection.

Next, Cas9-2×NLS-ProteinA was complexed with guide RNA by incubating 50 uM Cas9-2×NLS-ProteinA with 25 uM refolded single guide RNA targeting the CD47 gene (CD47SG2) in Cas9 Buffer at 37° C. for 10 minutes to prepare a Cas9-2×NLS-ProteinA:gRNA RNP.

To assess the capacity of Cas9-2×NLS-ProteinA (Cas9-pA) RNP to edit DNA ex vivo, 25 pM of Cas9-2×NLS-ProteinA RNP or Cas9 (C80A) RNP were introduced into stimulated human T cells by nucleofection. Following nucleofection, CD47 downregulation was assessed using a phenotypic readout measuring the loss of surface CD47 using flow cytometry. Finally, DNA was isolated from cells and analyzed by amplicon sequencing. As shown in FIG. 3, the Cas9-2×NLS-ProteinA RNP displayed editing ex vivo in stimulated human T cells.

### Example 4. In Vitro Binding Assay to Assess Formation of Cas9-pA:Antibody Agent

To assess the ability of Cas9-2×NLS-ProteinA (“Cas9-pA”) to complex with an antibody, Cas9-proteinA (pA) was mixed with an anti-CD3 antibody at a 2:1 antibody:Cas9 ratio. Cas9 pA alone, anti-CD3 antibody alone, or a mixture of Cas9-pA and an anti-CD3 antibody were analyzed by size exclusion chromatography on a S200 size exclusion column.

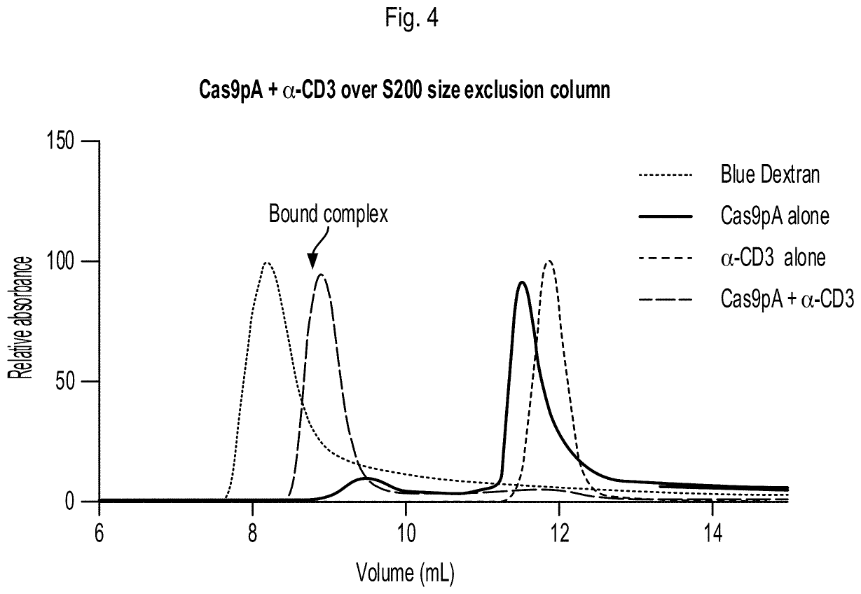

As shown in FIG. 4, Cas9-pA can bind the anti-CD3 antibody, thereby forming a Cas9 pA:antibody agent.

### Example 5. Antibody and Cas9-pA:Antibody Internalization Assays

Antibody and TAGE agent internalization was assessed by a FACS-based internalization assay.

### FACS-Based Internalization Assay

The FACS-based internalization assay is based on the detection of Alexa-488 labeled molecules (e.g., protein or RNA guides) following incubation of the labeled molecule with cells for a given period of time and comparing the results achieved with or without quenching with an anti-A488 antibody. Labeled molecules that are internalized by the cell are protected from quenching by the anti-A488 antibody and therefore retain a stronger Alexa488 signal relative to a control following quenching. In contrast, labeled molecules that are not internalized, and therefore remain on the cell surface, are susceptible to quenching by the anti-A488 antibody and therefore display a reduced Alexa488 signal relative to an unquenched control.

Alexa-488-labeled proteins (e.g., Cas9 or antibodies) described herein were prepared using NHSester-Alexa488 sold by Thermofisher (item #A37563) to conjugate to accessible lysines on the protein. To prepare the Alexa-488 labeled protein, 16000 pmol of NHS ester-Alexa488 was incubated with 1000 pmol of protein in Size Exclusion buffer (20 mM HEPES pH 7.5, 200 mM KCl, and 10% glycerol) supplemented with 10% Sodium Bicarbonate pH8.5 for 1 hr at room temperature. Excess unconjugated NHS ester was quenched with 10 mM Tris pH 8, and excess dye was removed using a HiTrap Desalting column.

Alexa-488-labeled guide RNA was prepared by purchasing custom tracrRNA from IDT with a 5′ labeled Alexa488. tracrRNA is complexed to crisprRNA, First, refolded guide RNA is prepared by combining 1× refolding buffer, 25 uM crisprRNA, and 25 uM Alexa-488-tracrRNA. The refolding reaction is heated to 70 C for 5 min and then equilibrated to room temperature. Subsequently, 20 mM MgCL2 is added to the reaction and heated at 50C for 5 min and then equilibrated to room temperature. The labeled guide RNA is then complexed with Cas9 (1.3:1 cr/trRNA:Cas9 ratio).

Once the labeled molecule is prepared, a titration curve with the molecule of interest was performed to find the optimal amount to achieve good staining without background on irrelevant cells. Cells were then prepared in accordance with the following method. Cells were collected and resuspended so they were at 500,000-1 million cells/100 uL (5 million-10 million/mL). Fc block was added to cells (1:100 for mouse, 5 uL per sample for human) and incubated for 15 minutes on ice. 100 uL of cells were added to each well, spun down at 300×g for 3 minutes, and cells were resuspended in 80 uL of 10% RPMI. If needed, cells were stimulated to cause an upregulation of surface markers. Cells were then exposed to the labelled molecule in accordance with the wash-off method below or the continuous labeling method in the next section.

The “wash-off” method involved first incubating all samples with the 488-labeled molecule at 4C to allow for surface binding. The molecule was then washed off before moving the cells to 37C. This way, only the molecules that initially bound to the surface were internalized. For the wash-off method, 20 uL of the A488-molecule was added to the cells in 80 uL of RPMI/FBS, and incubated for 30 minutes on ice. Then, 100 uL of PBS was added on top of wells and cells were spun at 300×g for 3 minutes. Cells were resuspended in 100 uL of RPMI+10% FBS. 4C sample and controls were kept on ice, while 37C samples were moved to separate plate(s) and incubated for a set amount of time (e.g., 15 min, 60 min, or longer (e.g., 3 hr)). After the first time point is done (i.e., 15 min), the plate or the cells were removed and kept on ice.

In contrast, the continuous method involved moving the cells to 37C (or keeping them at 4C) and adding the 488-labeled molecule from the beginning. This allowed for continual uptake of the molecule during the entire 37C incubation period. 4C samples were kept on ice while 37C samples were incubated at 37C. The A488-labeled molecule was then added to samples at the appropriate time, starting with the longest time point sample (i.e., the 488-labeled molecule wad added to the 3 hour sample first; after 2 hours (1 hour remaining), the 488 molecule was added to the 60 minute sample, and then after 2.75 hours (0.25 hour remaining), the 488 molecule was added to the 15 minute sample. After the final time point, all samples were moved back to ice. The continuous method was utilized in the following experiments.

Finally, the samples were quenched with an anti-A488 antibody and stained for FACS analysis. Before spinning down, each sample was split in half, providing two 50 uL samples for each time point. Plates were spun down for 300×g for 3 minutes. Then, 50 uL of MACS buffer (PBS, 2% FBS, 2 mM EDTA) was added to all wells that were UNQUENCHED. Next, 50 uL of anti-A488 quenching master mix was added to all wells that were QUENCHED. Finally, 50 uL of FACS mix was added to all samples. Samples were then incubated on ice for 30 minutes. 100 uL of MACS buffer was then added to each well, after which cells were spun down at 300×g for 3 minutes at 4C. Cells were resuspended in 170 uL MACS buffer and 10 uL 7AAD. After incubation for 5 minutes, the samples were run on the Attune NxT Flow Cytometer. Alternatively, cells can be fixed before analysis by resuspending cells in 100 uL of 4% PFA in PBS, incubating for 10 minutes at room temperature, adding 100 uL of PBS on top, spinning down, and resuspending cells in 180 uL of PBS. Following this, cells can be analyzed the next day.

### Antibody Internalization

To identify candidate antibodies that may function in a Cas9-2×NLS-ProteinA (“Cas9-pA”):antibody agent, antibodies were first assessed for their internalization capacity without Cas9-pA. The internalization of a variety of antibodies having different targets (i.e., CD22, CD33, CD3, CXCR4, CD25, CD54, CD44, and EGFR) was assessed in mouse and human cell populations (e.g., B cells, myeloid cells, T cells, activated T cells, epithelial cells). As shown in Table 2, anti-CD22, anti-CD33, anti-CD3, anti-CXCR4, anti-CD54, and anti-CD44 antibodies were identified that are internalized by a wide range of human mouse immune cells.

TABLE 2
Antibody Internalization

| Antibody Target | Population Targeted | Does it Internalize? |
|---|---|---|
| CD22 | B cell | Yes |
| CD33 | Myeloid Cells | Yes |
| CD3 | T Cells | Yes, slowly (7-24 hrs) |
| CXCR4 | Precursors, T cells, myeloid | Yes |
| CD25 | Activated T cells | Not tested |
| CD54(ICAM1) | Many cells | Yes |
| CD44 | Many cells | Yes |

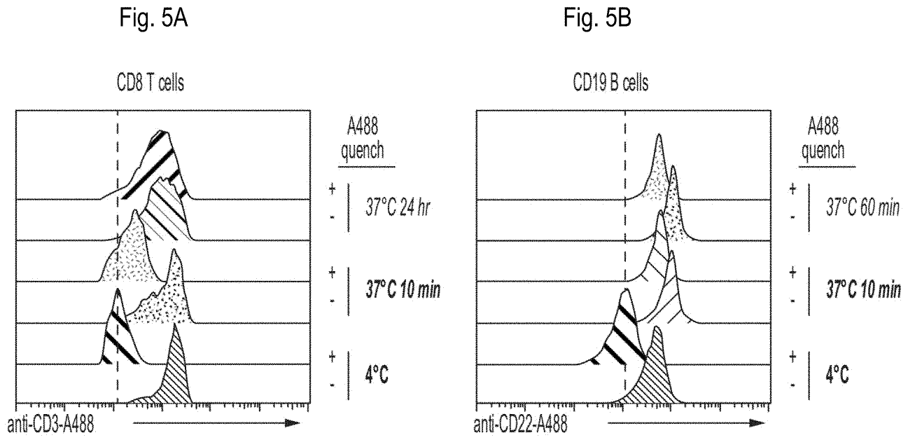

In particular, the rate of internalization of anti-CD3 (18 nM) or anti-CD22 (100 nM) antibodies was assessed by adding each antibody to PBMCs. After the indicated times at the indicated temperatures, the external A488 signal was quenched with an anti-A488 antibody. Specific cell populations were identified by FACS. As shown in FIGS. 5A and 5B, antibodies recognizing CD3 and CD22 internalize at different rates.

### TAGE Agent Internalization

Next, internalization of candidate antibodies complexed with Cas9-2×NLS-proteinA (Cas9-pA) was assessed in a FACS-based internalization assay. Cas9-pA complexed to human IgG1 or an anti-CD22 antibody was assessed with A488 labeling either on the Cas9-pA or on the antibody. An anti-CD22 antibody alone was assessed as a control.

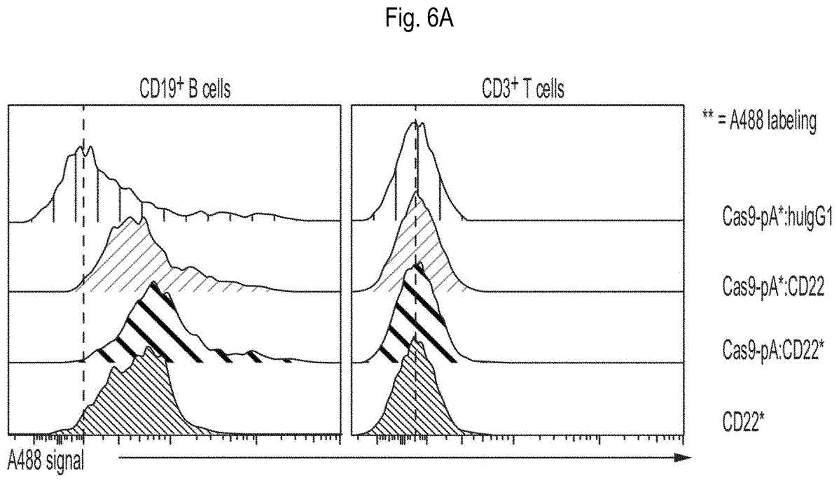

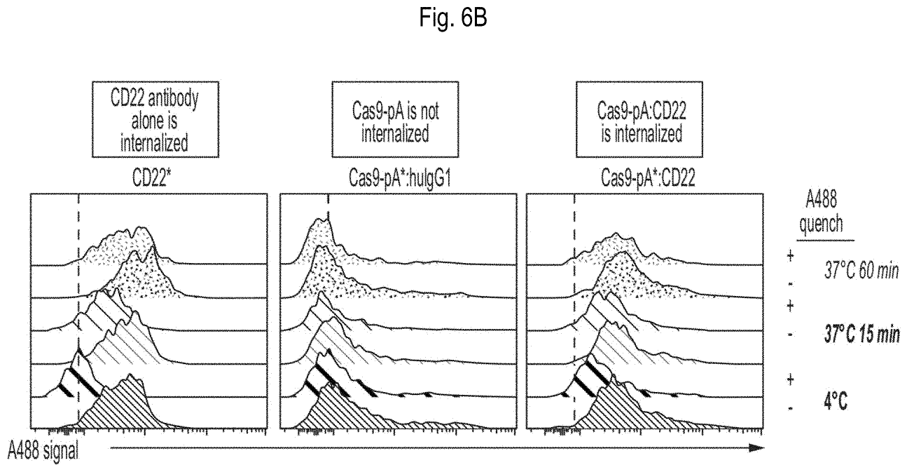

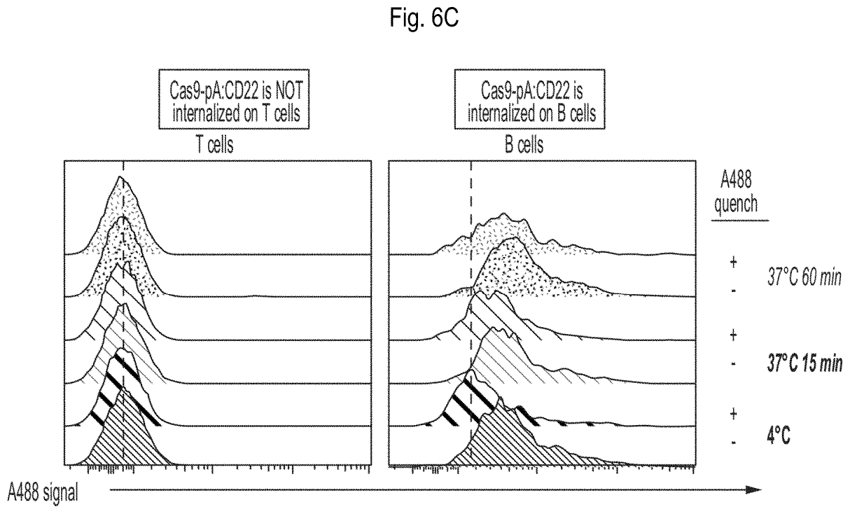

First, cell binding was assessed by adding 10 nM of each protein to PBMCs and staining for 30 minutes on ice. As shown in FIG. 6A, complexing Cas9-pA with anti-CD22 increases binding on B cells but not on T cells.

Next, after adding 10 nM of each protein to PBMCs and quenching, cells were stained for CD45, CD3, and CD19. As shown in FIG. 6B, Cas9-pA can be internalized when complexed with anti-CD22 while Cas9-pA is not internalized. As shown in FIG. 6C, Cas9-pA:anti-CD22 only binds and is internalized on B cells, but not T cells in the same cell pool. Therefore, Cas9-pA:antibody agents display effective internalization by B cells when delivered to bulk PBMCs.

### Example 6. Antibody and Cas9-pA:Antibody Internalization Assays

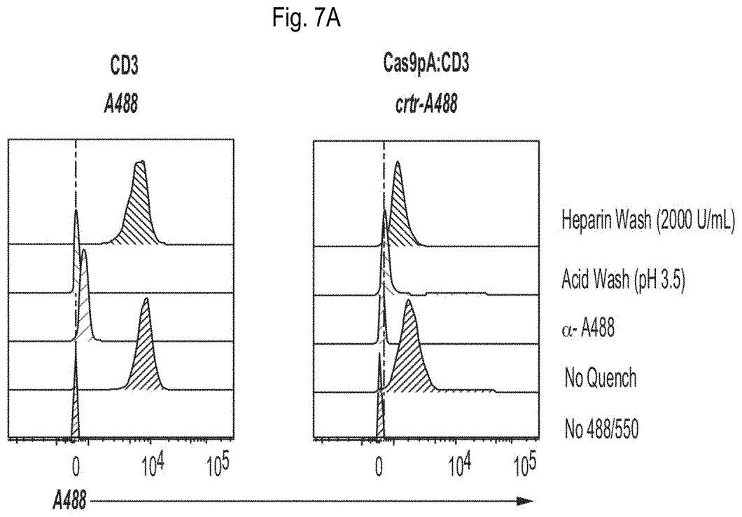

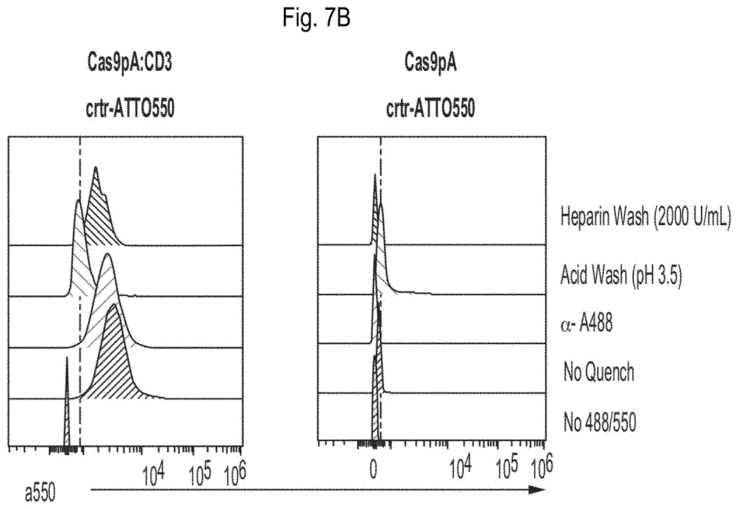

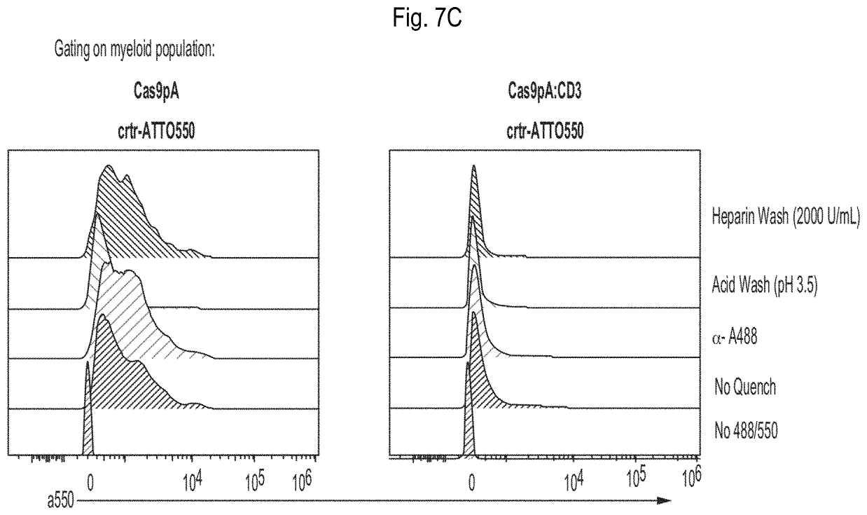

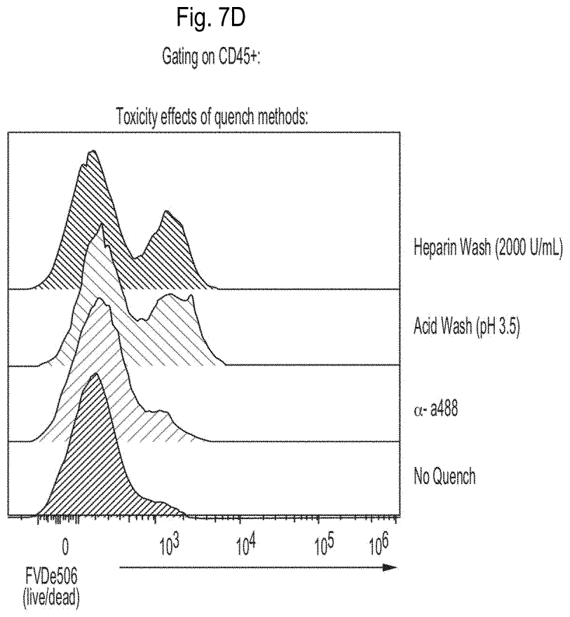

To assess the efficacy of different quenching methods in the FACS-based internalization assay described in Example 5, antibody (anti-CD3 antibody), Cas9-2×NLS-ProteinA (“Cas9-pA”) RNP, or TAGE agent (Cas9-pA:anti-CD3 antibody RNP (“Cas9 pA:CD3”) internalization was assessed by a FACS-based internalization assay in which the reporter signal (A488 or ATTO550) was quenched by heparin wash (2000 U/mL), acid wash (pH 3.5), or anti-A488 antibody. For each RNP, the reporter signal (A488 or ATTO500) was conjugated to guide RNA. The toxicity of each quenching method was further assessed on CD45+ cells by a FACS-based live/dead assay, in which the level of FVDe506+ cells (dead cells) was determined by FACS (FIG. 7D). As indicated in FIG. 7D, acid quenching and heparin quenching had more toxicity than regular quenching.

Internalization of Cas9-pA, an anti-CD3 antibody, or Cas9-pA complexed with an anti-CD3 antibody was assessed in T cells (FIGS. 7A and 7B) or myeloid cells (FIG. 7C). As shown in, FIGS. 7A and 7B, an acid wash was as effective for quenching in the internalization assay as an anti-A488 antibody. For myeloid populations, the acid wash was more effective for quenching than the anti-A488 antibody for Cas9-pA staining (FIG. 7C).

### Example 7. In Vitro DNA Cleavage by Cas9-2×NLS-ProteinA

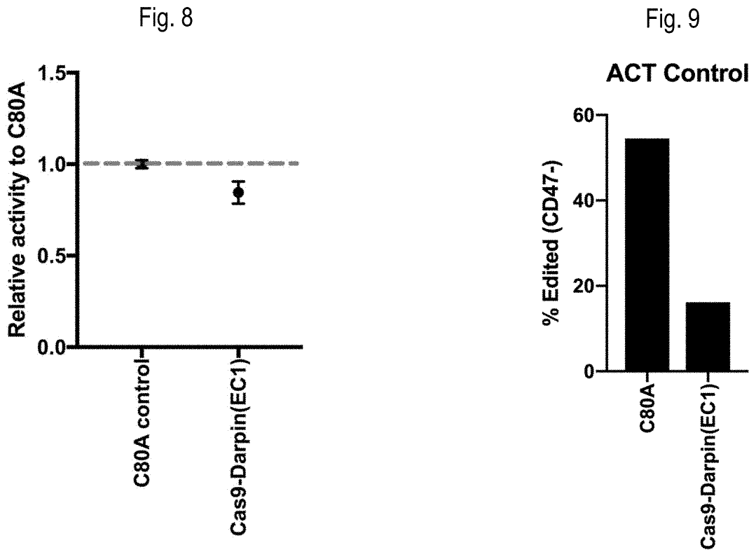

DNA cleavage by the TAGE agent Cas9-Darpin(EC1) was assessed by an in vitro DNA-cleavage assay, as described in Example 2. As shown in FIG. 8, Cas9-2×NLS-Darpin(EC1) achieved similar levels of DNA cleavage as Cas9 (C80A)-2×NLS.

### Example 8. Ex Vivo DNA Editing by Cas9-Darpin(EC1) Following Nucleofection

To assess the capacity of the TAGE agent Cas9-2×NLS-Darpin(EC1) (“Cas9-Darpin(EC1)”) to edit DNA ex vivo, stimulated human T cells (see Example 3) were nucleofected with 25 pM of Cas9-Darpin(EC1) RNP or Cas9 (C80A) RNP. A guide RNA targeting CD47 was associated with the respective TAGE agents to form ribonucleoproteins, and the ribonucleoproteins were nucleofected into T cells to test for editing. Editing was measured using a phenotypic readout measuring the loss of surface CD47 using flow cytometry. Following nucleofection, CD47 downregulation was assessed using a phenotypic readout measuring the loss of surface CD47 using flow cytometry. Finally, DNA was isolated from cells and analyzed by amplicon sequencing. As shown in FIG. 9, the Cas9-Darpin(EC1) RNP displayed editing ex vivo in stimulated human T cells.

### Example 9. Binding of Cas9-DARPin(EpCAM) on EpCAM+ Cells

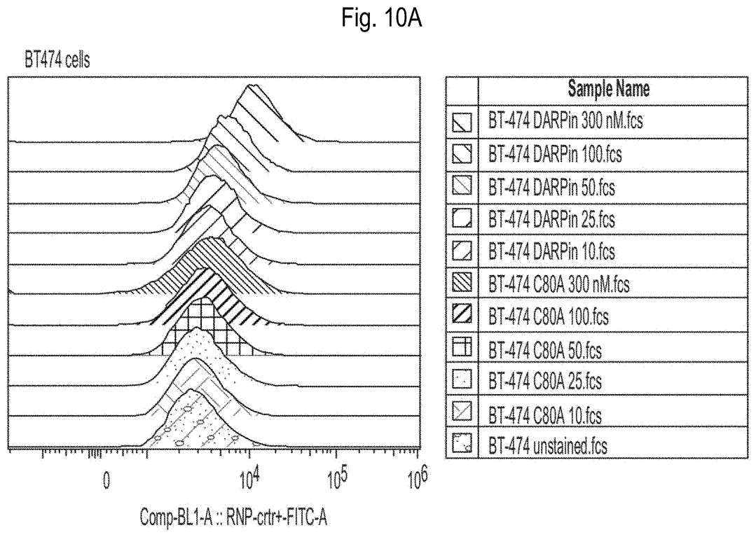

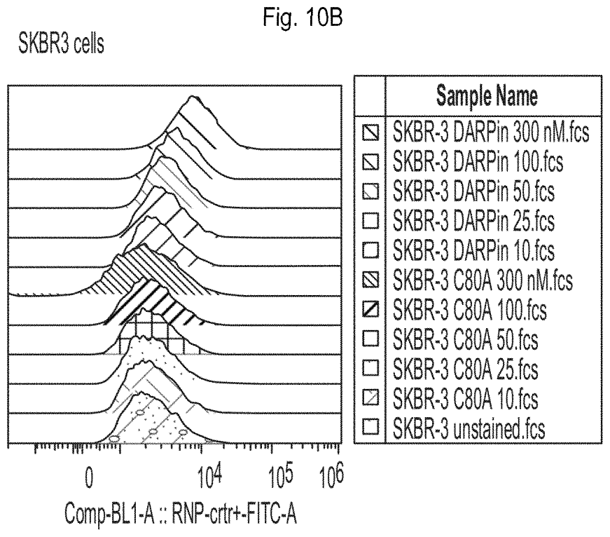

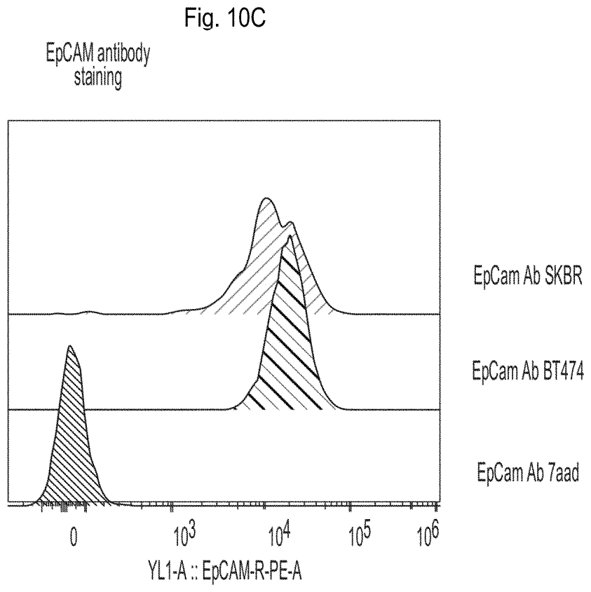

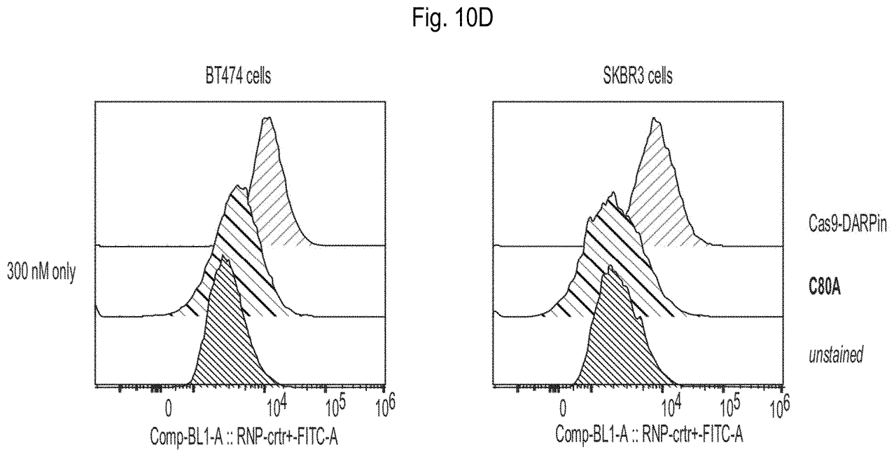

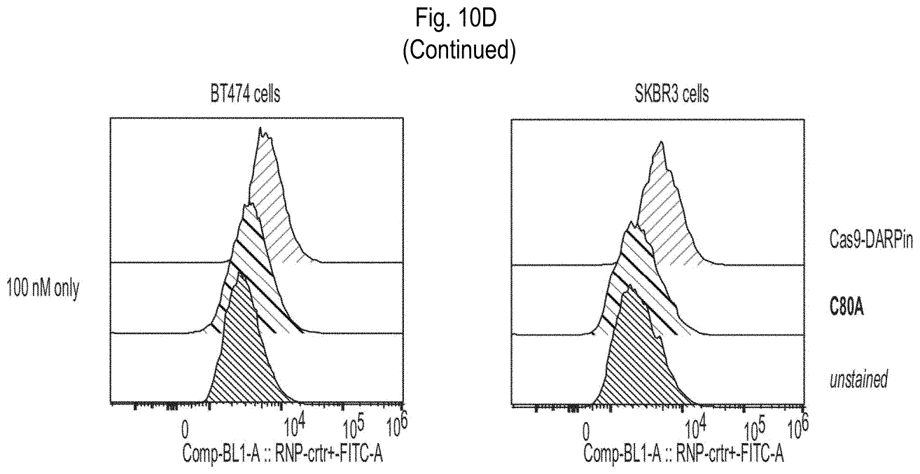

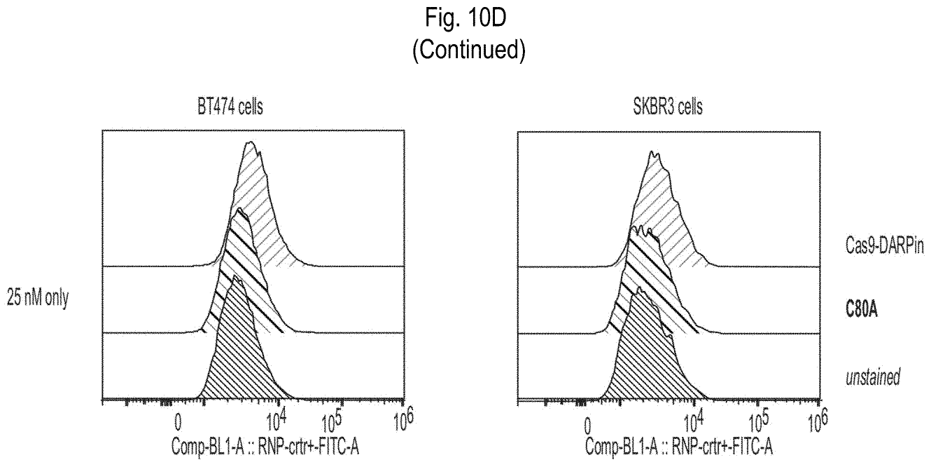

To assess the ability of the TAGE agent Cas9-2×NLS-DARPin(EpCAM) (“Cas9-DARPin(EpCAM)”) to bind EpCAM+ cells, Cas9-DARPin(EpCAM) RNP or a Cas9(C80A)2×NLS control at 10, 25, 50, 100, or 300 nM in PBS were incubated with two different human epithelial breast cancer cell lines SKBR-3 and BT474. As shown in FIG. 10C, SKBR-3 and BT474 cells express EpCAM, as detected by EpCAM antibody staining. The indicated RNPs were complexed with HBB cr/tr guides labelled with A488 and incubated with the SKBR-3 or BT474 cell line for 30 minutes on ice. The cells were then washed and analyzed by FACS.

As shown in FIGS. 10A and 10B, EpCAM targeted Cas9-DARPin binds EpCAM+ cells. Binding is detected particularly when cells are incubated with high concentrations of Cas9-DARPin(EpCAM), as shown in FIG. 10D.

### Example 10. Internalization of Cas9-DARPin(EpCAM)

Internalization of the TAGE agent Cas9-2×NLS-DARPin(EpCAM) (“Cas9-DARPin(EpCAM)”) in EpCAM+ BT-474 cells or SKBR3 cells was assessed using a FACS-based internalization assay, the protocol for which is further described in Example 5. 100 nM or 300 nM of Cas9-DARPin (EpCAM) was incubated with BT474 cells or SKBR3 cells for the indicated time (60 min or 30 min) at 37° C. or 4° C. prior to quenching.

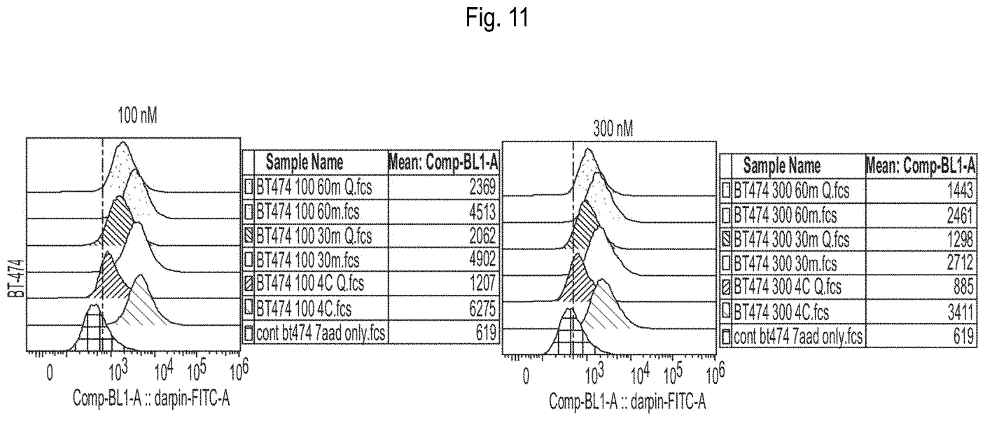

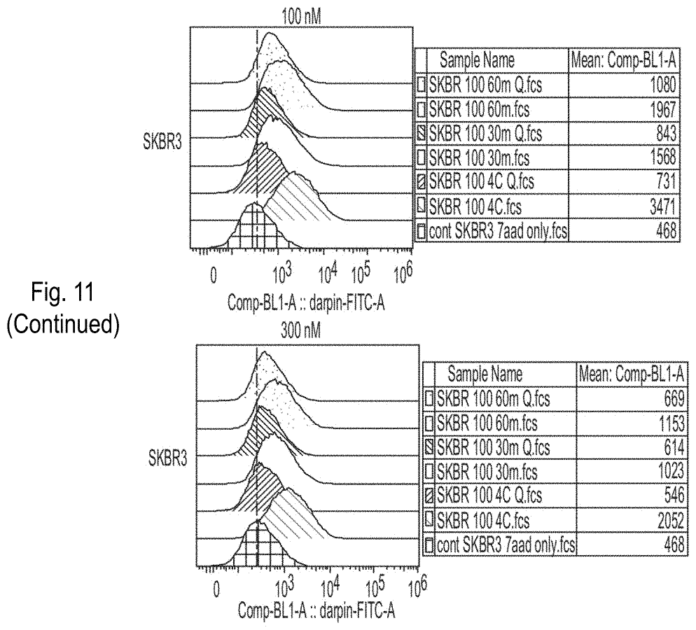

As shown in FIG. 11, Cas9-DARPin(EpCAM) is internalized in BT474 cells.

### Example 11. Ex Vivo Editing by Cas9-DARPin(EpCAM) Following Co-Incubation or Nucleofection

The TAGE agent Cas9-2×NLS-DARPin(EpCAM) (“Cas9-DARPin(EpCAM)”) was assessed by an ex vivo editing assay comparing the level of editing achieved with co-incubation in BT474 cells verses that achieved in SKBR3 cells.

Ex Vivo Editing of Adherent Cells by Co-Incubation—Editing while Cells are in Suspension

RNP complexes were prepared by combining Cas9-DARPin(EpCAM) and huCD47g2 guide RNA targeting CD47. Cells grown on tissue culture plates were lifted by brief trypsinization. Trypsinization was quenched by adding at least a 5× excess of complete cell culture medium. Cells were then counted and washed with cell culture medium. Cell culture medium contained 0-10% fetal bovine serum, as appropriate for desired editing condition. Cells were then pelleted by centrifugation and resuspended at high density in cell culture medium. Concentrated cells (˜500,000 cells) were mixed with 3.75 uM RNP in an Eppendorf tube. Cells were then placed in a 37° C. incubator for 1 hour. After 1 hour, cells with RNP were transferred to a tissue culture plate that was pre-loaded with complete cell culture medium.

On the following day, cells were split when they reached 80-100% confluence (optimal cell density depends on the cell type being used). Day 4 and Day 7 post-co-incubation, cells were harvested to measure the degree of gene editing using flow cytometry.

### Results

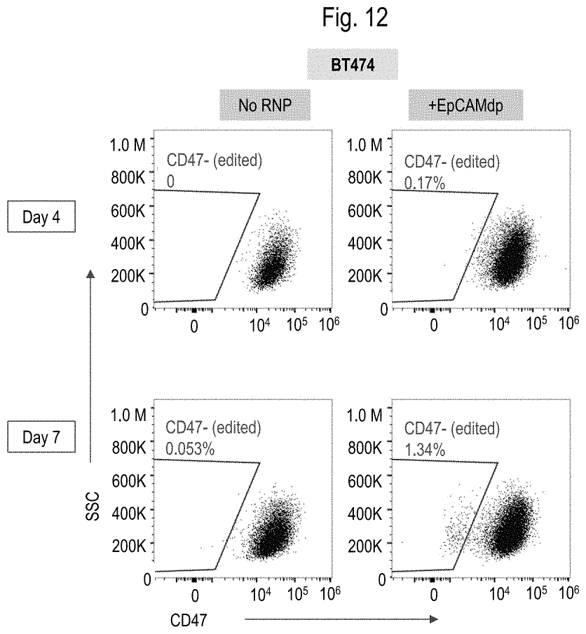

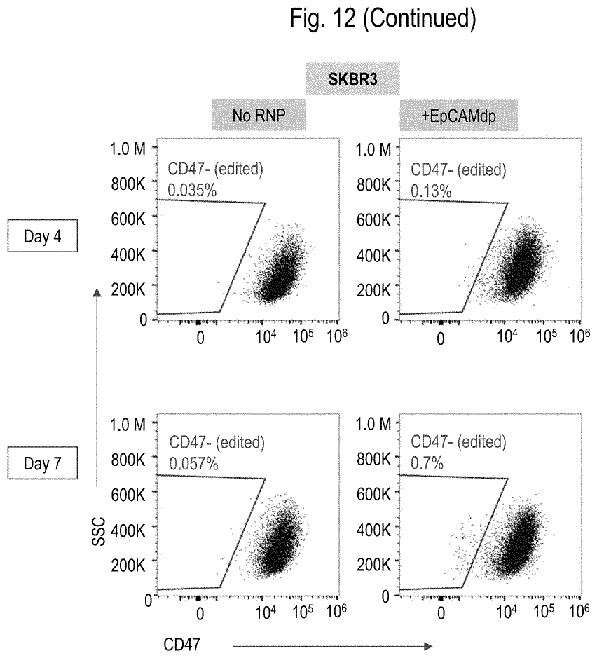

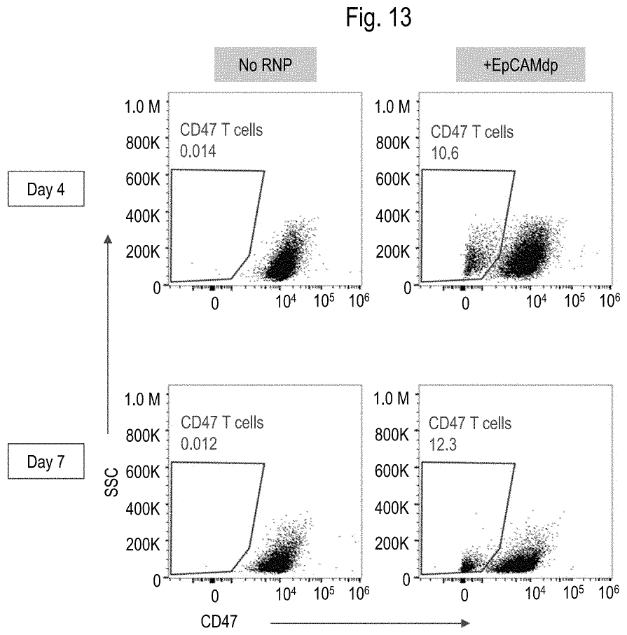

As shown in FIG. 12, Cas9-DARPin (EpCAM) exhibited approximately 1.34% editing following co-incubation in BT474 cells and 0.7% editing following co-incubation in SKBR3 cells. Results achieved in a RNP-free condition are shown for comparison. As a control, editing by Cas9-DARPin (EpCAM) introduced by nucleofection was confirmed in human T cells (FIG. 13).

### Example 12. Production of Cas9-Halo:Antibody Conjugates

A TAGE agent including Cas9 linked to a Halo tag (Cas9-2×NLS-Halo) (“Cas9-Halo”) was constructed and purified from E. coli according to a similar protocol used to produce Cas9-2×NLS-proteinA, as outlined in Example 1. Cas9-Halo can be conjugated to antibodies of any isotype (or any other protein) using a succinimidyl ester linked to a Halo ligand (promega P6751). In this example, an anti-CD22 antibody was complexed with Cas9-Halo.

First, the anti-CD22 antibody was linked to the Halo-succinimidyl ester via the amine reactive group to lysines on the antibody, as follows. Sodium bicarbonate pH8.5 to 100 mM was added to the antibody. Then, 8 molar excess NHSester-Halo ligand was added to the antibody. The conjugation was quenched with 10 mM Tris pH7.5. Increasing or decreasing the molar excess of halo ligand in relation to antibody can be used to change Cas9:antibody conjugation ratio. Next, the antibody conjugation reaction was run over a desalting column, and the antibody was concentrated to >50 uM.

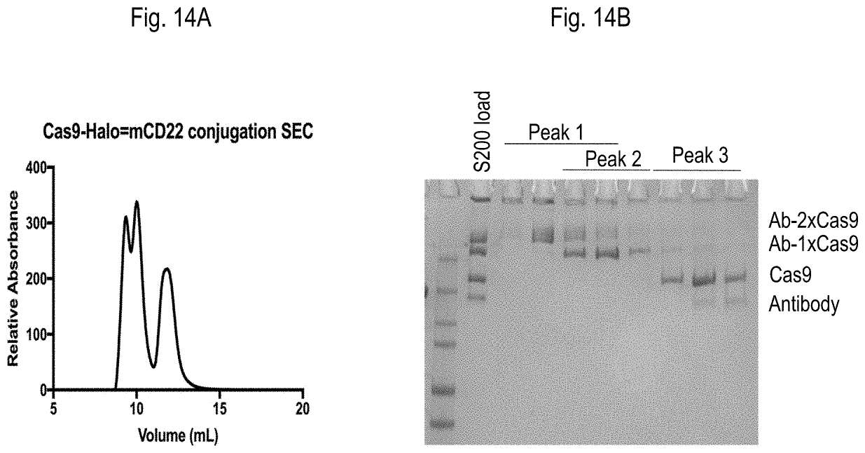

To conjugate the anti-CD22 antibody linked to the Halo ligand with Cas9-Halo, the antibody and Cas9-Halo were combined at a 1:1.5 molar ratio and incubated for 1 hour at room temperature. An S200 10/300 Increase sizing column in SEC buffer (20 mM HEPES, pH 7.5, 200 mM KCL, and 10% glycerol) was used to separate antibody-cas9 conjugates from unconjugated material (FIG. 14A). Peaks between 8.5-11 mL contained conjugated material. SDS-PAGE was used to identify the ratio of Cas9-Antibody conjugation (FIG. 14B).

### Example 13. Internalization of Cas9-Halo:Anti-CD22 Antibody

Internalization of TAGE agents including Cas9-2×NLS-Halo (“Cas9-Halo”):anti-CD22 antibody in mouse B cells from healthy spleen or B116 tumors was assessed using a FACS-based internalization assay (using a wash-off method), the protocol for which is further described in Example 5. 20 nM of the indicated RNP (Cas9-Halo:anti-CD22 antibody, Cas9-Halo:IgG1, or Cas9-Halo) with an A488 guide RNA was incubated with total splenocytes or tumor infiltrating lymphocytes for the indicated time (15 min or 60 min) at 37° C. or 4° C. Samples from each condition with and without quenching were assessed by FACS analysis gated on CD19+ B cells.

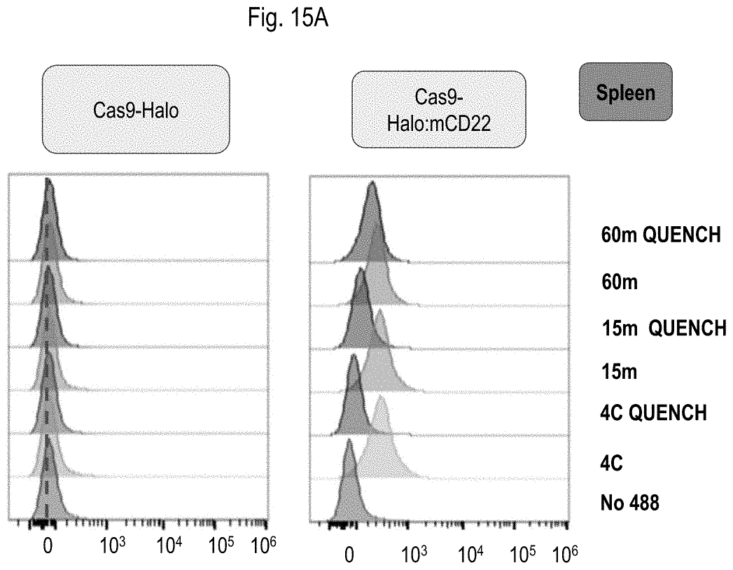

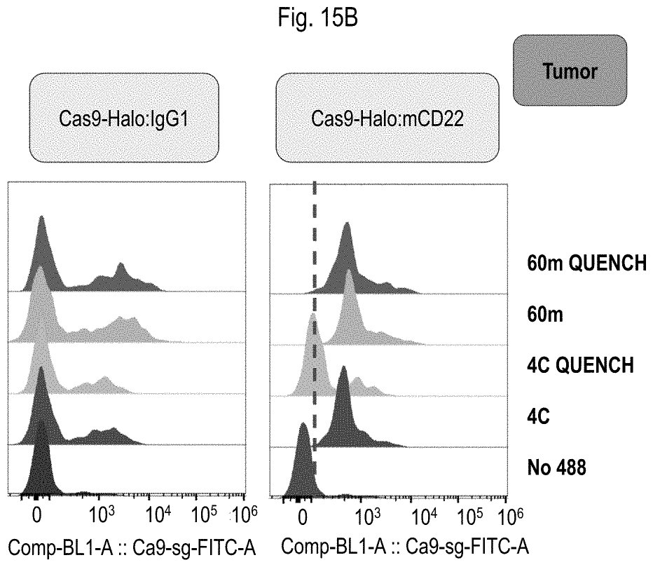

As shown in FIGS. 15A and 15B, Cas9-Halo:anti-CD22 is internalized into mouse B cells from healthy spleen and B116 tumors.

### Example 14. In Vitro DNA Cleavage and Ex Vivo Editing by Cas9-Halo:Anti-CD22 Antibody

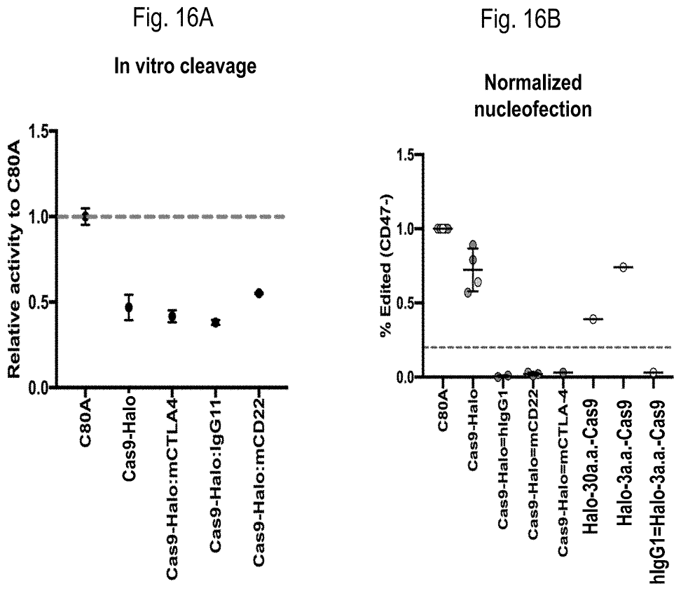

TAGE agents including Cas9-2×NLS-Halo (“Cas9-Halo”) were assessed in an in vitro DNA cleavage and ex vivo nucleofection editing activity was assessed, as outlined in Examples 2 and 3, respectively. In particular, Cas9-halo (20181209), Cas9-Halo:anti-mCTLA4, Cas9-Halo:IgG1, Cas9-Halo:anti-CD22, Halo-30aa-Cas9, Halo-3aa-Cas9 were assessed for activity in vitro by incubating with dsDNA. Each construct displayed DNA cleavage activity in vitro (FIG. 16A).

Next, 25 pM of each RNP was introduced by nucleofection into stimulated human T cells. A guide RNA targeting CD47 was associated with the respective TAGE agents to form ribonucleoproteins, and the ribonucleoproteins were nucleofected into T cells to test for editing. Editing was measured using a phenotypic readout measuring the loss of surface CD47 using flow cytometry. FIG. 16B shows relative efficiency of editing by Halo complexed antibodies as compared to Cas9(C80A)-2×NLS.

### Example 15. Cas9-Halo:Antibody RNP Differential Internalization in a Mixed Cell Population

Internalization of TAGE agents including Cas9-2×NLS-Halo (“Cas9-Halo”) complexed with an antibody (“Cas9-Halo:Antibody”), TAGE agent RNP internalization was assessed in a mixed cell population. Live cells isolated from pooled B16F10 tumors were mixed with Cas9-Halo TAGE agent RNPs complexed with different antibodies (anti-CTLA4 antibody, anti-CD22 antibody, IgG1, MHCII-Nb). Cas9(C80A) RNP and Cas9-Halo RNP alone were also assessed as controls. Each RNP with A488-labelled guide RNA was incubated with tumor cells at 4° C. and 37° C. for 1 hour, after which samples were assessed by FACS-analysis with or without quenching. Internalization of each RNP was assessed in gated DC cells, non-DC myeloid cells, B cells, T cells, non-T/B cells, or CD45− PDPN+ cells.

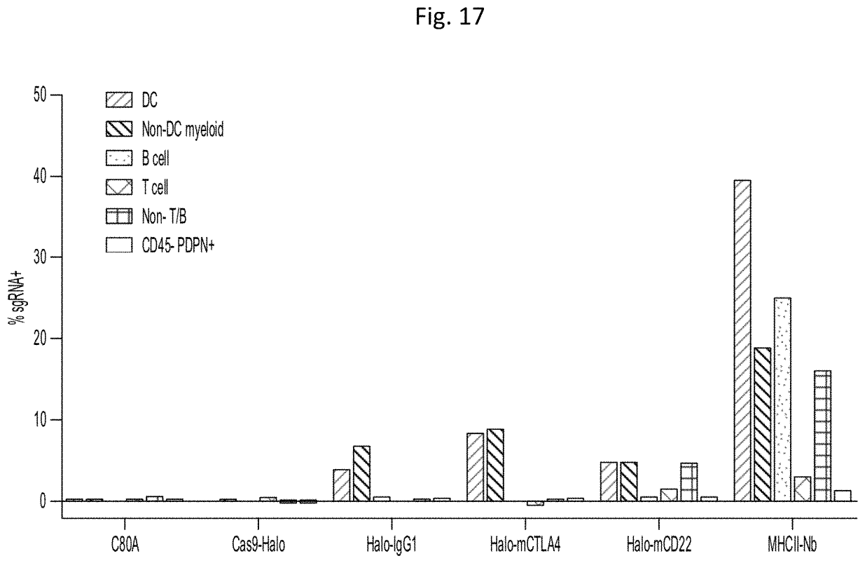

As shown in FIG. 17, Cas9-Halo:Antibody RNPs displayed differential internalization patterns in DC cells, non-DC myeloid cells, B cells, T cells, non-T/B cells, and CD45− PDPN+ cells.

### Example 16. Antibody TAGE Agent with ProteinA Conjugation—Internalization and Editing Assays

TAGE agents containing Cas9-2×NLS-proteinA (“Cas9-pA”) linked to one of five different antibodies (an anti-CD33 antibody, an anti-EGFR antibody, an anti-CD25 antibody, anti-FAP antibody, or an anti-CTLA-4 antibody) were tested in different cell types for both internalization and editing.

First, a FACS-based internalization assay was performed to assess cellular internalization of Cas9-pA:antibody complexes including an anti-CD33 antibody, an anti-EGFR antibody, or an anti-FAP antibody (data not shown; see also Example 4). A TAGE agent containing Cas9-pA complexed with an anti-CD33 antibody increased internalization of Cas9-pA in US937 cells compared with Cas9 pA:huIgG1, but not to levels of internalization of the antibody alone. Cas9-pA complexed with an anti-EGFR antibody mediated binding and internalization in A431 epithelial cells compared with pA:huIgG1. Similarly, in human fibroblasts, Cas9-pA:FAP binds more than pA:huIgG1 (isotype control) and can drive Cas9-pA internalization on human fibroblasts.

Cas9-pA editing (without an antibody) showed consistently less editing than with Cas9 alone (C80A) (data not shown). Further, no detectable editing was observed when Cas9-pA was conjugated to an antibody across the five different cell types and antibodies tested (Table 3). The results from Table 3 suggest that, despite having the capacity to bind and internalize within cells, Cas9-pA constructs—regardless of the antigen the TAGE agent is targeted to—has reduced editing relative to controls. Accordingly, alternative conjugation moieties other than protein A were assessed, as described in Examples 17-21.

TABLE 3

| Cell | Antigen | Results |
|---|---|---|
| U937 | CD33 | No editing |
| A431 | EGFR | pA edited <50% as well as Cas9(C80A); |
| | | nothing with pA + Ab |
| HEK Blue | CD25 (IL- | pA edited <50% as well as Cas9(C80A); |
| | 2Ra) | nothing with pA + Iso; cells died with |
| | | pA + Ab |
| fibroblasts | FAP | pA edited <50% as well as Cas9(C80A); |
| | | nothing with pA + Ab |
| T cells | CTLA4 | No editing |
| (human) | | |

### Example 17. Antibody TAGE Agent with Halo Conjugation—Binding and Ex Vivo Editing Assays

Conjugation of antibodies to Cas9 via a Halo/Halo tag appeared to affect antibody binding in the context of a Cas9 TAGE agent in some antibody/cell type pairs, as shown in the following Example.

The antibodies described in the present Example were linked to a Halo Tag (HT) for conjugation to Cas9-Halo to form a Cas9-Halo:HT-Antibody conjugate (alternatively referred to as a Cas9-Halo:Antibody conjugate).

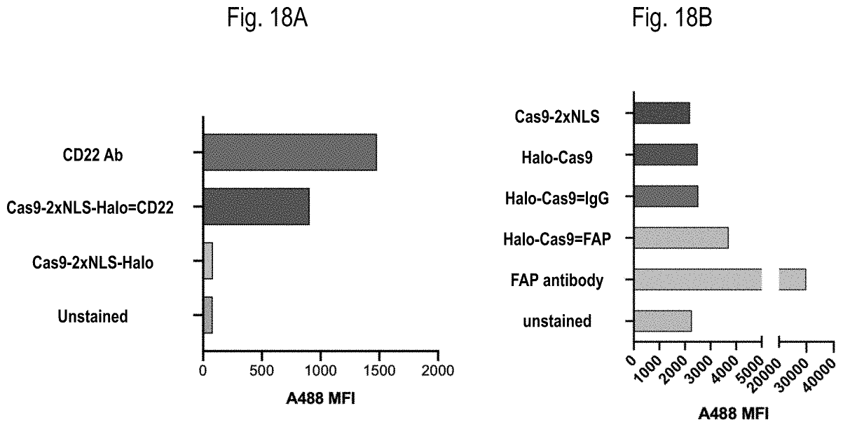

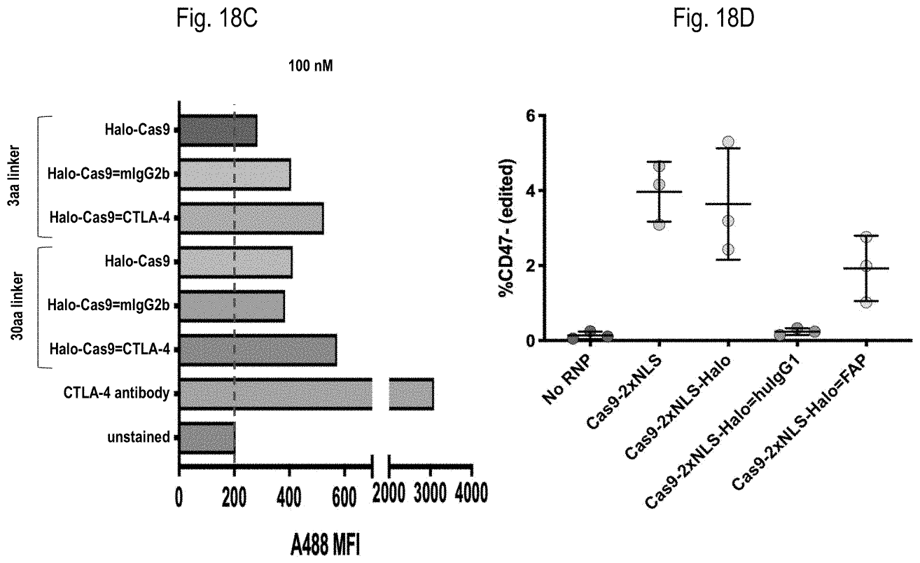

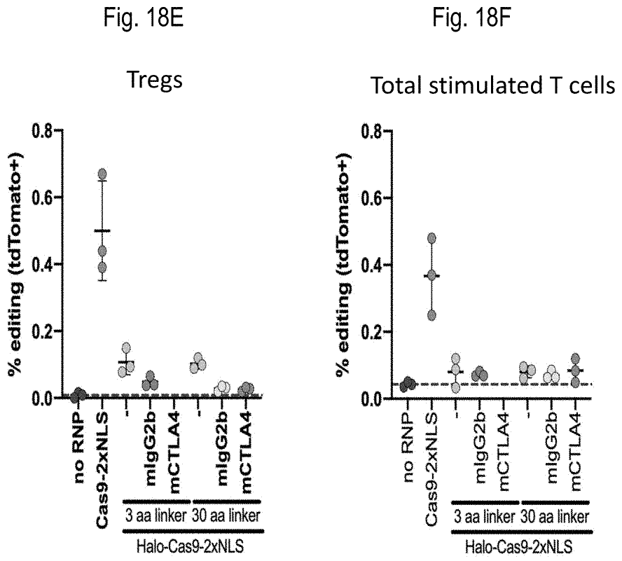

Initial tests with mouse anti-CD22 antibodies demonstrated equivalent B cell binding between a TAGE agent including Cas9-Halo conjugated to an antiCD22 antibody as compared to the anti-CD22 antibody alone (FIG. 18A). A subsequent fibroblast binding assay with an anti-FAP antibody conjugated to Cas9-Halo, or a T cell binding assay withan anti-mouse CTLA-4 antibody conjugated to Cas9-Halo (3 different clones tested) revealed less cellular binding of the Cas9-Halo:Antibody conjugates compared with antibody alone but increased binding over the negative controls (FIGS. 18B and 18C). Further testing indicated that the position of the Halo Tag from the N-terminus to the C-terminus of Cas9 did not impact binding, nor did the number of Halo Tags.

A TAGE agent including Cas9-Halo also showed variable editing depending on the cell type in which the Cas9-Halo was internalized. An ex vivo editing assay demonstrated that Cas9-Halo conjugated to an anti-FAP antibody (CD47 guide RNA; editing assessed by using a phenotypic readout measuring the loss of surface CD47 using flow cytometry.) was able to edit human fibroblasts via co-incubation at a similar level as Cas9(C80A)-2×NLS (a CPP-based TAGE agent used as a positive control) (FIG. 18D). However, a TAGE agent including Halo-Cas9 conjugated to an anti-CTLA-4 antibody and co-incubated with mouse T cells displayed lower editing levels (as measured by the TdTomato fluorescence reporter system) compared to Cas9(C80A)-2×NLS (about 20% of the editing observed with Cas9(C80A)-2×NLS; FIGS. 18E and 18F).

The results from the above example demonstrate binding and editing of fibroblasts using a TAGE agent targeting these cells (i.e., an anti-FAP TAGE agent), and suggests that such editing results in T cells may depend on the target or antibody.

### Example 18. Anti-FAP Antibody TAGE Agent Internalization and Ex Vivo Editing Assays

Internalization and ex vivo editing of a TAGE agent including a human anti-Fibroblast Activation Protein (FAP) antibody conjugated to Cas9 was assessed in this Example.

An anti-FAP antibody linked to a SpyTag (ST) and Cas9 linked to a spycatcher (SC) moiety were expressed using standard methods for expression of antibodies in mammalian cells (see, Vazquez-Lombardi et. al., (2018). Nature protocols, 13(1), 99). The SpyTag was genetically fused to the C-terminal of the light chain of the antibody, while SpyCatcher was genetically fused ot the N-terminus of Cas9-2×NLS to form SpyCatcher-Cas9(WT)-2×NLS. Anti-FAP-SpyTag was conjugated to SpyCatcher-Cas9 to form anti-FAP antibody/Cas9 conjugates (“FAP=SC-Cas9”). A portion of complexes included one SpyCatcher-Cas9 per antibody (FAP-SpyTag=SpyCatcher-Cas9), while another portion of complexes included two SpyCatcher-Cas9 moieties per antibody (FAP-SpyTag=(SpyCatcher-Cas9)₂). Complexes with two Cas9 molecules on a single antibody formed due to the presence of two light chains and two SpyTags per antibody.

To assess binding of the conjugates, adherent human dermal fibroblasts were incubated with 270 nM of protein for 1 hour at 4° C. or 37° C. and then analyzed by FACS. A488 signal comes from labeled antibody or A488-labeled guide (where Cas9 is present). FAP=SC-Cas9 bound comparably to the anti-FAP antibody alone. Further, internalization of the FAP=SC-Cas9 conjugate was evaluated in a variety of a cell types using the FACS-based internalization assay described herein. FAP antibody and SC-Cas9 conjugates internalized quickly in human fibroblasts.

Subsequently, ex vivo editing of fibroblasts by an anti-FAP antibody was assessed. A guide RNA targeting CD47 was associated with the respective TAGE agents to form ribonucleoproteins, and the ribonucleoproteins were co-incubated with fibroblasts to test for editing. Editing was measured using a phenotypic readout measuring the loss of surface CD47 using flow cytometry. Editing was compared to Cas9(C80A)-2×NLS with and without spycatcher (SC). Further, anti-FAP antibody linked to a spy tag (ST) with a long linker (LL) or short linker (SL) was assessed. Human dermal fibroblasts were incubated with 3750 nM of indicated molecules for 1 hour, then kept in 375 nM RNP for an additional 5 days. Edited cells were detected by a loss of CD47 surface protein. Editing values were determined as the mean of technical triplicates for each group.

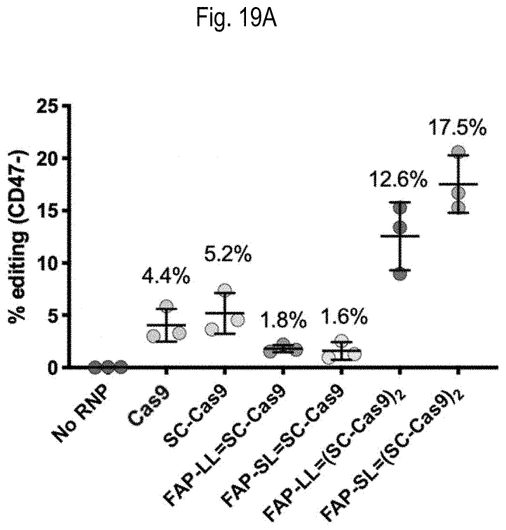

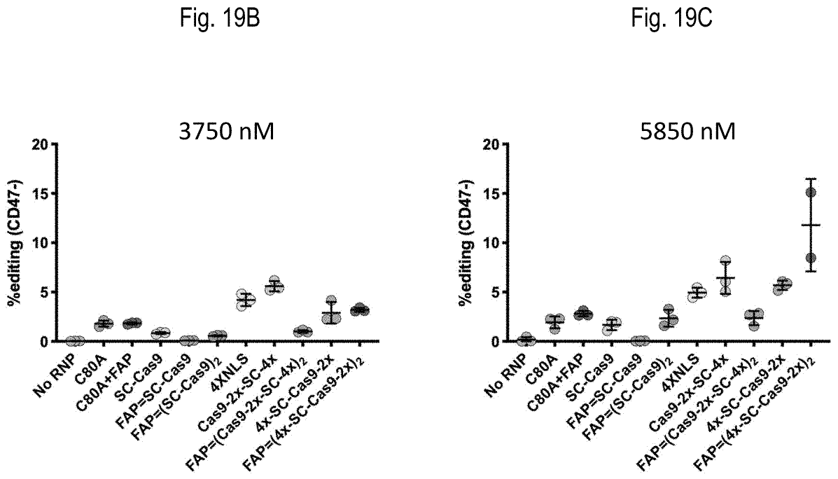

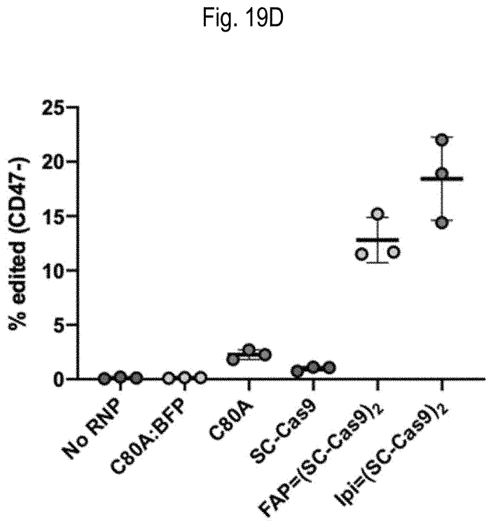

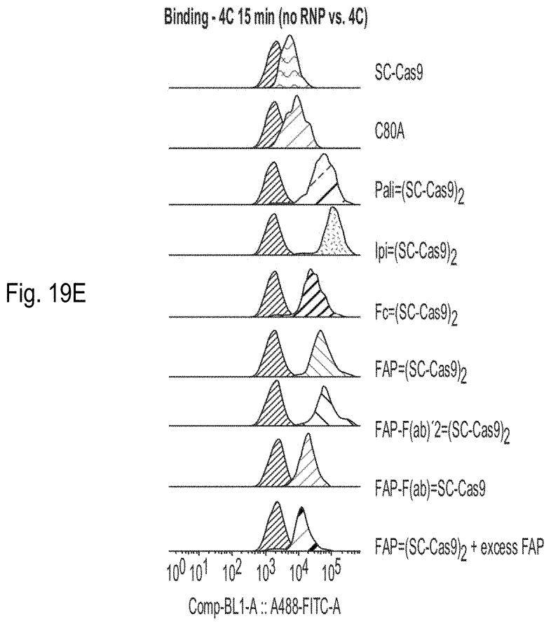

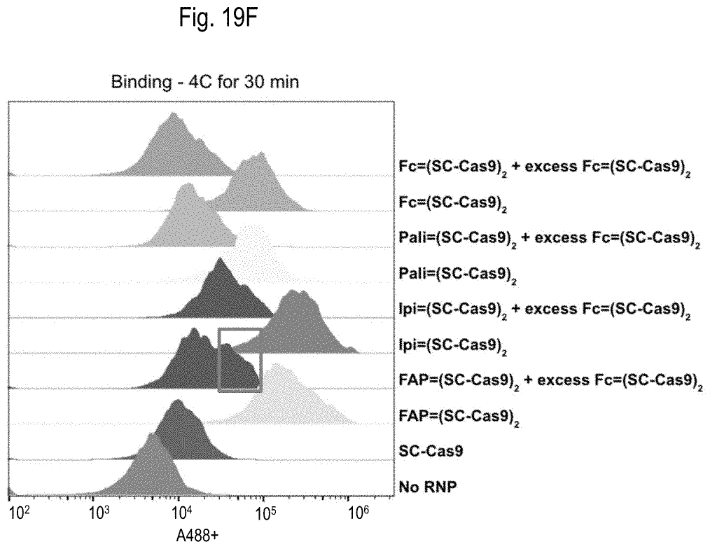

As shown in FIG. 19A, the conjugation of the FAP-ST antibody to SC-Cas9 showed higher levels of editing compared with SC-Cas9 (naked control). To rule out effects of an unconjugated antibody, an anti-FAP-ST antibody was added in trans during editing of Cas9(C80A)-2×NLS (C80A+FAP). Despite binding and internalizing similarly to conjugates with a single Cas9 moiety per antibody, only 2:1 Cas9:Ab conjugates (2 Cas9 per 1 Ab) edited better than controls. In particular, FAP=(4×NLS-SC-Cas9-2×NLS)₂ showed enhanced editing over 4×NLS-SC-Cas9-2×NLS alone at high concentrations (FIGS. 19B and 19C). Further, 2:1 Cas9:Ab conjugates edited better than the condition in which the anti-FAP antibody was delivered in trans with an unconjugated Cas9.

Editing of fibroblasts by an anti-CTLA4 antibody (ipilimumab, Ipi=(SC-Cas9)₂) was assessed as a negative (isotype) control for the FAP=(SC-Cas9)₂, as fibroblasts do not express CTLA-4. Further, a variety of antibody=SC-Cas9 conjugates bound fibroblasts similarly (FIG. 19E). All constructs were tested on human dermal fibroblasts (donor 8194) at 50 nM concentration. Anti-CTLA4 control constructs edited similarly to the FAP=Cas9 conjugate and bound to fibroblasts, suggesting that there are additional mechanisms enabling uptake and editing in fibroblasts. For example, while SC-Cas9 and Cas9(C80A) did not show substantial cell binding, Cas9 conjugates including various non-specific (to fibroblasts) antibodies (e.g., ipilimumabj, palivizumab, or an Fc portion of an antibody with two Cas9s linked together) exhibited binding to fibroblasts (FIG. 19E). Excess FAP blocked binding of the anti-FAP antibody=SC-Cas9 conjugate to fibroblasts, indicating that there was specificity of the anti-FAP antibody=SC-Cas9 TAGE agent for FAP expressed on the cell surface of fibroblasts (FIG. 19E).

Next, a competition assay with excess Fc=SC-Cas9 (an antibody Fc domain conjugated to a SC-Cas9) was performed to determine whether binding by TAGE agents including an anti-FAP-antibody and SC-Cas9 was mediated by the Fc region of the anti-FAP antibody. Fc addition blocked binding of the anti-FAP antibody=(SC-Cas9)₂ TAGE agent, along with ipilimumab and palivizumab, to fibroblasts. This indicates that binding of the anti-FAP antibody=(SC-Cas9)₂ conjugate to fibroblasts may be mediated by the Fc domain of the anti-FAP antibody or by Cas9 itself. However, as shown in FIG. 19F, there was residual anti-FAP antibody=(SC-Cas9)₂ binding that could not be blocked by Fc=SC-Cas9, which is consistent with FAP-mediated binding (see box in FIG. 19F).

### Example 19. Antibody TAGE Agent Screen in Human T Cells

The goal of this study was to identify antibodies for engineering T cell targeting TAGE agents. In this screen, antibodies were collected against targets on human T cells that are clinically validated. Antibodies were generated with a SpyTag on a human IgG1 backbone so that they could be conjugated to SpyCatcher(SC)-Cas9 and validated for binding and editing.

Antibody Screening

For this screening assay, spytagged human T-cell binding antibodies were cloned and expressed in Expi293 cells. Expi293 cell cultures were grown in 24-well plate format in 4 mL of media. On Day 0, cells at 3×10⁶/mL and at least 95% viability were transfected with 0.5 ug per mL of cells of a vector expressing the heavy chain of the antibody and 0.5 ug per mL of cells of a vector expressing the light chain of the antibody. Cells were harvested on day 4, or when viability dropped below 85%, whichever came first. The cells were pelleted at 3000×g for 10 min, the supernatant was diluted 1:1 volume:volume with PBS and filtered with a 0.44 uM filter. The supernatant was kept at 4C overnight if not using that day.

To purify the antibodies, each antibody was expressed in 25 mLs of Expi293 cells prepared as noted above. Cell lysate from the antibody-expressing cells was then applied to a 5 mL MabSelect SuRe 5 mL HiTrap column and washed with PBS. Antibodies were eluted with 50 mM Citrate buffer and peak fractions were pooled. The pH of the pooled elution was adjusted to pH 7.5 with 1 M HEPES pH8. The final antibody solution was concentrated using a 30 kD concentrator.

To produce F(ab′)2 fragments, a Genovis FragT Midi-Spin resin was equilibrated in digestion buffer (150 mM NaCl and 10 mM Na3PO4, pH7.5). Antibody was added at twice the desired amount of final F(ab′)2 to account for any loss, and digested with shaking at room temperature for 2 hours. Digestion was confirmed by SDS-PAGE analysis.

Purified spytagged antibodies (Ab-ST) were mixed with Cas9(WT)-2×NLS-Spycatcher-HTN (“AC28”), alternatively referred to as “SC-Cas9”) in Expi293 media to form an Ab-ST=SC-Cas9 conjugate. Unconjugated excess Cas9 was ‘quenched’ with spytag (5× SpyTag solution at room temperature for 1-2 hours) to enable blocking without excess Cas9 creating noise. Experiments with PBMCs were performed by treating PBMCs with up to 10% Expi293 media. Conjugating Ab-ST=SC-Cas9 in Expi293 media thereby obviated the need for full conjugate purification.

For this assay, 45 antibodies that bound to receptors on human T cells were identified and were selected for cloning. 31 spytagged antibodies were expressed for further testing.

T Cell Binding of Antibodies

31 spytagged T-cell binding antibodies were tested for binding to human T cells. Palivizumab (“Pali”) and a no RNP condition with unstained cells were assessed as negative controls. Total PBMCs activated for zero, two, or seven days were stained with antibodies against indicated target for 4C for 1 hour at 70 nM. An A488-labeled anti-human secondary was used to detect binding. An ANOVA with multiple comparisons was conducted to compare each antibody to Pali; Antibodies were moved to the next step if they had significantly more staining than Pali.

14 out of the 31 tested antibodies bound human T cells significantly above background. The identified antibodies targeted the following antigens: CD11a, CD25, CD27, CD44, CD52, CD54(ICAM), CD59, GITR, HLA-DR, ICOS, OX40, PD-L1, and PD-1. TAGE agents containing anti-CTLA-4 mouse and human antibodies (including ipilimumab and tremelimumab) using the SpyCatcher conjugation system were previously tested on splenocytes and stimulated PBMCs. While these TAGE agents were able to internalize, editing was not observed. Further investigation of ipilimumab and tremelimumab is described below in addition to the new antigens identified in the T cell antibody screen.

T Cell Binding of Ab=Cas9 Conjugates

14 antibodies identified in the previous step, along with ipilimumab (“Ipi”) and tremelimumab (“Trem”) (16 antibodies total), were then selected for conjugation to Cas9. Total PBMCs were activated for two days and were then stained with Ab=Cas9 conjugates at 7 or 70 nM. Binding was detected based on the presence of A550-labeled gRNA. palivizumab (Pali) was used as a negative control. An ANOVA with multiple comparisons was conducted to compare each antibody to Pali, and antibodies were moved to next step if they had significantly more staining than Pali.

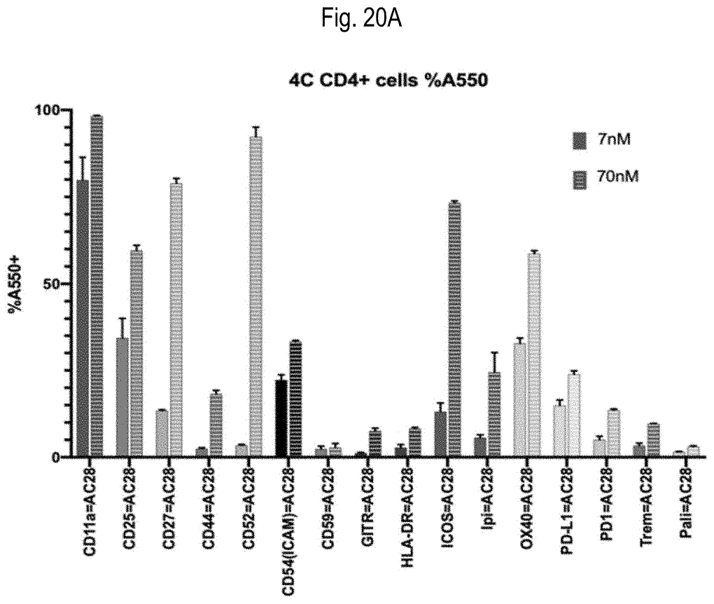

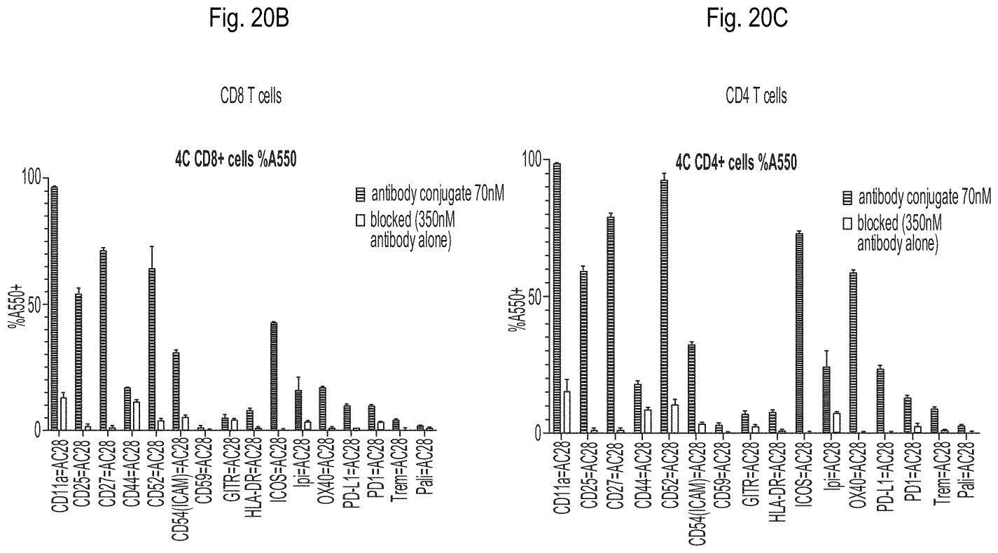

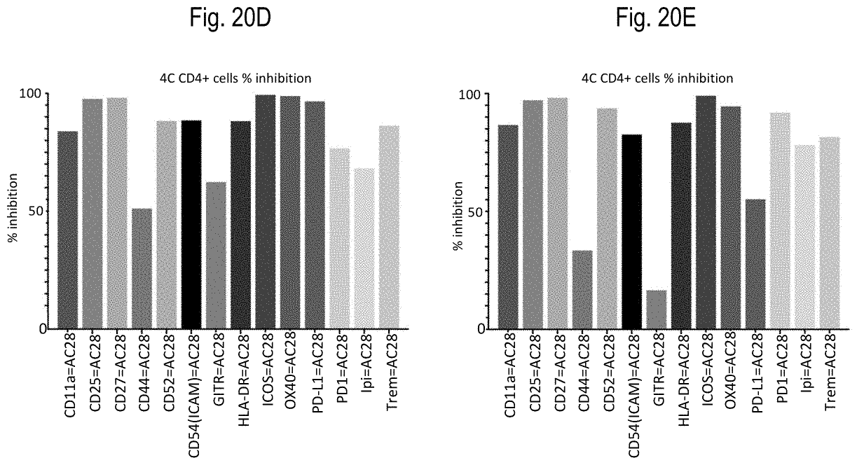

As shown in FIG. 20A, 14 of the tested Ab=Cas9 conjugates (antibody=Cas9(WT)-2×NLS-Spycatcher-HTN (“AC28”)) bound T cells significantly more than the negative control (Pali).

Binding of Ab=Cas9 conjugates to human T cells was further assessed in a 70 nM blocking assay with 5× “cold” antibody to assess whether excess unconjugated antibody blocks binding of Ab=Cas9 conjugates. Total PBMCs were activated for 2 days and were first blocked with 350 nM of unconjugated, SpyTagged antibody for 30 minutes. Then cells were stained with Ab=Cas9 conjugates at 70 nM. The A550 signal comes from an A550-labeled guide. Based on the A550 signal, percent blocking was determined by comparing the amount of binding of the antibody conjugate with and without blocking.

As shown in FIGS. 20B-20E, 14 out of the 15 tested TAGE agents (Ab=Cas9) bound human T cells and were blocked by an unconjugated antibody in the blocking assay. These results indicate that TAGE agents (Ab=Cas9) including antibodies that target CD11a, CD25, CD27, CD44, CD52, CD54(ICAM), GITR, HLA-DR, ICOS, OX40, PD-L1, or PD-1 can specifically bind human T cells.

### Example 20. Anti-CD11a and Anti-CD25 Antibody TAGE Agent Ex Vivo Editing of Human T Cells

Anti-CD11a and anti-CD25a antibodies (as identified in the T cell screen described in Example 19), or antigen-binding fragments thereof, were conjugated to Cas9 to form antibody-based TAGE agents (CD11a=Cas9 and CD25a=Cas9). In particular, anti-CD11a and anti-CD25a antibodies were conjugated to Cas9(WT)-2×NLS-SpyCatcher-4×NLS (“AC26”) or Cas9(WT)-2×NLS-SpyCatcher-HTN (“AC28”). Conjugates were purified and tested for editing of human T cells by co-incubation. A guide RNA targeting CD47 was associated with the respective TAGE agents, and the TAGE agents were co-incubated with T cells to test for editing. Editing was measured using a phenotypic readout measuring the loss of surface CD47 using flow cytometry. Editing of human T cells from two different donors was assessed. A full length antibody and an antibody fragment without the Fc domain were tested to determine whether a smaller molecule had higher editing. Palivizumab was used as a negative control.

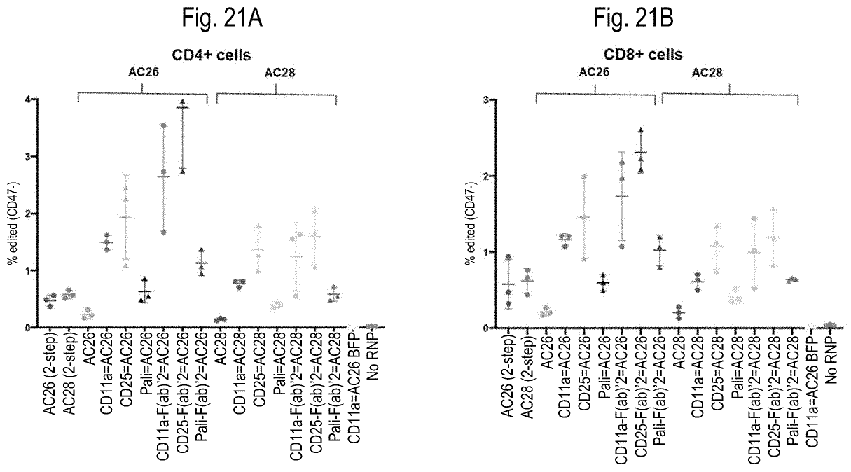

As shown in FIGS. 21A and 21B, Cas9 (e.g., AC26 or AC28) conjugated to either an anti-CD11a antibody or an anti-CD25 antibody, or antigen-binding fragments thereof, displayed increased editing of human T cells relative to an isotype control antibody. Similar editing was achieved in human T cells obtained from a second donor.

### Example 21. Comparison of Ex Vivo Editing Measurements by Flow Cytometry Vs. Amplicon Sequencing

In previous Examples, ex vivo editing was, in some cases, assessed by a phenotypic readout using flow cytometry (see, e.g., Examples 3, 8, 14, 17, 18, 20, 23, 27, 28, 39, 45, or 47). Flow cytometry offers a fast way to detect editing as compared to standard amplicon sequencing approaches. To determine the degree to which editing measurements obtained by flow cytometry correlate with editing measurements obtained by sequencing, T cells or fibroblasts edited by TAGE agents (via co-incubation or nucleofection) were assessed by both flow cytometry and next generation sequencing (NGS).

TAGE agents comprising Cas9(C80A)-2×NLS or 4×NLS-Cas9(C80A)-2×NLS were complexed with a sgRNA targeting CD47 or CD44 to form ribonucleoproteins (RNPs). A non-targeting sgRNA was used as a negative control (sgBFP; BFP is a gene not present in the human genome).

Editing of fibroblasts by TAGE agents was assessed by co-incubation or nucleofection with each TAGE agent.

To assess editing of fibroblasts by co-incubation, human dermal fibroblasts were grown on tissue culture plates. RNPs were added to the wells of a 96-well round-bottomed Ultra-Low Attachment tissue culture plate. 30 uL of the appropriate RNP was added to reach an RNP concentration of 5 uM. Human dermal fibroblasts were harvested from tissue culture plates and resuspended in fibroblast growth media at 20×10⁶ cells/mL. 10 uL of fibroblasts were added to the wells containing RNP. The final conditions in each well were: 40 uL volume; 3.75 uM RNP; 200,000 cells/well at 5×10⁶ cells/mL. The plate was incubated for 1 hour at 37 degrees Celsius. After the incubation, each sample was transferred to one well of a 12-well tissue culture plate containing 960 uL of fibroblast growth medium, for a final volume of 1 mL per well. Three days later, cells were lifted from the plates and transferred to the wells of 6-well tissue culture plates. An additional three days later (six days after co-incubation), cells were harvested and divided in half. Half of the cells were used for genomic DNA isolation and processed for Next-Gen Sequencing (NGS), and half of the cells were processed for flow cytometry, as outlined below.

To assess editing of fibroblasts by nucleofection, Human dermal fibroblasts were grown on tissue culture plates. RNPs were added to the wells of a 96-well round-bottomed Ultra-Low Attachment tissue culture plate. 5 uL of the appropriate RNP was added to each well to reach an RNP concentration of 5 uM. Human dermal fibroblasts were harvested from tissue culture plates and resuspended in Lonza Nucleofection Buffer P4 at 10×10⁶ cells/mL. 20 uL of fibroblasts were added to the wells containing RNP. The final conditions in each well were: 25 uL volume; 1 uM RNP; 200,000 cells/well at 8×10⁶ cells/mL. Cells mixed with RNP were transferred to the wells of nucleofection cassettes for the Lonza 4D Nucleofector. Cells were nucleofected using the Lonza 4D Nucleofector using the instrument code DS-137. After the nucleofection, each sample was transferred to one well of a 12-well tissue culture plate containing 975 uL of fibroblast growth medium, for a final volume of 1 mL per well. Three days later, cells were lifted from the plates and transferred to the wells of 6-well tissue culture plates. An additional three days later (six days after co-incubation), the cells were harvested and divided in half. Half of the cells were used for genomic DNA isolation and processed for Next-Gen Sequencing (NGS), and half of the cells were processed for flow cytometry, as described below.

Editing of T cells by TAGE agents was assessed by co-incubation with each TAGE agent. Human T cells were cultured for 4 days in T cell culture medium containing CD3 and CD28 cross-linking antibodies for T cell stimulation. After 4 days of stimulation, cells were harvested and resuspended in T cell growth media at 20×10⁶ cells/mL. RNPs were added to the wells of a 96-well round-bottomed Ultra-Low Attachment tissue culture plate. 30 uL of the appropriate RNP was added to each well to reach an RNP concentration of 5 uM. 10 uL of T cells were added to the wells containing RNP. The final conditions in each well were: 40 uL volume; 3.75 uM RNP; 200,000 cells/well at 5×10⁶ cells/mL. The plate was incubated for 1 hour at 37 degrees Celsius. After the incubation, each sample was diluted with 160 uL of T cell growth media. Over the next six days, cells were fed fresh media and expanded to larger well volume as appropriate for standard T cell growth conditions. An additional six days after co-incubation, the cells were harvested and divided in half. Half of the cells were used for genomic DNA isolation and processed for Next-Gen Sequencing (NGS), and half of the cells were processed for flow cytometry.

First, editing was measured using a phenotypic readout measuring the loss of surface CD47 or CD44 using flow cytometry. Cells were processed by standard flow cytometry methods and stained with antibodies against the human CD44 and CD47 proteins. Samples were analyzed on a flow cytometer. Gene editing was measured by analyzing the frequency of cells with decreased CD44 or CD47 staining. Cells edited with CD44-targeting RNPs were analyzed for CD44 staining in comparison to cells treated with a non-targeting (sgBFP) RNP. Cells edited with CD47-targeting RNPs were analyzed for CD47 staining in comparison to cells treated with a non-targeting (sgBFP) RNP.

Next, editing was measured using next-generation sequencing. Genomic DNA isolated from at least 10,000 cells/sample was amplified by PCR. PCR primers contained a gene-specific region and a region containing adapters to enable Illumina-based sequencing. Each sample was sequenced using an Illumina sequencing instrument. Sequencing reads for each sample were aligned to the genomic DNA sequence of the target region of the human genome. Unmodified sequences and sequences containing insertion and deletion mutations (indels) were counted for each sample. Gene editing was measured as the frequency of indel mutations at the corresponding RNP target site for each sample.

For each sample, gene editing as measured by flow cytometry was compared to gene editing as measured by NGS.

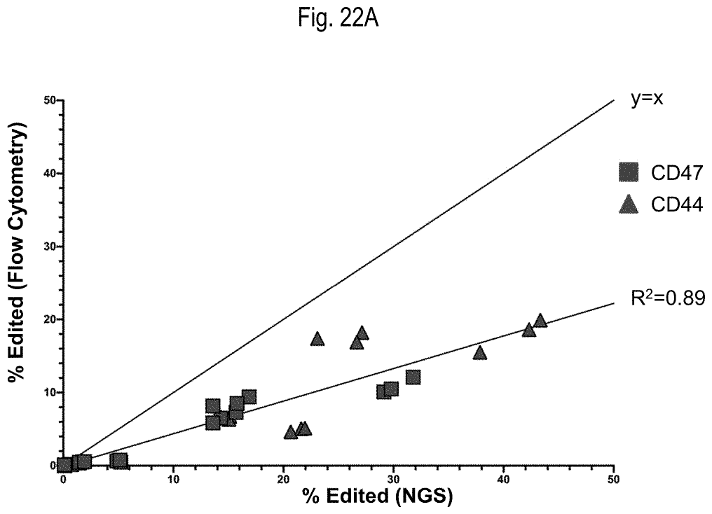

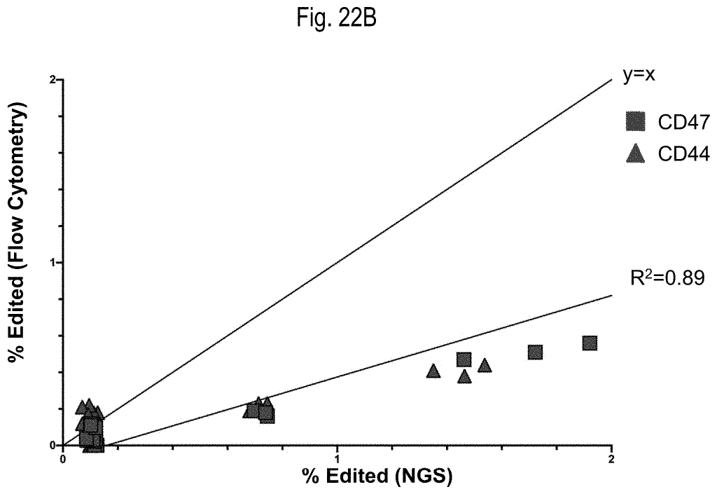

As shown in FIG. 22A, the percentage of editing as measured by flow cytometry correlated with amplicon sequencing across genes and cell types. Editing measurements obtained by flow cytometry and sequencing also correlated in cells with a lower degree of editing (FIG. 22B).

These results indicate that the phenotypic flow cytometry readout is representative of the amplicon sequencing assay, where either can be used to determine efficacy of a TAGE agent for gene editing. The results provided in FIGS. 22A and 22B also suggest that the flow cytometry assay may underestimate the level of gene editing by 2- to 4-fold as compared to editing measurements obtained by sequencing across different cell types, sgRNA, and editing efficiencies.

TABLE 4
Sequence Table

| SEQ ID | | |
|---|---|---|
| NO: | DESCRIPTION | SEQUENCE |
| SEQ ID | Cas9(C80A) | MDKKYSIGLDIGTNSVGWAVITDEYKVPSKKFKVLGNTDR |
| NO: 1 | (amino acid | HSIKKNLIGALLFDSGETAEATRLKRTARRRYTRRKNRIAY |
| | sequence) | LQEIFSNEMAKVDDSFFHRLEESFLVEEDKKHERHPIFGN |
| | | IVDEVAYHEKYPTIYHLRKKLVDSTDKADLRLIYLALAHMIK |
| | | FRGHFLIEGDLNPDNSDVDKLFIQLVQTYNQLFEENPINA |
| | | SGVDAKAILSARLSKSRRLENLIAQLPGEKKNGLFGNLIAL |
| | | SLGLTPNFKSNFDLAEDAKLQLSKDTYDDDLDNLLAQIGD |
| | | QYADLFLAAKNLSDAILLSDILRVNTEITKAPLSASMIKRYD |
| | | EHHQDLTLLKALVRQQLPEKYKEIFFDQSKNGYAGYIDG |
| | | GASQEEFYKFIKPILEKMDGTEELLVKLNREDLLRKQRTF |
| | | DNGSIPHQIHLGELHAILRRQEDFYPFLKDNREKIEKILTF |
| | | RIPYYVGPLARGNSRFAWMTRKSEETITPWNFEEVVDKG |
| | | ASAQSFIERMTNFDKNLPNEKVLPKHSLLYEYFTVYNELT |
| | | KVKYVTEGMRKPAFLSGEQKKAIVDLLFKTNRKVTVKQL |
| | | KEDYFKKIECFDSVEISGVEDRFNASLGTYHDLLKIIKDKD |
| | | FLDNEENEDILEDIVLTLTLFEDREMIEERLKTYAHLFDDK |
| | | VMKQLKRRRYTGWGRLSRKLINGIRDKQSGKTILDFLKS |
| | | DGFANRNFMQLIHDDSLTFKEDIQKAQVSGQGDSLHEHI |
| | | ANLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHIV |
| | | PQSFLKDDSIDNKVLTRSDKNRGKSDNVPSEEVVKKMKN |
| | | YWRQLLNAKLITQRKFDNLTKAERGGLSELDKAGFIKRQL |
| | | VETRQITKHVAQILDSRMNTKYDENDKLIREVKVITLKSKL |
| | | VSDFRKDFQFYKVREINNYHHAHDAYLNAVVGTALIKKYP |
| | | KLESEFVYGDYKVYDVRKMIAKSEQEIGKATAKYFFYSNI |
| | | MNFFKTEITLANGEIRKRPLIETNGETGEIVWDKGRDFAT |
| | | VRKVLSMPQVNIVKKTEVQTGGFSKESILPKRNSDKLIAR |
| | | KKDWDPKKYGGFDSPTVAYSVLVVAKVEKGKSKKLKSV |
| | | KELLGITIMERSSFEKNPIDFLEAKGYKEVKKDLIIKLPKYS |
| | | LFELENGRKRMLASAGELQKGNELALPSKYVNFLYLASH |
| | | YEKLKGSPEDNEQKQLFVEQHKHYLDEIIEQISEFSKRVIL |
| | | ADANLDKVLSAYNKHRDKPIREQAENIIHLFTLTNLGAPAA |
| | | FKYFDTTIDRKRYTSTKEVLDATLIHQSITGLYETRIDLSQL |
| | | GGDGSPKKKRKVEDPKKKRKVD |
| SEQ ID | Protein A | VDNKFNKEQQNAFYEILHLPNLNEEQRNAFIQSLKDDPS |
| NO: 2 | (amino acid | QSANLLAEAKKLNDAQAPKVDNKFNKEQQNAFYEILHLP |
| | sequence) | NLNEEQRNAFIQSLKDDPSQSANLLAEAKKLNGAQAPK |
| SEQ ID | Cas9(WT)-2xNL5- | MDKKYSIGLDIGTNSVGWAVITDEYKVPSKKFKVLGNTDR |
| NO: 3 | proteinA | HSIKKNLIGALLFDSGETAEATRLKRTARRRYTRRKNRIC |
| | (amino acid | YLQEIFSNEMAKVDDSFFHRLEESFLVEEDKKHERHPIFG |
| | sequence) | NIVDEVAYHEKYPTIYHLRKKLVDSTDKADLRLIYLALAHM |
| | Protein A is | IKFRGHFLIEGDLNPDNSDVDKLFIQLVQTYNQLFEENPIN |
| | underlined | ASGVDAKAILSARLSKSRRLENLIAQLPGEKKNGLFGNLIA |
| | | LSLGLTPNFKSNFDLAEDAKLQLSKDTYDDDLDNLLAQIG |
| | | DQYADLFLAAKNLSDAILLSDILRVNTEITKAPLSASMIKRY |
| | | DEHHQDLTLLKALVRQQLPEKYKEIFFDQSKNGYAGYID |
| | | GGASQEEFYKFIKPILEKMDGTEELLVKLNREDLLRKQRT |
| | | FDNGSIPHQIHLGELHAILRRQEDFYPFLKDNREKIEKILTF |
| | | RIPYYVGPLARGNSRFAWMTRKSEETITPWNFEEVVDKG |
| | | ASAQSFIERMTNFDKNLPNEKVLPKHSLLYEYFTVYNELT |
| | | KVKYVTEGMRKPAFLSGEQKKAIVDLLFKTNRKVTVKQL |
| | | KEDYFKKIECFDSVEISGVEDRFNASLGTYHDLLKIIKDKD |
| | | FLDNEENEDILEDIVLTLTLFEDREMIEERLKTYAHLFDDK |
| | | VMKQLKRRRYTGWGRLSRKLINGIRDKQSGKTILDFLKS |
| | | DGFANRNFMQLIHDDSLTFKEDIQKAQVSGQGDSLHEHI |
| | | ANLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHIV |
| | | PQSFLKDDSIDNKVLTRSDKNRGKSDNVPSEEVVKKMKN |
| | | YWRQLLNAKLITQRKFDNLTKAERGGLSELDKAGFIKRQL |
| | | VETRQITKHVAQILDSRMNTKYDENDKLIREVKVITLKSKL |
| | | VSDFRKDFQFYKVREINNYHHAHDAYLNAVVGTALIKKYP |
| | | KLESEFVYGDYKVYDVRKMIAKSEQEIGKATAKYFFYSNI |
| | | MNFFKTEITLANGEIRKRPLIETNGETGEIVWDKGRDFAT |
| | | VRKVLSMPQVNIVKKTEVQTGGFSKESILPKRNSDKLIAR |
| | | KKDWDPKKYGGFDSPTVAYSVLVVAKVEKGKSKKLKSV |
| | | KELLGITIMERSSFEKNPIDFLEAKGYKEVKKDLIIKLPKYS |
| | | LFELENGRKRMLASAGELQKGNELALPSKYVNFLYLASH |
| | | YEKLKGSPEDNEQKQLFVEQHKHYLDEIIEQISEFSKRVIL |
| | | ADANLDKVLSAYNKHRDKPIREQAENIIHLFTLTNLGAPAA |
| | | FKYFDTTIDRKRYTSTKEVLDATLIHQSITGLYETRIDLSQL |
| | | GGDGSPKKKRKVEDPKKKRKVDNGSSGSELVDNKFNKE |
| | | QQNAFYEILHLPNLNEEQRNAFIQSLKDDPSQSANLLAEA |
| | | KKLNDAQAPKVDNKFNKEQQNAFYEILHLPNLNEEQRNA |
| | | FIQSLKDDPSQSANLLAEAKKLNGAQAPK |
| SEQ ID | Cas9(C80A)- | MDKKYSIGLDIGTNSVGWAVITDEYKVPSKKFKVLGNTDR |
| NO: 4 | MHCiiNb-2XNLS | HSIKKNLIGALLFDSGETAEATRLKRTARRRYTRRKNRIAY |
| | (amino acid | LQEIFSNEMAKVDDSFFHRLEESFLVEEDKKHERHPIFGN |
| | sequence) | IVDEVAYHEKYPTIYHLRKKLVDSTDKADLRLIYLALAHMIK |
| | | FRGHFLIEGDLNPDNSDVDKLFIQLVQTYNQLFEENPINA |
| | | SGVDAKAILSARLSKSRRLENLIAQLPGEKKNGLFGNLIAL |
| | | SLGLTPNFKSNFDLAEDAKLQLSKDTYDDDLDNLLAQIGD |
| | | QYADLFLAAKNLSDAILLSDILRVNTEITKAPLSASMIKRYD |
| | | EHHQDLTLLKALVRQQLPEKYKEIFFDQSKNGYAGYIDG |
| | | GASQEEFYKFIKPILEKMDGTEELLVKLNREDLLRKQRTF |
| | | DNGSIPHQIHLGELHAILRRQEDFYPFLKDNREKIEKILTF |
| | | RIPYYVGPLARGNSRFAWMTRKSEETITPWNFEEVVDKG |
| | | ASAQSFIERMTNFDKNLPNEKVLPKHSLLYEYFTVYNELT |
| | | KVKYVTEGMRKPAFLSGEQKKAIVDLLFKTNRKVTVKQL |
| | | KEDYFKKIECFDSVEISGVEDRFNASLGTYHDLLKIIKDKD |
| | | FLDNEENEDILEDIVLTLTLFEDREMIEERLKTYAHLFDDK |
| | | VMKQLKRRRYTGWGRLSRKLINGIRDKQSGKTILDFLKS |
| | | DGFANRNFMQLIHDDSLTFKEDIQKAQVSGQGDSLHEHI |
| | | ANLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHIV |
| | | PQSFLKDDSIDNKVLTRSDKNRGKSDNVPSEEVVKKMKN |
| | | YWRQLLNAKLITQRKFDNLTKAERGGLSELDKAGFIKRQL |
| | | VETRQITKHVAQILDSRMNTKYDENDKLIREVKVITLKSKL |
| | | VSDFRKDFQFYKVREINNYHHAHDAYLNAVVGTALIKKYP |
| | | KLESEFVYGDYKVYDVRKMIAKSEQEIGKATAKYFFYSNI |
| | | MNFFKTEITLANGEIRKRPLIETNGETGEIVWDKGRDFAT |
| | | VRKVLSMPQVNIVKKTEVQTGGFSKESILPKRNSDKLIAR |
| | | KKDWDPKKYGGFDSPTVAYSVLVVAKVEKGKSKKLKSV |
| | | KELLGITIMERSSFEKNPIDFLEAKGYKEVKKDLIIKLPKYS |
| | | LFELENGRKRMLASAGELQKGNELALPSKYVNFLYLASH |
| | | YEKLKGSPEDNEQKQLFVEQHKHYLDEIIEQISEFSKRVIL |
| | | ADANLDKVLSAYNKHRDKPIREQAENIIHLFTLTNLGAPAA |
| | | FKYFDTTIDRKRYTSTKEVLDATLIHQSITGLYETRIDLSQL |
| | | GGDGASGASAQVQLVESGGGLVQAGDSLRLSCAASGR |
| | | TFSRGVMGWFRRAPGKEREFVAIFSGSSWSGRSTYYSD |
| | | SVKGRFTISRDNAKNTVYLQMNGLKPEDTAVYYCAAGYP |
| | | EAYSAYGRESTYDYWGQGTQVTVSSEPKTPKPQPARQA |
| | | CTSGASGASGSPKKKRKVEDPKKKRKVD |
| SEQ ID | 6xHis-3C-Cas9 | HHHHHHLEVLFQGPMDKKYSIGLDIGTNSVGWAVITDEY |
| NO: 5 | C80A | KVPSKKFKVLGNTDRHSIKKNLIGALLFDSGETAEATRLK |
| | (amino acid | RTARRRYTRRKNRIAYLQEIFSNEMAKVDDSFFHRLEESF |
| | sequence) | LVEEDKKHERHPIFGNIVDEVAYHEKYPTIYHLRKKLVDST |
| | | DKADLRLIYLALAHMIKFRGHFLIEGDLNPDNSDVDKLFIQ |
| | | LVQTYNQLFEENPINASGVDAKAILSARLSKSRRLENLIAQ |
| | | LPGEKKNGLFGNLIALSLGLTPNFKSNFDLAEDAKLQLSK |
| | | DTYDDDLDNLLAQIGDQYADLFLAAKNLSDAILLSDILRVN |
| | | TEITKAPLSASMIKRYDEHHQDLTLLKALVRQQLPEKYKEI |
| | | FFDQSKNGYAGYIDGGASQEEFYKFIKPILEKMDGTEELL |
| | | VKLNREDLLRKQRTFDNGSIPHQIHLGELHAILRRQEDFY |
| | | PFLKDNREKIEKILTFRIPYYVGPLARGNSRFAWMTRKSE |
| | | ETITPWNFEEVVDKGASAQSFIERMTNFDKNLPNEKVLP |
| | | KHSLLYEYFTVYNELTKVKYVTEGMRKPAFLSGEQKKAIV |
| | | DLLFKTNRKVTVKQLKEDYFKKIECFDSVEISGVEDRFNA |
| | | SLGTYHDLLKIIKDKDFLDNEENEDILEDIVLTLTLFEDREM |
| | | IEERLKTYAHLFDDKVMKQLKRRRYTGWGRLSRKLINGIR |
| | | DKQSGKTILDFLKSDGFANRNFMQLIHDDSLTFKEDIQKA |
| | | QVSGQGDSLHEHIANLAGSPAIKKGILQTVKVVDELVKVM |
| | | GRHKPENIVIEMARENQTTQKGQKNSRERMKRIEEGIKE |
| | | LGSQILKEHPVENTQLQNEKLYLYYLQNGRDMYVDQELD |
| | | INRLSDYDVDHIVPQSFLKDDSIDNKVLTRSDKNRGKSDN |
| | | VPSEEVVKKMKNYWRQLLNAKLITQRKFDNLTKAERGGL |
| | | SELDKAGFIKRQLVETRQITKHVAQILDSRMNTKYDENDK |
| | | LIREVKVITLKSKLVSDFRKDFQFYKVREINNYHHAHDAYL |
| | | NAVVGTALIKKYPKLESEFVYGDYKVYDVRKMIAKSEQEI |
| | | GKATAKYFFYSNIMNFFKTEITLANGEIRKRPLIETNGETG |
| | | EIVWDKGRDFATVRKVLSMPQVNIVKKTEVQTGGFSKES |
| | | ILPKRNSDKLIARKKDWDPKKYGGFDSPTVAYSVLVVAKV |
| | | EKGKSKKLKSVKELLGITIMERSSFEKNPIDFLEAKGYKEV |
| | | KKDLIIKLPKYSLFELENGRKRMLASAGELQKGNELALPS |
| | | KYVNFLYLASHYEKLKGSPEDNEQKQLFVEQHKHYLDEII |
| | | EQISEFSKRVILADANLDKVLSAYNKHRDKPIREQAENIIH |
| | | LFTLTNLGAPAAFKYFDTTIDRKRYTSTKEVLDATLIHQSIT |
| | | GLYETRIDLSQLGGDGSPKKKRKVEDPKKKRKVD |
| SEQ ID | Spycatcher-Cas9 | MVTTLSGLSGEQGPSGDMTTEEDSATHIKFSKRDEDGR |
| NO: 6 | (WT) | ELAGATMELRDSSGKTISTWISDGHVKDFYLYPGKYTFV |
| | (amino acid | ETAAPDGYEVATAITFTVNEQGQVTVNGEATKGDAHTGS |
| | sequence) | SGSNGSSGSELDKKYSIGLDIGTNSVGWAVITDEYKVPS |
| | SpyCatcher | KKFKVLGNTDRHSIKKNLIGALLFDSGETAEATRLKRTAR |
| | sequence is | RRYTRRKNRICYLQEIFSNEMAKVDDSFFHRLEESFLVEE |
| | underlined | DKKHERHPIFGNIVDEVAYHEKYPTIYHLRKKLVDSTDKA |
| | | DLRLIYLALAHMIKFRGHFLIEGDLNPDNSDVDKLFIQLVQ |
| | | TYNQLFEENPINASGVDAKAILSARLSKSRRLENLIAQLPG |
| | | EKKNGLFGNLIALSLGLTPNFKSNFDLAEDAKLQLSKDTY |
| | | DDDLDNLLAQIGDQYADLFLAAKNLSDAILLSDILRVNTEIT |
| | | KAPLSASMIKRYDEHHQDLTLLKALVRQQLPEKYKEIFFD |
| | | QSKNGYAGYIDGGASQEEFYKFIKPILEKMDGTEELLVKL |
| | | NREDLLRKQRTFDNGSIPHQIHLGELHAILRRQEDFYPFL |
| | | KDNREKIEKILTFRIPYYVGPLARGNSRFAWMTRKSEETIT |
| | | PWNFEEVVDKGASAQSFIERMTNFDKNLPNEKVLPKHSL |
| | | LYEYFTVYNELTKVKYVTEGMRKPAFLSGEQKKAIVDLLF |
| | | KTNRKVTVKQLKEDYFKKIECFDSVEISGVEDRFNASLGT |
| | | YHDLLKIIKDKDFLDNEENEDILEDIVLTLTLFEDREMIEER |
| | | LKTYAHLFDDKVMKQLKRRRYTGWGRLSRKLINGIRDKQ |
| | | SGKTILDFLKSDGFANRNFMQLIHDDSLTFKEDIQKAQVS |
| | | GQGDSLHEHIANLAGSPAIKKGILQTVKVVDELVKVMGRH |
| | | KPENIVIEMARENQTTQKGQKNSRERMKRIEEGIKELGS |
| | | QILKEHPVENTQLQNEKLYLYYLQNGRDMYVDQELDINR |
| | | LSDYDVDHIVPQSFLKDDSIDNKVLTRSDKNRGKSDNVP |
| | | SEEVVKKMKNYWRQLLNAKLITQRKFDNLTKAERGGLSE |
| | | LDKAGFIKRQLVETRQITKHVAQILDSRMNTKYDENDKLIR |
| | | EVKVITLKSKLVSDFRKDFQFYKVREINNYHHAHDAYLNA |
| | | VVGTALIKKYPKLESEFVYGDYKVYDVRKMIAKSEQEIGK |
| | | ATAKYFFYSNIMNFFKTEITLANGEIRKRPLIETNGETGEIV |
| | | WDKGRDFATVRKVLSMPQVNIVKKTEVQTGGFSKESILP |
| | | KRNSDKLIARKKDWDPKKYGGFDSPTVAYSVLVVAKVEK |
| | | GKSKKLKSVKELLGITIMERSSFEKNPIDFLEAKGYKEVKK |
| | | DLIIKLPKYSLFELENGRKRMLASAGELQKGNELALPSKY |
| | | VNFLYLASHYEKLKGSPEDNEQKQLFVEQHKHYLDEIIEQ |
| | | ISEFSKRVILADANLDKVLSAYNKHRDKPIREQAENIIHLFT |
| | | LTNLGAPAAFKYFDTTIDRKRYTSTKEVLDATLIHQSITGL |
| | | YETRIDLSQLGGDGSPKKKRKVEDPKKKRKVD |
| SEQ ID | Cas9 (WT)- | MDKKYSIGLDIGTNSVGWAVITDEYKVPSKKFKVLGNTDR |
| NO: 7 | Spycatcher | HSIKKNLIGALLFDSGETAEATRLKRTARRRYTRRKNRIC |
| | (amino acid | YLQEIFSNEMAKVDDSFFHRLEESFLVEEDKKHERHPIFG |
| | sequence) | NIVDEVAYHEKYPTIYHLRKKLVDSTDKADLRLIYLALAHM |
| | SpyCatcher | IKFRGHFLIEGDLNPDNSDVDKLFIQLVQTYNQLFEENPIN |
| | Sequence | ASGVDAKAILSARLSKSRRLENLIAQLPGEKKNGLFGNLIA |
| | Underlined | LSLGLTPNFKSNFDLAEDAKLQLSKDTYDDDLDNLLAQIG |
| | | DQYADLFLAAKNLSDAILLSDILRVNTEITKAPLSASMIKRY |
| | | DEHHQDLTLLKALVRQQLPEKYKEIFFDQSKNGYAGYID |
| | | GGASQEEFYKFIKPILEKMDGTEELLVKLNREDLLRKQRT |
| | | FDNGSIPHQIHLGELHAILRRQEDFYPFLKDNREKIEKILTF |
| | | RIPYYVGPLARGNSRFAWMTRKSEETITPWNFEEVVDKG |
| | | ASAQSFIERMTNFDKNLPNEKVLPKHSLLYEYFTVYNELT |
| | | KVKYVTEGMRKPAFLSGEQKKAIVDLLFKTNRKVTVKQL |
| | | KEDYFKKIECFDSVEISGVEDRFNASLGTYHDLLKIIKDKD |
| | | FLDNEENEDILEDIVLTLTLFEDREMIEERLKTYAHLFDDK |
| | | VMKQLKRRRYTGWGRLSRKLINGIRDKQSGKTILDFLKS |
| | | DGFANRNFMQLIHDDSLTFKEDIQKAQVSGQGDSLHEHI |
| | | ANLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHIV |
| | | PQSFLKDDSIDNKVLTRSDKNRGKSDNVPSEEVVKKMKN |
| | | YWRQLLNAKLITQRKFDNLTKAERGGLSELDKAGFIKRQL |
| | | VETRQITKHVAQILDSRMNTKYDENDKLIREVKVITLKSKL |
| | | VSDFRKDFQFYKVREINNYHHAHDAYLNAVVGTALIKKYP |
| | | KLESEFVYGDYKVYDVRKMIAKSEQEIGKATAKYFFYSNI |
| | | MNFFKTEITLANGEIRKRPLIETNGETGEIVWDKGRDFAT |
| | | VRKVLSMPQVNIVKKTEVQTGGFSKESILPKRNSDKLIAR |
| | | KKDWDPKKYGGFDSPTVAYSVLVVAKVEKGKSKKLKSV |
| | | KELLGITIMERSSFEKNPIDFLEAKGYKEVKKDLIIKLPKYS |
| | | LFELENGRKRMLASAGELQKGNELALPSKYVNFLYLASH |
| | | YEKLKGSPEDNEQKQLFVEQHKHYLDEIIEQISEFSKRVIL |
| | | ADANLDKVLSAYNKHRDKPIREQAENIIHLFTLTNLGAPAA |
| | | FKYFDTTIDRKRYTSTKEVLDATLIHQSITGLYETRIDLSQL |
| | | GGDGSPKKKRKVEDPKKKRKVDNGSSGSELMVTTLSGL |
| | | SGEQGPSGDMTTEEDSATHIKFSKRDEDGRELAGATME |
| | | LRDSSGKTISTWISDGHVKDFYLYPGKYTFVETAAPDGY |
| | | EVATAITFTVNEQGQVTVNGEATKGDAHTGSSGS |
| SEQ ID | NLS | PKKKRKV |
| NO: 8 | (amino acid | |
| | sequence) | |
| SEQ ID | Tat | RKKRRQRRR |
| NO: 9 | (amino acid | |
| | sequence) | |
| SEQ ID | HIS-4XNLS | HHHHHHLEVLFQGPMNATPKKKRKVGGSPKKKRKVGGS |
| NO: 10 | (amino acid | PKKKRKVGGSPKKKRKVGIHGVPAAT |
| | sequence) | |
| SEQ ID | Tat-NLS | GAYGRKKRRQRRRPPAGTSVSLKKKRKVG |
| NO: 11 | (amino acid | |
| | sequence) | |
| SEQ ID | HIS_TAT-NLS | HHHHHHGMGAAGRKKRRQRRRPPAGTSVSLKKKRKV |
| NO: 12 | (HTN) | |
| | (amino acid | |
| | sequence) | |
| SEQ ID | HIV-REV | RQARRNRRRRWR |
| NO: 13 | (amino acid | |
| | sequence) | |
| SEQ ID | Anti-CD45 | EVKLLESGGGLVQPGGSLKLSCAASGFDFSRYWMSWV |
| NO: 14 | antibody heavy | RQAPGKGLEWIGEINPTSSTINFTPSLKDKVFISRDNAKN |
| | chain variable | TLYLQMSKVRSEDTALYYCARGNYYRYGDAMDYWGQG |
| | region | TSVTVSSA |
| | (amino acid | |
| | sequence) | |
| SEQ ID | Anti-CD45 | DIALTQSPASLAVSLGQRATISCRASKSVSTSGYSYLHWY |
| NO: 15 | antibody light | QQKPGQPPKLLIYLASNLESGVPARFSGSGSGTDFTLNIH |
| | chain variable | PVEEEDAATYYCQHSRELPFTFGSGTKLEIKR |
| | region | |
| | (amino acid | |
| | sequence) | |
| SEQ ID | Anti-CD48 | EVQLLESGGGLVHPGGSLRLSCAASGFTFGGYAMSWVR |
| NO: 16 | antibody heavy | QAPGKGLEWVSLISGSGGSTYYADSVKGRFTIFRDNSKN |
| | chain variable | TLYLQMISLRAEDSAVYYCAKYSNYDYFDPWGQGTLVTV |
| | region | SSA |
| | (amino acid | |
| | sequence) | |
| SEQ ID | Anti-CD48 | EIVLTQSPGTLSLSPGERVTLSCRASQSVSSSYLAWYQQ |
| NO: 17 | antibody light | KPGQAPRLLIYGASSRATGIPDRFSGSGSGTDFTLTISRL |
| | chain variable | EPEDFAVYYCQQYGSSPRTFGQGTKVEIK |
| | region | |
| | (amino acid | |
| | sequence) | |
| SEQ ID | Anti-TIM3 | EVQLLESGGGLVQPGGSLRLSCAAASGFTFSSYDMSWV |
| NO: 18 | antibody heavy | RQAPGKGLDWVSTISGGGTYTYYQDSVKGRFTISRDNS |
| | chain variable | KNTLYLQMNSLRAEDTAVYYCASMDYWGQGTTVTVSSA |
| | region | |
| | (amino acid | |
| | sequence) | |
| SEQ ID | Anti-TIM3 | DIQMTQSPSSLSASVGDRVTITCRASQSIRRYLNWYHQK |
| NO: 19 | antibody light | PGKAPKLLIYGASTLQSGVPSRFSGSGSGTDFTLTISSLQ |
| | chain variable | PEDFAVYYCQQSHSAPLTFGGGTKVEIKR |
| | region | |
| | (amino acid | |
| | sequence) | |
| SEQ ID | Anti-CD73 | EVQLLESGGGLVQPGGSLRLSCAASGFTFSSYAYSWVR |
| NO: 20 | antibody heavy | QAPGKGLEWVSAISGSGGRTYYADSVKGRFTISRDNSK |
| | chain variable | NTLYLQMNSLRAEDTAVYYCARLGYGRVDEWGRGTLVT |
| | region | VSSA |
| | (amino acid | |
| | sequence) | |
| SEQ ID | Anti-CD73 | QSVLTQPPSASGTPGQRVTISCSGSLSNIGRNPVNWYQ |
| NO: 21 | antibody light | QLPGTAPKLLIYLDNLRLSGVPDRFSGSKSGTSASLAISG |
| | chain variable | LQSEDEADYYCATWDDSHPGWTFGGGTKLTVLGQPKA |
| | region | APS |
| | (amino acid | |
| | sequence) | |
| SEQ ID | Anti-TIGIT | EVQLQQSGPGLVKPSQTLSLTCAISGDSVSSNSAAWNWI |
| NO: 22 | antibody heavy | RQSPSRGLEWLGKTYYRFKWYSDYAVSVKGRITINPDTS |
| | chain variable | KNQFSLQLNSVTPEDTAVFYCTRESTTYDLLAGPFDYWG |
| | region | QGTLVTVSSA |
| | (amino acid | |
| | sequence) | |
| SEQ ID | Anti-TIGIT | DIVMTQSPDSLAVSLGERATINCKSSQTVLYSSNNKKYLA |
| NO: 23 | antibody light | WYQQKPGQPPNLLIYWASTRESGVPDRESGSGSGTDFT |
| | chain variable | LTISSLQAEDVAVYYCQQYYSTPFTFGPGTKVEIKR |
| | region | |
| | (amino acid | |
| | sequence) | |
| SEQ ID | Anti-CCR4 | EVQLVESGGDLVQPGRSLRLSCAASGFIFSNYGMSWVR |
| NO: 24 | antibody heavy | QAPGKGLEWVATISSASTYSYYPDSVKGRFTISRDNAKN |
| | chain variable | SLYLQMNSLRVEDTALYYCGRHSDGNFAFGYWGQGTLV |
| | region | TVSSA |
| | (amino acid | |
| | sequence) | |
| SEQ ID | Anti-CCR4 | DVLMTQSPLSLPVTPGEPASISCRSSRNIVHINGDTYLEW |
| NO: 25 | antibody light | YLQKPGQSPQLLIYKVSNRFSGVPDRFSGSGSGTDFTLKI |
| | chain variable | SRVEAEDVGVYYCFQGSLLPWTFGQGTKVEIKR |
| | region | |
| | (amino acid | |
| | sequence) | |
| SEQ ID | Anti-IL-4R | EVQLVESGGGLEQPGGSLRLSCAGSGFTFRDYAMTWV |
| NO: 26 | antibody heavy | RQAPGKGLEWVSSISGSGGNTYYADSVKGRFTISRDNS |
| | chain variable | KNTLYLQMNSLRAEDTAVYYCAKDRLSITIRPRYYGLDV |
| | region | WGQGTTVTVSSA |
| | (amino acid | |
| | sequence) | |
| SEQ ID | Anti-IL-4R | DIVMTQSPLSLPVTPGEPASISCRSSQSLLYSIGYNYLDW |
| NO: 27 | antibody light | YLQKSGQSPQLLIYLGSNRASGVPDRFSGSGSGTDFTLK |
| | chain variable | ISRVEAEDVGFYYCMQALQTPYTFGQGTKLEIKR |
| | region | |
| | (amino acid | |
| | sequence) | |
| SEQ ID | Anti-CCR2 | EVQLVESGGGLVKPGGSLRLSCAASGFTFSAYAMNWVR |
| NO: 28 | antibody heavy | QAPGKGLEWVGRIRTKNNNYATYYADSVKDRFTISRDDS |
| | chain variable | KNTLYLQMNSLKTEDTAVYYCTTFYGNGVWGQGTLVTV |
| | region | SSA |
| | (amino acid | |
| | sequence) | |
| SEQ ID | Anti-CCR2 | DVVMTQSPLSLPVTLGQPASISCKSSQSLLDSDGKTFLN |
| NO: 29 | antibody light | WFQQRPGQSPRRLIYLVSKLDSGVPDRFSGSGSGTDFT |
| | chain variable | LKISRVEAEDVGVYYCWQGTHFPYTFGQGTRLEIKR |
| | region | |
| | (amino acid | |
| | sequence) | |
| SEQ ID | Anti-CD44 | EVQLVESGGGLVKPGGSLRLSCAASGFTFSSYDMSWVR |
| NO: 30 | antibody heavy | QAPGKGLEWVSTISSGGSYTYYLDSIKGRFTISRDNAKNS |
| | chain variable | LYLQMNSLRAEDTAVYYCARQGLDYWGRGTLVTVSSA |
| | region | |
| | (amino acid | |
| | sequence) | |
| SEQ ID | Anti-CD44 | EIVLTQSPATLSLSPGERATLSCSASSSINYIYWYQQKPG |
| NO: 31 | antibody light | QAPRLLIYLTSNLASGVPARFSGSGSGTDFTLTISSLEPE |
| | chain variable | DFAVYYCLQWSSNPLTFGGGTKVEIKR |
| | region | |
| | (amino acid | |
| | sequence) | |
| SEQ ID | Anti-CCR5 | EVQLVESGGGLVKPGGSLRLSCAASGYTFSNYWIGWVR |
| NO: 32 | antibody heavy | QAPGKGLEWIGDIYPGGNYIRNNEKFKDKTTLSADTSKN |
| | chain variable | TAYLQMNSLKTEDTAVYYCGSSFGSNYVFAWFTYWGQG |
| | region | TLVTVSSA |
| | (amino acid | |
| | sequence) | |
| SEQ ID | Anti-CCR5 | DIVMTQSPLSLPVTPGEPASISCRSSQRLLSSYGHTYLH |
| NO: 33 | antibody light | WYLQKPGQSPQLLIYEVSNRFSGVPDRFSGSGSGTDFTL |
| | chain variable | KISRVEAEDVGVYYCSQSTHVPLTFGQGTKVEIKR |
| | region | |
| | (amino acid | |
| | sequence) | |
| SEQ ID | Anti-CXCR4 | EVQLVESGGGLVQPGGSLRLSCAAAGFTFSSYSMNWVR |
| NO: 34 | antibody heavy | QAPGKGLEWVSYISSRSRTIYYADSVKGRFTISRDNAKN |
| | chain variable | SLYLQMNSLRDEDTAVYYCARDYGGQPPYYYYYGMDV |
| | region | WGQGTTVTVSSA |
| | (amino acid | |
| | sequence) | |
| SEQ ID | Anti-CXCR4 | DIQMTQSPSSLSASVGDRVTITCRASQGISSWLAWYQQK |
| NO: 35 | antibody light | PEKAPKSLIYAASSLQSGVPSRFSGSGSGTDFTLTISSLQ |
| | chain variable | PEDFVTYYCQQYNSYPRTFGQGTKVEIKR |
| | region | |
| | (amino acid | |
| | sequence) | |
| SEQ ID | Anti-SLAMF7 | EVQLVESGGGLVQPGGSLRLSCAASGFDFSRYWMSWV |
| NO: 36 | antibody heavy | RQAPGKGLEWIGEINPDSSTINYAPSLKDKFIISRDNAKNS |
| | chain variable | LYLQMNSLRAEDTAVYYCARPDGNYWYFDVWGQGTLV |
| | region | TVSSA |
| | (amino acid | |
| | sequence) | |
| SEQ ID | Anti-SLAMF7 | DIQMTQSPSSLSASVGDRVTITCKASQDVGIAVAWYQQK |
| NO: 37 | antibody light | PGKVPKLLIYWASTRHTGVPDRFSGSGSGTDFTLTISSLQ |
| | chain variable | PEDVATYYCQQYSSYPYTFGQGTKVEIKR |
| | region | |
| | (amino acid | |
| | sequence) | |
| SEQ ID | Anti-ICOS | EVQLVESGGGLVQPGGSLRLSCAASGFTFSDYWMDWV |
| NO: 38 | antibody heavy | RQAPGKGLVWVSNIDEDGSITEYSPFVKGRFTISRDNAK |
| | chain variable | NTLYLQMNSLRAEDTAVYYCTRWGRFGFDSWGQGTLVT |
| | region | VSSA |
| | (amino acid | |
| | sequence) | |
| SEQ ID | Anti-ICOS | DIVMTQSPDSLAVSLGERATINCKSSQSLLSGSFNYLTWY |
| NO: 39 | antibody light | QQKPGQPPKLLIFYASTRHTGVPDRFSGSGSGTDFTLTIS |
| | chain variable | SLQAEDVAVYYCHHHYNAPPTFGPGTKVDIKR |
| | region | |
| | (amino acid | |
| | sequence) | |
| SEQ ID | Anti-PD-L1 | EVQLVESGGGLVQPGGSLRLSCAASGFTFSRYWMSWV |
| NO: 40 | antibody heavy | RQAPGKGLEWVANIKQDGSEKYYVDSVKGRFTISRDNA |
| | chain variable | KNSLYLQMNSLRAEDTAVYYCAREGGWFGELAFDYWG |
| | region | QGTLVTVSSA |
| | (amino acid | |
| | sequence) | |
| SEQ ID | Anti-PD-L1 | EIVLTQSPGTLSLSPGERATLSCRASQRVSSSYLAWYQQ |
| NO: 41 | antibody light | KPGQAPRLLIYDASSRATGIPDRFSGSGSGTDFTLTISRL |
| | chain variable | EPEDFAVYYCQQYGSLPWTFGQGTKVEIKR |
| | region | |
| | (amino acid | |
| | sequence) | |
| SEQ ID | Anti-OX40 | EVQLVESGGGLVQPGGSLRLSCAASGFTFSSYSMNWVR |
| NO: 42 | antibody heavy | QAPGKGLEWVSYISSSSSTIDYADSVKGRFTISRDNAKNS |
| | chain variable | LYLQMNSLRDEDTAVYYCARESGWYLFDYWGQGTLVTV |
| | region | SSA |
| | (amino acid | |
| | sequence) | |
| SEQ ID | Anti-OX40 | DIQMTQSPSSLSASVGDRVTITCRASQGISSWLAWYQQK |
| NO: 43 | antibody light | PEKAPKSLIYAASSLQSGVPSRFSGSGSGTDFTLTISSLQ |
| | chain variable | PEDFATYYCQQYNSYPPTFGGGTKVEIKR |
| | region | |
| | (amino acid | |
| | sequence) | |
| SEQ ID | Anti-CD11a | EVQLVESGGGLVQPGGSLRLSCAASGYSFTGHWMNWV |
| NO: 44 | antibody heavy | RQAPGKGLEWVGMIHPSDSETRYNQKFKDRFTISVDKSK |
| | chain variable | NTLYLQMNSLRAEDTAVYYCARGIYFYGTTYFDYWGQG |
| | region | TLVTVSSA |
| | (amino acid | |
| | sequence) | |
| SEQ ID | Anti-CD11a | DIQMTQSPSSLSASVGDRVTITCRASKTISKYLAWYQQKP |
| NO: 45 | antibody light | GKAPKLLIYSGSTLQSGVPSRFSGSGSGTDFTLTISSLQP |
| | chain variable | EDFATYYCQQHNEYPLTFGQGTKVEIKR |
| | region | |
| | (amino acid | |
| | sequence) | |
| SEQ ID | Anti-CD40L | EVQLVESGGGLVQPGGSLRLSCAVSGFSSTNYHVHWVR |
| NO: 46 | antibody heavy | QAPGKGLEWMGVIWGDGDTSYNSVLKSRFTISRDTSKN |
| | chain variable | TVYLQMNSLRAEDTAVYYCARQLTHYYVLAAWGQGTLV |
| | region | TVSSA |
| | (amino acid | |
| | sequence) | |
| SEQ ID | Anti-CD40L | DIQMTQSPSSLSASVGDRVTITCRASEDLYYNLAWYQRK |
| NO: 47 | antibody light | PGKAPKLLIYDTYRLADGVPSRFSGSGSGTDYTLTISSLQ |
| | chain variable | PEDFASYYCQQYYKFPFTFGQGTKVEIKR |
| | region | |
| | (amino acid | |
| | sequence) | |
| SEQ ID | Anti-IFNAR1 | EVQLVQSGAEVKKPGESLKISCKGSGYIFTNYWIAWVRQ |
| NO: 48 | antibody heavy | MPGKGLESMGIIYPGDSDIRYSPSFQGQVTISADKSITTA |
| | chain variable | YLQWSSLKASDTAMYYCARHDIEGFDYWGRGTLVTVSS |
| | region | A |
| | (amino acid | |
| | sequence) | |
| SEQ ID | Anti-IFNAR1 | EIVLTQSPGTLSLSPGERATLSCRASQSVSSSFFAWYQQ |
| NO: 49 | antibody light | KPGQAPRLLIYGASSRATGIPDRLSGSGSGTDFTLTITRL |
| | chain variable | EPEDFAVYYCQQYDSSAITFGQGTRLEIKR |
| | region | |
| | (amino acid | |
| | sequence) | |
| SEQ ID | Anti-Transferin | QVQLQESGGGVVQPGRSLRLSCAASRFTFSSYAMHWV |
| NO: 50 | receptor antibody | RQAPGKGLEWVAVISYDGSNKYYADSVKGRTISRDNSK |
| | heavy chain | NTLYLQMNSLRAEDTAVYYCARDLSGYGSYPDYWGQGT |
| | variable region | LVGVS |
| | (amino acid | |
| | sequence) | |
| SEQ ID | Anti-Transferin | SSELTQDPAVSVALGQTVRITCQGDSLRSYYASWYQQK |
| NO: 51 | receptor antibody | PGQAPVLVMYGRNERPSGVPDRFSGSKSGTSASLAISG |
| | light chain | LQPEDEANYYCAGWDDSLTGPVFGGGTKLTVLG |
| | variable region | |
| | (amino acid | |
| | sequence) | |
| SEQ ID | Anti-CD80 | QVQLQESGPGLVKPSETLSLTCAVSGGSISGGYGWGWI |
| NO: 52 | antibody heavy | RQPPGKGLEWIGSFYSSSGNTYYNPSLKSQVTISTDTSK |
| | chain variable | NQFSLKLNSMTAADTAVYYCVRDRLFSVVGMVYNNWFD |
| | region | VWGPGVLVTVSSA |
| | (amino acid | |
| | sequence) | |
| SEQ ID | Anti-CD80 | ESALTQPPSVSGAPGQKVTISCTGSTSNIGGYDLHWYQQ |
| NO: 53 | antibody light | LPGTAPKLLIYDINKRPSGISDRFSGSKSGTAASLAITGLQ |
| | chain variable | TEDEADYYCQSYDSSLNAQVFGGGTRLTVLG |
| | region | |
| | (amino acid | |
| | sequence) | |
| SEQ ID | Anti-IL6-R | QVQLQESGPGLVRPSQTLSLTCTVSGYSITSDHAWSWV |
| NO: 54 | antibody heavy | RQPPGRGLEWIGYISYSGITTYNPSLKSRVTMLRDTSKN |
| | chain variable | QFSLRLSSVTAADTAVYYCARSLARTTAMDYWGQGSLV |
| | region | TVSSA |
| | (amino acid | |
| | sequence) | |
| SEQ ID | Anti-IL6-R | DIQMTQSPSSLSASVGDRVTITCRASQDISSYLNWYQQK |
| NO: 55 | antibody light | PGKAPKLLIYYTSRLHSGVPSRFSGSGSGTDFTFTISSLQ |
| | chain variable | PEDIATYYCQQGNTLPYTFGQGTKVEIKR |
| | region | |
| | (amino acid | |
| | sequence) | |
| SEQ ID | Anti-TCRb | QVQLQQSGAELARPGASVKMSCKASGYTFTSYTMHWV |
| NO: 56 | antibody heavy | KQRPGQGLEWIGYINPSSGYTNYNQKFKDKATLTADKSS |
| | chain variable | STAYMQLSSLTSEDSAVYYCARWRDAYYAMDYWGQGT |
| | region | SVTVSSA |
| | (amino acid | |
| | sequence) | |
| SEQ ID | Anti-TCRb | QIVLTQSPAIMSASPGEKVTMTCSASSSVSYMHWYQQK |
| NO: 57 | antibody light | SGTSPKRWIYDTSKLASGVPARFSGSGSGTSYSLTISSM |
| | chain variable | EAEDAATYYCQQWSSNPFTFGSGTKLEIKR |
| | region | |
| | (amino acid | |
| | sequence) | |
| SEQ ID | Anti-CD59 | QVQLQQSGGGVVQPGRSLGLSCAASGFTFSSYGMNWV |
| NO: 58 | antibody heavy | RQAPGKGLEWVSYISSSSSTIYYADSVKGRFTISRDNSKN |
| | chain variable | TLYLQMNSLRAEDTAVYYCARGPGMDVWGQGTTVTVSS |
| | region | A |
| | (amino acid | |
| | sequence) | |
| SEQ ID | Anti-CD59 | DIVLTQSPDSLAVSLGERATINCKSSQSVLYSSNNKNYLA |
| NO: 59 | antibody light | WYQQKPGQPPKLLIYWASTRESGVPDRFSGSGSGTDFT |
| | chain variable | PAISSLQAEDVAVYYCQQYYSTPQLTFGGGTKVDIKR |
| | region | |
| | (amino acid | |
| | sequence) | |
| SEQ ID | Anti-CD4 antibody | QVQLQQSGPEVVKPGASVKMSCKASGYTFTSYVIHWVR |
| NO: 60 | heavy chain | QKPGQGLDWIGYINPYNDGTDYDEKFKGKATLTSDTSTS |
| | variable region | TAYMELSSLRSEDTAVYYCAREKDNYATGAWFAYWGQ |
| | (amino acid | GTLVTVSSA |
| | sequence) | |
| SEQ ID | Anti-CD4 antibody | DIVMTQSPDSLAVSLGERVTMNCKSSQSLLYSTNQKNYL |
| NO: 61 | light chain | AWYQQKPGQSPKLLIYWASTRESGVPDRFSGSGSGTDF |
| | variable region | TLTISSVQAEDVAVYYCQQYYSYRTFGGGTKLEIKR |
| | (amino acid | |
| | sequence) | |
| SEQ ID | Anti-HLA-DR | QVQLQQSGSELKKPGASVKVSCKASGFTFTNYGMNWV |
| NO: 62 | antibody heavy | KQAPGQGLKWMGWINTYTREPTYADDFKGRFAFSLDTS |
| | chain variable | VSTAYLQISSLKADDTAVYFCARDITAVVPTGFDYWGQG |
| | region | SLVTVSSA |
| | (amino acid | |
| | sequence) | |
| SEQ ID | Anti-HLA-DR | DIQLTQSPSSLSASVGDRVTITCRASENIYSNLAWYRQKP |
| NO: 63 | antibody light | GKAPKLLVFAASNLADGVPSRFSGSGSGTDYTFTISSLQ |
| | chain variable | PEDIATYYCQHFWTTPWAFGGGTKLQIKR |
| | region | |
| | (amino acid | |
| | sequence) | |
| SEQ ID | Anti-LAG3 | QVQLQQWGAGLLKPSETLSLTCAVYGGSFSDYYWNWIR |
| NO: 64 | antibody heavy | QPPGKGLEWIGEINHRGSTNSNPSLKSRVTLSLDTSKNQ |
| | chain variable | FSLKLRSVTAADTAVYYCAFGYSDYEYNWFDPWGQGTL |
| | region | VTVSSA |
| | (amino acid | |
| | sequence) | |
| SEQ ID | Anti-LAG3 | EIVLTQSPATLSLSPGERATLSCRASQSISSYLAWYQQKP |
| NO: 65 | antibody light | GQAPRLLIYDASNRATGIPARFSGSGSGTDFTLTISSLEP |
| | chain variable | EDFAVYYCQQRSNWPLTFGQGTNLEIKR |
| | region | |
| | (amino acid | |
| | sequence) | |
| SEQ ID | Anti-4-1BB | QVQLQQWGAGLLKPSETLSLTCAVYGGSFSGYYWSWIR |
| NO: 66 | antibody heavy | QSPEKGLEWIGEINHGGYVTYNPSLESRVTISVDTSKNQF |
| | chain variable | SLKLSSVTAADTAVYYCARDYGPGNYDWYFDLWGRGTL |
| | region | VTVSSA |
| | (amino acid | |
| | sequence) | |
| SEQ ID | Anti-4-1BB | EIVLTQSPATLSLSPGERATLSCRASQSVSSYLAWYQQK |
| NO: 67 | antibody light | PGQAPRLLIYDASNRATGIPARFSGSGSGTDFTLTISSLE |
| | chain variable | PEDFAVYYCQQRSNWPPALTFCGGTKVEIKR |
| | region | |
| | (amino acid | |
| | sequence) | |
| SEQ ID | Anti-GITR | QVQLVESGGGVVQPGRSLRLSCAASGFTFSSYAMHWV |
| NO: 68 | antibody heavy | RQAPGKGLEWVAVISYDGSNKYYADSVKGRFTISRDNSK |
| | chain variable | NTLYLQMNSLRAEDTAVYYCARGIAAAGPPYYYYYYYMD |
| | region | VWGKGTTVTVSSA |
| | (amino acid | |
| | sequence) | |
| SEQ ID | Anti-GITR | DIQMTQSPSSLSASVGDRVTITCRASQTIYNYLNWYQQK |
| NO: 69 | antibody light | PGKAPKLLIYAASSLQSGVPSRFGGRGYGTDFTLTINSLQ |
| | chain variable | PEDFATYFCQQSYTSPLTFGQGTKVDIKR |
| | region | |
| | (amino acid | |
| | sequence) | |
| SEQ ID | Anti-CD27 | QVQLVESGGGVVQPGRSLRLSCAASGFTFSSYDMHWV |
| NO: 70 | antibody heavy | RQAPGKGLEWVAVIWYDGSNKYYADSVKGRFTISRDNS |
| | chain variable | KNTLYLQMNSLRAEDTAVYYCARGSGNWGFFDYWGQG |
| | region | TLVTVSSA |
| | (amino acid | |
| | sequence) | |
| SEQ ID | Anti-CD27 | DIQMTQSPSSLSASVGDRVTITCRASQGISRWLAWYQQK |
| NO: 71 | antibody light | PEKAPKSLIYAASSLQSGVPSRFSGSGSGTDFTLTISSLQ |
| | chain variable | PEDFATYYCQQYNTYPRTFGQGTKVEIKR |
| | region | |
| | (amino acid | |
| | sequence) | |
| SEQ ID | Anti-nkg2a | QVQLVQSGAEVKKPGASVKVSCKASGYTFTSYWMNWV |
| NO: 72 | antibody heavy | RQAPGQGLEWMGRIDPYDSETHYAQKLQGRVTMTTDTS |
| | chain variable | TSTAYMELRSLRSDDTAVYYCARGGYDFDVGTLYWFFD |
| | region | VWGQGTTVTVSSA |
| | (amino acid | |
| | sequence) | |
| SEQ ID | Anti-nkg2a | DIQMTQSPSSLSASVGDRVTITCRASENIYSYLAWYQQK |
| NO: 73 | antibody light | PGKAPKLLIYNAKTLAEGVPSRFSGSGSGTDFTLTISSLQ |
| | chain variable | PEDFATYYCQHHYGTPRTFGGGTKVEIKR |
| | region | |
| | (amino acid | |
| | sequence) | |
| SEQ ID | Anti-CD25 | QVQLVQSGAEVKKPGSSVKVSCKASGYTFTSYRMHWV |
| NO: 74 | antibody heavy | RQAPGQGLEWIGYINPSTGYTEYNQKFKDKATITADEST |
| | chain variable | NTAYMELSSLRSEDTAVYYCARGGGVFDYWGQGTLVTV |
| | region | SSA |
| | (amino acid | |
| | sequence) | |
| SEQ ID | Anti-CD25 | DIQMTQSPSTLSASVGDRVTITCSASSSISYMHWYQQKP |
| NO: 75 | antibody light | GKAPKLLIYTTSNLASGVPARFSGSGSGTEFTLTISSLQP |
| | chain variable | DDFATYYCHQRSTYPLTFGQGTKVEVKR |
| | region | |
| | (amino acid | |
| | sequence) | |
| SEQ ID | Anti-CD3 antibody | QVQLQQSGAELARPGASVKMSCKASGYTFTRYTMHWV |
| NO: 76 | heavy chain | KQRPGQGLEWIGYINPSRGYTNYNQKFKDKATLTTDKSS |
| | variable region | STAYMQLSSLTSEDSAVYYCARYYDDHYCLDYWGQGTT |
| | (amino acid | LTVSSA |
| | sequence) | |
| SEQ ID | Anti-CD3 antibody | QIVLTQSPAIMSASPGEKVTMTCSASSSVSYMNWYQQK |
| NO: 77 | light chain | SGTSPKRWIYDTSKLASGVPAHFRGSGSGTSYSLTISGM |
| | variable region | EAEDAATYYCQQWSSNPFTFGSGTKLEINR |
| | (amino acid | |
| | sequence) | |
| SEQ ID | Anti-TLR2 | QVQLVQSGSELKKPGASVKLSCKASGFTFTTYGINWVRQ |
| NO: 78 | antibody heavy | APGQGLEWIGWIYPRDGSTNFNENFKDRATITVDTSAST |
| | chain variable | AYMELSSLRSEDTAVYFCARLTGGTFLDYWGQGTTVTV |
| | region | SSA |
| | (amino acid | |
| | sequence) | |
| SEQ ID | Anti-TLR2 | DIVLTQSPATLSLSPGERATLSCRASESVEYYGTSLMQW |
| NO: 79 | antibody light | YQQKPGQPPKLLIFGASNVESGVPDRFSGSGSGTDFTLK |
| | chain variable | ISRVEAEDVGMYFCQQSRKLPWTFGGGTKVEIKR |
| | region | |
| | (amino acid | |
| | sequence) | |
| SEQ ID | Anti-PD1 antibody | QVQLVQSGVEVKKPGASVKVSCKASGYTFTNYYMYWVR |
| NO: 80 | heavy chain | QAPGQGLEWMGGINPSNGGTNFNEKFKNRVTLTTDSST |
| | variable region | TTAYMELKSLQFDDTAVYYCARRDYRFDMGFDYWGQG |
| | (amino acid | TTVTVSSA |
| | sequence) | |
| SEQ ID | Anti-PD1 antibody | EIVLTQSPATLSLSPGERATLSCRASKGVSTSGYSYLHW |
| NO: 81 | light chain | YQQKPGQAPRLLIYLASYLESGVPARFSGSGSGTDFTLTI |
| | variable region | SSLEPEDFAVYYCQHSRDLPLTFGGGTKVEIKR |
| | (amino acid | |
| | sequence) | |
| SEQ ID | Anti-CD2 antibody | QVQLVQSGAEVKKPGASVKVSCKASGYTFTGYYMHWV |
| NO: 82 | heavy chain | RQAPGQGLEWMGRINPNSGGTNYAQKFQGRVTMTRDT |
| | variable region | SISTAYMELSRLRSDDTAVYYCARGRTEYIVVAEGFDYW |
| | (amino acid | GQGTLVTVSSA |
| | sequence) | |
| SEQ ID | Anti-CD2 antibody | DVVMTQSPPSLLVTLGQPASISCRSSQSLLHSSGNTYLN |
| NO: 83 | light chain | WLLQRPGQSPQPLIYLVSKLESGVPDRFSGSGSGTDFTL |
| | variable region | KISGVEAEDVGVYYCMQFTHYPYTFGQGTKLEIKR |
| | (amino acid | |
| | sequence) | |
| SEQ ID | Anti-CD52 | QVQLQESGPGLVRPSQTLSLTCTVSGFTFTDFYMNWVR |
| NO: 84 | antibody heavy | QPPGRGLEWIGFIRDKAKGYTTEYNPSVKGRVTMLVDTS |
| | chain variable | KNQFSLRLSSVTAADTAVYYCAREGHTAAPFDYWGQGS |
| | region | LVTVSSA |
| | (amino acid | |
| | sequence) | |
| SEQ ID | Anti-CD52 | DIQMTQSPSSLSASVGDRVTITCKASQNIDKYLNWYQQK |
| NO: 85 | antibody light | PGKAPKLLIYNTNNLQTGVPSRFSGSGSGTDFTFTISSLQ |
| | chain variable | PEDIATYYCLQHISRPRTFGQGTKVEIKR |
| | region | |
| | (amino acid | |
| | sequence) | |
| SEQ ID | Anti-CD54 (ICAM) | EVQLLESGGGLVQPGGSLRLSCAASGFTFSNAWMSWV |
| NO: 86 | antibody heavy | RQAPGKGLEWVAFIWYDGSNKYYADSVKGRFTISRDNS |
| | chain variable | KNTLYLQMNSLRAEDTAVYYCARYSGWYFDYWGQGTLV |
| | region | TVSSA |
| | (amino acid | |
| | sequence) | |
| SEQ ID | Anti-CD54 (ICAM) | QSVLTQPPSASGTPGQRVTISCTGSSSNIGAGYDVHWY |
| NO: 87 | antibody light | QQLPGTAPKLLIYDNNNRPSGVPDRFSGSKSGTSASLAIS |
| | chain variable | GLRSEDEADYYCQSYDSSLSAWLFGGGTKLTV |
| | region | |
| | (amino acid | |
| | sequence) | |
| SEQ ID | Anti-EGFR | QVQLKQSGPGLVQPSQSLSITCTVSGFSLTNYGVHWVR |
| NO: 88 | antibody heavy | QSPGKGLEWLGVIWSGGNTDYNTPFTSRLSINKDNSKS |
| | chain variable | QVFFKMNSLQSNDTAIYYCARALTYYDYEFAYWGQGTLV |
| | region | TVSAA |
| | (amino acid | |
| | sequence) | |
| SEQ ID | Anti-EGFR | DILLTQSPVILSVSPGERVSFSCRASQSIGTNIHWYQQRT |
| NO: 89 | antibody light | NGSPRLLIKYASESISGIPSRFSGSGSGTDFTLSINSVESE |
| | chain variable | DIADYYCQQNNNWPTTFGAGTKLELKR |
| | region | |
| | (amino acid | |
| | sequence) | |
| SEQ ID | Anti-IGF-1R | EVQLLESGGGLVQPGGSLRLSCTASGFTFSSYAMNWVR |
| NO: 90 | antibody heavy | QAPGKGLEWVSAISGSGGTTFYADSVKGRFTISRDNSRT |
| | chain variable | TLYLQMNSLRAEDTAVYYCAKDLGWSDSYYYYYGMDV |
| | region | WGQGTTVTVSSA |
| | (amino acid | |
| | sequence) | |
| SEQ ID | Anti-IGF-1R | DIQMTQFPSSLSASVGDRVTITCRASQGIRNDLGWYQQK |
| NO: 91 | antibody light | PGKAPKRLIYAASRLHRGVPSRFSGSGSGTEFTLTISSLQ |
| | chain variable | PEDFATYYCLQHNSYPCSFGQGTKLEIKR |
| | region | |
| | (amino acid | |
| | sequence) | |
| SEQ ID | Anti-CD30 | QIQLQQSGPEVVKPGASVKISCKASGYTFTDYYITWVKQ |
| NO: 92 | antibody heavy | KPGQGLEWIGWIYPGSGNTKYNEKFKGKATLTVDTSSST |
| | chain variable | AFMQLSSLTSEDTAVYFCANYGNYWFAYWGQGTQVTVS |
| | region | AA |
| | (amino acid | |
| | sequence) | |
| SEQ ID | Anti-CD30 | DIVLTQSPASLAVSLGQRATISCKASQSVDFDGDSYMNW |
| NO: 93 | antibody light | YQQKPGQPPKVLIYAASNLESGIPARFSGSGSGTDFTLNI |
| | chain variable | HPVEEEDAATYYCQQSNEDPWTFGGGTKLEIKR |
| | region | |
| | (amino acid | |
| | sequence) | |
| SEQ ID | Anti-CD19 | QVQLQQSGAELVRPGSSVKISCKASGYAFSSYWMNWVK |
| NO: 94 | antibody heavy | QRPGQGLEWIGQIWPGDGDTNYNGKFKGKATLTADESS |
| | chain variable | STAYMQLSSLASEDSAVYFCARRETTTVGRYYYAMDYW |
| | region | GQGTTVTVSSG |
| | (amino acid | |
| | sequence) | |
| SEQ ID | Anti-CD19 | DIQLTQSPASLAVSLGQRATISCKASQSVDYDGDSYLNW |
| NO: 95 | antibody light | YQQ1PGQPPKWYDASNLVSGIPPRFSGSGSGTDFTLNIH |
| | chain variable | PVEKVDAATYHCQQSTEDPWTFGGGTKLEIKR |
| | region | |
| | (amino acid | |
| | sequence) | |
| SEQ ID | Anti-CD34 | EVQLQQSGPELVKPGASVKISCKASGYSFIGYFMNWVM |
| NO: 96 | antibody heavy | QSHGRSLEWIGRINPYNGYTFYNQKFKGKATLTVDKSSS |
| | chain variable | TAHMELRSLASEDSAVYYCARHFRYDGVFYYAMDYWG |
| | region | QGTSVTVSSA |
| | (amino acid | |
| | sequence) | |
| SEQ ID | Anti-CD34 | QLVLTQSSSASFSLGASAKLTCTLSSQHSTFTIEWYQQQ |
| NO: 97 | antibody light | PLKPPKYVMDLKKDGSHSTGDGVPDRFSGSSSGADRYL |
| | chain variable | SISNIQPEDEATYICGVGDTIKEQFVYVFGGGTKVTVLG |
| | region | |
| | (amino acid | |
| | sequence) | |
| SEQ ID | Anti-CD59 | QVQLQQSGGGVVQPGRSLGLSCAASGFTFSSYGMNWV |
| NO: 98 | antibody heavy | RQAPGKGLEWVSYISSSSSTIYYADSVKGRFTISRDNSKN |
| | chain variable | TLYLQMNSLRAEDTAVYYCARGPGMDVWGQGTTVTVSS |
| | region | A |
| | (amino acid | |
| | sequence) | |
| SEQ ID | Anti-CD59 | DIVLTQSPDSLAVSLGERATINCKSSQSVLYSSNNKNYLA |
| NO: 99 | antibody light | WYQQKPGQPPKLLIYWASTRESGVPDRFSGSGSGTDFT |
| | chain variable | PAISSLQAEDVAVYYCQQYYSTPQLTFGGGTKVDIKR |
| | region | |
| | (amino acid | |
| | sequence) | |
| SEQ ID | Anti-FAP antibody | EVQLLESGGGLVQPGGSLRLSCAASGFTFSSHAMSWVR |
| NO: 100 | heavy chain | QAPGKGLEWVSAIWASGEQYYADSVKGRFTISRDNSKN |
| | variable region | TLYLQMNSLRAEDTAVYYCAKGWLGNFDYWGQGTLVTV |
| | (amino acid | SSA |
| | sequence) | |
| SEQ ID | Anti-FAP antibody | EIVLTQSPGTLSLSPGERATLSCRASQSVSRSYLAWYQQ |
| NO: 101 | light chain | KPGQAPRLLIIGASTRATGIPDRFSGSGSGTDFTLTISRLE |
| | variable region | PEDFAVYYCQQGQVIPPTFGQGTKVEIKR |
| | (amino acid | |
| | sequence) | |
| SEQ ID | Anti-hCTLA4 | QVQLVESGGGVVQPGRSLRLSCAASGFTFSSYTMHWV |
| NO: 102 | antibody | RQAPGKGLEWVTFISYDGNNKYYADSVKGRFTISRDNSK |
| | (ipilumimab) | NTLYLQMNSLRAEDTAIYYCARTGWLGPFDYWGQGTLV |
| | heavy chain | TVSSA |
| | variable region | |
| | (amino acid | |
| | sequence) | |
| SEQ ID | Anti-hCTLA4 | EIVLTQSPGTLSLSPGERATLSCRASQSVGSSYLAWYQQ |
| NO: 103 | (ipilumimab) | KPGQAPRLLIYGAFSRATGIPDRFSGSGSGTDFTLTISRL |
| | antibody light | EPEDFAVYYCQQYGSSPWTFGQGTKVEIKR |
| | chain variable | |
| | region | |
| | (amino acid | |
| | sequence) | |
| SEQ ID | Anti-hCTLA4 | QVQLVESGGGVVQPGRSLRLSCAASGFTFSSYGMHWV |
| NO: 104 | antibody | RQAPGKGLEWVAVIWYDGSNKYYADSVKGRFTISRDNS |
| | (tremelimumab) | KNTLYLQMNSLRAEDTAVYYCARDPRGATLYYYYYGMD |
| | heavy chain | VWGQGTTVTVSSA |
| | variable region | |
| | (amino acid | |
| | sequence) | |
| SEQ ID | Anti-hCTLA4 | DIQMTQSPSSLSASVGDRVTITCRASQSINSYLDWYQQK |
| NO: 105 | (tremelimumab) | PGKAPKLLIYAASSLQSGVPSRFSGSGSGTDFTLTISSLQ |
| | antibody light | PEDFATYYCQQYYSTPFTFGPGTKVEIKR |
| | chain variable | |
| | region | |
| | (amino acid | |
| | sequence) | |
| SEQ ID | Anti-mCTLA4 | EVQLQQSGPVLVKPGASVKMSCKASGYTFTDYYMNWV |
| NO: 106 | antibody heavy | KQSHGKSLEWIGVINPYNGDTSYNQKFKGKATLTVDKSS |
| | chain variable | STAYMELNSLTSEDSAVYYCARYYGSWFAYWGQGTLIT |
| | region | VST |
| | (amino acid | |
| | sequence) | |
| SEQ ID | Anti-mCTLA4 | DVLMTQTPLSLPVSLGDQASISCRSSQSIVHSNGNTYLE |
| NO: 107 | antibody light | WYLQKPGQSPKLLIYKVSNRFSGVPDRFSGSGSGTDFTL |
| | chain variable | KISRVEAEDLGVYYCFQGSHVPYTFGGGTKLEIKR |
| | region | |
| | (amino acid | |
| | sequence) | |
| SEQ ID | Anti-hCD22 | QVQLVQSGAEVKKPGSSVKVSCKASGYTFTSYWLHWVR |
| NO: 108 | antibody heavy | QAPGQGLEWIGYINPRNDYTEYNQNFKDKATITADESTN |
| | chain variable | TAYMELSSLRSEDTAFYFCARRDITTFYWGQGTTVTVSS |
| | region | A |
| | (amino acid | |
| | sequence) | |
| SEQ ID | Anti-hCD22 | DVLMTQTPLSLPVSLGDQASISCRSSQSIVHSNGNTYLE |
| NO: 109 | antibody light | WYLQKPGQSPKLLIYKVSNRFSGVPDRFSGSGSGTDFTL |
| | chain variable | KISRVEAEDLGVYYCFQGSHVPYTFGGGTKLEIKR |
| | region | |
| | (amino acid | |
| | sequence) | |
| SEQ ID | Anti-MHCII | QVQLQESGGGLVQPGGSLRLSCAASGKMSSRRCMAWF |
| NO: 110 | nanobody | RQAPGKERERVAKLLTTSGSTYLADSVKGRFTISQNNAK |
| | (amino acid | STVYLQMNSLKPEDTAMYYCAADSFEDPTCTLVTSSGAF |
| | sequence) | QYWGQGTQVTVSS |
| SEQ ID | Anti-EGFR | QVKLEESGGGSVQTGGSLRLTCAASGRTSRSYGMGWF |
| NO: 111 | nanobody | RQAPGKEREFVSGISWRGDSTGYADSVKGRFTISRDNA |
| | (amino acid | KNTVDLQMNSLKPEDTAIYYCAAAAGSAWYGTLYEYDY |
| | sequence) | WGQGTQVTVSS |
| SEQ ID | Anti-HER2 | QVQLQESGGGSVQAGGSLKLTCAASGYIFNSCGMGWY |
| NO: 112 | nanobody | RQSPGRERELVSRISGDGDTWHKESVKGRFTISQDNVK |
| | (amino acid | KTLYLQMNSLKPEDTAVYFCAVCYNLETYWGQGTQVTV |
| | sequence) | SS |
| SEQ ID | Anti-mCD47 | QVQLVESGGGLVEPGGSLRLSCAASGIIFKINDMGWYRQ |
| NO: 113 | nanobody | APGKRREWVAASTGGDEAIYRDSVKDRFTISRDAKNSVF |
| | (amino acid | LQMNSLKPEDTAVYYCTAVISTDRDGTEWRRYWGQGTQ |
| | sequence) | VYVSS |
| SEQ ID | Anti-CD47 | QVQLVQSGAEVKKPGASVKVSCKASGYTFTNYNMHWV |
| NO: 114 | antibody heavy | RQAPGQRLEWMGTIYPGNDDTSYNQKFKDRVTITADTS |
| | chain variable | ASTAYMELSSLRSEDTAVYYCARGGYRAMDYWGQGTLV |
| | region | TVSSA |
| | (amino acid | |
| | sequence) | |
| SEQ ID | Anti-CD47 | DIVMTQSPLSLPVTPGEPASISCRSSQSIVYSNGNTYLGW |
| NO: 115 | antibody light | YLQKPGQSPQLLIYKVSNRFSGVPDRFSGSGSGTDFTLKI |
| | chain variable | SRVEAEDVGVYYCFQGSHVPYTFGQGTKLEIKR |
| | region | |
| | (amino acid | |
| | sequence) | |
| SEQ ID | SpyTag | VPTIVMVDAYKRYK |
| NO: 116 | (amino acid | |
| | sequence) | |
| SEQ ID | SpyCatcher | MVTTLSGLSGEQGPSGDMTTEEDSATHIKFSKRDEDGR |
| NO: 117 | (amino acid | ELAGATMELRDSSGKTISTWISDGHVKDFYLYPGKYTFV |
| | sequence0 | ETAAPDGYEVATAITFTVNEQGQVTVNGEATKGDAHTGS |
| | | SGS |
| SEQ ID | T5pr-6xHis-MBP- | MHHHHHHKTEEGKLVIWINGDKGYNGLAEVGKKFEKDT |
| NO: 118 | HRV_3C-spCas9- | GIKVTVEHPDKLEEKFPQVAATGDGPDIIFWAHDRFGGY |
| | 2xSV40NLS- | AQSGLLAEITPDKAFQDKLYPFTWDAVRYNGKLIAYPIAV |
| | Link8-DarpinEc1 | EALSLIYNKDLLPNPPKTWEEIPALDKELKAKGKSALMFN |
| | (amino acid | LQEPYFTWPLIAADGGYAFKYENGKYDIKDVGVDNAGAK |
| | sequence) | AGLTFLVDLIKNKHMNADTDYSIAEAAFNKGETAMTINGP |
| | (Also referred to | WAWSNIDTSKVNYGVTVLPTFKGQPSKPFVGVLSAGINA |
| | as Cas9- | ASPNKELAKEFLENYLLTDEGLEAVNKDKPLGAVALKSYE |
| | Darpin(EpCam) or | EELAKDPRIAATMENAQKGEIMPNIPQMSAFWYAVRTAVI |
| | Cas9-Darpin(Ec1)) | NAASGRQTVDEALKDAQTNLEVLFNSSSNNNNNNNNNN |
| | Darpin(EpCam) | LGIEGRISHMLEVLFQGPMDKKYSIGLDIGTNSVGWAVIT |
| | sequence is | DEYKVPSKKFKVLGNTDRHSIKKNLIGALLFDSGETAEAT |
| | Underlined | RLKRTARRRYTRRKNRICYLQEIFSNEMAKVDDSFFHRL |
| | | EESFLVEEDKKHERHPIFGNIVDEVAYHEKYPTIYHLRKKL |
| | | VDSTDKADLRLIYLALAHMIKFRGHFLIEGDLNPDNSDVD |
| | | KLFIQLVQTYNQLFEENPINASGVDAKAILSARLSKSRRLE |
| | | NLIAQLPGEKKNGLFGNLIALSLGLTPNFKSNFDLAEDAKL |
| | | QLSKDTYDDDLDNLLAQIGDQYADLFLAAKNLSDAILLSDI |
| | | LRVNTEITKAPLSASMIKRYDEHHQDLTLLKALVRQQLPE |
| | | KYKEIFFDQSKNGYAGYIDGGASQEEFYKFIKPILEKMDG |
| | | TEELLVKLNREDLLRKQRTFDNGSIPHQIHLGELHAILRRQ |
| | | EDFYPFLKDNREKIEKILTFRIPYYVGPLARGNSRFAWMT |
| | | RKSEETITPWNFEEVVDKGASAQSFIERMTNFDKNLPNE |
| | | KVLPKHSLLYEYFTVYNELTKVKYVTEGMRKPAFLSGEQ |
| | | KKAIVDLLFKTNRKVTVKQLKEDYFKKIECFDSVEISGVED |
| | | RFNASLGTYHDLLKIIKDKDFLDNEENEDILEDIVLTLTLFE |
| | | DREMIEERLKTYAHLFDDKVMKQLKRRRYTGWGRLSRK |
| | | LINGIRDKQSGKTILDFLKSDGFANRNFMQLIHDDSLTFKE |
| | | DIQKAQVSGQGDSLHEHIANLAGSPAIKKGILQTVKVVDE |
| | | LVKVMGRHKPENIVIEMARENQTTQKGQKNSRERMKRIE |
| | | EGIKELGSQILKEHPVENTQLQNEKLYLYYLQNGRDMYV |
| | | DQELDINRLSDYDVDHIVPQSFLKDDSIDNKVLTRSDKNR |
| | | GKSDNVPSEEVVKKMKNYWRQLLNAKLITQRKFDNLTKA |
| | | ERGGLSELDKAGFIKRQLVETRQITKHVAQILDSRMNTKY |
| | | DENDKLIREVKVITLKSKLVSDFRKDFQFYKVREINNYHH |
| | | AHDAYLNAVVGTALIKKYPKLESEFVYGDYKVYDVRKMIA |
| | | KSEQEIGKATAKYFFYSNIMNFFKTEITLANGEIRKRPLIET |
| | | NGETGEIVWDKGRDFATVRKVLSMPQVNIVKKTEVQTG |
| | | GFSKESILPKRNSDKLIARKKDWDPKKYGGFDSPTVAYS |
| | | VLVVAKVEKGKSKKLKSVKELLGITIMERSSFEKNPIDFLE |
| | | AKGYKEVKKDLIIKLPKYSLFELENGRKRMLASAGELQKG |
| | | NELALPSKYVNFLYLASHYEKLKGSPEDNEQKQLFVEQH |
| | | KHYLDEIIEQISEFSKRVILADANLDKVLSAYNKHRDKPIRE |
| | | QAENIIHLFTLTNLGAPAAFKYFDTTIDRKRYTSTKEVLDA |
| | | TLIHQSITGLYETRIDLSQLGGDGSPKKKRKVEDPKKKRK |
| | | VDNGSSGSELDLGKKLLEAARAGQDDEVRILVANGADVN |
| | | AYFGTTPLHLAAAHGRLEIVEVLLKNGADVNAQDVWGITP |
| | | LHLAAYNGHLEIVEVLLKYGADVNAHDTRGWTPLHLAAIN |
| | | GHLEIVEVLLKNVADVNAQDRSGKTPFDLAIDNGNEDIAE |
| | | VLQKAAKLN |
| SEQ ID | Darpin(EpCam) | DNGSSGSELDLGKKLLEAARAGQDDEVRILVANGADVNA |
| NO: 119 | (amino acid | YFGTTPLHLAAAHGRLEIVEVLLKNGADVNAQDVWGITPL |
| | sequence) | HLAAYNGHLEIVEVLLKYGADVNAHDTRGWTPLHLAAIN |
| | | GHLEIVEVLLKNVADVNAQDRSGKTPFDLAIDNGNEDIAE |
| | | VLQKAAKLN |
| SEQ ID | Cas9(WT) | MDKKYSIGLDIGTNSVGWAVITDEYKVPSKKFKVLGNTDR |
| NO: 120 | Streptococcus | HSIKKNLIGALLFDSGETAEATRLKRTARRRYTRRKNRIC |
| | pyogenes | YLQEISNEMAKVDDSFFHRLEESFLVEEDKKHERHPIFG |
| | (amino acid | NIVDEVAYHEKYPTIYHLRKKLVDSTDKADLRLIYLALAHM |
| | sequence) | IKFRGHFLIEGDLNPDNSDVDKLFIQLVQTYNQLFEENPIN |
| | | ASGVDAKAILSARLSKSRRLENLIAQLPGEKKNGLFGNLIA |
| | | LSLGLTPNFKSNFDLAEDAKLQLSKDTYDDDLDNLLAQIG |
| | | DQYADLFLAAKNLSDAILLSDILRVNTEITKAPLSASMIKRY |
| | | DEHHQDLTLLKALVRQQLPEKYKEIFFDQSKNGYAGYID |
| | | GGASQEEFYKFIKPILEKMDGTEELLVKLNREDLLRKQRT |
| | | FDNGSIPHQIHLGELHAILRRQEDFYPFLKDNREKIEKILTF |
| | | RIPYYVGPLARGNSRFAWMTRKSEETITPWNFEEVVDKG |
| | | ASAQSFIERMTNFDKNLPNEKVLPKHSLLYEYFTVYNELT |
| | | KVKYVTEGMRKPAFLSGEQKKAIVDLLFKTNRKVTVKQL |
| | | KEDYFKKIECFDSVEISGVEDRFNASLGTYHDLLKIIKDKD |
| | | FLDNEENEDILEDIVLTLTLFEDREMIEERLKTYAHLFDDK |
| | | VMKQLKRRRYTGWGRLSRKLINGIRDKQSGKTILDFLKS |
| | | DGFANRNFMQLIHDDSLTFKEDIQKAQVSGQGDSLHEHI |
| | | ANLAGSPAIKKGILQTVKVVDELVKVMGRHKPENIVIEMA |
| | | RENQTTQKGQKNSRERMKRIEEGIKELGSQILKEHPVEN |
| | | TQLQNEKLYLYYLQNGRDMYVDQELDINRLSDYDVDHIV |
| | | PQSFLKDDSIDNKVLTRSDKNRGKSDNVPSEEVVKKMKN |
| | | YWRQLLNAKLITQRKFDNLTKAERGGLSELDKAGFIKRQL |
| | | VETRQITKHVAQILDSRMNTKYDENDKLIREVKVITLKSKL |
| | | VSDFRKDFQFYKVREINNYHHAHDAYLNAVVGTALIKKYP |
| | | KLESEFVYGDYKVYDVRKMIAKSEQEIGKATAKYFFYSNI |
| | | MNFFKTEITLANGEIRKRPLIETNGETGEIVWDKGRDFAT |
| | | VRKVLSMPQVNIVKKTEVQTGGFSKESILPKRNSDKLIAR |
| | | KKDWDPKKYGGFDSPTVAYSVLVVAKVEKGKSKKLKSV |
| | | KELLGITIMERSSFEKNPIDFLEAKGYKEVKKDLIIKLPKYS |
| | | LFELENGRKRMLASAGELQKGNELALPSKYVNFLYLASH |
| | | YEKLKGSPEDNEQKQLFVEQHKHYLDEIIEQISEFSKRVIL |
| | | ADANLDKVLSAYNKHRDKPIREQAENIIHLFTLTNLGAPAA |
| | | FKYFDTTIDRKRYTSTKEVLDATLIHQSITGLYETRIDLSQL |
| | | GGD |
| SEQ ID | Cas12a | MTQFEGFTNLYQVSKTLRFELIPQGKTLKHIQEQGFIEED |
| NO: 121 | Acidaminococcus | KARNDHYKELKPIIDRIYKTYADQCLQLVOLDWENLSAAI |
| | sp. (strain BV3L6) | DSYRKEKTEETRNALIEEQATYRNAIHDYFIGRTDNLTDAI |
| | (amino acid | NKRHAEIYKGLFKAELFNGKVLKQLGTVTTTEHENALLRS |
| | sequence) | FDKFTTYFSGFYENRKNVFSAEDISTAIPHRIVQDNFPKFK |
| | | ENCHIFTRLITAVPSLREHFENVKKAIGIFVSTSIEEVFSFP |
| | | FYNQLLTQTQIDLYNQLLGGISREAGTEKIKGLNEVLNLAI |
| | | QKNDETAHIIASLPHRFIPLFKQILSDRNTLSFILEEFKSDE |
| | | EVIQSFCKYKTLLRNENVLETAEALFNELNSIDLTHIFISHK |
| | | KLETISSALCDHWDTLRNALYERRISELTGKITKSAKEKVQ |
| | | RSLKHEDINLQEIISAAGKELSEAFKQKTSEILSHAHAALD |
| | | QPLPTTLKKQEEKEILKSQLDSLLGLYHLLDWFAVDESNE |
| | | VDPEFSARLTGIKLEMEPSLSFYNKARNYATKKPYSVEKF |
| | | KLNFQMPTLASGWDVNKEKNNGAILFVKNGLYYLGIMPK |
| | | QKGRYKALSFEPTEKTSEGFDKMYYDYFPDAAKMIPKCS |
| | | TQLKAVTAHFQTHTTPILLSNNFIEPLEITKEIYDLNNPEKE |
| | | PKKFQTAYAKKTGDQKGYREALCKWIDFTRDFLSKYTKT |
| | | TSIDLSSLRPSSQYKDLGEYYAELNPLLYHISFQRIAEKEI |
| | | MDAVETGKLYLFQIYNKDFAKGHHGKPNLHTLYWTGLFS |
| | | PENLAKTSIKLNGQAELFYRPKSRMKRMAHRLGEKMLNK |
| | | KLKDQKTPIPDTLYQELYDYVNHRLSHDLSDEARALLPNV |
| | | ITKEVSHEIIKDRRFTSDKFFFHVPITLNYQAANSPSKFNQ |
| | | RVNAYLKEHPETPIIGIDRGERNLIYITVIDSTGKILEQRSLN |
| | | TIQQFDYQKKLDNREKERVAARQAWSVVGTIKDLKQGYL |
| | | SQVIHEIVDLMIHYQAVVVLENLNFGFKSKRTGIAEKAVYQ |
| | | QFEKMLIDKLNCLVLKDYPAEKVGGVLNPYQLTDQFTSF |
| | | AKMGTQSGFLFYVPAPYTSKIDPLTGFVDPFVWKTIKNHE |
| | | SRKHFLEGFDFLHYDVKTGDFILHFKMNRNLSFQRGLPG |
| | | FMPAWDIVFEKNETQFDAKGTPFIAGKRIVPVIENHRFTG |
| | | RYRDLYPANELIALLEEKGIVFRDGSNILPKLLENDDSHAI |
| | | DTMVALIRSVLQMRNSNAATGEDYINSPVRDLNGVCFDS |
| | | RFQNPEWPMDADANGAYHIALKGQLLLNHLKESKDLKLQ |
| | | NGISNQDWLAYIQELRN |
| SEQ ID | c-Myc-NLS | PAAKRVKLD |
| NO: 122 | | |
| SEQ ID | TDP-KDEL | GDAHTGSSGSEFGGGSGGGSGGGSEGGSLAALTAHQA |
| NO: 123 | (“KDEL” disclosed | CHLPLETFTRHRQPRGWEQLEQCGYPVQRLVALYLAAR |
| | as SEQ ID NO: | LSWNQVDQVIRNALASPGSGGDLGEAIREQPEQARLALT |
| | 40; underlined) | LAAAESERFVRQGTGNDEAGAANGGGSGGGSKLNGSS |
| | (amino acid | GSELDKKDEL |
| | sequence) | |
| SEQ ID | “KDEL” | KDEL |
| NO: 124 | (amino acid | |
| | sequence) | |
| SEQ ID | Hexahistidine | HHHHHH |
| NO: 125 | (amino acid | |
| | sequence) | |
| SEQ ID | Dodecahistidine | HHHHHHHHHHHH |
| NO: 126 | (amino acid | |
| | sequence) | |
| SEQ ID | Homing | LAGLIDADG |
| NO: 127 | endonuclease | |
| | motif | |
| | (amino acid | |
| | sequence) | |
| SEQ ID | Poly R (R × 17) | RRRRRRRRRRRRRRRR |
| NO: 128 | (amino acid | |
| | sequence) | |

What is claimed:

1. A targeted active gene editing (TAGE) agent comprising
an antigen binding polypeptide that specifically binds to an extracellular cell membrane-bound molecule, and
a site-directed modifying polypeptide that recognizes a nucleic acid sequence,
wherein the antigen binding polypeptide and the site-directed modifying polypeptide are stably associated such that the site-directed modifying polypeptide can be internalized into a cell displaying the extracellular cell membrane-bound molecule.

2. The TAGE agent of claim 1, wherein the antigen binding polypeptide is an antibody, an antigen-binding portion of an antibody, or an antibody-mimetic.

3. The TAGE agent of claim 1 or 2, wherein the site-directed modifying polypeptide comprises a nuclease or a nickase.

4. The TAGE agent of claim 3, wherein the nuclease is a DNA endonuclease.

5. The TAGE agent of claim 4, wherein the DNA endonuclease is Cas9.

6. The TAGE agent of claim 4, wherein the DNA endonuclease is Cas12.

7. The TAGE agent of any one of claims 1 to 6, further comprising a guide RNA that specifically hybridizes to a target region of the genome of the cell, wherein the guide RNA and the site-directed modifying polypeptide form a ribonucleoprotein.

8. A targeted active gene editing (TAGE) agent comprising
an antigen binding polypeptide which specifically binds to an extracellular cell membrane-bound molecule, and
a site-directed modifying polypeptide comprising an RNA-guided DNA endonuclease that recognizes a CRISPR sequence,
wherein the antigen binding polypeptide and the site-directed modifying polypeptide are stably associated such that the site-directed modifying polypeptide can be internalized into a cell displaying the extracellular cell membrane-bound molecule, and
wherein the antigen binding polypeptide is an antibody, an antigen-binding portion of an antibody, or an antibody-mimetic.

9. The TAGE agent of claim 8, further comprising a guide RNA that specifically hybridizes to a target region of the genome of the cell, wherein the guide RNA and the site-directed modifying polypeptide form a ribonucleoprotein.

10. The TAGE agent of claim 8 or 9, wherein the RNA-guided DNA endonuclease is a Cas9 nuclease.

11. The TAGE agent of any one of claims 8 to 10, wherein the site-directed modifying polypeptide further comprises at least one nuclear localization signal (NLS).

12. The TAGE agent of any one of claims 1 to 11, wherein the site-directed modifying polypeptide further comprises a conjugation moiety that binds to the antigen binding polypeptide.

13. The TAGE agent of claim 12, wherein the conjugation moiety is a protein.

14. The TAGE agent of claim 13, wherein the protein is Protein A, SpyCatcher, or a Halo-Tag.

15. The TAGE agent of any one of claims 1 to 11, wherein the site-directed modifying polypeptide and the antigen binding polypeptide are conjugated via a linker.

16. The TAGE agent of claim 15, wherein the linker is cleavable.

17. The TAGE agent of any one of claims 1 to 16, wherein the antibody mimetic is an adnectin (i.e., fibronectin based binding molecules), an affilin, an affimer, an affitin, an alphabody, an affibody, a DARPin, an anticalin, an avimer, a fynomer, a Kunitz domain peptide, a monobody, a nanoCLAMP, a unibody, a versabody, an aptamer, or a peptidic molecule.

18. The TAGE agent of any one of claims 2 to 16, wherein the antigen-binding portion of the antibody is a nanobody, a domain antibody, an scFv, a Fab, a diabody, a BiTE, a diabody, a DART, a minibody, a F(ab′)₂, or an intrabody.

19. The TAGE agent of any one of claims 2 to 16, wherein the antibody is an intact antibody or a bispecific antibody.

20. A targeted active gene editing (TAGE) agent comprising
an antigen binding polypeptide comprising an antibody, or an antigen-binding portion thereof, which specifically binds to an extracellular cell membrane-bound protein, and
a site-directed modifying polypeptide comprising a Cas9 nuclease,
wherein the antibody, or antigen-binding portion thereof, and the site-directed modifying polypeptide are stably associated via a conjugation moiety such that the site-directed modifying polypeptide can be internalized into the cell expressing the extracellular cell membrane-bound protein via the antibody, or the antigen binding portion thereof.

21. The TAGE agent of claim 20, wherein the site-directed modifying polypeptide further comprises at least one nuclear localization signal (NLS).

22. The TAGE agent of claim 21, wherein the at least one NLS comprises an SV40 NLS.

23. The TAGE agent of claim 22, wherein the SV40 NLS comprises the amino acid sequence PKKKRKV (SEQ ID NO: 8).

24. The TAGE agent of any one of claims 20 to 23, wherein the at least one NLS is at the C-terminus, the N-terminus, or both of the site-directed modifying polypeptide.

25. The TAGE agent of any one of claims 20 to 24, comprising at least two NLSs.

26. The TAGE agent of any one of claims 20 to 25, further comprising a guide RNA that specifically hybridizes to a target region of the genome of a cell expressing the extracellular cell membrane-bound protein, wherein the guide RNA and the site-directed modifying polypeptide form a nucleoprotein.

27. The TAGE agent of any one of claims 20 to 26, wherein the site-directed modifying polypeptide further comprises a conjugation moiety that can bind to the antibody, or antigen-binding portion thereof.

28. The TAGE agent of claim 27, wherein the conjugation moiety is a protein.

29. The TAGE of claim 28, wherein the protein is Protein A, SpyCatcher, or a Halo-Tag.

30. The TAGE agent of any one of claims 20 to 29, wherein the Cas9 nuclease comprises the amino acid substitution C80A.

31. The TAGE agent of any one of claims 20 to 29, wherein the Cas9 nuclease has an amino acid sequence that is at least 85%, 90%, 95%, 97%, 98%, 99%, or 100% identical to Cas9 as described in the Sequence Table.

32. The TAGE agent of any one of claims 20 to 29, wherein the antigen-binding portion of the antibody is a nanobody, a domain antibody, an scFv, a Fab, a diabody, a BiTE, a diabody, a DART, a minibody, a F(ab′)2, or an intrabody.

33. The TAGE agent of any one of claims 20 to 29, wherein the antibody is an intact antibody or a bispecific antibody.

34. The TAGE agent of any one of claims 1 to 33, wherein the extracellular cell membrane-bound molecule or protein is HLA-DR, CD44, CD11a, CD22, CD3, CD20, CD33, CD32, CD44, CD47, CD59, CD54, CD25, AchR, CD70, CD74, CTLA4, EGFR, HER2, EpCam, OX40, PD-1, PD-L1, GITR, CD52, CD34, CD27, CD30, ICOS, or RSV.

35. The TAGE agent of any one of claims 1 to 33, wherein the extracellular cell membrane-bound molecule or protein is CD11a.

36. The TAGE agent of claim 35, wherein the antigen binding polypeptide is an anti-CD11a antibody, or antigen binding fragment thereof.

37. The TAGE agent of claim 36, wherein the anti-CD11a antibody is efalizumab.

38. The TAGE agent of any one of claims 1 to 33, wherein the extracellular cell membrane-bound molecule or protein is CD25.

39. The TAGE agent of claim 38, wherein the antigen binding polypeptide is an anti-CD25 antibody, or antigen binding fragment thereof.

40. The TAGE agent of claim 39, wherein the anti-CD25 antibody is daclizumab.

41. A site-directed modifying polypeptide comprising an RNA-guided DNA endonuclease that recognizes a CRISPR sequence and a conjugation moiety that binds to an antibody, an antigen-binding portion of an antibody, or an antibody mimetic that specifically binds to an extracellular cell membrane-bound molecule.

42. The site-directed modifying polypeptide of claim 41, further comprising a guide RNA that specifically hybridizes to a target region of the genome of a cell.

43. The site-directed modifying polypeptide of claim 41 or 42, wherein the RNA-guided DNA endonuclease is a Cas9 nuclease.

44. The site-directed modifying polypeptide of claim 43, wherein the Cas9 nuclease comprises the amino acid substitution C80A.

45. The site-directed modifying polypeptide of claim 43, wherein the Cas9 nuclease comprises an amino acid sequence having at least 85%, 90%, 95%, 97%, 98%, 99%, or 100% identity to Cas9 as described in the Sequence Table.

46. The site-directed modifying polypeptide of claim 41 or 42, wherein the RNA-guided DNA endonuclease is a Cas12 nuclease.

47. The site-directed modifying polypeptide of any one of claims 41 to 46, further comprising at least one nuclear localization signal (NLS).

48. The site-directed modifying polypeptide of claim 46, wherein the at least one NLS comprises an SV40 NLS.

49. The site-directed modifying polypeptide of claim 47, wherein the SV40 NLS comprises PKKKRKV (SEQ ID NO: 8).

50. The site-directed modifying polypeptide of any one of claims 41 to 49, comprising at least two NLSs.

51. The site-directed modifying polypeptide of any one of claims 41 to 50, wherein the at least one NLS is at the C-terminus, the N-terminus, or both of the site-directed modifying polypeptide.

52. The site-directed modifying polypeptide of any one of claims 41 to 51, wherein the site-directed modifying polypeptide further comprises a conjugation moiety that can bind to the antibody, antigen-binding portion thereof, or antibody mimetic.

53. The site-directed modifying polypeptide of claim 52, wherein the conjugation moiety is a protein.

54. The site-directed modifying polypeptide claim 53, wherein the protein is Protein A, SpyCatcher, or a Halo-Tag.

55. The site-directed modifying polypeptide of any one of claims 41 to 54, wherein the extracellular cell membrane-bound molecule is a protein selected from the group consisting of HLA-DR, CD44, CD11a, CD22, CD3, CD20, CD33, CD32, CD44, CD47, CD59, CD54, CD25, AchR, CD70, CD74, CTLA4, EGFR, HER2, EpCam, OX40, PD-1, PD-L1, GITR, CD52, CD34, CD27, CD30, ICOS, or RSV.

56. The site-directed modifying polypeptide of any one of claims 41 to 55, wherein the extracellular cell membrane-bound molecule or protein is CD11a.

57. The site-directed modifying polypeptide of claim 56, wherein the antigen binding polypeptide is an anti-CD11a antibody, or antigen binding fragment thereof.

58. The site-directed modifying polypeptide of claim 57, wherein the anti-CD11a antibody is efalizumab.

59. The site-directed modifying polypeptide of any one of claims 41 to 55, wherein the extracellular cell membrane-bound molecule or protein is CD25.

60. The site-directed modifying polypeptide of claim 59, wherein the antigen binding polypeptide is an anti-CD25 antibody, or antigen binding fragment thereof.

61. The site-directed modifying polypeptide of claim 60, wherein the anti-CD25 antibody is daclizumab.

62. A nucleoprotein comprising the site-directed modifying polypeptide of any one of claims 41 to 61 and a guide RNA, wherein the guide RNA specifically hybridizes to a target region of the genome of a cell displaying the extracellular cell membrane-bound protein.

63. An isolated nucleic acid encoding the site-directed modifying polypeptide of any one of claims 41 to 61.

64. A vector comprising the nucleic acid of claim 63.

65. A cell comprising the site-directed modifying polypeptide of any one of claims 41 to 61.

66. A method of modifying the genome of a target cell, the method comprising contacting the target cell with a targeted active gene editing (TAGE) agent of any one of claims 1 to 40.

67. The method of claim 66, wherein the target cell is a eukaryotic cell.

68. The method of claim 67, wherein the eukaryotic cell is a mammalian cell.

69. The method of claim 68, wherein the mammalian cell is a mouse cell, a non-human primate cell, or a human cell.

70. The method of any one of claims 66 to 69, wherein the site-directed modifying polypeptide produces a cleavage site at the target region of the genome, thereby modifying the genome.

71. The method of any one of claims 66 to 70, wherein the target region of the genome is a target gene.

72. The method of claim 71, wherein said method is effective to modify expression of the target gene.

73. The method of claim 72, wherein said method is effective to increase expression of the target gene relative to a reference level.

74. The method of claim 60, wherein said method is effective to decrease expression of the target gene relative to a reference level.

75. A method of modifying a nucleic acid sequence within a target cell in a mammalian subject, the method comprising contacting the target cell in the subject with a targeted active gene editing (TAGE) agent comprising
an antigen binding polypeptide that specifically binds to an extracellular cell membrane-bound molecule, and
a site-directed modifying polypeptide that recognizes the nucleic acid sequence within the target cell, such that the nucleic acid sequence of the target cell is modified.

76. A method of modifying a nucleic acid sequence within a target cell in a mammalian subject, the method comprising locally administering to the subject a targeted active gene editing (TAGE) agent comprising
an antigen binding polypeptide that specifically binds to an extracellular cell membrane-bound molecule, and
a site-directed modifying polypeptide that recognizes the nucleic acid sequence within the target cell, such that the nucleic acid sequence of the target cell is modified.

77. The method of claim 75 or 76, wherein the method comprises locally administering the TAGE agent to the subject by intramuscular injection, intraosseous injection, intraocular injection, intratumoral injection, or intradermal injection.

78. The method of any one of claims 75 to 77, wherein the method is effective to increase the number of genetically modified target cells in the subject following administration of the TAGE agent.

79. The method of any one of claims 75 to 77, wherein the mammalian subject is a human subject.

80. The method of any one of claims 75 to 79, wherein the subject has a disease selected from an eye disease, a stem cell disorder, and a cancer, and wherein the method is effective to treat the disease.

81. A method of modifying a nucleic acid sequence within a target mammalian cell, the method comprising contacting the target mammalian cell with a targeted active gene editing (TAGE) agent under conditions in which the TAGE agent is internalized into the target cell, such that the nucleic acid sequence is modified, wherein the TAGE agent comprises
an antigen binding polypeptide that specifically binds to an extracellular cell membrane-bound molecule, and
a site-directed modifying polypeptide that recognizes the nucleic acid sequence within the target cell,
wherein the internalization of the TAGE agent is not dependent on electroporation.

82. The method of claim 81, wherein the target mammalian cell is a hematopoietic cell (HSC), a neutrophil, a T cell, a B cell, a dendritic cell, a macrophage, or a fibroblast.

83. The method of claim 81, wherein the target mammalian cell is a hematopoietic stem cell (HSC) or a bone marrow cell that is not an HSC.

84. The method of claim 83, wherein the antigen binding polypeptide specifically binds an extracellular cell membrane-bound molecule on a human HSC.

85. The method of claim 84, wherein the extracellular cell membrane-bound molecule on the HSC is CD34, EMCN, CD59, CD90, ckit, CD45, or CD49F.

86. The method of any one of claims 81 to 85, wherein the target mammalian cell is contacted with the TAGE agent by co-incubation ex vivo.

87. The method of claim 81 to 86, wherein the method provides a genetically-modified target cell which is administered to a subject in need thereof.

88. The method of any one of claims 81 to 85, wherein the target mammalian cell is contacted with the TAGE agent in situ by injection into a tissue of a subject.

89. The method of claim 88, wherein the TAGE agent is administered to the subject by intramuscular injection, intraosseous injection, intraocular injection, intratumoral injection, or intradermal injection.

90. The method of any one of claims 75 to 89, wherein the nucleic acid is a gene in the genome of the target cell, wherein the expression of said gene is altered following said modification.

91. The method of any one of claims 75 to 90, wherein the target mammalian cell is a mouse cell, a non-human primate cell, or a human cell.

92. The method of any one of claims 75 to 91, wherein the antigen binding polypeptide is an antibody, an antigen-binding portion of an antibody, or an antibody-mimetic.

93. The method of claim 92, wherein the antibody mimetic is an adnectin (i.e., fibronectin based binding molecules), an affilin, an affimer, an affitin, an alphabody, an aptamer, an affibody, a DARPin, an anticalin, an avimer, a fynomer, a Kunitz domain peptide, a monobody, a nanoCLAMP, a unibody, a versabody, an aptamer, or a peptidic molecule.

94. The method of claim 92, wherein the antigen-binding portion of the antibody is a nanobody, a domain antibody, an scFv, a Fab, a diabody, a BiTE, a diabody, a DART, a mnibody, a F(ab′)2, or an intrabody.

95. The method of claim 92, wherein the antibody is an intact antibody or a bispecific antibody.

96. The method of any one of claims 75 to 95, wherein the extracellular cell membrane-bound molecule bound by the antigen binding polypeptide is HLA-DR, CD44, CD11a, CD22, CD3, CD20, CD33, CD32, CD44, CD47, CD59, CD54, CD25, AchR, CD70, CD74, CTLA4, EGFR, HER2, EpCam, OX40, PD-1, PD-L1, GITR, CD52, CD34, CD27, CD30, ICOS, or RSV.

97. The method of any one of claims 75 to 95, wherein the extracellular cell membrane-bound molecule or protein is CD11a.

98. The method of claim 97, wherein the antigen binding polypeptide is an anti-CD11a antibody, or antigen binding fragment thereof.

99. The method of claim 98, wherein the anti-CD11a antibody is efalizumab.

100. The method of any one of claims 75 to 95, wherein the extracellular cell membrane-bound molecule or protein is CD25.

101. The method of claim 100, wherein the antigen binding polypeptide is an anti-CD25 antibody, or antigen binding fragment thereof.

102. The method of claim 101, wherein the anti-CD25 antibody is daclizumab.

103. The method of any one of claims 75 to 102, wherein the TAGE agent further comprises at least one nuclear localization signal (NLS).

104. The method of any one of claims 75 to 103, wherein the TAGE agent comprises at least two nuclear localization signals (NLSs).

105. The method of claim 104, wherein the TAGE agent comprises four nuclear localization signals (NLSs).

106. The method of claim 104, wherein the TAGE agent comprises six nuclear localization signals (NLSs).

107. The method of claim 104, wherein the TAGE agent comprises seven nuclear localization signals (NLSs).

108. The method of claim 104, wherein the TAGE agent comprises eight nuclear localization signals (NLSs).

109. The method of any one of claims 103 to 108, wherein the NLS comprises an SV40 NLS.

110. The method of claim 109, wherein the SV40 NLS comprises the amino acid sequence PKKKRKV (SEQ ID NO: 8).

111. The method of any one of claims 75 to 110, wherein the target mammalian cell is a population of target mammalian cells.

112. The method of claim 111, wherein the method is effective to increase the number of genetically modified target mammalian cells.

113. The method of any one of claims 75 to 112, wherein the site-directed modifying polypeptide of the TAGE agent has increased cellular internalization in the target mammalian cell.

114. The method of any one of claims 75 to 113, wherein the site-directed modifying polypeptide of the TAGE agent has increased nuclear internalization in the target mammalian cell.

115. The method of any one of claims 75 to 114, wherein the site-directed modifying polypeptide comprises a nuclease or a nickase.

116. The method of any one of claims 75 to 115, wherein the site-directed modifying polypeptide is a nucleic acid-guided nuclease, and the TAGE agent further comprises a guide nucleic acid that specifically hybridizes to a target region of the nucleic acid sequence of the target mammalian cell, wherein the guide nucleic acid and the nucleic acid-guided nuclease form a nucleoprotein.

117. The method of claim 116, wherein the site-directed modifying polypeptide is a RNA-guided nuclease, and the TAGE agent further comprises a guide RNA that specifically hybridizes to a target region of the nucleic acid sequence of the target mammalian cell, wherein the guide RNA and the RNA-guided nuclease form a ribonucleoprotein.

118. The method of claim 117, wherein the guide RNA is a single guide RNA (sgRNA) or a cr:trRNA.

119. The method of claim 117, wherein the RNA-guided nuclease is a Class 2 Cas polypeptide.

120. The method of claim 119, wherein the Class 2 Cas polypeptide is a Type II Cas polypeptide.

121. The method of claim 120, wherein the Type II Cas polypeptide is Cas9.

122. The method of claim 119, wherein the Class 2 Cas polypeptide is a Type V Cas polypeptide.

123. The method of claim 122, wherein the Type V Cas polypeptide is Cas12.

124. The method of any one of claims 75 to 123, wherein the site-directed modifying polypeptide further comprises a conjugation moiety that binds to the antigen binding polypeptide or a complementary binding moiety attached thereto.

125. The method of claim 124, wherein the conjugation moiety is a protein.

126. The method of claim 125, wherein the protein is SpyCatcher or a Halo-Tag.

127. The method of any one of claims 75 to 126, wherein the site-directed modifying polypeptide and the antigen binding polypeptide are conjugated via a linker.

128. The method of claim 127, wherein the linker is a cleavable linker.

129. The method of any one of claims 75 to 128, wherein the TAGE agent further comprises an endosomal escape agent.

130. The method of claim 129, wherein the endosomal escape agent is TDP or TDP-KDEL (SEQ ID NO: 123).


SEQUENCE LISTING (128 sequences)

SEQ ID NO: 1 - protein, 1387 aa, Artificial Sequence
  Description of Artificial Sequence Synthetic polypeptide
MDKKYSIGLDIGTNSVGWAVITDEYKVPSKKFKVLGNTDRHSIKKNLIGALLFDSGETAEATRLKRTARRRYTRRKNRIAYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVD

SEQ ID NO: 2 - protein, 116 aa, Artificial Sequence
  Description of Artificial Sequence Synthetic polypeptide
VDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPK

SEQ ID NO: 3 - protein, 1511 aa, Artificial Sequence
  Description of Artificial Sequence Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDNGSSGSELVDNKFNKEQQNAFYEILHLPNLNEEQRNAFIQSLKDDPSQSANLLAEAKKLNDAQAPKVDNKFNKEQQNAFYEILHLPNLNEEQRNAFIQSLKDDPSQSANLLAEAKKLNGAQAPK

SEQ ID NO: 4 - protein, 1545 aa, Artificial Sequence
  Description of Artificial Sequence Synthetic polypeptide
MDKKYSIGLDIGTNSVGWAVITDEYKVPSKKFKVLGNTDRHSIKKNLIGALLFDSGETAEATRLKRTARRRYTRRKNRIAYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGASGASAQVQLVESGGGLVQAGDSLRLSCAASGRTFSRGVMGWFRRAPGKEREFVAIFSGSSWSGRSTYYSDSVKGRFTISRDNAKNTVYLQMNGLKPEDTAVYYCAAGYPEAYSAYGRESTYDYWGQGTQVTVSSEPKTPKPQPARQACTSGASGASGSPKKKRKVEDPKKKRKVD

SEQ ID NO: 5 - protein, 1401 aa, Artificial Sequence
  Description of Artificial Sequence Synthetic polypeptide
HHHHHHLEVLFQGPMDKKYSIGLDIGTNSVGWAVITDEYKVPSKKFKVLGNTDRHSIKKNLIGALLFDSGETAEATRLKRTARRRYTRRKNRIAYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVD

SEQ ID NO: 6 - protein, 1513 aa, Artificial Sequence
  Description of Artificial Sequence Synthetic polypeptide
MVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGSNGSSGSELDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVD

SEQ ID NO: 7 - protein, 1514 aa, Artificial Sequence
  Description of Artificial Sequence Synthetic polypeptide
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDNGSSGSELMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 8 - protein, 7 aa, Simian virus 40
PKKKRKV

SEQ ID NO: 9 - protein, 9 aa, Human immunodeficiency virus
RKKRRQRRR

SEQ ID NO: 10 - protein, 64 aa, Artificial Sequence
  Description of Artificial Sequence Synthetic polypeptide
HHHHHHLEVLFQGPMNATPKKKRKVGGSPKKKRKVGGSPKKKRKVGGSPKKKRKVGIHGVPAAT

SEQ ID NO: 11 - protein, 29 aa, Artificial Sequence
  Description of Artificial Sequence Synthetic peptide
GAYGRKKRRQRRRPPAGTSVSLKKKRKVG

SEQ ID NO: 12 - protein, 36 aa, Artificial Sequence
  Description of Artificial Sequence Synthetic polypeptide
HHHHHHGMGAAGRKKRRQRRRPPAGTSVSLKKKRKV

SEQ ID NO: 13 - protein, 12 aa, Human immunodeficiency virus
RQARRNRRRRWR

SEQ ID NO: 14 - protein, 122 aa, Artificial Sequence
  Description of Artificial Sequence Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSA

SEQ ID NO: 15 - protein, 112 aa, Artificial Sequence
  Description of Artificial Sequence Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKR

SEQ ID NO: 16 - protein, 119 aa, Artificial Sequence
  Description of Artificial Sequence Synthetic polypeptide
EVQLLESGGGLVHPGGSLRLSCAASGFTFGGYAMSWVRQAPGKGLEWVSLISGSGGSTYYADSVKGRFTIFRDNSKNTLYLQMISLRAEDSAVYYCAKYSNYDYFDPWGQGTLVTVSSA

SEQ ID NO: 17 - protein, 108 aa, Artificial Sequence
  Description of Artificial Sequence Synthetic polypeptide
EIVLTQSPGTLSLSPGERVTLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 18 - protein, 114 aa, Artificial Sequence
  Description of Artificial Sequence Synthetic polypeptide
EVQLLESGGGLVQPGGSLRLSCAAASGFTFSSYDMSWVRQAPGKGLDWVSTISGGGTYTYYQDSVKGRFTISRDNSKNTLYLQMNSLRAEDTAVYYCASMDYWGQGTTVTVSSA

SEQ ID NO: 19 - protein, 108 aa, Artificial Sequence
  Description of Artificial Sequence Synthetic polypeptide
DIQMTQSPSSLSASVGDRVTITCRASQSIRRYLNWYHQKPGKAPKLLIYGASTLQSGVPSRFSGSGSGTDFTLTISSLQPEDFAVYYCQQSHSAPLTFGGGTKVEIKR

SEQ ID NO: 20 - protein, 118 aa, Artificial Sequence
  Description of Artificial Sequence Synthetic polypeptide
EVQLLESGGGLVQPGGSLRLSCAASGFTFSSYAYSWVRQAPGKGLEWVSAISGSGGRTYYADSVKGRFTISRDNSKNTLYLQMNSLRAEDTAVYYCARLGYGRVDEWGRGTLVTVSSA

SEQ ID NO: 21 - protein, 118 aa, Artificial Sequence
  Description of Artificial Sequence Synthetic polypeptide
QSVLTQPPSASGTPGQRVTISCSGSLSNIGRNPVNWYQQLPGTAPKLLIYLDNLRLSGVPDRFSGSKSGTSASLAISGLQSEDEADYYCATWDDSHPGWTFGGGTKLTVLGQPKAAPS

SEQ ID NO: 22 - protein, 127 aa, Artificial Sequence
  Description of Artificial Sequence Synthetic polypeptide
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSSA

SEQ ID NO: 23 - protein, 114 aa, Artificial Sequence
  Description of Artificial Sequence Synthetic polypeptide
DIVMTQSPDSLAVSLGERATINCKSSQTVLYSSNNKKYLAWYQQKPGQPPNLLIYWASTRESGVPDRESGSGSGTDFTLTISSLQAEDVAVYYCQQYYSTPFTFGPGTKVEIKR

SEQ ID NO: 24 - protein, 120 aa, Artificial Sequence
  Description of Artificial Sequence Synthetic polypeptide
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSSA

SEQ ID NO: 25 - protein, 113 aa, Artificial Sequence
  Description of Artificial Sequence Synthetic polypeptide
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIKR

SEQ ID NO: 26 - protein, 126 aa, Artificial Sequence
  Description of Artificial Sequence Synthetic polypeptide
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSA

SEQ ID NO: 27 - protein, 113 aa, Artificial Sequence
  Description of Artificial Sequence Synthetic polypeptide
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKR

SEQ ID NO: 28 - protein, 118 aa, Artificial Sequence
  Description of Artificial Sequence Synthetic polypeptide
EVQLVESGGGLVKPGGSLRLSCAASGFTFSAYAMNWVRQAPGKGLEWVGRIRTKNNNYATYYADSVKDRFTISRDDSKNTLYLQMNSLKTEDTAVYYCTTFYGNGVWGQGTLVTVSSA

SEQ ID NO: 29 - protein, 113 aa, Artificial Sequence
  Description of Artificial Sequence Synthetic polypeptide
DVVMTQSPLSLPVTLGQPASISCKSSQSLLDSDGKTFLNWFQQRPGQSPRRLIYLVSKLDSGVPDRFSGSGSGTDFTLKISRVEAEDVGVYYCWQGTHFPYTFGQGTRLEIKR

SEQ ID NO: 30 - protein, 115 aa, Artificial Sequence
  Description of Artificial Sequence Synthetic polypeptide
EVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSA

SEQ ID NO: 31 - protein, 107 aa, Artificial Sequence
  Description of Artificial Sequence Synthetic polypeptide
EIVLTQSPATLSLSPGERATLSCSASSSINYIYWYQQKPGQAPRLLIYLTSNLASGVPARFSGSGSGTDFTLTISSLEPEDFAVYYCLQWSSNPLTFGGGTKVEIKR

SEQ ID NO: 32 - protein, 123 aa, Artificial Sequence
  Description of Artificial Sequence Synthetic polypeptide
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSA

SEQ ID NO: 33 - protein, 113 aa, Artificial Sequence
  Description of Artificial Sequence Synthetic polypeptide
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKR

SEQ ID NO: 34 - protein, 126 aa, Artificial Sequence
  Description of Artificial Sequence Synthetic polypeptide
EVQLVESGGGLVQPGGSLRLSCAAAGFTFSSYSMNWVRQAPGKGLEWVSYISSRSRTIYYADSVKGRFTISRDNAKNSLYLQMNSLRDEDTAVYYCARDYGGQPPYYYYYGMDVWGQGTTVTVSSA

SEQ ID NO: 35 - protein, 108 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFVTYYCQQYNSYPRTFGQGTKVEIKR

SEQ ID NO: 36 - protein, 120 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSA

SEQ ID NO: 37 - protein, 108 aa, Artificial Sequence
  Description of Artificial Sequence Synthetic polypeptide
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 38 - protein, 118 aa, Artificial Sequence
  Description of Artificial Sequence Synthetic polypeptide
EVQLVESGGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSSA

SEQ ID NO: 39 - protein, 112 aa, Artificial Sequence
  Description of Artificial Sequence Synthetic polypeptide
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIKR

SEQ ID NO: 40 - protein, 122 aa, Artificial Sequence
  Description of Artificial Sequence Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSA

SEQ ID NO: 41 - protein, 109 aa, Artificial Sequence
  Description of Artificial Sequence Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKR

SEQ ID NO: 42 - protein, 119 aa, Artificial Sequence
  Description of Artificial Sequence Synthetic polypeptide
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSA

SEQ ID NO: 43 - protein, 108 aa, Artificial Sequence
  Description of Artificial Sequence Synthetic polypeptide
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKR

SEQ ID NO: 44 - protein, 122 aa, Artificial Sequence
  Description of Artificial Sequence Synthetic polypeptide
EVQLVESGGGLVQPGGSLRLSCAASGYSFTGHWMNWVRQAPGKGLEWVGMIHPSDSETRYNQKFKDRFTISVDKSKNTLYLQMNSLRAEDTAVYYCARGIYFYGTTYFDYWGQGTLVTVSSA

SEQ ID NO: 45 - protein, 108 aa, Artificial Sequence
  Description of Artificial Sequence Synthetic polypeptide
DIQMTQSPSSLSASVGDRVTITCRASKTISKYLAWYQQKPGKAPKLLIYSGSTLQSGVPSRFSGSGSGTDFTLTISSLQPEDFATYYCQQHNEYPLTFGQGTKVEIKR

SEQ ID NO: 46 - protein, 119 aa, Artificial Sequence
  Description of Artificial Sequence Synthetic polypeptide
EVQLVESGGGLVQPGGSLRLSCAVSGFSSTNYHVHWVRQAPGKGLEWMGVIWGDGDTSYNSVLKSRFTISRDTSKNTVYLQMNSLRAEDTAVYYCARQLTHYYVLAAWGQGTLVTVSSA

SEQ ID NO: 47 - protein, 108 aa, Artificial Sequence
  Description of Artificial Sequence Synthetic polypeptide
DIQMTQSPSSLSASVGDRVTITCRASEDLYYNLAWYQRKPGKAPKLLIYDTYRLADGVPSRFSGSGSGTDYTLTISSLQPEDFASYYCQQYYKFPFTFGQGTKVEIKR

SEQ ID NO: 48 - protein, 118 aa, Artificial Sequence
  Description of Artificial Sequence Synthetic polypeptide
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSA

SEQ ID NO: 49 - protein, 109 aa, Artificial Sequence
  Description of Artificial Sequence Synthetic polypeptide
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKR

SEQ ID NO: 50 - protein, 118 aa, Artificial Sequence
  Description of Artificial Sequence Synthetic polypeptide
QVQLQESGGGVVQPGRSLRLSCAASRFTFSSYAMHWVRQAPGKGLEWVAVISYDGSNKYYADSVKGRTISRDNSKNTLYLQMNSLRAEDTAVYYCARDLSGYGSYPDYWGQGTLVGVS

SEQ ID NO: 51 - protein, 109 aa, Artificial Sequence
  Description of Artificial Sequence Synthetic polypeptide
SSELTQDPAVSVALGQTVRITCQGDSLRSYYASWYQQKPGQAPVLVMYGRNERPSGVPDRFSGSKSGTSASLAISGLQPEDEANYYCAGWDDSLTGPVFGGGTKLTVLG

SEQ ID NO: 52 - protein, 128 aa, Artificial Sequence
  Description of Artificial Sequence Synthetic polypeptide
QVQLQESGPGLVKPSETLSLTCAVSGGSISGGYGWGWIRQPPGKGLEWIGSFYSSSGNTYYNPSLKSQVTISTDTSKNQFSLKLNSMTAADTAVYYCVRDRLFSVVGMVYNNWFDVWGPGVLVTVSSA

SEQ ID NO: 53 - protein, 111 aa, Artificial Sequence
  Description of Artificial Sequence Synthetic polypeptide
ESALTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVLG

SEQ ID NO: 54 - protein, 120 aa, Artificial Sequence
  Description of Artificial Sequence Synthetic polypeptide
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSA

SEQ ID NO: 55 - protein, 108 aa, Artificial Sequence
  Description of Artificial Sequence Synthetic polypeptide
DIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIKR

SEQ ID NO: 56 - protein, 120 aa, Artificial Sequence
  Description of Artificial Sequence Synthetic polypeptide
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARWRDAYYAMDYWGQGTSVTVSSA

SEQ ID NO: 57 - protein, 107 aa, Artificial Sequence
  Description of Artificial Sequence Synthetic polypeptide
QIVLTQSPAIMSASPGEKVTMTCSASSSVSYMHWYQQKSGTSPKRWIYDTSKLASGVPARFSGSGSGTSYSLTISSMEAEDAATYYCQQWSSNPFTFGSGTKLEIKR

SEQ ID NO: 58 - protein, 116 aa, Artificial Sequence
  Description of Artificial Sequence Synthetic polypeptide
QVQLQQSGGGVVQPGRSLGLSCAASGFTFSSYGMNWVRQAPGKGLEWVSYISSSSSTIYYADSVKGRFTISRDNSKNTLYLQMNSLRAEDTAVYYCARGPGMDVWGQGTTVTVSSA

SEQ ID NO: 59 - protein, 115 aa, Artificial Sequence
  Description of Artificial Sequence Synthetic polypeptide
DIVLTQSPDSLAVSLGERATINCKSSQSVLYSSNNKNYLAWYQQKPGQPPKLLIYWASTRESGVPDRFSGSGSGTDFTPAISSLQAEDVAVYYCQQYYSTPQLTFGGGTKVDIKR

SEQ ID NO: 60 - protein, 123 aa, Artificial Sequence
  Description of Artificial Sequence Synthetic polypeptide
QVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSA

SEQ ID NO: 61 - protein, 113 aa, Artificial Sequence
  Description of Artificial Sequence Synthetic polypeptide
DIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKR

SEQ ID NO: 62 - protein, 122 aa, Artificial Sequence
  Description of Artificial Sequence Synthetic polypeptide
QVQLQQSGSELKKPGASVKVSCKASGFTFTNYGMNWVKQAPGQGLKWMGWINTYTREPTYADDFKGRFAFSLDTSVSTAYLQISSLKADDTAVYFCARDITAVVPTGFDYWGQGSLVTVSSA

SEQ ID NO: 63 - protein, 108 aa, Artificial Sequence
  Description of Artificial Sequence Synthetic polypeptide
DIQLTQSPSSLSASVGDRVTITCRASENIYSNLAWYRQKPGKAPKLLVFAASNLADGVPSRFSGSGSGTDYTFTISSLQPEDIATYYCQHFWTTPWAFGGGTKLQIKR

SEQ ID NO: 64 - protein, 121 aa, Artificial Sequence
  Description of Artificial Sequence Synthetic polypeptide
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSA

SEQ ID NO: 65 - protein, 108 aa, Artificial Sequence
  Description of Artificial Sequence Synthetic polypeptide
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKR

SEQ ID NO: 66 - protein, 122 aa, Artificial Sequence
  Description of Artificial Sequence Synthetic polypeptide
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSA

SEQ ID NO: 67 - protein, 110 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKR

SEQ ID NO: 68 - protein, 128 aa, Artificial Sequence
  Description of Artificial Sequence Synthetic polypeptide
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGIAAAGPPYYYYYYYMDVWGKGTTVTVSSA

SEQ ID NO: 69 - protein, 108 aa, Artificial Sequence
  Description of Artificial Sequence Synthetic polypeptide
DIQMTQSPSSLSASVGDRVTITCRASQTIYNYLNWYQQKPGKAPKLLIYAASSLQSGVPSRFGGRGYGTDFTLTINSLQPEDFATYFCQQSYTSPLTFGQGTKVDIKR

SEQ ID NO: 70 - protein, 120 aa, Artificial Sequence
  Description of Artificial Sequence Synthetic polypeptide
QVQLVESGGGVVQPGRSLRLSCAASGFTFSSYDMHWVRQAPGKGLEWVAVIWYDGSNKYYADSVKGRFTISRDNSKNTLYLQMNSLRAEDTAVYYCARGSGNWGFFDYWGQGTLVTVSSA

SEQ ID NO: 71 - protein, 108 aa, Artificial Sequence
  Description of Artificial Sequence Synthetic polypeptide
DIQMTQSPSSLSASVGDRVTITCRASQGISRWLAWYQQKPEKAPKSLIYAASSLQSGVPSRFSGSGSGTDFTLTISSLQPEDFATYYCQQYNTYPRTFGQGTKVEIKR

SEQ ID NO: 72 - protein, 126 aa, Artificial Sequence
  Description of Artificial Sequence Synthetic polypeptide
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSA

SEQ ID NO: 73 - protein, 108 aa, Artificial Sequence
  Description of Artificial Sequence Synthetic polypeptide
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKR

SEQ ID NO: 74 - protein, 117 aa, Artificial Sequence
  Description of Artificial Sequence Synthetic polypeptide
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSSA

SEQ ID NO: 75 - protein, 107 aa, Artificial Sequence
  Description of Artificial Sequence Synthetic polypeptide
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVKR

SEQ ID NO: 76 - protein, 120 aa, Artificial Sequence
  Description of Artificial Sequence Synthetic polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSA

SEQ ID NO: 77 - protein, 107 aa, Artificial Sequence
  Description of Artificial Sequence Synthetic polypeptide
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 78 - protein, 119 aa, Artificial Sequence
  Description of Artificial Sequence Synthetic polypeptide
QVQLVQSGSELKKPGASVKLSCKASGFTFTTYGINWVRQAPGQGLEWIGWIYPRDGSTNFNENFKDRATITVDTSASTAYMELSSLRSEDTAVYFCARLTGGTFLDYWGQGTTVTVSSA

SEQ ID NO: 79 - protein, 112 aa, Artificial Sequence
  Description of Artificial Sequence Synthetic polypeptide
DIVLTQSPATLSLSPGERATLSCRASESVEYYGTSLMQWYQQKPGQPPKLLIFGASNVESGVPDRFSGSGSGTDFTLKISRVEAEDVGMYFCQQSRKLPWTFGGGTKVEIKR

SEQ ID NO: 80 - protein, 121 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSA

SEQ ID NO: 81 - protein, 112 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 82 - protein, 124 aa, Artificial Sequence
  Description of Artificial Sequence Synthetic polypeptide
QVQLVQSGAEVKKPGASVKVSCKASGYTFTGYYMHWVRQAPGQGLEWMGRINPNSGGTNYAQKFQGRVTMTRDTSISTAYMELSRLRSDDTAVYYCARGRTEYIVVAEGFDYWGQGTLVTVSSA

SEQ ID NO: 83 - protein, 113 aa, Artificial Sequence
  Description of Artificial Sequence Synthetic polypeptide
DVVMTQSPPSLLVTLGQPASISCRSSQSLLHSSGNTYLNWLLQRPGQSPQPLIYLVSKLESGVPDRFSGSGSGTDFTLKISGVEAEDVGVYYCMQFTHYPYTFGQGTKLEIKR

SEQ ID NO: 84 - protein, 122 aa, Artificial Sequence
  Description of Artificial Sequence Synthetic polypeptide
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSA

SEQ ID NO: 85 - protein, 108 aa, Artificial Sequence
  Description of Artificial Sequence Synthetic polypeptide
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKR

SEQ ID NO: 86 - protein, 118 aa, Artificial Sequence
  Description of Artificial Sequence Synthetic polypeptide
EVQLLESGGGLVQPGGSLRLSCAASGFTFSNAWMSWVRQAPGKGLEWVAFIWYDGSNKYYADSVKGRFTISRDNSKNTLYLQMNSLRAEDTAVYYCARYSGWYFDYWGQGTLVTVSSA

SEQ ID NO: 87 - protein, 110 aa, Artificial Sequence
  Description of Artificial Sequence Synthetic polypeptide
QSVLTQPPSASGTPGQRVTISCTGSSSNIGAGYDVHWYQQLPGTAPKLLIYDNNNRPSGVPDRFSGSKSGTSASLAISGLRSEDEADYYCQSYDSSLSAWLFGGGTKLTV

SEQ ID NO: 88 - protein, 120 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAA

SEQ ID NO: 89 - protein, 108 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKR

SEQ ID NO: 90 - protein, 126 aa, Artificial Sequence
  Description of Artificial Sequence Synthetic polypeptide
EVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSA

SEQ ID NO: 91 - protein, 108 aa, Artificial Sequence
  Description of Artificial Sequence Synthetic polypeptide
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKR

SEQ ID NO: 92 - protein, 118 aa, Artificial Sequence
  Description of Artificial Sequence Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAA

SEQ ID NO: 93 - protein, 112 aa, Artificial Sequence
  Description of Artificial Sequence Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKR

SEQ ID NO: 94 - protein, 125 aa, Artificial Sequence
  Description of Artificial Sequence Synthetic polypeptide
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSG

SEQ ID NO: 95 - protein, 112 aa, Artificial Sequence
  Description of Artificial Sequence Synthetic polypeptide
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKR

SEQ ID NO: 96 - protein, 124 aa, Artificial Sequence
  Description of Artificial Sequence Synthetic polypeptide
EVQLQQSGPELVKPGASVKISCKASGYSFIGYFMNWVMQSHGRSLEWIGRINPYNGYTFYNQKFKGKATLTVDKSSSTAHMELRSLASEDSAVYYCARHFRYDGVFYYAMDYWGQGTSVTVSSA

SEQ ID NO: 97 - protein, 116 aa, Artificial Sequence
  Description of Artificial Sequence Synthetic polypeptide
QLVLTQSSSASFSLGASAKLTCTLSSQHSTFTIEWYQQQPLKPPKYVMDLKKDGSHSTGDGVPDRFSGSSSGADRYLSISNIQPEDEATYICGVGDTIKEQFVYVFGGGTKVTVLG

SEQ ID NO: 98 - protein, 116 aa, Artificial Sequence; identical to SEQ ID NO: 58
  Description of Artificial Sequence Synthetic polypeptide

SEQ ID NO: 99 - protein, 115 aa, Artificial Sequence; identical to SEQ ID NO: 59
  Description of Artificial Sequence Synthetic polypeptide

SEQ ID NO: 100 - protein, 117 aa, Artificial Sequence
  Description of Artificial Sequence Synthetic polypeptide
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSA

SEQ ID NO: 101 - protein, 109 aa, Artificial Sequence
  Description of Artificial Sequence Synthetic polypeptide
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKR

SEQ ID NO: 102 - protein, 119 aa, Artificial Sequence
  Description of Artificial Sequence Synthetic polypeptide
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSA

SEQ ID NO: 103 - protein, 109 aa, Artificial Sequence
  Description of Artificial Sequence Synthetic polypeptide
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKR

SEQ ID NO: 104 - protein, 126 aa, Artificial Sequence
  Description of Artificial Sequence Synthetic polypeptide
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSA

SEQ ID NO: 105 - protein, 108 aa, Artificial Sequence
  Description of Artificial Sequence Synthetic polypeptide
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKR

SEQ ID NO: 106 - protein, 117 aa, Artificial Sequence
  Description of Artificial Sequence Synthetic polypeptide
EVQLQQSGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVST

SEQ ID NO: 107 - protein, 113 aa, Artificial Sequence
  Description of Artificial Sequence Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 108 - protein, 117 aa, Artificial Sequence
  Description of Artificial Sequence Synthetic polypeptide
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSA

SEQ ID NO: 109 - protein, 113 aa, Artificial Sequence; identical to SEQ ID NO: 107
  Description of Artificial Sequence Synthetic polypeptide

SEQ ID NO: 110 - protein, 128 aa, Artificial Sequence
  Description of Artificial Sequence Synthetic polypeptide
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 111 - protein, 124 aa, Artificial Sequence
  Description of Artificial Sequence Synthetic polypeptide
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 112 - protein, 115 aa, Artificial Sequence
  Description of Artificial Sequence Synthetic polypeptide
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 113 - protein, 121 aa, Artificial Sequence
  Description of Artificial Sequence Synthetic polypeptide
QVQLVESGGGLVEPGGSLRLSCAASGIIFKINDMGWYRQAPGKRREWVAASTGGDEAIYRDSVKDRFTISRDAKNSVFLQMNSLKPEDTAVYYCTAVISTDRDGTEWRRYWGQGTQVYVSS

SEQ ID NO: 114 - protein, 118 aa, Artificial Sequence
  Description of Artificial Sequence Synthetic polypeptide
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSSA

SEQ ID NO: 115 - protein, 113 aa, Artificial Sequence
  Description of Artificial Sequence Synthetic polypeptide
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIKR

SEQ ID NO: 116 - protein, 14 aa, Artificial Sequence
  Description of Artificial Sequence Synthetic peptide
VPTIVMVDAYKRYK

SEQ ID NO: 117 - protein, 119 aa, Artificial Sequence
  Description of Artificial Sequence Synthetic polypeptide
MVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS

SEQ ID NO: 118 - protein, 1964 aa, Artificial Sequence
  Description of Artificial Sequence Synthetic polypeptide
MHHHHHHKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNLEVLFNSSSNNNNNNNNNNLGIEGRISHMLEVLFQGPMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDNGSSGSELDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLN

SEQ ID NO: 119 - protein, 167 aa, Artificial Sequence
  Description of Artificial Sequence Synthetic polypeptide
DNGSSGSELDLGKKLLEAARAGQDDEVRILVANGADVNAYFGTTPLHLAAAHGRLEIVEVLLKNGADVNAQDVWGITPLHLAAYNGHLEIVEVLLKYGADVNAHDTRGWTPLHLAAINGHLEIVEVLLKNVADVNAQDRSGKTPFDLAIDNGNEDIAEVLQKAAKLN

SEQ ID NO: 120 - protein, 1368 aa, Streptococcus pyogenes [sequence omitted]

SEQ ID NO: 121 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 122 - protein, 9 aa, Artificial Sequence
  Description of Artificial Sequence C-myc NLS sequence
PAAKRVKLD

SEQ ID NO: 123 - protein, 161 aa, Artificial Sequence
  Description of Artificial Sequence Synthetic polypeptide
GDAHTGSSGSEFGGGSGGGSGGGSEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGGGSGGGSKLNGSSGSELDKKDEL

SEQ ID NO: 124 - protein, 4 aa, Artificial Sequence
  Description of Artificial Sequence “KDEL” motif peptide
KDEL

SEQ ID NO: 125 - protein, 6 aa, Artificial Sequence
  Description of Artificial Sequence Synthetic 6xHis tag
HHHHHH

SEQ ID NO: 126 - protein, 12 aa, Artificial Sequence
  Description of Artificial Sequence Synthetic 12xHis tag
HHHHHHHHHHHH

SEQ ID NO: 127 - protein, 9 aa, Artificial Sequence
  Description of Artificial Sequence “LAGLIDADG” family peptide motif sequence
LAGLIDADG

SEQ ID NO: 128 - protein, 16 aa, Artificial Sequence
  Description of Artificial Sequence Synthetic peptide
RRRRRRRRRRRRRRRR